(19) United States
(12) Patent Application Publication
(10) Pub. No.: US 2023/0295244 A1
(43) Pub. Date: Sep. 21, 2023

(54) COMPOSITIONS AND METHODS FOR TREATING AND PREVENTING CORONAVIRUSES

(71) Applicant: Svenska Vaccinfabriken Produktion AB, Sollentuna (SE)

(72) Inventors: Matti Sällberg, Stockholm (SE); Lars Frelin, Älvsjö (SE); Gustaf Ahlén, Järfälla (SE)

(21) Appl. No.: 17/936,273

(22) Filed: Sep. 28, 2022

Related U.S. Application Data
(60) Provisional application No. 63/332,406, filed on Apr. 19, 2022.
(60) Provisional application No. 63/261,808, filed on Sep. 29, 2021.

(51) Int. Cl. C07K 14/165; A61K 39/12; C12N 15/86

(52) U.S. Cl. CPC C07K 14/165; A61K 39/12; C12N 15/86; A61K 2039/545

(57) ABSTRACT

Disclosed herein are immunogenic compositions or product combinations of engineered SARS-CoV nucleic acids, genes, peptides, or proteins that can be used to elicit an immune response against a SARS-CoV infection or infection by another coronavirus, including SARS-CoV-2 and variants thereof. Also disclosed are methods of using the immunogenic compositions or product combinations in subjects to generate immune responses and neutralizing antibodies against SARS-CoV or another coronavirus by administering the compositions or combinations with a nucleic acid prime and polypeptide boost approach.

### CROSS-REFERENCE TO RELATED APPLICATIONS

This application claims the benefit of priority of U.S. Provisional Pat. Application US 63/332,406, filed Apr. 19, 2022, and U.S. Provisional Pat. Application US 63/261,808, filed Sep. 29, 2021 which are hereby expressly incorporated by reference in its entirety.

### REFERENCE TO SEQUENCE LISTING

The present application is being filed along with a Sequence Listing in electronic format. The Sequence Listing is provided in a file entitled SeqListingSVF007AUS.xml, which was created on Sep. 26, 2022 and is 300,146 bytes in size. The information in the electronic Sequence Listing is hereby expressly incorporated by reference in its entirety.

### FIELD

Aspects of the present disclosure relate generally to immunogenic compositions or product combinations of engineered SARS-CoV-2 nucleic acids, genes, peptides, or proteins that can be used to elicit an immune response against a SARS-CoV-2 infection or infection by another coronavirus. This immune response includes activation of cytotoxic immune cells and immune cells that produce neutralizing antibodies against SARS-CoV-2 or another coronavirus, including variants thereof. The disclosure also relates generally to methods of using or administering the immunogenic compositions or product combinations described herein to subjects to generate immune responses including but not limited to the production of neutralizing antibodies against SARS-CoV-2 or another coronavirus, for example by administering the compositions or combinations with a homologous or heterologous nucleic acid and/or polypeptide prime and nucleic acid and/or polypeptide boost approach.

### BACKGROUND

The COVID-19 coronavirus pandemic caused by the SARS-CoV-2 (2019-nCoV) virus has resulted in devastating losses of human life, impact on the global economy, and pressure on the public health infrastructure around the world. Although human coronavirus immunotherapies or vaccines directed to the SARS-CoV-2 virus have been approved, long-term efficacy and safety profiles are still not well understood. Furthermore, additional variants or mutants of the SARS-CoV-2 virus, some of which have been shown to be more contagious or virulent than the originally identified strain, have emerged. As such, there is a great need for new treatments and prophylaxes against SARS-CoV-2, including variants thereof, and other coronaviruses.

### SUMMARY

Speed in therapeutic and vaccine development against SARS-CoV-2 and other potential new coronavirus strains or mutants is of utmost importance. Genetic analysis of the virus shows that the most variable components of SARS-CoV-2 and coronaviruses in general is the spike (S) protein, which includes the receptor binding domains (RBD). The RBD of SARS-CoV-2 has approximately 75% homology with the SARS virus of 2003 (SARS-CoV-1) and other coronaviruses (Wu A et al. “Genome Compositions and Divergence of the Novel Coronavirus (2019-nCoV) Originating in China” Cell Host Microbe. (2020); 27(3):325-328). This suggests that existing immunotherapies and vaccine candidates against other coronaviruses such as SARS-CoV-1 are not useful in protecting against SARS-CoV-2. A number of vaccines against SARS-CoV-2 developed around the world have shown effectiveness in preventing severe complications and hospitalization, but the eradication of the SARS-CoV-2 virus has been hindered by worldwide supply shortages and distribution logistics limitations, resulting in the emergence of variants, some of which have been shown to be more infectious and/or less affected by current vaccine products.

Disclosed herein are unique candidates for use as immunogenic compositions against SARS-CoV-2 that allow for rapid validation and large-scale production. Described herein is the use of a heterologous prime-boost immunization approach using a nucleic acid (DNA or RNA) prime and a polypeptide boost administration schedule. A nucleic acid prime allows for detection of neutralizing antibodies within one or two weeks from a single dose. This is due to better T cell priming, as compared to a protein/adjuvant mix.

In some embodiments, the immunogenic compositions or product compositions described herein are nucleic acids and/or polypeptides. In some embodiments, the nucleic acids are DNA or RNA. In some embodiments, the immunogenic compositions or product compositions are intended to be administered to an animal, such as a mammal, mouse, rabbit, cat, dog, primate, monkey, or human, to induce an immunogenic response against the SARS-CoV-2 virus or other coronavirus. In some embodiments, the immunogenic response comprises, consists essentially of, or consist of formation of active immune cells, such as cytotoxic T cells or immune cells that produce inactivating antibodies against the SARS-CoV-2 virus, other coronavirus, or any antigen, polypeptide, protein, nucleic acid, or genome component of the virus. In some embodiments, the immunogenic compositions or product compositions are intended to be administered to an animal, such as a mammal, mouse, rabbit, cat, dog, primate, monkey, or human, to generate neutralizing antibodies against the SARS-CoV-2 virus or other coronavirus, including variants of SARS-CoV-2, in the animal. In some embodiments, the immunogenic compositions or product compositions are administered to individuals that are at risk of contracting SARS-CoV-2 (or variant thereof) or are not currently infected with SARS-CoV-2 (or variant thereof). In some embodiments, the immunogenic compositions or product combinations provide lasting immunogenic protection against a SARS-CoV-2 (or variant thereof) infection.

Some alternatives described herein concern nucleic acids comprising, consisting essentially of, or consisting of at least one SARS-CoV-2 nucleic acid component, preferably joined with a nucleic acid encoding an IgE leader sequence (e.g., a nucleic acid encoding the amino acid sequence MDWTWILFLVAAATRVHS (SEQ ID NO: 44), or an IgE leader nucleic acid sequence having at least 90%, 91%, 92%, 93%, 94%, 95%, 96%, 97%, 98%, 99%, or 100% homology or sequence identity to SEQ ID NO: 43, as well as, use of such nucleic acids and/or the proteins encoded thereby as a medicament, including medicaments that treat or inhibit SARS-CoV-2 infection. In some embodiments, SARS-CoV-2 includes the wild-type strain or variants thereof.

In some alternatives, the nucleic acids or polypeptides also may comprise at least one autocatalytic peptide cleavage site. In some alternatives, the at least one autocatalytic peptide cleavage site is a P2A autocatalytic peptide cleavage site. In some alternatives, the at least one SARS-CoV-2 nucleic acid component or the at least one SARS-CoV-2 polypeptide component are separated by the at least one autocatalytic peptide cleavage site.

In some alternatives, the nucleic acids or polypeptides described herein may encode or comprise a CC40.8 epitope, which is an epitope that is conserved in the spike protein among coronaviruses. In some alternatives, the polypeptides described herein comprise an amino acid sequence of the CC40.8 epitope for SARS-CoV-2 represented as SEQ ID NO: 91 or an amino acid sequence comprising at least 85%, 86%, 87%, 88%, 89%, 90%, 91%, 92%, 93%, 94%, 95%, 96%, 97%, 98%, 99% or 100% sequence identity to SEQ ID NO: 91. In some alternatives, the nucleic acids described herein encode an amino acid sequence of the CC40.8 epitope for SARS-CoV-2 represented as SEQ ID NO: 91 or encode an amino acid sequence comprising at least 85%, 86%, 87%, 88%, 89%, 90%, 91%, 92%, 93%, 94%, 95%, 96%, 97%, 98%, 99% or 100% sequence identity to SEQ ID NO: 91. In some alternatives, a nucleic acid that encodes for the CC40.8 epitope for SARS-CoV-2 is represented as SEQ ID NO: 90 or a nucleic acid encoding the CC40.8 epitope for SARS-CoV-2 represented as having at least 85%, 86%, 87%, 88%, 89%, 90%, 91%, 92%, 93%, 94%, 95%, 96%, 97%, 98%, 99% or 100% sequence identity to SEQ ID NO: 90.

In some alternatives, at least one hepatitis D antigen (HDAg) strain sequence is provided in the nucleic acids or polypeptides referenced above, such as 1, 2, 3, 4, 5, 6, 7, 8, 9, or 10 HDAg strain sequences selected from HDAg genotype 1A, HDAg genotype 1B, HDAg genotype 2A, or HDAg genotype 2B or any combination thereof. In some alternatives, four HDAg strain sequences are provided in the nucleic acids or polypeptides referenced thereof. In some alternatives, the four HDAg strain sequences comprise one copy each of HDAg genotype 1A, HDAg genotype 1B, HDAg genotype 2A, and HDAg genotype 2B. In some alternatives, there are less than four HDAg strain sequences in the nucleic acids or polypeptides. In some alternatives, the HDAg strain sequences are found in tandem in the nucleic acids or polypeptides. In some alternatives, the HDAg strain sequences are separated by autocatalytic peptide cleavage sites. In other alternatives, the HDAg strain sequences are found in tandem with no linker, a linker of at least 1 nucleotide or amino acid, or without an autocatalytic peptide cleavage site in between. In some alternatives, the SARS-CoV-2 or other coronavirus sequences are found either upstream or downstream of the HDAg strain sequences. In some alternatives, the SARS-CoV-2 or other coronavirus sequences are separated from the HDAg strain sequences with an autocatalytic peptide cleavage site. In some alternatives, the autocatalytic peptide cleavage site is a P2A autocatalytic peptide cleavage site. In some alternatives, the constructs SVF-8 (OC-8) and SVF-9 (OC-9) comprise, consist essentially of, or consist of HDAg strain sequences.

In some alternatives, the at least one SARS-CoV-2 nucleic acid component comprises, consists essentially of, or consists of an S protein sequence, RBD sequence, M protein sequence, NP protein sequence, E protein sequence, or HE protein sequence. In some alternatives, the at least one SARS-CoV-2 nucleic acid component is found as the wild-type sequence. Some alternatives concern nucleic acids and the use thereof, wherein the nucleic acids share or comprise at least 50%, 60%, 70%, 80%, 85%, 86%, 87%, 88%, 89%, 90%, 91%, 92%, 93%, 94%, 95%, 96%, 97%, 98%, 99%, or 100% homology or sequence identity to any one or more of SEQ ID NO: 1-12, 77, or 80 or an amount of sequence identity to any one or more of SEQ ID NO: 1-12, 77, or 80 that is within a range defined by any two of the aforementioned percentages. In some alternatives, the at least one SARS-CoV-2 nucleic acid component contemplated for inclusion in the compositions and the uses described herein are human codon optimized sequences of the aforementioned wild-type sequences. In some alternatives, for example, the nucleic acids share or comprise 50%, 60%, 70%, 80%, 85%, 86%, 87%, 88%, 89%, 90%, 91%, 92%, 93%, 94%, 95%, 96%, 97%, 98%, 99%, or 100% homology or sequence identity to any one or more of SEQ ID NO: 13-24, 39-40, 57-63, 71, 73, 75, 78, 81, 84, 86, 88, 92, 94, 96, or 98, or an amount of sequence identity to any one or more of SEQ ID NO: 13-24, 39-40, 57-63, 71, 73, 75, 78, 81, 84, 86, 88, 92, 94, 96, or 98, that is within a range defined by any two of the aforementioned percentages. In some alternatives, the nucleic acids referenced above are used for the prevention, treatment or inhibition of a SARS-CoV-2 infection in a subject, such as a mammal, preferably a human. Accordingly, some alternatives include the use of a nucleic acid having at least 50%, 60%, 70%, 80%, 85%, 86%, 87%, 88%, 89%, 90%, 91%, 92%, 93%, 94%, 95%, 96%, 97%, 98%, 99%, or 100% homology or sequence identity to any one or more of SEQ ID NO: 1-24, 39-40, 57-63, 71, 73, 75, 78, 81, 84, 86, 88, 92, 94, 96, or 98, or an amount of sequence identity to any one or more of SEQ ID NO: 1-24, 39-40, 57-63, 71, 73, 75, 78, 81, 84, 86, 88, 92, 94, 96, or 98, that is within a range defined by any two of the aforementioned percentages as a medicament, such as for the prevention, treatment, amelioration, or inhibition of a SARS-CoV-2 infection in a subject, such as a mammal, preferably a human, which may, optionally, be selected or identified to receive a medicament for the prevention, treatment, amelioration, or inhibition of a SARS-CoV-2 infection. Such subjects can be selected or identified by clinical evaluation or diagnostic evaluation or both. In some embodiments, SARS-CoV-2 includes the wild-type strain or variants thereof.

Some alternatives provided herein concern polypeptides comprising, consisting essentially of, or consisting of at least one SARS-CoV-2 polypeptide component. In some alternatives, the at least one SARS-CoV-2 polypeptide component comprises, consists essentially of, or consists of an S protein sequence, RBD sequence, M protein sequence, NP protein sequence, E protein sequence, or HE protein sequence. In some embodiments, the polypeptides, which may be provided in a composition or method described herein share or comprise at least 50%, 60%, 70%, 80%, 85%, 86%, 87%, 88%, 89%, 90%, 91%, 92%, 93%, 94%, 95%, 96%, 97%, 98%, 99%, or 100% homology or sequence identity to any one or more of SEQ ID NO: 25-36, 41-42, 64-70, 72, 74, 76, 79, 82, 85, 87, 89, 93, 95, 97, or 99, or an amount of sequence identity to any one or more of SEQ ID NO: 25-36, 41-42, 64-70, 72, 74, 76, 79, 82, 85, 87, 89, 93, 95, 97, or 99, that is within a range defined by any two of the aforementioned percentages. In some alternatives, the polypeptides are used as a medicament, such as for the prevention, treatment or inhibition of SARS-CoV-2 in a subject such as a mammal, preferably a human, which may, optionally, be selected or identified to receive a medicament for the prevention, treatment, amelioration, or inhibition of a SARS-CoV-2 infection. Such subjects can be selected or identified by clinical evaluation or diagnostic evaluation or both. In some embodiments, the polypeptides are translated from the wild-type or codon optimized sequences referenced above. In some embodiments, the polypeptides are recombinantly expressed. In some embodiments, the polypeptides are recombinantly expressed in a mammalian, bacterial, yeast, insect, or cell-free system. Accordingly, some alternatives include the use of a polypeptide having at least 50%, 60%, 70%, 80%, 85%, 86%, 87%, 88%, 89%, 90%, 91%, 92%, 93%, 94%, 95%, 96%, 97%, 98%, 99%, or 100% homology or sequence identity to any one or more of SEQ ID NO: 25-36, 41-42, 64-70, 72, 74, 76, 79, 82, 85, 87, 89, 93, 95, 97, or 99, or an amount of sequence identity to any one or more of SEQ ID NO: 25-36, 41-42, 64-70, 72, 74, 76, 79, 82, 85, 87, 89, 93, 95, 97, or 99, that is within a range defined by any two of the aforementioned percentages as a medicament, such as for the prevention, treatment, amelioration, or inhibition of a SARS-CoV-2 infection in a subject, such as a mammal, preferably a human, which may, optionally, be selected or identified to receive a medicament for the prevention, treatment, amelioration, or inhibition of a SARS-CoV-2 infection. In some embodiments, SARS-CoV-2 includes the wild-type strain or variants thereof.

In some alternatives, the immunogenic compositions or product compositions comprise, consist essentially of, or consist of a nucleic acid, described above (e.g., any one or more of SEQ ID NO: 1-24, 39-40, 57-63, 71, 73, 75, 77-78, 80-81, 84, 86, 88, 92, 94, 96, or 98 or a sequence having 85%, 86%, 87%, 88%, 89%, 90%, 91%, 92%, 93%, 94%, 95%, 96%, 97%, 98%, 99%, or 100% sequence identity thereto), and a polypeptide, described above (e.g., any one or more of SEQ ID NO: 25-36, 41-42, 64-70, 72, 74, 76, 79, 82, 85, 87, 89, 93, 95, 97, or 99 or a sequence having 85%, 86%, 87%, 88%, 89%, 90%, 91%, 92%, 93%, 94%, 95%, 96%, 97%, 98%, 99%, or 100% sequence identity thereto). In some alternatives, the immunogenic compositions or product compositions are administered to a subject in a heterologous prime-boost approach. In some alternatives, the prime dose comprises the nucleic acid and the boost dose comprises the polypeptide. In some alternatives, the prime dose comprises any one or more of the aforementioned polypeptides and the boost dose comprises any one or more of the aforementioned nucleic acids. In some alternatives, the immunogenic compositions or product compositions are administered to a subject as a homologous prime-boost approach. In some alternatives, the prime dose comprises any one or more of the aforementioned nucleic acids and the boost dose comprises either the same nucleic acid or a different nucleic acid. In some alternatives, the prime dose comprises any one or more of the aforementioned polypeptides and the boost dose comprises either the same polypeptide or a different polypeptide. In some alternatives, the immunogenic compositions or product compositions further comprise an adjuvant. In some embodiments, the adjuvant is Matrix-M, alum and/or QS21. In some alternatives, the nucleic acid is provided as a recombinant vector. In some alternatives, the recombinant vector is pVAX1. In some alternatives, the immunogenic compositions or product compositions are used for the prevention, treatment or inhibition of SARS-CoV-2 in a subject, such as a mammal, preferably a human, which may, optionally, be selected or identified to receive a medicament for the prevention, treatment, amelioration, or inhibition of a SARS-CoV-2 infection. Such subjects can be selected or identified by clinical evaluation or diagnostic evaluation or both. In some embodiments, SARS-CoV-2 includes the wild-type strain or variants thereof.

Some alternatives described herein concern methods of generating an immune response in a subject, preferably a human, using the immunogenic compositions, product compositions, nucleic acids, or polypeptides described above (e.g., any one or more of SEQ ID NO: 1-36, 39-42, 57-89, or 92-99 or a sequence having 85%, 86%, 87%, 88%, 89%, 90%, 91%, 92%, 93%, 94%, 95%, 96%, 97%, 98%, 99%, or 100% sequence identity thereto). In some alternatives, the methods comprise a heterologous prime-boost approach. In some alternatives, at least one prime dose is administered to the subject and at least one boost dose is administered to the subject. In some alternatives, the at least one prime dose is a nucleic acid. In some alternatives, the at least one boost dose is a polypeptide. In some alternatives, the at least one boost dose comprises an adjuvant, such as Matrix-M, alum and/or QS21. In some alternatives, the at least one boost dose is administered at least 1, 2, 3, 4, 5, 6, 7, 8, 9, 10, 11, 12, 24, 36, or 48 days or weeks after the at least one prime dose is administered or within a range of time defined by any two of the aforementioned time points. In some alternatives, the methods comprise a homologous prime-boost approach. In some alternatives, the method further comprises administration of an antiviral therapy, such as dexamethasone, favipiravir, favilavir, remdesivir, tocilizumab, galidesivir, sarilumab, lopinavir, ritonavir, darunavir, ribavirin, interferon-α, pegylated interferon-α, interferon alfa-2b, convalescent serum, AT-100, or TJM2, or a stem cell therapy, or any combination thereof.

Additional alternatives concern an injection device comprising any one or more of the compositions described herein, such as any one or more of the nucleic acids or polypeptides set forth in any one or more of SEQ ID NO: 1-36, 39-42, 57-89, or 92-99 or a sequence having 85%, 86%, 87%, 88%, 89%, 90%, 91%, 92%, 93%, 94%, 95%, 96%, 97%, 98%, 99%, or 100% sequence identity thereto. Such injection devices can comprise a single dose of such nucleic acid or polypeptide and such injection devices can have modified needle designs configured to enhance delivery of the nucleic acid or polypeptide or both. Such injection devices can be used with or without electroporation. Contemplated injection devices, which can include any one or more of the nucleic acids or polypeptides of SEQ ID NO: 1-36, 39-42, 57-89, or 92-99 (or a sequence having 85%, 86%, 87%, 88%, 89%, 90%, 91%, 92%, 93%, 94%, 95%, 96%, 97%, 98%, 99%, or 100%sequence identity thereto) are described in U.S. Pat. App. Pub. No. 2016/0235928; PCT App. Pub. No. WO2014064534; U.S. Pat. Nos. 6,610,044; 6,132,419; 6,379,966; 6,897,068; 7,015,040; 7,214,369; 7,473,419; and 7,589,059, all of which are hereby expressly incorporated by reference in their entireties.

Some aspects of the present invention are related to the following numbered alternatives:

1. A nucleic acid comprising at least one nucleic acid sequence encoding aSARS-CoV-2 polypeptide, wherein the at least one nucleic acid sequence encoding a SARS-CoV-2 polypeptide comprises:
i) one or more nucleic acid sequences encoding a SARS-CoV-2 spike receptor binding domain (RBD) polypeptide;
ii) a nucleic acid sequence encoding a SARS-CoV-2 nucleocapsid protein (NP) polypeptide;
iii) a nucleic acid sequence encoding a SARS-CoV-2 membrane (M) polypeptide;
iv) a nucleic acid sequence encoding a hepatitis D antigen (HDAg) polypeptide;
v) a nucleic acid sequence encoding an autocatalytic polypeptide cleavage site;
vi) a nucleic acid sequence encoding an IgE leader polypeptide;
vii) a nucleic acid sequence encoding a SARS-CoV-2 spike (S) polypeptide; or
viii) a nucleic acid sequence encoding a CC40.8 epitope;
or any combination thereof.

2. The nucleic acid of alternative 1, wherein one or more of the RBD polypeptide, NP polypeptide, M polypeptide, or S polypeptide is derived from the wild-type SARS-CoV-2 strain (Wuhan-hu-1) or a SARS-CoV-2 variant, optionally the Alpha, Beta, Gamma, Delta, Theta, Iota, Kappa, Lambda, Mu, or Omicron variant.

3. The nucleic acid of alternative 1 or 2, wherein one or more of the RBD polypeptide, NP polypeptide, M polypeptide, or S polypeptide comprises one or more mutations found in a SARS-CoV-2 variant, optionally the Alpha, Beta, Gamma, Delta, Theta, Iota, Kappa, Lambda, Mu, or Omicron variant, relative to the wild-type SARS-CoV-2 strain (Wuhan-hu-1).

4. The nucleic acid of any one of alternatives 1-3, wherein the RBD polypeptide comprises one or more mutations found in a SARS-CoV-2 variant, optionally the Alpha, Beta, Gamma, Delta, Theta, Iota, Kappa, Lambda, Mu, or Omicron variant, relative to the wild-type SARS-CoV-2 strain (Wuhan-hu-1).

5. The nucleic acid of any one of alternatives 1-4, wherein each of the one or more nucleic acid sequences encoding the RBD polypeptide comprise one or more mutations corresponding to K417, N439, L452, T478, E484, N501, G339, S371, S373, S375, T376, D405, R408, N440, G446, S447, Q493, G496, Q498, or Y505 with reference to the full SARS-CoV-2 S protein (e.g. as set forth in NCBI Accession No. YP_009724390 (SEQ ID NO: 83)), or none of these mutations.

6. The nucleic acid of any one of alternatives 1-5, wherein each of the one or more nucleic acid sequences encoding the RBD polypeptide comprise one or more mutations corresponding to K417, N439, L452, T478, E484, or N501 with reference to the full SARS-CoV-2 S protein (e.g. as set forth in NCBI Accession No. YP_009724390 (SEQ ID NO: 83)), or none of these mutations.

7. The nucleic acid of any one of alternatives 1-6, wherein at least one of the one or more nucleic acid sequence encoding the RBD polypeptide comprises mutations corresponding to N439 and N501 with reference to the full SARS-CoV-2 S protein (e.g. as set forth in NCBI Accession No. YP_009724390 (SEQ ID NO: 83)).

8. The nucleic acid of any one of alternatives 1-7, wherein at least one of the one or more nucleic acid sequence encoding the RBD polypeptide comprises mutations corresponding to K417, E484, and N501 with reference to the full SARS-CoV-2 S protein (e.g. as set forth in NCBI Accession No. YP_009724390 (SEQ ID NO: 83)).

9. The nucleic acid of any one of alternatives 1-8, wherein at least one of the one or more nucleic acid sequence encoding the RBD polypeptide comprises mutations corresponding to L452 and T478 with reference to the full SARS-CoV-2 S protein (e.g. as set forth in NCBI Accession No. YP_009724390 (SEQ ID NO: 83)).

10. The nucleic acid of any one of alternatives 1-9, wherein at least one of the one or more nucleic acid sequences encoding the RBD polypeptide comprise one or more mutations corresponding to L452R or T478K with reference to the full SARS-CoV-2 S protein (e.g. as set forth in NCBI Accession No. YP_009724390 (SEQ ID NO: 83)), or none of these mutations.

11. The nucleic acid of any one of alternatives 1-10, wherein at least one of the one or more nucleic acid sequence encoding the RBD polypeptide comprises mutations corresponding to K417, N439, L452, T478, E484, or N501 with reference to the full SARS-CoV-2 S protein (e.g. as set forth in NCBI Accession No. YP_009724390 (SEQ ID NO: 83)).

12. The nucleic acid of any one of alternatives 1-11, wherein at least one of the one or more nucleic acid sequences encoding the RBD polypeptide comprise one or more mutations corresponding to K417N, N439K, E484K, or N501Y with reference to the full SARS-CoV-2 S protein (e.g. as set forth in NCBI Accession No. YP_009724390 (SEQ ID NO: 83)), or none of these mutations.

13. The nucleic acid of any one of alternatives 1-12, wherein at least one of the one or more nucleic acid sequences encoding the RBD polypeptide comprise one or more mutations corresponding to K417N, N439K, L452R, T478K, E484K, or N501Y with reference to the full SARS-CoV-2 S protein (e.g. as set forth in NCBI Accession No. YP_009724390 (SEQ ID NO: 83)), or none of these mutations.

14. The nucleic acid of any one of alternatives 1-13, wherein at least one of the one or more nucleic acid sequence encoding the RBD polypeptide comprises one or more mutations corresponding to K417, L452, or T478 with reference to the full SARS-CoV-2 S protein (e.g. as set forth in NCBI Accession No. YP_009724390 (SEQ ID NO: 83)).

15. The nucleic acid of any one of alternatives 1-14, wherein at least one of the one or more nucleic acid sequences encoding the RBD polypeptide comprise one or more mutations corresponding to K417N, L452R, or T478K with reference to the full SARS-CoV-2 S protein (e.g. as set forth in NCBI Accession No. YP_009724390 (SEQ ID NO: 83)), or none of these mutations.

16. The nucleic acid of any one of alternatives 1-15, wherein at least one of the one or more nucleic acid sequence encoding the RBD polypeptide comprise one or more mutations corresponding to G339, S371, S373, S375, K417, N440, G446, S477, T478, E484, Q493, G496, Q498, N501, or Y505 with reference to the full SARS-CoV-2 S protein (e.g. as set forth in NCBI Accession No. YP_009724390 (SEQ ID NO: 83)), or none of these mutations.

17. The nucleic acid of any one of alternatives 1-16, wherein at least one of the one or more nucleic acid sequence encoding the RBD polypeptide comprise one or more mutations corresponding to G339D, S371L, S373P, S375F, K417N, N440K, G446S, S477N, T478K, E484A, Q493R, G496S, Q498R, N501Y, or Y505H with reference to the full SARS-CoV-2 S protein (e.g. as set forth in NCBI Accession No. YP_009724390 (SEQ ID NO: 83)), or none of these mutations.

18. The nucleic acid of any one of alternatives 1-15, wherein at least one of the one or more nucleic acid sequence encoding the RBD polypeptide comprise one or more mutations corresponding to G339, S371, S373, S375, T376, D405, R408, K417, N440, S477, T478, E484, Q493, Q498, N501, or Y505 with reference to the full SARS-CoV-2 S protein (e.g. as set forth in NCBI Accession No. YP_009724390 (SEQ ID NO: 83)), or none of these mutations.

19. The nucleic acid of any one of alternatives 1-16, wherein at least one of the one or more nucleic acid sequence encoding the RBD polypeptide comprise one or more mutations corresponding to G339D, S371F, S373P, S375F, T376A, D405N, R408S, K417N, N440K, S477N, T478K, E484A, Q493R, Q498R, N501Y, or Y505H with reference to the full SARS-CoV-2 S protein (e.g. as set forth in NCBI Accession No. YP_009724390 (SEQ ID NO: 83)), or none of these mutations.

20. The nucleic acid of any one of alternatives 1-19, wherein the nucleic acid comprises three tandem nucleic acid sequences encoding an RBD polypeptide.

21. The nucleic acid of alternative 20, wherein the three tandem nucleic acid sequences encoding the RBD polypeptide each comprise one or more mutations corresponding to K417, N439, L452, T478, E484, N501, G339, S371, S373, S375, T376, D405, R408, N440, G446, S447, Q493, G496, Q498, or Y505 with reference to the full SARS-CoV-2 S protein (e.g. as set forth in NCBI Accession No. YP_009724390 (SEQ ID NO: 83)), or none of these mutations.

22. The nucleic acid of alternative 20 or 21, wherein the three tandem nucleic acid sequences encoding the RBD polypeptide each comprise one or more mutations corresponding to K417, N439, L452, T478, E484, or N501 with reference to the full SARS-CoV-2 S protein (e.g. as set forth in NCBI Accession No. YP_009724390 (SEQ ID NO: 83)), or none of these mutations.

23. The nucleic acid of any one of alternatives 20-22, wherein at least one of the three tandem nucleic acid sequences encoding the RBD polypeptide comprises mutations corresponding to N439 and N501 with reference to the full SARS-CoV-2 S protein (e.g. as set forth in NCBI Accession No. YP_009724390 (SEQ ID NO: 83)).

24. The nucleic acid of any one of alternatives 20-23, wherein at least one of the three tandem nucleic acid sequences encoding the RBD polypeptide comprises mutations corresponding to K417, E484, and N501 with reference to the full SARS-CoV-2 S protein (e.g. as set forth in NCBI Accession No. YP_009724390 (SEQ ID NO: 83)).

25. The nucleic acid of any one of alternatives 20-24, wherein at least one of the three tandem nucleic acid sequences encoding the RBD polypeptide comprises mutations corresponding to L452 and T478 with reference to the full SARS-CoV-2 S protein (e.g. as set forth in NCBI Accession No. YP_009724390 (SEQ ID NO: 83)).

26. The nucleic acid of any one of alternatives 20-25, wherein at least one of the three tandem nucleic acid sequences encoding the RBD polypeptide comprises mutations corresponding to K417, N439, L452, T478, and N501 with reference to the full SARS-CoV-2 S protein (e.g. as set forth in NCBI Accession No. YP_009724390 (SEQ ID NO: 83)).

27. The nucleic acid of any one of alternatives 20-26, wherein each of the three tandem nucleic acid sequences encoding the RBD polypeptide comprises one or more mutations corresponding to K417N, N439K, L452R, T478K, E484K, or N501Y with reference to the full SARS-CoV-2 S protein (e.g. as set forth in NCBI Accession No. YP_009724390 (SEQ ID NO: 83)), or none of these mutations.

28. The nucleic acid of any one of alternatives 20-27, wherein at least one of the three tandem nucleic acid sequences encoding the RBD polypeptide comprises mutations corresponding to K417, L452, or T478 with reference to the full SARS-CoV-2 S protein (e.g. as set forth in NCBI Accession No. YP_009724390 (SEQ ID NO: 83)).

29. The nucleic acid of any one of alternatives 20-28, wherein each of the three tandem nucleic acid sequences encoding the RBD polypeptide comprises one or more mutations corresponding to K417N, L452R, or T478K with reference to the full SARS-CoV-2 S protein (e.g. as set forth in NCBI Accession No. YP_009724390 (SEQ ID NO: 83)), or none of these mutations.

30. The nucleic acid of any one of alternatives 20-29, wherein at least one of the three tandem nucleic acid sequences encoding the RBD polypeptide comprises mutations corresponding to G339, S371, S373, S375, K417, N440, G446, S477, T478, E484, Q493, G496, Q498, N501, or Y505 with reference to the full SARS-CoV-2 S protein (e.g. as set forth in NCBI Accession No. YP_009724390 (SEQ ID NO: 83)).

31. The nucleic acid of any one of alternatives 20-30, wherein each of the three tandem nucleic acid sequences encoding the RBD polypeptide comprises one or more mutations corresponding to G339D, S371L, S373P, S375F, K417N, N440K, G446S, S477N, T478K, E484A, Q493R, G496S, Q498R, N501Y, or Y505H with reference to the full SARS-CoV-2 S protein (e.g. as set forth in NCBI Accession No. YP_009724390 (SEQ ID NO: 83)), or none of these mutations.

32. The nucleic acid of any one of alternatives 20-31, wherein at least one of the three tandem nucleic acid sequences encoding the RBD polypeptide comprises mutations corresponding to G339, S371, S373, S375, T376, D405, R408, K417, N440, S477, T478, E484, Q493, Q498, N501, or Y505 with reference to the full SARS-CoV-2 S protein (e.g. as set forth in NCBI Accession No. YP_009724390 (SEQ ID NO: 83)).

33. The nucleic acid of any one of alternatives 20-32, wherein each of the three tandem nucleic acid sequences encoding the RBD polypeptide comprises one or more mutations corresponding to G339D, S371F, S373P, S375F, T376A, D405N, R408S, K417N, N440K, S477N, T478K, E484A, Q493R, Q498R, N501Y, or Y505H with reference to the full SARS-CoV-2 S protein (e.g. as set forth in NCBI Accession No. YP_009724390 (SEQ ID NO: 83)), or none of these mutations.

34. The nucleic acid of any one of alternatives 20-33, wherein the three tandem nucleic acid sequences encoding the RBD polypeptide comprise 1) one nucleic acid encoding for the RBD polypeptide from the wild-type SARS-CoV-2 strain (Wuhan-hu-1), 2) one nucleic acid with mutations corresponding to N439 and N501, and 3) one nucleic acid with mutations corresponding to K417, E484, and N501.

35. The nucleic acid of any one of alternatives 20-33, wherein the three tandem nucleic acid sequences encoding the RBD polypeptide comprise 1) one nucleic acid encoding for the RBD polypeptide from the wild-type SARS-CoV-2 strain (Wuhan-hu-1), 2) one nucleic acid with mutations corresponding to K417, N439, L452, T478, and N501, and 3) one nucleic acid with mutations corresponding to K417, E484, and N501.

36. The nucleic acid of any one of alternatives 20-33, wherein the three tandem nucleic acid sequences encoding the RBD polypeptide comprise 1) one nucleic acid with mutations corresponding to G339, S371, S373, S375, K417, N440, G446, S477, T478, E484, Q493, G496, Q498, N501, or Y505, 2) one nucleic acid with mutations corresponding to G339, S371, S373, S375, T376, D405, R408, K417, N440, S477, T478, E484, Q493, Q498, N501, or Y505, and 3) one nucleic acid with mutations corresponding to K417, L452, or T478.

37. The nucleic acid of any one of alternatives 20-33, wherein the three tandem nucleic acid sequences encoding the RBD polypeptide comprise 1) one nucleic acid with mutations corresponding to G339, S371, S373, S375, K417, N440, G446, S477, T478, E484, Q493, G496, Q498, N501, or Y505, 2) one nucleic acid with mutations corresponding to G339, S371, S373, S375, T376, D405, R408, K417, N440, S477, T478, E484, Q493, Q498, N501, or Y505, and 3) one nucleic acid with a mutation corresponding to N501.

38. The nucleic acid of any one of alternatives 1-37, wherein each of the one or more nucleic acid sequences encoding the RBD polypeptide comprise one or more mutations corresponding to C336, C361, C379, C391, C432, C480, C488, or C525 with reference to the full SARS-CoV-2 S protein (e.g. as set forth in NCBI Accession No. YP_009724390 (SEQ ID NO: 83)), or none of these mutations.

39. The nucleic acid of any one of alternatives 1-38, wherein the nucleic acid comprises the one or more nucleic acid sequences encoding the RBD polypeptide and the nucleic acid sequence encoding an NP polypeptide.

40. The nucleic acid of alternative 39, wherein the nucleic acid further comprises the nucleic acid sequence encoding an IgE leader polypeptide.

41. The nucleic acid of alternative 40, wherein the nucleic acid consists essentially of the one or more nucleic acid sequences encoding the RBD polypeptide, the nucleic acid sequence encoding the NP polypeptide, and the nucleic acid sequence encoding the IgE leader polypeptide.

42. The nucleic acid of alternative 41, wherein the nucleic acid encodes for a polypeptide having at least 90%, 91%, 92%, 93%, 94%, 95%, 96%, 97%, 98%, 99%, or 100% sequence identity to SEQ ID NO: 79, 87, 93, or 95, optionally wherein the variance in sequence identity is not in the RBD polypeptide at K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein, optionally wherein the variance in sequence identity is not in the RBD polypeptide at K417, N439, L452, T478, E484, or N501, or any combination thereof, with reference to the full SARS-CoV-2 S protein, optionally wherein the variance in sequence identity is in the RBD polypeptide at C336, C361, C379, C391, C432, C480, C488, or C525, or any combination thereof, with reference to the full SARS-CoV-2 S protein.

43. The nucleic acid of alternative 41 or 42, wherein the nucleic acid shares or comprises at least 90%, 91%, 92%, 93%, 94%, 95%, 96%, 97%, 98%, 99%, or 100% sequence identity to SEQ ID NO: 77, 78, 86, 92, or 94, optionally wherein the variance in sequence identity is not in the RBD polypeptide at sequences encoding for K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein, optionally wherein the variance in sequence identity is not in the RBD polypeptide at sequences encoding for K417, N439, L452, T478, E484, or N501, or any combination thereof, with reference to the full SARS-CoV-2 S protein, optionally wherein the variance in sequence identity is in the RBD polypeptide at sequences encoding for C336, C361, C379, C391, C432, C480, C488, or C525, or any combination thereof, with reference to the full SARS-CoV-2 S protein.

44. The nucleic acid of alternative 40, wherein the nucleic acid further comprises the nucleic acid sequence encoding the autocatalytic polypeptide cleavage site, optionally wherein the autocatalytic polypeptide cleavage site is a P2A autocatalytic polypeptide cleavage site.

45. The nucleic acid of alternative 44, wherein the nucleic acid consists essentially of the one or more nucleic acid sequences encoding the RBD polypeptide, the nucleic acid sequence encoding the NP polypeptide, the nucleic acid sequence encoding the IgE leader polypeptide, and the nucleic acid sequence encoding the autocatalytic polypeptide cleavage site.

46. The nucleic acid of alternative 45, wherein the nucleic acid encodes for a polypeptide having at least 90%, 91%, 92%, 93%, 94%, 95%, 96%, 97%, 98%, 99%, or 100% sequence identity to SEQ ID NO: 82 or 89, optionally wherein the variance in sequence identity is not in the RBD polypeptide at K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein, optionally wherein the variance in sequence identity is not in the RBD polypeptide at K417, N439, L452, T478, E484, or N501, or any combination thereof, with reference to the full SARS-CoV-2 S protein, optionally wherein the variance in sequence identity is in the RBD polypeptide at C336, C361, C379, C391, C432, C480, C488, or C525, or any combination thereof, with reference to the full SARS-CoV-2 S protein.

47. The nucleic acid of alternative 45 or 46, wherein the nucleic acid shares or comprises at least 90%, 91%, 92%, 93%, 94%, 95%, 96%, 97%, 98%, 99%, or 100% sequence identity to SEQ ID NO: 80, 81, or 88, optionally wherein the variance in sequence identity is not in the RBD polypeptide at sequences encoding for K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein, optionally wherein the variance in sequence identity is not in the RBD polypeptide at sequences encoding for K417, N439, L452, T478, E484, or N501, or any combination thereof, with reference to the full SARS-CoV-2 S protein, optionally wherein the variance in sequence identity is in the RBD polypeptide at sequences encoding for C336, C361, C379, C391, C432, C480, C488, or C525, or any combination thereof, with reference to the full SARS-CoV-2 S protein.

48. The nucleic acid of alternative 44, wherein the nucleic acid further comprises the nucleic acid sequence encoding the M polypeptide.

49. The nucleic acid of alternative 48, wherein the nucleic acid consists essentially of the one or more nucleic acid sequences encoding the RBD polypeptide, the nucleic acid sequence encoding the NP polypeptide, the nucleic acid sequence encoding the IgE leader polypeptide, the nucleic acid sequence encoding the autocatalytic polypeptide cleavage site, and the nucleic acid sequence encoding the M polypeptide.

50. The nucleic acid of alternative 49, wherein the nucleic acid encodes for a polypeptide having at least 90%, 91%, 92%, 93%, 94%, 95%, 96%, 97%, 98%, 99%, or 100% sequence identity to SEQ ID NO: 85, optionally wherein the variance in sequence identity is not in the RBD polypeptide at K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein, optionally wherein the variance in sequence identity is not in the RBD polypeptide at K417, N439, L452, T478, E484, or N501, or any combination thereof, with reference to the full SARS-CoV-2 S protein, optionally wherein the variance in sequence identity is in the RBD polypeptide at C336, C361, C379, C391, C432, C480, C488, or C525, or any combination thereof, with reference to the full SARS-CoV-2 S protein.

51. The nucleic acid of alternative 49 or 50, wherein the nucleic acid shares or comprises at least 90%, 91%, 92%, 93%, 94%, 95%, 96%, 97%, 98%, 99%, or 100% sequence identity to SEQ ID NO: 84, optionally wherein the variance in sequence identity is not in the RBD polypeptide at sequences encoding for K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein, optionally wherein the variance in sequence identity is not in the RBD polypeptide at sequences encoding for K417, N439, L452, T478, E484, or N501, or any combination thereof, with reference to the full SARS-CoV-2 S protein, optionally wherein the variance in sequence identity is in the RBD polypeptide at sequences encoding for C336, C361, C379, C391, C432, C480, C488, or C525, or any combination thereof, with reference to the full SARS-CoV-2 S protein.

52. The nucleic acid of alternative 40, wherein the nucleic acid further comprises a nucleic acid sequence encoding a CC40.8 epitope.

53. The nucleic acid of alternative 52, wherein the nucleic acid consists essentially of the one or more nucleic acid sequences encoding the RBD polypeptide, the nucleic acid sequence encoding the NP polypeptide, the nucleic acid sequence encoding the IgE leader polypeptide, and the nucleic acid sequence encoding the CC40.8 epitope.

54. The nucleic acid of alternative 53, wherein the nucleic acid encodes for a polypeptide having at least 90%, 91%, 92%, 93%, 94%, 95%, 96%, 97%, 98%, 99%, or 100% sequence identity to SEQ ID NO: 97 or 99, optionally wherein the variance in sequence identity is not in the RBD polypeptide at K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein, optionally wherein the variance in sequence identity is not in the RBD polypeptide at K417, N439, L452, T478, E484, or N501, or any combination thereof, with reference to the full SARS-CoV-2 S protein, optionally wherein the variance in sequence identity is in the RBD polypeptide at C336, C361, C379, C391, C432, C480, C488, or C525, or any combination thereof, with reference to the full SARS-CoV-2 S protein.

55. The nucleic acid of alternative 53 or 54, wherein the nucleic acid shares or comprises at least 90%, 91%, 92%, 93%, 94%, 95%, 96%, 97%, 98%, 99%, or 100% sequence identity to SEQ ID NO: 96 or 98, optionally wherein the variance in sequence identity is not in the RBD polypeptide at sequences encoding for K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein, optionally wherein the variance in sequence identity is not in the RBD polypeptide at sequences encoding for K417, N439, L452, T478, E484, or N501, or any combination thereof, with reference to the full SARS-CoV-2 S protein, optionally wherein the variance in sequence identity is in the RBD polypeptide at sequences encoding for C336, C361, C379, C391, C432, C480, C488, or C525, or any combination thereof, with reference to the full SARS-CoV-2 S protein.

56. A polypeptide comprising at least one SARS-CoV-2 polypeptide, wherein the SARS-CoV-2 polypeptide comprises:
i) one or more SARS-CoV-2 spike receptor binding domain (RBD) polypeptide sequences;
ii) a SARS-CoV-2 nucleocapsid protein (NP) polypeptide sequence;
iii) a SARS-CoV-2 membrane protein (M) polypeptide sequence;
iv) a hepatitis D antigen (HDAg) polypeptide sequence;
v) an autocatalytic polypeptide cleavage site sequence;
vi) an IgE leader polypeptide sequence; or
vii) a SARS-CoV-2 spike (S) polypeptide sequence; or
viii) a CC40.8 epitope;
or any combination thereof.

57. The polypeptide of alternative 56, wherein one or more of the RBD polypeptide, NP polypeptide, M polypeptide, or S polypeptide is derived from the wild-type SARS-CoV-2 strain (Wuhan-hu-1) or a SARS-CoV-2 variant, optionally the Alpha, Beta, Gamma, Delta, Theta, Iota, Kappa, Lambda, Mu, or Omicron variant.

58. The polypeptide of alternative 56 or 57, wherein one or more of the RBD polypeptide, NP polypeptide, M polypeptide, or S polypeptide comprises one or more mutations found in a SARS-CoV-2 variant, optionally the Alpha, Beta, Gamma, Delta, Theta, Iota, Kappa, Lambda, Mu, or Omicron variant, relative to the wild-type SARS-CoV-2 strain (Wuhan-hu-1).

59. The polypeptide of any one of alternatives 56-58, wherein each of the one or more RBD polypeptide comprises one or more mutations found in a SARS-CoV-2 variant, optionally the Alpha, Beta, Gamma, Delta, Theta, Iota, Kappa, Lambda, Mu, or Omicron variant, relative to the wild-type SARS-CoV-2 strain (Wuhan-hu-1).

60. The polypeptide of any one of alternatives 56-59, wherein each of the one or more RBD polypeptide sequences comprise one or more mutations at K417, N439, L452, T478, E484, N501, G339, S371, S373, S375, T376, D405, R408, N440, G446, S447, Q493, G496, Q498, or Y505 with reference to the full SARS-CoV-2 S protein (e.g. as set forth in NCBI Accession No. YP_009724390 (SEQ ID NO: 83)), or none of these mutations.

61. The polypeptide of any one of alternatives 56-60, wherein each of the one or more RBD polypeptide sequences comprise one or more mutations at K417, N439, L452, T478, E484, or N501 with reference to the full SARS-CoV-2 S protein (e.g. as set forth in NCBI Accession No. YP_009724390 (SEQ ID NO: 83)), or none of these mutations.

62. The polypeptide of any one of alternatives 56-61, wherein at least one of the one or more RBD polypeptide sequences comprises mutations at N439 and N501 with reference to the full SARS-CoV-2 S protein (e.g., as set forth in NCBI Accession No. YP_009724390 (SEQ ID NO: 83)).

63. The polypeptide of any one of alternatives 56-62, wherein at least one of the one or more RBD polypeptide sequences comprises mutations at K417, E484, and N501 with reference to the full SARS-CoV-2 S protein (e.g., as set forth in NCBI Accession No. YP_009724390 (SEQ ID NO: 83)).

64. The polypeptide of any one of alternatives 56-63, wherein at least one of the one or more RBD polypeptide sequences comprises mutations corresponding to L452 and T478 with reference to the full SARS-CoV-2 S protein (e.g., as set forth in NCBI Accession No. YP_009724390 (SEQ ID NO: 83)).

65. The polypeptide of any one of alternatives 56-64, wherein at least one of the one or more RBD polypeptide sequences comprise one or more mutations corresponding to L452R or T478K with reference to the full SARS-CoV-2 S protein (e.g., as set forth in NCBI Accession No. YP_009724390 (SEQ ID NO: 83)).

66. The polypeptide of any one of alternatives 56-65, wherein at least one of the one or more RBD polypeptide sequences comprise one or more mutations corresponding to K417, N439, L452, T478, E484, or N501 with reference to the full SARS-CoV-2 S protein (e.g., as set forth in NCBI Accession No. YP_009724390 (SEQ ID NO: 83)).

67. The polypeptide of any one of alternatives 56-66, wherein at least one of the one or more RBD polypeptide sequences comprise one or more mutations from K417N, N439K, E484K, or N501Y with reference to the full SARS-CoV-2 S protein (e.g., as set forth in NCBI Accession No. YP_009724390 (SEQ ID NO: 83)), or none of these mutations.

68. The polypeptide of any one of alternatives 56-67, wherein at least one of the one or more RBD polypeptide sequences comprise one or more mutations corresponding to K417N, N439K, L452R, T478K, E484K, or N501Y with reference to the full SARS-CoV-2 S protein (e.g., as set forth in NCBI Accession No. YP_009724390 (SEQ ID NO: 83)).

69. The polypeptide of any one of alternatives 56-68, wherein at least one of the one or more RBD polypeptide sequences comprises one or more mutations corresponding to K417, L452, or T478 with reference to the full SARS-CoV-2 S protein (e.g. as set forth in NCBI Accession No. YP_009724390 (SEQ ID NO: 83)).

70. The polypeptide of any one of alternatives 56-69, wherein at least one of the one or more RBD polypeptide sequences comprise one or more mutations corresponding to K417N, L452R, or T478K with reference to the full SARS-CoV-2 S protein (e.g. as set forth in NCBI Accession No. YP_009724390 (SEQ ID NO: 83)), or none of these mutations.

71. The polypeptide of any one of alternatives 56-70, wherein at least one of the one or more RBD polypeptide sequences comprise one or more mutations corresponding to G339, S371, S373, S375, K417, N440, G446, S477, T478, E484, Q493, G496, Q498, N501, or Y505 with reference to the full SARS-CoV-2 S protein (e.g. as set forth in NCBI Accession No. YP_009724390 (SEQ ID NO: 83)), or none of these mutations.

72. The polypeptide of any one of alternatives 56-71, wherein at least one of the one or more RBD polypeptide sequences comprise one or more mutations corresponding to G339D, S371L, S373P, S375F, K417N, N440K, G446S, S477N, T478K, E484A, Q493R, G496S, Q498R, N501Y, or Y505H with reference to the full SARS-CoV-2 S protein (e.g. as set forth in NCBI Accession No. YP_009724390 (SEQ ID NO: 83)), or none of these mutations.

73. The polypeptide of any one of alternatives 56-72, wherein at least one of the one or more RBD polypeptide sequences comprise one or more mutations corresponding to G339, S371, S373, S375, T376, D405, R408, K417, N440, S477, T478, E484, Q493, Q498, N501, or Y505 with reference to the full SARS-CoV-2 S protein (e.g. as set forth in NCBI Accession No. YP_009724390 (SEQ ID NO: 83)), or none of these mutations.

74. The polypeptide of any one of alternatives 56-73, wherein at least one of the one or more RBD polypeptide sequences comprise one or more mutations corresponding to G339D, S371F, S373P, S375F, T376A, D405N, R408S, K417N, N440K, S477N, T478K, E484A, Q493R, Q498R, N501Y, or Y505H with reference to the full SARS-CoV-2 S protein (e.g. as set forth in NCBI Accession No. YP_009724390 (SEQ ID NO: 83)), or none of these mutations.

75. The polypeptide of any one of alternatives 56-74, wherein the polypeptide comprises three tandem RBD polypeptide sequences.

76. The nucleic acid of alternative 75, wherein the three tandem RBD polypeptide sequences each comprise one or more mutations at K417, N439, L452, T478, E484, N501, G339, S371, S373, S375, T376, D405, R408, N440, G446, S447, Q493, G496, Q498, or Y505 with reference to the full SARS-CoV-2 S protein (e.g. as set forth in NCBI Accession No. YP_009724390 (SEQ ID NO: 83)), or none of these mutations.

77. The polypeptide of alternative 75 or 76, wherein the three tandem RBD polypeptide sequences each comprise one or more mutations at K417, N439, L452, T478, E484, or N501 with reference to the full SARS-CoV-2 S protein (e.g., as set forth in NCBI Accession No. YP_009724390 (SEQ ID NO: 83)), or none of these mutations.

78. The polypeptide of any one of alternatives 75-77, wherein at least one of the three tandem RBD polypeptide sequences comprises mutations at N439 and N501 with reference to the full SARS-CoV-2 S protein (e.g., as set forth in NCBI Accession No. YP_009724390 (SEQ ID NO: 83)).

79. The polypeptide of any one of alternatives 75-78, wherein at least one of the three tandem RBD polypeptide sequences comprises mutations at K417, E484, and N501 with reference to the full SARS-CoV-2 S protein (e.g. as set forth in NCBI Accession No. YP_009724390 (SEQ ID NO: 83)).

80. The polypeptide of any one of alternatives 75-79, wherein at least one of the three tandem RBD polypeptide sequences comprises mutations corresponding to L452 and T478 with reference to the full SARS-CoV-2 S protein (e.g. as set forth in NCBI Accession No. YP_009724390 (SEQ ID NO: 83)).

81. The polypeptide of any one of alternatives 75-80, wherein at least one of the three tandem RBD polypeptide sequences comprises mutations corresponding to K417, N439, L452, T478, and N501 with reference to the full SARS-CoV- S protein (e.g. as set forth in NCBI Accession No. YP_009724390 (SEQ ID NO: 83)).

82. The polypeptide of any one of alternatives 75-81, wherein each of the three tandem RBD polypeptide sequences comprises one or more mutations from K417N, N439K, L452R, T478K, E484K, or N501Y with reference to the full SARS-CoV-2 S protein (e.g. as set forth in NCBI Accession No. YP_009724390 (SEQ ID NO: 83)), or none of these mutations.

83. The polypeptide of any one of alternatives 75-82, wherein at least one of the three tandem RBD polypeptide sequences comprises mutations corresponding to K417, L452, or T478 with reference to the full SARS-CoV-2 S protein (e.g. as set forth in NCBI Accession No. YP_009724390 (SEQ ID NO: 83)).

84. The polypeptide of any one of alternatives 75-83, wherein each of the three tandem RBD polypeptide sequences comprises one or more mutations from K417N, L452R, or T478K with reference to the full SARS-CoV-2 S protein (e.g. as set forth in NCBI Accession No. YP_009724390 (SEQ ID NO: 83)), or none of these mutations.

85. The polypeptide of any one of alternatives 75-84, wherein at least one of the three tandem RBD polypeptide sequences comprises mutations corresponding to G339, S371, S373, S375, K417, N440, G446, S477, T478, E484, Q493, G496, Q498, N501, or Y505 with reference to the full SARS-CoV-2 S protein (e.g. as set forth in NCBI Accession No. YP_009724390 (SEQ ID NO: 83)).

86. The polypeptide of any one of alternatives 75-85, wherein each of the three tandem RBD polypeptide sequences comprises one or more mutations from G339D, S371L, S373P, S375F, K417N, N440K, G446S, S477N, T478K, E484A, Q493R, G496S, Q498R, N501Y, or Y505H with reference to the full SARS-CoV-2 S protein (e.g. as set forth in NCBI Accession No. YP_009724390 (SEQ ID NO: 83)), or none of these mutations.

87. The polypeptide of any one of alternatives 75-86, wherein at least one of the three tandem RBD polypeptide sequences comprises mutations corresponding to G339, S371, S373, S375, T376, D405, R408, K417, N440, S477, T478, E484, Q493, Q498, N501, or Y505with reference to the full SARS-CoV-2 S protein (e.g. as set forth in NCBI Accession No. YP_009724390 (SEQ ID NO: 83)).

88. The polypeptide of any one of alternatives 75-87, wherein each of the three tandem RBD polypeptide sequences comprises one or more mutations from G339D, S371F, S373P, S375F, T376A, D405N, R408S, K417N, N440K, S477N, T478K, E484A, Q493R, Q498R, N501Y, or Y505H with reference to the full SARS-CoV-2 S protein (e.g. as set forth in NCBI Accession No. YP_009724390 (SEQ ID NO: 83)), or none of these mutations.

89. The polypeptide of any one of alternatives 75-88, wherein the three tandem RBD polypeptide sequences comprise 1) one RBD polypeptide corresponding to the RBD polypeptide from the wild-type SARS-CoV-2 strain (Wuhan-hu-1), 2) one RBD polypeptide with mutations at N439 and N501, and 3) one RBD polypeptide with mutations at K417, E484, and N501.

90. The polypeptide of any one of alternatives 75-89, wherein the three tandem RBD polypeptide sequences comprise 1) one RBD polypeptide corresponding to the RBD polypeptide from the wild-type SARS-CoV-2 strain (Wuhan-hu-1), 2) one RBD polypeptide with mutations at K417, N439, L452, T478, and N501, and 3) one RBD polypeptide with mutations at K417, E484, and N501.

91. The polypeptide of any one of alternatives 75-90, wherein the three tandem RBD polypeptide sequences comprise 1) one RBD polypeptide with mutations at G339, S371, S373, S375, K417, N440, G446, S477, T478, E484, Q493, G496, Q498, N501, or Y505, 2) one RBD polypeptide with mutations at G339, S371, S373, S375, T376, D405, R408, K417, N440, S477, T478, E484, Q493, Q498, N501, or Y505, and 3) one RBD polypeptide with mutations at K417, L452, or T478.

92. The polypeptide of any one of alternatives 75-91, wherein the three tandem RBD polypeptide sequences comprise 1) one RBD polypeptide with mutations at G339, S371, S373, S375, K417, N440, G446, S477, T478, E484, Q493, G496, Q498, N501, or Y505, 2) one RBD polypeptide with mutations at G339, S371, S373, S375, T376, D405, R408, K417, N440, S477, T478, E484, Q493, Q498, N501, or Y505, and 3) one RBD polypeptide with a mutation at N501.

93. The polypeptide of any one of alternatives 75-92, wherein each of the one or more RBD polypeptide sequences comprise one or more mutations at C336, C361, C379, C391, C432, C480, C488, or C525 with reference to the full SARS-CoV-2 S protein (e.g. as set forth in NCBI Accession No. YP_009724390 (SEQ ID NO: 83)), or none of these mutations.

94. The polypeptide of any one of alternatives 75-93, wherein the polypeptide comprises the one or more RBD polypeptide sequences and the NP polypeptide sequence.

95. The polypeptide of alternative 94, wherein the polypeptide further comprises the IgE leader polypeptide sequence.

96. The polypeptide of alternative 95, wherein the polypeptide consists essentially of the one or more RBD polypeptide sequences, the NP polypeptide sequence, and the IgE leader polypeptide sequence.

97. The polypeptide of alternative 96, wherein the polypeptide comprises a sequence having at least 90%, 91%, 92%, 93%, 94%, 95%, 96%, 97%, 98%, 99%, or 100% sequence identity to SEQ ID NO: 79, 87, 93, or 95, optionally wherein the variance in sequence identity is not in the RBD polypeptide at K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein, optionally wherein the variance in sequence identity is not in the RBD polypeptide at K417, N439, L452, T478, E484, or N501, or any combination thereof, with reference to the full SARS-CoV-2 S protein, optionally wherein the variance in sequence identity is in the RBD polypeptide at C336, C361, C379, C391, C432, C480, C488, or C525, or any combination thereof, with reference to the full SARS-CoV-2 S protein.

98. The polypeptide of alternative 96 or 97, wherein the polypeptide is encoded by a nucleic acid comprising at least 90%, 91%, 92%, 93%, 94%, 95%, 96%, 97%, 98%, 99%, or 100% sequence identity to SEQ ID NO: 77, 78, 86, 92, or 94, optionally wherein the variance in sequence identity is not in the RBD polypeptide at sequences encoding for K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein, optionally wherein the variance in sequence identity is not in the RBD polypeptide at sequences encoding for K417, N439, L452, T478, E484, or N501, or any combination thereof, optionally wherein the variance in sequence identity is in the RBD polypeptide at sequences encoding for C336, C361, C379, C391, C432, C480, C488, or C525, or any combination thereof, with reference to the full SARS-CoV-2 S protein.

99. The polypeptide of alternative 95, wherein the polypeptide further comprises the autocatalytic polypeptide cleavage site sequence, optionally wherein the autocatalytic polypeptide cleavage site sequence is a P2A autocatalytic polypeptide cleavage site sequence.

100. The polypeptide of alternative 99, wherein the polypeptide consists essentially of the one or more RBD polypeptide sequences, the NP polypeptide sequence, the IgE leader polypeptide sequence, and the autocatalytic polypeptide cleavage site sequence.

101. The polypeptide of alternative 100, wherein the polypeptide comprises a sequence having at least 90%, 91%, 92%, 93%, 94%, 95%, 96%, 97%, 98%, 99%, or 100% sequence identity to SEQ ID NO: 82 or 89, optionally wherein the variance in sequence identity is not in the RBD polypeptide at K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein, optionally wherein the variance in sequence identity is not in the RBD polypeptide at K417, N439, L452, T478, E484, or N501, or any combination thereof, with reference to the full SARS-CoV-2 S protein, optionally wherein the variance in sequence identity is in the RBD polypeptide at C336, C361, C379, C391, C432, C480, C488, or C525, or any combination thereof, with reference to the full SARS-CoV-2 S protein.

102. The polypeptide of alternative 100 or 101, wherein the polypeptide is encoded by a nucleic acid comprising at least 90%, 91%, 92%, 93%, 94%, 95%, 96%, 97%, 98%, 99%, or 100% sequence identity to SEQ ID NO: 80, 81, or 88, optionally wherein the variance in sequence identity is not in the RBD polypeptide at sequences encoding for K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein, optionally wherein the variance in sequence identity is not in the RBD polypeptide at sequences encoding for K417, N439, L452, T478, E484, or N501, or any combination thereof, with reference to the full SARS-CoV-2 S protein, optionally wherein the variance in sequence identity is in the RBD polypeptide at sequences encoding for C336, C361, C379, C391, C432, C480, C488, or C525, or any combination thereof, with reference to the full SARS-CoV-2 S protein.

103. The polypeptide of alternative 99, wherein the polypeptide further comprises the M polypeptide sequence.

104. The polypeptide of alternative 103, wherein the polypeptide consists essentially of the one or more RBD polypeptide sequences, the NP polypeptide sequence, the IgE leader polypeptide sequence, the autocatalytic polypeptide cleavage site sequence, and the M polypeptide sequence.

105. The polypeptide of alternative 104, wherein the polypeptide comprises a sequence having at least 90%, 91%, 92%, 93%, 94%, 95%, 96%, 97%, 98%, 99%, or 100% sequence identity to SEQ ID NO: 85, optionally wherein the variance in sequence identity is not in the RBD polypeptide at K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein, optionally wherein the variance in sequence identity is not in the RBD polypeptide at K417, N439, L452, T478, E484, or N501, or any combination thereof, with reference to the full SARS-CoV-2 S protein, optionally wherein the variance in sequence identity is in the RBD polypeptide at C336, C361, C379, C391, C432, C480, C488, or C525, or any combination thereof, with reference to the full SARS-CoV-2 S protein.

106. The polypeptide of alternative 104 or 105, wherein the polypeptide is encoded by a nucleic acid comprising at least 90%, 91%, 92%, 93%, 94%, 95%, 96%, 97%, 98%, 99%, or 100% sequence identity to SEQ ID NO: 84, optionally wherein the variance in sequence identity is not in the RBD polypeptide at sequences encoding for K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein, optionally wherein the variance in sequence identity is not in the RBD polypeptide at sequences encoding for K417, N439, L452, T478, E484, or N501, or any combination thereof, with reference to the full SARS-CoV-2 S protein, optionally wherein the variance in sequence identity is in the RBD polypeptide at sequences encoding for C336, C361, C379, C391, C432, C480, C488, or C525, or any combination thereof, with reference to the full SARS-CoV-2 S protein.

107. The polypeptide of alternative 95, wherein the polypeptide further comprises a CC40.8 epitope.

108. The polypeptide of alternative 107, wherein the polypeptide comprises, consists of, or consists essentially of the one or more RBD polypeptide sequences, the NP polypeptide sequence, the IgE leader polypeptide sequence, and the CC40.8 epitope.

109. The polypeptide of alternative 108, wherein the polypeptide comprises a sequence having at least 90%, 91%, 92%, 93%, 94%, 95%, 96%, 97%, 98%, 99%, or 100% sequence identity to SEQ ID NO: 97 or 99, optionally wherein the variance in sequence identity is not in the RBD polypeptide at K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein, optionally wherein the variance in sequence identity is not in the RBD polypeptide at K417, N439, L452, T478, E484, or N501, or any combination thereof, with reference to the full SARS-CoV-2 S protein, optionally wherein the variance in sequence identity is in the RBD polypeptide at C336, C361, C379, C391, C432, C480, C488, or C525, or any combination thereof, with reference to the full SARS-CoV-2 S protein.

110. The polypeptide of alternative 108 or 109, wherein the polypeptide is encoded by a nucleic acid comprising at least 90%, 91%, 92%, 93%, 94%, 95%, 96%, 97%, 98%, 99%, or 100% sequence identity to SEQ ID NO: 96 or 98, optionally wherein the variance in sequence identity is not in the RBD polypeptide at sequences encoding for K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein, optionally wherein the variance in sequence identity is not in the RBD polypeptide at sequences encoding for K417, N439, L452, T478, E484, or N501, or any combination thereof, with reference to the full SARS-CoV-2 S protein, optionally wherein the variance in sequence identity is in the RBD polypeptide at C336, C361, C379, C391, C432, C480, C488, or C525, or any combination thereof, with reference to the full SARS-CoV-2 S protein.

111. The polypeptide of any one of alternatives 56-110, wherein the polypeptide is recombinantly expressed, optionally in a mammalian, bacterial, yeast, insect, or cell-free system.

112. An immunogenic composition or product combination comprising a nucleic acid and a polypeptide, wherein the nucleic acid is the nucleic acid of any one of alternatives 1-55 and the polypeptide is the polypeptide of any one of alternatives 56-111.

113. The immunogenic composition or product combination of alternative 112, further comprising an adjuvant, optionally wherein the adjuvant is Matrix-M, alum and/or QS21.

114. The immunogenic composition or product combination of alternative 112 or 113, wherein the nucleic acid is provided as a recombinant vector.

115. A method of generating an immune response and/or generating neutralizing antibodies in a subject, comprising administering the nucleic acid of any one of alternatives 1-55 and/or the polypeptide of any one of alternatives 56-111 to the subject.

116. A method of generating an immune response and/or generating neutralizing antibodies in a subject, comprising administering the immunogenic composition or product combination of any one of alternatives 112-114 to the subject.

117. The method of alternative 116, wherein the administration step is done in prime/boost approach, comprising a) administering to the subject at least one prime dose comprising the nucleic acid of the immunogenic composition or product combination and b) administering to the subject at least one boost dose comprising the polypeptide of the immunogenic composition or product combination.

118. The method of alternative 117, wherein the at least one boost dose is administered at least 1, 2, 3, 4, 5, 6, 7, 8, 9, 10, 11, 12, 24, 36, or 48 days or weeks after the at least one prime dose is administered or within a range of time defined by any two of the aforementioned time points e.g., within 1-48 days or 1-48 weeks.

119. The method of any one of alternatives 116-118, wherein the administration is provided enterally, orally, intranasally, parenterally, subcutaneously, intramuscularly, intradermally, or intravenously or any combination thereof, and optionally with in vivo electroporation.

120. The method of any one of alternatives 116-119, wherein the administration is performed in conjunction with an antiviral therapy, optionally wherein the antiviral therapy comprises administration of dexamethasone, favipiravir, favilavir, remdesivir, tocilizumab, galidesivir, sarilumab, lopinavir, ritonavir, darunavir, ribavirin, interferon-α, pegylated interferon-α, interferon alfa-2b, convalescent serum, or any combination thereof.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments and are not intended to be limiting in scope.

FIG. 1 depicts exemplary recombinant immunogenic compositions that can be used as medicaments such as for the prevention, treatment, or inhibition of SARS-CoV-2 in a subject, for example utilizing a heterologous prime-boost approach. Any of the exemplary compositions shown herein may be used for any of the methods or uses disclosed herein.

FIGS. 2A-C depict additional exemplary recombinant immunogenic compositions that can be used as medicaments such as for the prevention, treatment, or inhibition of SARS-CoV-2, including different variants, in a subject, for example utilizing a heterologous prime-boost approach. Any of the exemplary compositions shown herein may be used for any of the methods or uses disclosed herein.

FIGS. 3A-B depict immunization of BALB/c and C57BL/6 mice using exemplary SARS-CoV-2 constructs disclosed herein. FIG. 3A shows end point ELISA of mice serum against RBD and S protein. FIG. 3B shows in vitro SARS-CoV-2 viral neutralization using serum from immunized mice.

FIG. 4 depicts T cell response of immunized mice against peptide pools covering the SARS-CoV-2 RBD, M, and NP proteins as detected by ELISpot.

FIG. 5A depicts anti-S protein antibody titers in mice immunized with a prime/boost approach using OC-2.3 DNA and recombinant S protein with QS21 adjuvant (rS/QS21). The combinations tested were: 1) OC-2.3 DNA prime and rS/QS21 protein boost; 2) OC-2.3 DNA prime and OC-2.3 DNA boost, 3) rS/QS21 protein prime and rS/QS21 protein boost; and 4) rS/QS21 protein prime and OC-2.3 DNA boost.

FIG. 5B depicts T cell response from mice immunized with the prime/boost approach of FIG. 5A against peptide pools covering the SARS-CoV-2 RBD, M, or NP proteins, or the full length RBD, M, or NP proteins.

FIG. 6A depicts anti-S protein antibody titers in rabbits immunized with OC-2.3 DNA tested two weeks after either the first dose (at week 2) or the second dose (at week 5), and administered either 500, 1000, or 1500 µg of the DNA.

FIG. 6B depicts anti-S or anti-NP (N) protein antibody titers in cynomolgus macaques immunized with OC-2.3 DNA tested at either week 0 or week 5 after two 1000 µg doses.

FIG. 6C depicts quantification of SARS-CoV-2 RNA in cynomolgus macaques immunized with either OC-2.3 DNA or control DNA at days 4 or 20 following a SARS-CoV-2 challenge.

FIG. 7A depicts S protein alignments between SARS-CoV strains and variants.

FIG. 7B depicts analysis of the fusion protein comprising the M and N proteins.

FIG. 7C depicts RBD, N and M protein alignments between SARS-CoV strains and variants.

FIGS. 8A-I depict immunogenicity of the universal SARS-CoV-2 vaccine. FIG. 8A depicts a schematic representation of the universal SARS-CoV-2 DNA vaccine OC-2.4 gene design has been given. FIGS. 8B-E depicts the ability of the SARS-CoV-2 DNA vaccine OC-2.4 to induce antibodies against S protein of the huCoV-19/WH01 (FIG. 8B) and Beta variants (FIG. 8D), and the ability to neutralize these viruses in vitro (FIG. 8C and FIG. 8E). FIGS. 8F, G depict the ability of the universal SARS-CoV-2 DNA vaccine OC-2.4 to prime and boost antibodies to S protein (huCoV-19/WH01) following priming with a recombinant S protein in adjuvant (FIG. 8F) and the ability of these antibodies to neutralize the Delta and Omicron variants in vitro (FIG. 8G). FIGS. 8H, I depict how three doses of the universal SARS-CoV-2 DNA vaccine OC-2.4 induce antibodies that cross react with S proteins from the huCoV-19/WH01, Beta, and Delta variants in mice (FIG. 8H), and four 840 µg doses of the same vaccine induces antibodies in female Zealand White rabbits that neutralize both the Delta and Omicron variants in vitro (FIG. 8I).

FIG. 9 depicts levels of anti N and ant S antibodies induced after one, two, and three doses of the OC-2.4 DNA vaccine.

FIGS. 10 depicts how the universal SARS-CoV-2 vaccine induces broadly cross-reactive T cell responses. FIGS. 10A-D depict T cell responses as determined by ELISpot in Balb/c mice immunized three times with either spike protein in QS21 adjuvant (rS/QS21) (FIG. 10A), 50 µg universal SARS-CoV-2 vaccine OC-2.4 (FIG. 10B), primed with rS/QS21 and boosted with two doses of OC-2.4 (FIG. 10C), or empty pVAX plasmid (FIG. 10D), using in vivo electroporation. Spleens were harvested and individual mice were analyzed for the presence of IFNy-producing T cells using the indicated antigens. Data has been given as the number of IFNy-producing (spot forming) cells (SFCs) per million splenocytes. Each color indicates an individual mouse. FIGS. 10E-H depict analysis of T cell responses in New Zealand White rabbits, which were immunized four times with either vehicle only (FIG. 10E), or 84 µg (FIG. 10F) or 840 µg (FIG. 10G) universal SARS-CoV-2 DNA vaccine OC-2.4 using in vivo electroporation. Peripheral blood mononuclear cells (PBMCs) were harvested and analyzed for presence of IFNy-producing T cells using the indicated antigens. Data has been given as the number of IFNγ SFCs per million PBMCs. The dotted line indicates the 50 SFC cut off. Also shown (FIG. 10H) is the cumulated IFNγ SFCs per million PBMCs to the RBD, M, and N peptide pools added together for each group of immunized rabbits (84 µg DNA, 840 1 g DNA, and vehicle only) at each time point (day). The numbers on the x-axis of graphs (FIGS. 10A-D) indicates the number of the respective peptide pool (WH1 variant) covering a part of the indicated protein.

FIGS. 11A-D depict how the universal SARS-CoV-2 vaccine protects against K18 mice against lethal challenge with SARS-CoV-2 Beta variant. FIG. 11A depicts the experimental design of evaluation of different vaccine strategies in the K18-hACE2 mice transgenic for the human ACE2 receptor. FIGS. 11B, C depicts three doses of respective vaccine fully or partially protected the mice against severe disease as determined by histological scoring of bronchial and alveolar lung tissues (FIG. 11B), percent weight loss (FIG. 11C). FIG. 11D depicts three doses of respective vaccine also protect against viral replication in the nose, lungs, and spleen. Values have been given as cycle times (CT), where lower values indicate a higher viral load. Statistical comparisons in the graph are shown with lines with one asterisk indicating P < 0.05 and two asterisks P < 0.01 (Mann-Whitney U-test, GraphPad Prism).

FIGS. 12A-E depicts an experiment where C57BL/6 mice were immunized twice with either a plasmid encoding N alone, rN in alumn, or rN/QS21. FIG. 12A depicts an analysis titers produced against the N protein by the methods described above. FIGS. 12B-E depict an assessment of the methods listed above in priming IFNy-producing N-specific T cells and priming IL-2-producing N-specific T cells.

FIG. 13 depicts a schematic of the vaccine gene and vaccine proteins and an explanation of the cross-reactive antibody and T cell responses.

FIG. 14A depicts a schematic design of universal SARS-CoV vaccine candidates based on SARS-CoV-2 derived sequences. FIG. 14B depicts vaccines used to immunize Balb/c and C57BL/6 mice twice three weeks apart and end antibody point titers at two weeks after the second dose to the receptor binding domain (RBD) of the spike (S) was determined by ELISA. FIG. 14B depicts the level of NAbs to both the Wuhan and B.1.351 VOC was determined by virus neutralization assay as the antibody titer that gives 50% neutralization of virus in a micro neutralization assay (VNT ID₅₀). FIG. 14C depicts the EPS gun (IGEA) handle device used for gene delivery and in vivo electroporation in ferret, rabbit and NHP. FIG. 14D depicts high levels of anti-Wuhan antibodies, after two doses of the OC-2.3 DNA vaccine, is required to induce neutralizing antibodies to both the Wuhan and B.1351 Beta variants in vitro.

FIGS. 15A-C depicts priming of SARS-CoV-2-specific T cells in Balb/c and C57BL/6 mice at two weeks after the second dose as detected reactivity to peptides and proteins corresponding to the RBD, S, M, and N antigens by an IFNy ELISPOT. FIG. 15A depicts an experiment where spleens were harvested and pools of mice splenocytes were analyzed for the presence of IL-2 and IFNy producing T cells using the indicated antigens. Data has been given as the number of IL-2 or IFNy producing (spot forming) cells (SFCs) per million splenocytes. FIG. 15B depicts the cross reactivity of T cells from Balb/c primed either by recombinant S in adjuvant or the RBD-M-N containing OC-2.3 DNA vaccine as determined by IL-2 production in ELISPOT. FIG. 15C depicts analysis of T cell responses in two NHP (385, 796) immunised two times intramuscularly in right quadriceps muscle with 1 mg SARS-CoV-2 OC-2.3 DNA followed by in vivo electroporation using EPSGun.

FIG. 16 depicts an assessment of the ability of the universal SARS-CoV vaccine OC-2.3 to broaden the response induced via rS/QS21-based vaccine as determined reactivity to peptides and proteins corresponding to the RBD, S, M, and N antigens by an IFNy ELISPOT. The right column shows antibody titers against S (Wuhan strain) after two immunizations.

FIGS. 17A-D depicts an experiment where groups of three ferrets were left uninfected or infected by 10⁵ pfu of SARS-CoV-2 and T cell responses were determined as reactivity to peptides and proteins corresponding to the RBD, S, M, and N antigens by an IFNy ELISPOT. FIGS. 17B, C depicts an experiment where groups of three ferrets were vaccinated, as indicated, and then infected by SARS-CoV-2. Antibodies to S were determined by ELISA prior to challenge, and NAbs and nasal SARS-CoV-2 levels were determined. SARS-CoV-2 RNA levels was measured from nasal washings at day 2, 4, 7, 9 and 10 post challenge 10 days post infection all animals were sacrificed, and histological evaluation were performed. SARS-CoV-2 RNA levels were evaluated from BAL sample (FIG. 17B) and histological scoring of nasal cavity and lung tissue were performed (FIG. 17D).

FIG. 18 depicts an experiment where T cells only partly protect against SARS-CoV-2 infection in hACE2 K18 disease model. Two vaccinations of T-cell vaccines partially protected the mice after homologous challenge with SARS-CoV-2 against severe disease as determined by percent weight loss. The group vaccinated with rS/QS21 was protected against lethal disease (100%). All other groups were only T cells were primed (OC-2, OC-2.3, OC-10.3 and OC-12) partially protected against lethal disease.

FIGS. 19A-C depicts an experiment where the universal SARS-CoV-2 vaccines protects against K18 mice against lethal challenge with SARS-CoV-2 Beta variant. Three doses of respective vaccine fully or partially protected the mice against severe disease as determined by percent weight loss (FIG. 19A), histological scoring of bronchial and alveolar lung tissues (FIG. 19C). The levels of SARS-CoV-2 RNA (FIG. 19B) in nasal washing has been given as the mean cycle time value from a duplicate determination of each sample. The levels of SARS-CoV-2 RNA in lung and spleen tissues has been given as the mean cycle time (CT) value from a duplicate determination. The histological scoring (FIG. 19C) was done by an independent pathologist unaware of the experimental groups. The data has been given as the individual histopathological score for each determination in each mouse ranging from 0 (none) to 4 (marked/severe) tissue damage.

FIGS. 20A-C depicts an experiment where groups of 3 NHP were immunized with OC-2.3 DNA or control DNA (HBV). The vaccinated animals were evaluated for anti-S, RBD and HBV PreS1 titers and NAbs two weeks after second vaccination (W5) (FIG. 20A). Genomic SARS-CoV-2 RNA were evaluated in BAL samples collected day 4 and day 10 post infection (FIG. 20A). Priming of SARS-CoV-2-specific antibodies against Wuhan and Beta were evaluated in serum samples collected prior (-35, -21, 0) and post (4, 10, 14, 21) infection (FIG. 20B). SARS-CoV-2-specific T cells against the Wuhan strain as detected by reactivity to peptides and proteins corresponding to the RBD, S, M, and N antigens by an interferon-y ELISpot (FIG. 20C).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

### Definitions

Unless defined otherwise, all technical and scientific terms used herein have the same meaning as is commonly understood by one of ordinary skill in the art. All patents, applications, published applications and other publications referenced herein are expressly incorporated by reference in their entireties unless stated otherwise. In the event that there are a plurality of definitions for a term herein, those in this section prevail unless stated otherwise.

The articles “a” and “an” are used herein to refer to one or to more than one (for example, at least one) of the grammatical object of the article. By way of example, “an element” means one element or more than one element.

The terms “about” or “around” as used herein refer to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Unless defined otherwise, all technical and scientific terms used herein have the same meaning as is commonly understood by one of ordinary skill in the art to which this disclosure belongs. If there is a plurality of definitions for a term herein, those in this section prevail unless stated otherwise. The practice of the present disclosure will employ, unless indicated specifically to the contrary, conventional methods of molecular biology and recombinant DNA techniques within the skill of the art, many of which are described below for the purpose of illustration.

The terms “individual”, “subject”, or “patient” as used herein, means a human or a non-human mammal, e.g., a dog, a cat, a mouse, a rat, a cow, a sheep, a pig, a goat, a non-human primate, or a bird, e.g., a chicken, as well as any other vertebrate or invertebrate.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

Some embodiments described herein relate to pharmaceutical compositions that comprise, consist essentially of, or consist of an effective amount of an oligonucleotide, a protein, or both, described herein and a pharmaceutically acceptable carrier, excipient, or combination thereof. A pharmaceutical composition described herein is suitable for human and/or veterinary applications.

The terms “function” and “functional” as used herein refer to a biological, enzymatic, or therapeutic function.

The term “isolated” as used herein refers to material that is substantially or essentially free from components that normally accompany it in its native state. For example, an “isolated cell,” as used herein, includes a cell that has been purified from the milieu or organisms in its naturally occurring state, a cell that has been removed from a subject or from a culture, for example, it is not significantly associated with in vivo or in vitro substances.

The terms “effective amount” or “effective dose” is used to indicate an amount of an active compound, or pharmaceutical agent, that elicits the biological or medicinal response indicated. For example, an effective amount of compound can be the amount needed to alleviate or ameliorate symptoms of disease or prolong the survival of the subject being treated This response may occur in a tissue, system, animal or human and includes alleviation of the signs or symptoms of the disease being treated. Determination of an effective amount is well within the capability of those skilled in the art, in view of the disclosure provided herein. The effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

The term “pharmaceutically acceptable salts” includes relatively non-toxic, inorganic and organic acid, or base addition salts of compositions, including without limitation, analgesic agents, therapeutic agents, other materials, and the like. Examples of pharmaceutically acceptable salts include those derived from mineral acids, such as hydrochloric acid, sulfuric acid, and those derived from organic acids, such as ethanesulfonic acid, benzenesulfonic acid, p-toluenesulfonic acid, and the like. Examples of suitable inorganic bases for the formation of salts include phosphates, hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; or trihydroxymethyl aminoethane.

“Formulation”, “pharmaceutical composition”, and “composition” as used interchangeably herein are equivalent terms referring to a composition of matter for administration to a subject.

The term “pharmaceutically acceptable” means compatible with therapy for a subject, and in particular, a human.

The terms “agent” refers to an active agent that has biological activity and may be used in a therapy. Also, an “agent” can be synonymous with “at least one agent,” “compound,” or “at least one compound,” and can refer to any form of the agent, such as a derivative, analog, salt or a prodrug thereof. The agent can be present in various forms, components of molecular complexes, and pharmaceutically acceptable salts (e.g., hydrochlorides, hydrobromides, sulfates, phosphates, nitrates, borates, acetates, maleates, tartrates, and salicylates). The term “agent” can also refer to any pharmaceutical molecules or compounds, therapeutic molecules or compounds, matrix forming molecules or compounds, polymers, synthetic molecules and compounds, natural molecules and compounds, and any combination thereof.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral, intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration. The pharmaceutical compositions described herein can also be administered to subjects along with other therapies, such as T cells, Natural Killer cells, B cells, macrophages, lymphocytes, stem cells, bone marrow cells, or hematopoietic stem cells.

The pharmaceutical compound can also be administered in a local rather than systemic manner, for example, via injection of the compound directly into an organ, tissue, or infected area, often in a depot or sustained release formulation. Furthermore, one may administer the compound in a targeted drug delivery system, for example, in a liposome coated with a tissue specific antibody. The liposomes may be targeted to and taken up selectively by the organ, tissue, cancer, tumor, or infected area.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. As described herein, compounds used in a pharmaceutical composition may be provided as salts with pharmaceutically compatible counterions.

As used herein, a “carrier” refers to a compound, particle, solid, semi-solid, liquid, or diluent that facilitates the passage, delivery and/or incorporation of a compound to cells, tissues and/or bodily organs. For example, without limitation, a lipid nanoparticle (LNP) is a type of carrier that can encapsulate an oligonucleotide to thereby protect the oligonucleotide from degradation during passage through the bloodstream and/or to facilitate delivery to a desired organ, such as to the liver.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the osmolarity and/or composition of human blood.

The term “excipient” has its ordinary meaning as understood in light of the specification, and refers to inert substances, compounds, or materials added to a pharmaceutical composition to provide, without limitation, bulk, consistency, stability, binding ability, lubrication, disintegrating ability etc., to the composition. Excipients with desirable properties, which may be incorporated into any one or more of the formulations set forth herein, include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, dextran, fructose, mannose, lactose, galactose, sucrose, sorbitol, cellulose, methyl cellulose, hydroxypropyl methyl cellulose (hypromellose), glycerin, polyvinyl alcohol, povidone, propylene glycol, serum, amino acids, polyethylene glycol, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. The amount of the excipient may be found in a pharmaceutical composition at a percentage of 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “adjuvant” as used herein refers to a substance, compound, or material that stimulates the immune response and increase the efficacy of protective immunity and is administered in conjunction with an immunogenic antigen, epitope, or composition. Adjuvants serve to improve immune responses by enabling a continual release of antigen, up-regulation of cytokines and chemokines, cellular recruitment at the site of administration, increased antigen uptake and presentation in antigen presenting cells, or activation of antigen presenting cells and inflammasomes. Commonly used adjuvants, which can be included in any one or more of the formulations set forth herein include but are not limited to alum, aluminum salts, aluminum sulfate, aluminum hydroxide, aluminum phosphate, calcium phosphate hydroxide, potassium aluminum sulfate, oils, mineral oil, paraffin oil, oil-in-water emulsions, detergents, Matrix-M, MF59®, squalene, AS03, α-tocopherol, polysorbate 80, AS04, monophosphoryl lipid A, virosomes, nucleic acids, polyinosinic:polycytidylic acid, saponins, QS-21, proteins, flagellin, cytokines, chemokines, IL-1, IL-2, IL-12, IL-15, IL-21, imidazoquinolines, CpG oligonucleotides, lipids, phospholipids, dioleoyl phosphatidylcholine (DOPC), trehalose dimycolate, peptidoglycans, bacterial extracts, lipopolysaccharides, or Freund’s Adjuvant, or any combination thereof.

The term “purity” of any given substance, compound, or material as used herein refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to side products, isomers, enantiomers, degradation products, solvent, carrier, vehicle, or contaminants, or any combination thereof. Purity can be measured technologies including but not limited to chromatography, liquid chromatography, gas chromatography, spectroscopy, UV-visible spectrometry, infrared spectrometry, mass spectrometry, nuclear magnetic resonance, gravimetry, or titration, or any combination thereof.

Some embodiments disclosed herein related to selecting a subject or patient in need. In some embodiments, a patient is selected who is in need of a treatment, inhibition, or amelioration of a viral infection such as SARS-CoV-2 or immunogenicity against a viral infection such as SARS-CoV-2. In some embodiments, a patient is selected as one identified as having aSARS-CoV-2 infection or as one in need of treatment of a viral infection such as SARS-CoV-2. In some embodiments, a patient is selected who has previously been treated for a viral infection, such as SARS-CoV-2. In some embodiments, a patient is selected who has previously been treated for being at risk of a viral infection, such as SARS-CoV-2. In some embodiments, a patient is selected who has developed a recurrence of a viral infection, such as SARS-CoV-2. In some embodiments, a patient is selected who has developed resistance to therapies for a viral infection, such as SARS-CoV-2. In some embodiments, a patient is selected who may have any combination of the aforementioned selection criteria. Such selections can be made by clinical and diagnostic evaluation of the subject or a combination of both. In some embodiments, the viral infection is a chronic viral infection. In some embodiments, the subject or patient is a “long-haul” patient or exhibits symptoms or sequela associated with SARS-CoV-2 infection but does not present an appreciable amount or no amount of detectable circulating virus. In some embodiments, SARS-CoV-2 includes the wild-type strain or variants thereof.

The terms “treat”, “treating”, “treatment”, “therapeutic”, or “therapy” as used herein has its ordinary meaning as understood in light of the specification, and do not necessarily mean total cure or abolition of the disease or condition. The term “treating” or “treatment” as used herein (and as well understood in the art) also means an approach for obtaining beneficial or desired results in a subject’s condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease’s transmission or spread, delaying or slowing of disease progression, amelioration or palliation of the disease state, diminishment of the reoccurrence of disease, and remission, whether partial or total and whether detectable or undetectable. “Treating” and “treatment” as used herein can in some but not all contexts include prophylactic treatment. Treatment methods comprise administering to a subject a therapeutically effective amount of an active agent. The administering step may consist of a single administration or may comprise a series of administrations. The compositions are administered to the subject in an amount and for a duration sufficient to treat the patient. The length of the treatment period depends on a variety of factors, such as the severity of the condition, the age and genetic profile of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from or developing a disease or condition.

The term “inhibit” as used herein has its ordinary meaning as understood in light of the specification, and may refer to the reduction of a viral infection, such as SARS-CoV-2. The reduction can be by 10%, 20%, 30%, 40%, 50%, 60%, 70%, 80%, 90%, or 100%, or an amount that is within a range defined by any two of the aforementioned values. As used herein, the term “delay” has its ordinary meaning as understood in light of the specification, and refers to a slowing, postponement, or deferment of an event, such as a viral infection, to a time which is later than would otherwise be expected. The delay can be a delay of 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

The term “immunogenic composition” as used herein refers to a substance or mixture of substances, including but not limited to antigens, epitopes, nucleic acids, peptides, polypeptides, proteins, polysaccharides, lipids, haptens, toxoids, inactivated organisms, or attenuated organisms, or any combination thereof, intended to elicit an immune response when administered to a host. The immune response includes both an innate and adaptive immune response, the latter of which establishes a lasting immunological memory through cells such as memory T cells and memory B cells. The antibodies created during the initial immune response to the immunogenic composition can be produced in subsequent challenges of the same antigens, epitopes, nucleic acids, peptides, polypeptides, proteins, polysaccharides, lipids, haptens, toxoids, inactivated organisms, or attenuated organisms, or a live organism or pathogen that exhibits the antigens, epitopes, nucleic acids, peptides, polypeptides, proteins, polysaccharides, lipids, haptens, or toxoids or any combination thereof. In this manner, the immunogenic composition may serve as a vaccine against a specific pathogen. Immunogenic compositions may also include one or more adjuvants to stimulate the immune response and increase the efficacy of protective immunity.

The term “product combination” as used herein refers to set of two or more individual compounds, substances, materials, or compositions that can be used together for a unified function. In some embodiments, a product combination comprises at least one nucleic acid composition and at least one polypeptide composition that are used together to elicit an immune response when administered to a host, optionally to a greater degree than would be elicited if only one composition type were to be administered.

The terms “nucleic acid” or “nucleic acid molecule” as used herein refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein refers to a sequence being after the 3′-end of a previous sequence, on the strand containing the encoding sequence (sense strand) if the nucleic acid is double stranded. The term “upstream” on a nucleic acid as used herein refers to a sequence being before the 5′-end of a subsequent sequence, on the strand containing the encoding sequence (sense strand) if the nucleic acid is double stranded. The term “grouped” on a nucleic acid as used herein refers to two or more sequences that occur in proximity either directly or with extra nucleic acids in between, e.g. linkers, repeats, or restriction enzyme sites, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The term “codon optimized” regarding a nucleic acid as used herein refers to the substitution of codons of the nucleic acid to enhance or maximize translation in a host of a particular species without changing the polypeptide sequence based on species-specific codon usage biases and relative availability of each aminoacyl-tRNA in the target cell cytoplasm. Codon optimization and techniques to perform such optimization is known in the art. Programs containing algorithms for codon optimization are known to those skilled in the art. Programs can include, for example, OptimumGene, GeneGPS® algorithms, etc. Additionally, synthetic codon optimized sequences can be obtained commercially for example from Integrated DNA Technologies and other commercially available DNA sequencing services. Those skilled in the art will appreciate that gene expression levels are dependent on many factors, such as promoter sequences and regulatory elements. As noted for most bacteria, small subsets of codons are recognized by tRNA species leading to translational selection, which can be an important limit on protein expression. In this aspect, many synthetic genes can be designed to increase their protein expression level.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein refers to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein refers to a sequence being after the C-terminus of a previous sequence. The term “upstream” on a polypeptide as used herein refers to a sequence being before the N-terminus of a subsequent sequence.

In some embodiments, the nucleic acid or peptide sequences presented herein and used in the examples are functional in various biological systems including but not limited to humans, mice, rabbits, E. coli, yeast, and mammalian cells. In other embodiments, nucleic acid or peptide sequences sharing at least or lower than 0%, 10%, 20%, 30%, 40%, 50%, 60%, 70%, 80%, 90%, 91%, 92%, 93%, 94%, 95%, 96%, 97%, 98%, 99%, or 100% similarity, or any percentage within a range defined by any two of the aforementioned percentages similarity to the nucleic acid or peptide sequences presented herein and used in the examples can also be used with no effect on the function of the sequences in biological systems. As used herein, the term “similarity” refers to a nucleic acid or peptide sequence having the same overall order of nucleotide or amino acids, respectively, as a template nucleic acid or peptide sequence with specific changes such as substitutions, deletions, repetitions, or insertions within the sequence. In some embodiments, two nucleic acid sequences sharing as low as 0%, 10%, 20%, 30%, 40%, 50%, 60%, 70%, 80%, 90%, 91%, 92%, 93%, 94%, 95%, 96%, 97%, 98%, or 99% similarity can encode for the same polypeptide by comprising different codons that encode for the same amino acid during translation.

The term “recombinantly expressed” as used herein refers to the production of proteins in optimized or adapted biological systems. These systems provide advantages over protein expression in a natural host, including but not limited to high expression (overexpression), ease of purification, ease of transformation, inducibility, low cost, or stability of the protein. In some embodiments, proteins are expressed in mammalian, bacteria, yeast, insect, or cell-free recombinant expression systems. Each system has its own advantages or disadvantages. For example, bacterial expression systems are highly optimized for overexpression, but may cause misfolding or aggregation of the produced protein, yeast systems are useful when post-translational modifications are necessary, and insect and mammalian systems are useful for proper RNA splicing that occurs in higher-order organisms. In some embodiments, recombinant polypeptides are produced and purified from mammalian, human, primary, immortalized, cancer, stem, fibroblasts, human embryonic kidney (HEK) 293, Chinese Hamster Ovary (CHO), bacterial, Escherichia coli, yeast, Saccharomyces cerevisiae, Pichia pastoris, insect, Spodoptera frugiperda Sƒ9, or S. frugiperda Sƒ21 cells, or in a cell-free system. In some embodiments, expression genes, vectors, or constructs are delivered to the recombinant expression systems in the form of plasmids, bacteriophages, viruses, adeno-associated viruses (AAVs), baculovirus, cosmids, fosmids, phagemids, BACs, YACs, or HACs. For more discussion on recombinant expression systems, see Gomes et al. “An Overview of Heterologous Expression Host Systems for the Production of Recombinant Proteins” ((2016) Adv. Anim. Vet. Sci. 4(7):346-356), hereby expressly incorporated by reference in its entirety.

The term “coronavirus” as used herein refers to the family of enveloped, positive-sense, single stranded RNA viruses that infect mammals and birds. In humans, coronavirus infections can cause mild symptoms as a common cold, or more severe respiratory conditions such as severe acute respiratory syndrome (SARS), acute respiratory distress syndrome (ARDS), coughing, congestion, sore throat, shortness of breath, pneumonia, bronchitis, and hypoxia. Other symptoms include but are not limited to fever, fatigue, myalgia, and gastrointestinal symptoms such as vomiting, diarrhea, and abdominal pain. The viral envelope comprises spike (“S”), envelope (“E”), membrane (“M”), and hemagglutinin esterase (“HE”) transmembrane structural proteins. The S protein comprises a receptor binding domain (“RBD”), a highly immunogenic region that determines the host receptor specificity of the virus strain. The viral nucleocapsid comprises multiple nucleocapsid (“N” or “NP”) proteins coating the RNA genome. During infection, the S protein attaches to a host cell receptor and initiate entry into the host cell through endocytosis or fusion of the envelope membrane. The RNA genome is translated by the host ribosome to produce new structural proteins and RNA-dependent RNA polymerases, which replicate the viral genome. Viral particles are assembled in the host endoplasmic reticulum and are shed by Golgi-mediated exocytosis. More information about the structure and infection cycle of coronaviruses can be found in Fehr AR & Perlman S. “Coronaviruses: An Overview of Their Replication and Pathogenesis” Methods Mol. Biol. (2015); 1282:1-23, hereby expressly incorporated by reference in its entirety.

The terms “SARS-CoV-2” and “2019-nCoV” as used herein refers to the coronavirus strain or strains responsible for the human coronavirus disease 2019 (COVID-19) pandemic. The contagiousness, long incubation period, and modern globalization has led to worldwide spread of the virus. Development of SARS and other respiratory issues in infected individuals has resulted in immense stress on medical infrastructure. Treatments and vaccines for SARS-CoV-2 and other coronaviruses in humans are starting to be approved, but additional testing is necessary. Reference sequences for the original wild-type (Wuhan-Hu-1) strain are available by NCBI GenBank accession number: MN908947.3 (e.g. complete genome), YP_009724390 (e.g. surface glycoprotein), YP_009724393.1 (e.g. membrane glycoprotein), and YP_009724397.2 (e.g. nucleocapsid phosphoprotein). Like the original SARS virus (SARS-CoV-1), SARS-CoV-2 infects human cells by binding to angiotensin-converting enzyme 2 (ACE2) through the RBD of the S protein. The RBD, M protein, and NP protein are good candidates for the development of treatments, prophylaxes, interventions, vaccines, or immunogenic compositions against SARS-CoV-2 and other coronaviruses. An embodiment of the SARS-CoV-2 RBD sequence is provided as SEQ ID NO: 100. As disclosed herein, the RBD sequence used in the embodiments provided herein may have one or more mutations, where the mutations may be those found in variants of SARS-CoV-2 relative to the original SARS-CoV-2 (Wuhan-Hu-1). Furthermore, in some embodiments, the RBD sequence used in the embodiments provided herein may have one or more mutations at cysteines in the sequence, for example, for the purpose of expressing the RBD sequence recombinantly or ex vivo as the presence of cysteines may interfere with protein folding or function. In some embodiments, the RBD sequence used in the embodiments provided herein comprise one or more mutations at C336, C361, C379, C391, C432, C480, C488, or C525 with reference to the full SARS-CoV-2 S protein (e.g. as set forth in NCBI Accession No. YP_009724390 (SEQ ID NO: 83)), or none of these mutations. The embodiments disclosed herein can be applied to other coronaviruses, including but not limited to HCoV-229E, HCoV-OC43, SARS-CoV-1, HCoV NL63, HCoV-HKU1, and MERS-CoV, or variants of SARS-CoV-2.

During the COVID-19 pandemic, emergent genetic variants were discovered. These variants may exhibit different host specificity or increased transmissibility, infectivity, and/or virulence. Furthermore, there are concerns that these variants or new variants may reduce the efficacy of currently approved vaccines. The primary genetic mutations of concern involve the S protein (and corresponding RBD), which the virus uses for host receptor binding; as the current vaccines are directed to immunogenicity against the wild-type (Wuhan-hu-1) S proteins, they may result in reduced efficacy against these mutant strains.

Three prominent Variants of Concern (VOC) are the Alpha variant (B.1.1.7, 20B/501Y.V1, VOC 20212/01, first identified in the United Kingdom), the Beta variant (B.1.351, 20C/501Y.V2, first identified in South Africa), the Gamma variant (P.1, 20J/501Y.V3, originating from Brazil and first identified in Japan). These variants have been found to exhibit rapid and wide-spread transmission throughout the world. A common mutation among these three strains is N501Y, which is at one of six contact residues of the RBD that interfaces with human ACE2 and has been shown to increase affinity towards ACE2 (Starr et al. “Deep Mutational Scanning of SARS-CoV-2 Receptor Binding Domain Reveals Constraints on Folding and ACE2 Binding” Cell; (2020) 182(5);1295-1310, hereby expressly incorporated by reference in its entirety). The Beta variant also comprises the mutations K417N and E484K. The Gamma variant has 17 unique amino acid changes and three deletions, including K417T, E484K, and N501Y mutations in the spike protein receptor binding domain. Other variants comprise the N439K mutation. These mutations have been suspected to interfere with antibody recognition.

The Delta variant (B.1.617.2) is one of the most transmissible VOC, and is believed to have been a dominant variant during the COVID-19 pandemic. The Delta variant is distinct from other VOC such as the Alpha, Beta, and Gamma variants, as it does not comprise the N501Y mutation within the RBD. Instead, among other mutations in other regions of the genome, the Delta variant comprises L452R and T478K mutations in the RBD. In addition to the enhanced transmissibility of this variant (which may be attributed to these mutations), this exacerbates significant concerns regarding efficacy of currently available vaccines. As the Delta variant remains prevalent in the world with the risk of new mutants emerging, even more potentially virulent strains may be identified. The related Delta plus variant (Delta with K417N) is also prevalent and exhibits the K417N mutation seen in the Beta and other variants.

The Omicron variant (B.1.1.529) has developed later during the COVID-19 pandemic as a dominant VOC. This variant comprises significantly more mutations than previous VOCs (60 mutations total), with 30 mutations in the spike protein. Therefore, there has been some concerns regarding the efficacy of current vaccines towards preventing infection by the Omicron variant. The Omicron variant includes the BA.1 and BA.2 sub-lineages. The mutations found in the BA.1 sub-lineage that are explored in the embodiments disclosed herein include G339D, S371L, S373P, S375F, K417N, N440K, G446S, S477N, T478K, E484A, Q493R, G496S, Q498R, N501Y, and Y505H. The mutations found in the BA.2 sub-lineage that are explored in the embodiments disclosed herein include G339D, S371F, S373P, S375F, T376A, D405N, R408S, K417N, N440K, S477N, T478K, E484A, Q493R, Q498R, N501Y, Y505H. However, the other mutations as well as any other combination of mutations found in the Omicron variant is envisioned for use herein.

In addition to these VOCs, other strains have been identified as Variants of Interest (VOI) or variants that are under monitoring, which potentially pose significant threats but perhaps has not been as prevalently transmitted compared to VOCs. Exemplary VOIs include the Theta, Iota, Kappa, Lambda, and Mu variants. Other variants of interest include B.1.427, B.1.429, R.1, B.1.466.2, B.1.1.318, B.1.1.519, C.36.3, B.1.214.2, B.1.1.523, B.1.619, B.1.620, C.1.2, B.1.617.1, B.1.526, and B.1.525.

Current lists of VOCs and VOIs and sequence information thereof are available from international regulatory bodies, such as the Centers for Disease Control and World Health Organization. One such example is available on the world wide web at who.int/en/activities/tracking-SARS-CoV-2-variants/. In addition to the mutations disclosed herein, it is envisioned that the embodiments provided herein may include any one or more of the mutations associated with these variants, in any conformation, in order to improve efficacy against wild-type SARS-CoV-2 and SARS-CoV-2 variants.

As disclosed herein, in some embodiments, the nucleic acids and polypeptides for use as immunogenic compositions may encode or comprise the mutations disclosed herein, or other mutations within the S protein or corresponding RBD, such as those identified in other SARS-CoV-2 variants. The incorporation of these immunogens into the formulations and methods described herein will produce an increased diversity of antibody and T cell response in the inoculated patient, which will provide for a robust protection against SARS-CoV-2 and SARS-CoV-2 variants.

In some embodiments, the RBD sequences used herein are tandem repeat single chain dimer variants. RBD dimers have been shown to improve immunogenicity and increase neutralizing antibody titers. Both disulfide-linked dimers and single chain (covalently linked) dimers are effective in this aspect. In some embodiments, the RBD tandem repeat single chain dimer is constructed by fusing two coronavirus RBD sequences with or without additional linkers or other amino acids. A non-limiting example of an RBD tandem repeat single chain dimer polypeptide is embodied in SEQ ID NO: 46. A non-limiting example of a nucleic acid sequence encoding an RBD tandem repeat single chain dimer polypeptide is embodied in SEQ ID NO: 45. In some embodiments, the RBD tandem repeat single chain dimers may comprise any one or more of the mutations disclosed herein and/or additional mutations associated with one or more SARS-CoV-2 variants (e.g., Alpha, Beta, Gamma, Delta, Theta, Iota, Kappa, Lambda, Mu, Omicron (including the BA.1 or BA.2 sub-lineage), or other known variants). For example, the RBD tandem repeat single chain dimer may comprise a mutation at K417, N439, L452, T478, E484, or N501, or any combination thereof, or none of these mutations, associated with a SARS-CoV-2 variant (where it is understood that these mutations are set forth with reference to the full SARS-CoV-2 S protein (e.g., as set forth in NCBI Accession No. YP_009724390). In some embodiments, the RBD tandem repeat single chain dimer may comprise a K417N, N439K, L452R, T478K, E484K, or N501Y mutation, or any combination thereof, or none of these mutations, associated with a SARS-CoV-2 variant (where it is understood that these mutations are set forth with reference to the full SARS-CoV-2 S protein (e.g., as set forth in NCBI Accession No. YP_009724390)). In some embodiments, the RBD tandem repeat single chain dimer may comprise mutations at one or more cysteines in the sequence (e.g., C336, C361, C379, C391, C432, C480, C488, or C525), for example, to enhance expression of the RBD tandem repeat single chain dimer (where it is understood that these mutations are set forth with reference to the full SARS-CoV-2 S protein (e.g., as set forth in NCBI Accession No. YP_009724390)). Throughout this disclosure, RBD tandem repeat single chain dimers may also be referred as RBD version 2 (RBDv2). Additional insight into RBD tandem repeat single chain dimers may be found in Dai et al. “A Universal Design of Betacoronavirus Vaccines against COVID-19, MERS, and SARS” Cell. (2020);182(3):722-733, which is hereby expressly incorporated by reference in its entirety.

In some embodiments, the RBD sequences are assembled in multimeric variants, such as variants with 3, 4, 5, 6, 7, 8, 9, or 10 copies of one or more RBD sequences. In some embodiments, the RBD sequences are assembled into trimeric variants. An example of a construct with a trimeric RBD variants is OC-2.4. In some embodiments, each of the RBD sequences in the multimeric variants may comprise any one or more of the mutations disclosed herein and/or additional mutations associated with one or more SARS-CoV-2 variants (e.g., Alpha, Beta, Gamma, Delta, Theta, Iota, Kappa, Lambda, Mu, Omicron (including the BA.1 or BA.2 sub-lineage), or other known variants). In some embodiments, one or more RBD sequences in the multimeric variants may comprises a K417N, N439K, L452R, T478K, E484K, N501Y, G339D, S371L, S371F, S373P, S375F, T376A, D405N, R408S, N440K, G446S, S447N, Q493R, G496S, Q498R, or Y505H mutation, or any combination thereof, or none of these mutations, associated with a SARS-CoV-2 variant (where it is understood that these mutations are set forth with reference to the full SARS-CoV-2 S protein (e.g., as set forth in NCBI Accession No. YP_009724390)). In some embodiments, one or more RBD sequences in the multimeric variants may comprise a K417N, N439K, L452R, T478K, E484K, or N501Y mutation, or any combination thereof, or none of these mutations, associated with a SARS-CoV-2 variant (where it is understood that these mutations are set forth with reference to the full SARS-CoV-2 S protein (e.g., as set forth in NCBI Accession No. YP_009724390)). In some embodiments, the one or more RBD sequences in the multimeric variants may comprise mutations at one or more cysteines in the sequence (e.g., C336, C361, C379, C391, C432, C480, C488, or C525), for example, to enhance expression of the RBD tandem repeat single chain dimer (where it is understood that these mutations are set forth with reference to the full SARS-CoV-2 S protein (e.g., as set forth in NCBI Accession No. YP_009724390)).

The terms “autocatalytic peptide cleavage site” or “2A peptide” as used herein refer to a peptide sequence that undergo cleavage of a peptide bond between two constituent amino acids, resulting in separation of the two proteins that flank the sequence. The cleavage is believed to be a result of a ribosomal “skipping” of the peptide bond formation between the C-terminal proline and glycine in the 2A peptide sequence. Four autocatalytic peptide cleavage site sequences identified to date have seen substantial use in biomedical research: foot-and-mouth disease virus 2A (F2A); equine rhinitis A virus (ERAV) 2A (E2A); porcine teschovirus-1 2A (P2A), and Thosea asigna virus 2A (T2A). In some embodiments, the P2A autocatalytic peptide cleavage site nucleic acid (SEQ ID NO: 37) and polypeptide (SEQ ID NO: 38) sequences are used. In some embodiments, the P2A nucleic acid or polypeptide used can be substituted with an F2A, E2A, or T2A nucleic acid or polypeptide.

In some embodiments, the nucleic acids or peptides used herein comprise sequences representing hepatitis D antigen (HDAg) variants. Hepatitis D is a virusoid that relies on hepatitis B coinfection or superinfection to replicate. The circular single-stranded RNA of hepatitis D is amplified using host RNA polymerases, but also contains a single hepatitis D antigen (HDAg) gene. During hepatitis B and D coinfection or superinfection, intact hepatitis D viruses are packaged with an envelope containing hepatitis B surface antigens surrounding the RNA genome that is coated with HDAg protein. Incorporation of the hepatitis B surface antigens is essential for hepatitis D infectivity, as hepatitis D does not encode its own receptor binding proteins. Coinfection or superinfection with hepatitis D causes more severe complications, with increased risk of liver failure, cirrhosis, and cancer. A small (24 kDa) and large (27 kDa, 213 amino acids excluding the start methionine) isoform exist for HDAg and are translated from the same open reading frame on the HDV genome. Deamination of the adenosine in a UAG stop codon at codon 196 of the coding sequence allows for translation to continue and produce the large isoform. Unless expressly stated otherwise, the embodiments described herein comprise the large isoform of HDAg. In some embodiments, the HDAg sequences comprise at least one of four different HDAg strain sequences: “HDAg genotype 1A”, “HDAg genotype 1B”, “HDAg genotype 2A”, or “HDAg genotype 2B”. Additional information about HDAg sequences and uses thereof can be found in PCT Publication WO 2017/132332, hereby expressly incorporated by reference in its entirety.

The term “IgE leader sequence” as used herein refers to the amino acid sequence MDWTWILFLVAAATRVHS (SEQ ID NO: 44), which can be appended to the N-terminus of a protein to both enhance translation and increase immunogenicity. Translation is particularly upregulated when the IgE leader sequence is used in combination with a functional Kozak sequence. An exemplary embodiment of a nucleic acid sequence that encodes for the amino acid IgE leader sequence is represented as SEQ ID NO: 43. However, it would be clearly apparent to one skilled in the art to develop alternative nucleic acid sequence that would result in the same amino acid sequence when translated. Additional insight into the use of an IgE leader sequence may be found in Vijayachari et al. “Immunogenicity of a novel enhanced consensus DNA vaccine encoding the leptospiral protein LipL45” Hum. Vaccin. Immunother. (2015);11(8):1945-53, which is hereby expressly incorporated by reference in its entirety.

The term “CC40.8 epitope” as used herein refers to a conserved epitope of the beta-coronavirus spike protein, wherein beta-coronavirus is the genus that includes SARS-CoV, SARS-CoV-2, MERS-CoV, and other prevalent coronavirus pathogens. This conserved epitope is found in the S2 stem-helix region of the spike protein. Antibodies raised against this epitope (including the original CC40.8 antibody described to bind to this region) can exhibit protective activity against infection by a broad range of beta-coronaviruses, including SARS-CoV-2 and its variants. The amino acid sequence of the CC40.8 epitope for SARS-CoV-2 is represented as SEQ ID NO: 91. An exemplary nucleic acid sequence that encodes for the CC40.8 epitope for SARS-CoV-2 and that is codon optimized for expression in humans is represented as SEQ ID NO: 90. In some embodiments, sequences representing the CC40.8 epitope from other related coronaviruses, or fragments of CC40.8 sequences, can be used herein. Additional information regarding the CC40.8 epitope can be found in Zhou et al. A human antibody reveals a conserved site on beta-coronavirus spike proteins and confers protection against SARS-CoV-2 infection. Science. Transl. Med. (2022) doi:10.1126/scitranslmed.abi9215, which is hereby expressly incorporated by reference in its entirety.

The terms “in vivo electroporation”, “electroporation”, and “EP” as used herein refers to the delivery of genes, nucleic acids, DNA, RNA, proteins, or vectors into cells of living tissues or organisms using electrical currents using techniques known in the art. Electroporation can be used as an alternative to other methods of gene transfer such as viruses (transduction), lipofection, gene gun (biolistics), microinjection, vesicle fusion, or chemical transformation. Electroporation limits the risk of immunogenicity and detrimental integration or mutagenesis of the cell genome. DNA vectors such as plasmids are able to access the cell nucleus, enabling transcription and translation of constituent genes. In some embodiments, the genes, nucleic acids, DNA, RNA, proteins, or vectors are added to the target tissue or organism by subcutaneous, intramuscular, or intradermal injection. An electroporator then delivers short electrical pulses via electrodes placed within or proximal to the injected sample. As used herein, the term “im/EP” refers to in vivo electroporation of a sample delivered intramuscularly (“im”).

The terms “K18-hACE2” or “B6.Cg-Tg(K18-ACE2)2Prlmn/J” as used herein refers to a transgenic mouse model expressing human ACE2, the receptor that coronaviruses such as SARS-CoV-1 and SARS-CoV-2 used to infect human cells. Expression of human ACE2 is driven by the human cytokeratin 18 promoter. These mice can be used as experimental models for SARS-CoV-2 viral infections. Other similar mouse models can be used as alternatives.

Where a range of values is provided, it is understood that the upper and lower limit, and each intervening value between the upper and lower limit of the range is encompassed within the embodiments.

The term “% w/w” or “% wt/wt” as used herein has its ordinary meaning as understood in light of the specification and refers to a percentage expressed in terms of the weight of the ingredient or agent over the total weight of the composition multiplied by 100. The term “% v/v” or “% vol/vol” as used herein has its ordinary meaning as understood in the light of the specification and refers to a percentage expressed in terms of the liquid volume of the compound, substance, ingredient, or agent over the total liquid volume of the composition multiplied by 100.

### Exemplary Nucleic Acids and Polypeptides

Disclosed herein are nucleic acids that can be used as immunogenic compositions or part of immunogenic product combinations, for example, to generate an immune response against SARS-CoV-2 or other coronavirus, and/or generate neutralizing antibodies against SARS-CoV-2 or other coronavirus in a subject.

In some embodiments, the nucleic acid comprises at least one nucleic acid sequence encoding a SARS-CoV-2 polypeptide and at least one nucleic acid sequence encoding a P2A autocatalytic polypeptide cleavage site. In some embodiments, the at least one nucleic acid sequence encoding a SARS-CoV-2 polypeptide comprises a nucleic acid sequence encoding a receptor binding domain (RBD) polypeptide and a nucleic acid encoding a nucleocapsid protein (NP) polypeptide. In some embodiments, the nucleic acid shares or comprises at least 90%, 91%, 92%, 93%, 94%, 95%, 96%, 97%, 98%, 99%, or 100% homology or sequence identity to SEQ ID NO: 1 or 13. In some embodiments, the at least one nucleic acid sequence encoding a SARS-CoV-2 polypeptide comprises a nucleic acid sequence encoding an RBD polypeptide, a nucleic acid sequence encoding an M polypeptide, and a nucleic acid sequence encoding an NP polypeptide. In some embodiments, the nucleic acid shares or comprises at least 90%, 91%, 92%, 93%, 94%, 95%, 96%, 97%, 98%, 99%, or 100% homology or sequence identity to any one of SEQ ID NO: 2-3, or 14-15. In some embodiments, the RBD polypeptide is an RBD tandem repeat single chain dimer polypeptide. In some embodiments, the RBD tandem repeat single chain dimer polypeptide comprises a K417N, N439K, L452R, T478K, E484K, or N501Y mutation, or any combination thereof, or none of these mutations with reference to the full SARS-CoV-2 S protein (e.g., as set forth in NCBI Accession No. YP_009724390)). In some embodiments, the RBD tandem repeat single chain dimer polypeptide comprises a C336, C361, C379, C391, C432, C480, C488, or C525 mutation, or any combination thereof, or none of these mutations, with reference to the full SARS-CoV-2 S protein (e.g., as set forth in NCBI Accession No. YP_009724390)). In some embodiments, the nucleic acid sequence encoding the RBD tandem repeat single chain dimer polypeptide shares or comprises at least 90%, 91%, 92%, 93%, 94%, 95%, 96%, 97%, 98%, 99%, or 100% homology or sequence identity to any one or more of SEQ ID NO: 45, or 47-50. In some embodiments, the nucleic acid shares or comprises at least 90%, 91%, 92%, 93%, 94%, 95%, 96%, 97%, 98%, 99%, or 100% homology or sequence identity to SEQ ID NO: 39. In some embodiments, the RBD polypeptide comprises three tandem copies of RBD (or RBDv2). In some embodiments, the three tandem copies of RBD each comprise a K417N, N439K, L452R, T478K, E484K, or N501Y mutation with reference to the full SARS-CoV-2 S protein (e.g., as set forth in NCBI Accession No. YP_009724390), or any combination thereof, or none of these mutations. In some embodiments, the three tandem copies of RBD each comprise a C336, C361, C379, C391, C432, C480, C488, or C525 mutation, or any combination thereof, or none of these mutations, with reference to the full SARS-CoV-2 S protein (e.g., as set forth in NCBI Accession No. YP_009724390)). In any of the embodiments disclosed herein, the RBD polypeptide, NP polypeptide, M polypeptide, or S polypeptide may be derived from the wild-type SARS-CoV-2 strain (Wuhan-hu-1) or a SARS-CoV-2 variant, optionally the Alpha, Beta, Gamma, Delta, Theta, Iota, Kappa, Lambda, Mu, or Omicron (including the BA.1 or BA.2 sub-lineage) variant. In some embodiments, the RBD polypeptide, NP polypeptide, M polypeptide, or S polypeptide comprises one or more mutations found in a SARS-CoV-2 variant, optionally the Alpha, Beta, Gamma, Delta, Theta, Iota, Kappa, Lambda, Mu, or Omicron (including the BA.1 or BA.2 sub-lineage) variant, relative to the wild-type SARS-CoV-2 strain (Wuhan-hu-1). In some embodiments, the RBD polypeptide comprises one or more mutations found in a SARS-CoV-2 variant, optionally the Alpha, Beta, Gamma, Delta, Theta, Iota, Kappa, Lambda, Mu, or Omicron (including the BA.1 or BA.2 sub-lineage) variant, relative to the wild-type SARS-CoV-2 strain (Wuhan-hu-1).

As applied to any of the nucleic acids disclosed herein, in some embodiments, the nucleic acid further comprises a 5′ IgE leader nucleic acid sequence. In some embodiments, the 5′ IgE leader nucleic acid sequence shares or comprises at least 90%, 91%, 92%, 93%, 94%, 95%, 96%, 97%, 98%, 99%, or 100% homology or sequence identity to SEQ ID NO: 43. In some embodiments, the RBD polypeptide is an RBD tandem repeat single chain dimer polypeptide. In some embodiments, the RBD tandem repeat single chain dimer polypeptide comprises a K417N, N439K, L452R, T478K, E484K, or N501Y mutation with reference to the full SARS-CoV-2 S protein (e.g., as set forth in NCBI Accession No. YP_009724390)), or any combination thereof, or none of these mutations. In some embodiments, the RBD tandem repeat single chain dimer polypeptide comprises a C336, C361, C379, C391, C432, C480, C488, or C525 mutation, or any combination thereof, or none of these mutations, with reference to the full SARS-CoV-2 S protein (e.g., as set forth in NCBI Accession No. YP_009724390)). In some embodiments, the nucleic acid sequence encoding the RBD tandem repeat single chain dimer polypeptide shares or comprises at least 90%, 91%, 92%, 93%, 94%, 95%, 96%, 97%, 98%, 99%, or 100% homology or sequence identity to any one or more of SEQ ID NO: 45, or 47-50. In some embodiments, the nucleic acid shares or comprises at least 90%, 91%, 92%, 93%, 94%, 95%, 96%, 97%, 98%, 99%, or 100% homology or sequence identity to any one or more of SEQ ID NO: 45, or 47-50. In some embodiments, the nucleic acid shares or comprises at least 90%, 91%, 92%, 93%, 94%, 95%, 96%, 97%, 98%, 99%, or 100% homology or sequence identity to any one or more of SEQ ID NO: 40, 57-60, or 62. In some embodiments, the RBD polypeptide comprise three tandem copies of RBD (or RBDv2). In some embodiments, the three tandem copies of RBD each comprise a K417N, N439K, L452R, T478K, E484K, or N501Y mutation with reference to the full SARS-CoV-2 S protein (e.g., as set forth in NCBI Accession No. YP_009724390), or any combination thereof, or none of these mutations. In some embodiments, the three tandem copies of RBD each comprise a C336, C361, C379, C391, C432, C480, C488, or C525 mutation, or any combination thereof, or none of these mutations, with reference to the full SARS-CoV-2 S protein (e.g., as set forth in NCBI Accession No. YP_009724390)). In some embodiments, the nucleic acid shares or comprises at least 90%, 91%, 92%, 93%, 94%, 95%, 96%, 97%, 98%, 99%, or 100% homology or sequence identity to SEQ ID NO: 61.

In some embodiments, the at least one nucleic acid sequence encoding a SARS-CoV-2 polypeptide comprises a nucleic acid sequence encoding an RBD polypeptide and a nucleic acid sequence encoding an M polypeptide. In some embodiments, the nucleic acid shares or comprises at least 90%, 91%, 92%, 93%, 94%, 95%, 96%, 97%, 98%, 99%, or 100% homology or sequence identity to SEQ ID NO: 4 or 16. In some embodiments, the variance in sequence identity is in the RBD polypeptide at sequences encoding for C336, C361, C379, C391, C432, C480, C488, or C525, or any combination thereof, with reference to the full SARS-CoV-2 S protein.

In some embodiments, the at least one nucleic acid sequence encoding the SARS-CoV-2 polypeptide comprises a nucleic acid sequence encoding for a spike (S) polypeptide. In some embodiments, the at least one nucleic acid sequence encoding the SARS-CoV-2 polypeptide comprises a nucleic acid sequence encoding for a membrane (M) polypeptide. In some embodiments, the at least one nucleic acid sequence encoding the SARS-CoV-2 polypeptide further comprises a nucleic acid sequence encoding for a nucleocapsid protein (NP) polypeptide. In some embodiments, the at least one nucleic acid sequence encoding the SARS-CoV-2 polypeptide comprises a nucleic acid sequence encoding for a S polypeptide, a nucleic acid sequence encoding for a M polypeptide, or a nucleic acid sequence encoding for a NP polypeptide, or any combination thereof. In some embodiments, the S polypeptide comprises mutations to facilitate improved expression, solubility, and/or immunogenicity. In some embodiments, the S polypeptide comprises a K968P or V987P mutation with reference to the full SARS-CoV-2 S protein (e.g., as set forth in NCBI Accession No. YP_009724390), or both. In some embodiments, the nucleic acid sequence encoding the S polypeptide shares or comprises at least 90%, 91%, 92%, 93%, 94%, 95%, 96%, 97%, 98%, 99%, or 100% homology or sequence identity to SEQ ID NO: 51. In some embodiments, the nucleic acid further comprises a 5′ IgE leader nucleic acid sequence. In some embodiments, the 5′ IgE leader nucleic acid sequence shares or comprises at least 90%, 91%, 92%, 93%, 94%, 95%, 96%, 97%, 98%, 99%, or 100% homology or sequence identity to SEQ ID NO: 43. In some embodiments, the nucleic acid shares or comprises at least 90%, 91%, 92%, 93%, 94%, 95%, 96%, 97%, 98%, 99%, or 100% homology or sequence identity to SEQ ID NO: 63. In some embodiments, the variance in sequence identity is in the RBD polypeptide at sequences encoding for C336, C361, C379, C391, C432, C480, C488, or C525, or any combination thereof, with reference to the full SARS-CoV-2 S protein.

In some embodiments, the nucleic acid comprises at least one nucleic acid sequence encoding a SARS-CoV-2 polypeptide. In some embodiments, the nucleic acid shares or comprises at least 90%, 91%, 92%, 93%, 94%, 95%, 96%, 97%, 98%, 99%, or 100% homology or sequence identity to any one or more of SEQ ID NO: 5-7, 17-19, 22-24, 73, or 75. In some embodiments, the variance in sequence identity is in the RBD polypeptide at sequences encoding for C336, C361, C379, C391, C432, C480, C488, or C525, or any combination thereof, with reference to the full SARS-CoV-2 S protein.

In some embodiments, the nucleic acid comprises at least one nucleic acid sequence encoding a SARS-CoV-2 polypeptide and at least one nucleic acid sequence encoding a hepatitis D antigen (HDAg). In some embodiments, the nucleic acid shares or comprises at least 90%, 91%, 92%, 93%, 94%, 95%, 96%, 97%, 98%, 99%, or 100% homology or sequence identity to SEQ ID NO: 8 or 20. In some embodiments, the nucleic acid further comprises at least one nucleic acid sequence encoding a P2A autocatalytic polypeptide cleavage site. In some embodiments, the nucleic acid shares or comprises at least 90%, 91%, 92%, 93%, 94%, 95%, 96%, 97%, 98%, 99%, or 100% homology or sequence identity to SEQ ID NO: 9 or 21. In some embodiments, the variance in sequence identity is in the RBD polypeptide at sequences encoding for C336, C361, C379, C391, C432, C480, C488, or C525, or any combination thereof, with reference to the full SARS-CoV-2 S protein.

In some embodiments of any one of the nucleic acids disclosed herein, the nucleic acid further comprises a 5′ IgE leader nucleic acid sequence. In some embodiments, the 5′ IgE leader nucleic acid sequence shares or comprises at least 90%, 91%, 92%, 93%, 94%, 95%, 96%, 97%, 98%, 99%, or 100% homology or sequence identity to SEQ ID NO: 43.

In any of the nucleic acids disclosed herein, the nucleic acid may encode for any one or more of the SARS-CoV-2 polypeptides disclosed herein or otherwise conventionally known in the art. In some embodiments, the one or more SARS-CoV-2 polypeptides comprise an RBD polypeptide. In some embodiments, the RBD polypeptide is from the SARS-CoV-2 virus or a variant thereof. In some embodiments, the RBD polypeptide comprises a K417N, N439K, L452R, T478K, E484K, or N501Y mutation with reference to the full SARS-CoV-2 S protein (e.g., as set forth in NCBI Accession No. YP_009724390), or any combination thereof, or none of these mutations. In some embodiments, a nucleic acid encoding for the RBD polypeptide is represented by SEQ ID NO: 10 or 22. In some embodiments, the RBD polypeptide is represented by SEQ ID NO: 34. In some embodiments, the RBD polypeptide is an RBD tandem repeat single chain dimer polypeptide. In some embodiments, the RBD tandem repeat single chain dimer polypeptide comprises a K417N, N439K, L452R, T478K, E484K, N501Y, G339D, S371L, S371F, S373P, S375F, T376A, D405N, R408S, N440K, G446S, S447N, Q493R, G496S, Q498R, or Y505H mutation with reference to the full SARS-CoV-2 S protein (e.g., as set forth in NCBI Accession No. YP_009724390), or any combination thereof, or none of these mutations. In some embodiments, the RBD tandem repeat single chain dimer polypeptide comprises a K417N, N439K, L452R, T478K, E484K, or N501Y mutation with reference to the full SARS-CoV-2 S protein (e.g., as set forth in NCBI Accession No. YP_009724390), or any combination thereof, or none of these mutations. In some embodiments, a nucleic acid encoding for the RBD polypeptide is represented by any one of SEQ ID NOs: 45, or 47-50. In some embodiments, the RBD polypeptide is represented by any one of SEQ ID NOs: 46, or 52-55. In some embodiments, a nucleic acid encoding for an M polypeptide is represented by SEQ ID NOs: 11 or 23. In some embodiments, the M polypeptide is represented by SEQ ID NO: 35. In some embodiments, a nucleic acid encoding for an NP polypeptide is represented by SEQ ID NOs: 12 or 24. In some embodiments, the NP polypeptide is represented by SEQ ID NO: 36.

In some embodiments, the nucleic acid comprises at least one nucleic acid sequence encoding a SARS-CoV-2 polypeptide, wherein the at least one nucleic acid sequence encoding a SARS-CoV-2 polypeptide comprises: i) one or more nucleic acid sequences encoding a SARS-CoV-2 spike receptor binding domain (RBD) polypeptide; ii) a nucleic acid sequence encoding a SARS-CoV-2 nucleocapsid protein (NP) polypeptide; iii) a nucleic acid sequence encoding a SARS-CoV-2 membrane (M) polypeptide; iv) a nucleic acid sequence encoding a hepatitis D antigen (HDAg) polypeptide; v) a nucleic acid sequence encoding an autocatalytic polypeptide cleavage site; vi) a nucleic acid sequence encoding an IgE leader polypeptide; vii) a nucleic acid sequence encoding a SARS-CoV-2 spike polypeptide; or viii) a nucleic acid sequence encoding a CC40.8 epitope; or any combination thereof. In some embodiments, one or more of the RBD polypeptide, NP polypeptide, M polypeptide, or S polypeptide is derived from the wild-type SARS-CoV-2 strain (Wuhan-hu-1) or a SARS-CoV-2 variant, optionally the Alpha, Beta, Gamma, Delta, Theta, Iota, Kappa, Lambda, Mu, or Omicron (including the BA.1 or BA.2 sub-lineage) variant. In some embodiments, one or more of the RBD polypeptide, NP polypeptide, M polypeptide, or S polypeptide comprises one or more mutations found in a SARS-CoV-2 variant, optionally the Alpha, Beta, Gamma, Delta, Theta, Iota, Kappa, Lambda, Mu, or Omicron (including the BA.1 or BA.2 sub-lineage) variant, relative to the wild-type SARS-CoV-2 strain (Wuhan-hu-1). In some embodiments, the RBD polypeptide comprises one or more mutations found in a SARS-CoV-2 variant, optionally the Alpha, Beta, Gamma, Delta, Theta, Iota, Kappa, Lambda, Mu, or Omicron (including the BA.1 or BA.2 sub-lineage) variant, relative to the wild-type SARS-CoV-2 strain (Wuhan-hu-1).

In some embodiments, each of the one or more nucleic acid sequences encoding the RBD polypeptide comprise one or more mutations corresponding to K417, N439, L452, T478, E484, N501, G339, S371, S373, S375, T376, D405, R408, N440, G446, S447, Q493, G496, Q498, or Y505 with reference to the full SARS-CoV-2 S protein (e.g. as set forth in NCBI Accession No. YP_009724390 (SEQ ID NO: 83)), or none of these mutations. In some embodiments, each of the one or more nucleic acid sequences encoding the RBD polypeptide comprise one or more mutations corresponding to K417, N439, E484, or N501 with reference to the full SARS-CoV-2 S protein (e.g., as set forth in NCBI Accession No. YP_009724390 (SEQ ID NO: 83)), or none of these mutations. In some embodiments, at least one of the one or more nucleic acid sequence encoding the RBD polypeptide comprises mutations corresponding to N439 and N501 with reference to the full SARS-CoV-2 S protein (e.g., as set forth in NCBI Accession No. YP_009724390 (SEQ ID NO: 83)). In some embodiments, at least one of the one or more nucleic acid sequence encoding the RBD polypeptide comprises mutations corresponding to K417, E484, and N501 with reference to the full SARS-CoV-2 S protein (e.g., as set forth in NCBI Accession No. YP_009724390 (SEQ ID NO: 83)). In some embodiments, at least one of the one or more nucleic acid sequence encoding the RBD polypeptide comprises mutations corresponding to L452 and T478 with reference to the full SARS-CoV-2 S protein (e.g. as set forth in NCBI Accession No. YP_009724390 (SEQ ID NO: 83)). In some embodiments, at least one of the one or more nucleic acid sequences encoding the RBD polypeptide comprise one or more mutations corresponding to L452R or T478K with reference to the full SARS-CoV-2 S protein (e.g. as set forth in NCBI Accession No. YP_009724390 (SEQ ID NO: 83)), or none of these mutations. In some embodiments, at least one of the one or more nucleic acid sequence encoding the RBD polypeptide comprises mutations corresponding to K417, N439, L452, T478, E484, or N501 with reference to the full SARS-CoV-2 S protein (e.g. as set forth in NCBI Accession No. YP_009724390 (SEQ ID NO: 83)). In some embodiments, at least one of the one or more nucleic acid sequences encoding the RBD polypeptide comprise one or more mutations corresponding to K417N, N439K, E484K, or N501Y with reference to the full SARS-CoV-2 S protein (e.g. as set forth in NCBI Accession No. YP_009724390 (SEQ ID NO: 83)), or none of these mutations. In some embodiments, at least one of the one or more nucleic acid sequences encoding the RBD polypeptide comprise one or more mutations corresponding to K417N, N439K, L452R, T478K, E484K, or N501Y with reference to the full SARS-CoV-2 S protein (e.g. as set forth in NCBI Accession No. YP_009724390 (SEQ ID NO: 83)), or none of these mutations. In some embodiments, at least one of the one or more nucleic acid sequence encoding the RBD polypeptide comprises one or more mutations corresponding to K417, L452, or T478 with reference to the full SARS-CoV-2 S protein (e.g. as set forth in NCBI Accession No. YP_009724390 (SEQ ID NO: 83)). In some embodiments, at least one of the one or more nucleic acid sequences encoding the RBD polypeptide comprise one or more mutations corresponding to K417N, L452R, or T478K with reference to the full SARS-CoV-2 S protein (e.g. as set forth in NCBI Accession No. YP_009724390 (SEQ ID NO: 83)), or none of these mutations. In some embodiments, at least one of the one or more nucleic acid sequences encoding the RBD polypeptide comprise one or more mutations corresponding to G339, S371, S373, S375, K417, N440, G446, S477, T478, E484, Q493, G496, Q498, N501, or Y505 with reference to the full SARS-CoV-2 S protein (e.g. as set forth in NCBI Accession No. YP_009724390 (SEQ ID NO: 83)), or none of these mutations. In some embodiments, at least one of the one or more nucleic acid sequences encoding the RBD polypeptide comprise one or more mutations corresponding to G339D, S371L, S373P, S375F, K417N, N440K, G446S, S477N, T478K, E484A, Q493R, G496S, Q498R, N501Y, or Y505H with reference to the full SARS-CoV-2 S protein (e.g. as set forth in NCBI Accession No. YP_009724390 (SEQ ID NO: 83)), or none of these mutations. In some embodiments, at least one of the one or more nucleic acid sequences encoding the RBD polypeptide comprise one or more mutations corresponding to G339, S371, S373, S375, T376, D405, R408, K417, N440, S477, T478, E484, Q493, Q498, N501, or Y505 with reference to the full SARS-CoV-2 S protein (e.g. as set forth in NCBI Accession No. YP_009724390 (SEQ ID NO: 83)), or none of these mutations. In some embodiments, at least one of the one or more nucleic acid sequence encoding the RBD polypeptide comprise one or more mutations corresponding to G339D, S371F, S373P, S375F, T376A, D405N, R408S, K417N, N440K, S477N, T478K, E484A, Q493R, Q498R, N501Y, or Y505H with reference to the full SARS-CoV-2 S protein (e.g. as set forth in NCBI Accession No. YP_009724390 (SEQ ID NO: 83)), or none of these mutations. In some embodiments, each of the one or more nucleic acid sequences encoding the RBD polypeptide comprise one or more mutations corresponding to C336, C361, C379, C391, C432, C480, C488, or C525 with reference to the full SARS-CoV-2 S protein (e.g. as set forth in NCBI Accession No. YP_009724390 (SEQ ID NO: 83)), or none of these mutations.

In some embodiments, the nucleic acid comprises three tandem nucleic acid sequences encoding an RBD polypeptide. In some embodiments, the three tandem nucleic acid sequences encoding the RBD polypeptide each comprise one or more mutations corresponding to K417, N439, L452, T478, E484, N501, G339, S371, S373, S375, T376, D405, R408, N440, G446, S447, Q493, G496, Q498, or Y505 with reference to the full SARS-CoV-2 S protein (e.g. as set forth in NCBI Accession No. YP_009724390 (SEQ ID NO: 83)), or none of these mutations. In some embodiments, the three tandem nucleic acid sequences encoding the RBD polypeptide each comprise one or more mutations corresponding to K417, N439, L452, T478, E484, or N501 with reference to the full SARS-CoV-2 S protein (e.g., as set forth in NCBI Accession No. YP_009724390 (SEQ ID NO: 83)), or none of these mutations. In some embodiments, at least one of the three tandem nucleic acid sequences encoding the RBD polypeptide comprises mutations corresponding to N439 and N501 with reference to the full SARS-CoV-2 S protein (e.g., as set forth in NCBI Accession No. YP_009724390 (SEQ ID NO: 83)). In some embodiments, at least one of the three tandem nucleic acid sequences encoding the RBD polypeptide comprises mutations corresponding to K417, E484, and N501 with reference to the full SARS-CoV-2 S protein (e.g. as set forth in NCBI Accession No. YP_009724390 (SEQ ID NO: 83)). In some embodiments, at least one of the three tandem nucleic acid sequences encoding the RBD polypeptide comprises mutations corresponding to L452 and T478 with reference to the full SARS-CoV-2 S protein (e.g. as set forth in NCBI Accession No. YP_009724390 (SEQ ID NO: 83)). In some embodiments, at least one of the three tandem nucleic acid sequences encoding the RBD polypeptide comprises mutations corresponding to K417, N439, L452, T478, and N501 with reference to the full SARS-CoV-2 S protein (e.g. as set forth in NCBI Accession No. YP_009724390 (SEQ ID NO: 83)). In some embodiments, each of the three tandem nucleic acid sequences encoding the RBD polypeptide comprises one or more mutations corresponding to K417N, N439K, L452R, T478K, E484K, or N501Y with reference to the full SARS-CoV-2 S protein (e.g., as set forth in NCBI Accession No. YP_009724390 (SEQ ID NO: 83)), or none of these mutations. In some embodiments, at least one of the three tandem nucleic acid sequences encoding the RBD polypeptide comprises mutations corresponding to K417, L452, or T478 with reference to the full SARS-CoV-2 S protein (e.g. as set forth in NCBI Accession No. YP_009724390 (SEQ ID NO: 83)). In some embodiments, each of the three tandem nucleic acid sequences encoding the RBD polypeptide comprises one or more mutations corresponding to K417N, L452R, or T478K with reference to the full SARS-CoV-2 S protein (e.g. as set forth in NCBI Accession No. YP_009724390 (SEQ ID NO: 83)), or none of these mutations. In some embodiments, at least one of the three tandem nucleic acid sequences encoding the RBD polypeptide comprises mutations corresponding to G339, S371, S373, S375, K417, N440, G446, S477, T478, E484, Q493, G496, Q498, N501, or Y505 with reference to the full SARS-CoV-2 S protein (e.g. as set forth in NCBI Accession No. YP_009724390 (SEQ ID NO: 83)). In some embodiments, each of the three tandem nucleic acid sequences encoding the RBD polypeptide comprises one or more mutations corresponding to G339D, S371L, S373P, S375F, K417N, N440K, G446S, S477N, T478K, E484A, Q493R, G496S, Q498R, N501Y, or Y505H with reference to the full SARS-CoV-2 S protein (e.g. as set forth in NCBI Accession No. YP_009724390 (SEQ ID NO: 83)), or none of these mutations. In some embodiments, at least one of the three tandem nucleic acid sequences encoding the RBD polypeptide comprises mutations corresponding to G339, S371, S373, S375, T376, D405, R408, K417, N440, S477, T478, E484, Q493, Q498, N501, or Y505 with reference to the full SARS-CoV-2 S protein (e.g. as set forth in NCBI Accession No. YP_009724390 (SEQ ID NO: 83)). In some embodiments, each of the three tandem nucleic acid sequences encoding the RBD polypeptide comprises one or more mutations corresponding to G339D, S371F, S373P, S375F, T376A, D405N, R408S, K417N, N440K, S477N, T478K, E484A, Q493R, Q498R, N501Y, or Y505H with reference to the full SARS-CoV-2 S protein (e.g. as set forth in NCBI Accession No. YP_009724390 (SEQ ID NO: 83)), or none of these mutations. In some embodiments, each of the one or more nucleic acid sequences encoding the RBD polypeptide comprise one or more mutations corresponding to C336, C361, C379, C391, C432, C480, C488, or C525 with reference to the full SARS-CoV-2 S protein (e.g. as set forth in NCBI Accession No. YP_009724390 (SEQ ID NO: 83)), or none of these mutations.

In some embodiments, the three tandem nucleic acid sequences encoding the RBD polypeptide comprise 1) one nucleic acid encoding for the RBD polypeptide from the wild-type SARS-CoV-2 strain (Wuhan-hu-1), 2) one nucleic acid with mutations corresponding to N439 and N501, and 3) one nucleic acid with mutations corresponding to K417, E484, and N501. In some embodiments, the three tandem nucleic acid sequences encoding the RBD polypeptide comprise 1) one nucleic acid encoding for the RBD polypeptide from the wild-type SARS-CoV-2 strain (Wuhan-hu-1), 2) one nucleic acid with mutations corresponding to K417, N439, L452, T478, and N501, and 3) one nucleic acid with mutations corresponding to K417, E484, and N501. In some embodiments, the three tandem nucleic acid sequences encoding the RBD polypeptide comprise 1) one nucleic acid with mutations corresponding to G339, S371, S373, S375, K417, N440, G446, S477, T478, E484, Q493, G496, Q498, N501, or Y505, 2) one nucleic acid with mutations corresponding to G339, S371, S373, S375, T376, D405, R408, K417, N440, S477, T478, E484, Q493, Q498, N501, or Y505, and 3) one nucleic acid with mutations corresponding to K417, L452, or T478. In some embodiments, the three tandem nucleic acid sequences encoding the RBD polypeptide comprise 1) one nucleic acid with mutations corresponding to G339, S371, S373, S375, K417, N440, G446, S477, T478, E484, Q493, G496, Q498, N501, or Y505, 2) one nucleic acid with mutations corresponding to G339, S371, S373, S375, T376, D405, R408, K417, N440, S477, T478, E484, Q493, Q498, N501, or Y505, and 3) one nucleic acid with a mutation corresponding to N501. In some embodiments, each of the one or more nucleic acid sequences encoding the RBD polypeptide comprise one or more mutations corresponding to C336, C361, C379, C391, C432, C480, C488, or C525 with reference to the full SARS-CoV-2 S protein (e.g. as set forth in NCBI Accession No. YP_009724390 (SEQ ID NO: 83)), or none of these mutations.

In some embodiments, the nucleic acid comprises the one or more nucleic acid sequences encoding the RBD polypeptide and the nucleic acid sequence encoding an NP polypeptide. In some embodiments, the nucleic acid further comprises the nucleic acid sequence encoding an IgE leader polypeptide. In some embodiments, the nucleic acid consists essentially of the one or more nucleic acid sequences encoding the RBD polypeptide, the nucleic acid sequence encoding the NP polypeptide, and the nucleic acid sequence encoding the IgE leader polypeptide. In some embodiments, the nucleic acid encodes for a polypeptide having at least 90%, 91%, 92%, 93%, 94%, 95%, 96%, 97%, 98%, 99%, or 100% sequence identity to SEQ ID NO: 79, 87, 93, or 95. In some embodiments, the variance in sequence identity is not in the RBD polypeptide at K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein. In some embodiments, the variance in sequence identity is not in the RBD polypeptide at K417, N439, L452, T478, E484, or N501, or any combination thereof, with reference to the full SARS-CoV-2 S protein. In some embodiments, the nucleic acid shares or comprises at least 90%, 91%, 92%, 93%, 94%, 95%, 96%, 97%, 98%, 99%, or 100% sequence identity to SEQ ID NO: 77, 78, 86, 92, or 94. In some embodiments, the variance in sequence identity is not in the RBD polypeptide at sequences encoding for K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein. In some embodiments, the variance in sequence identity is not in the RBD polypeptide at sequences encoding for K417, N439, L452, T478, E484, or N501, or any combination thereof, with reference to the full SARS-CoV-2 S protein. In some embodiments, the variance in sequence identity is in the RBD polypeptide at or at sequences encoding for C336, C361, C379, C391, C432, C480, C488, or C525, or any combination thereof, with reference to the full SARS-CoV-2 S protein.

In some embodiments, the nucleic acid further comprises the nucleic acid sequence encoding the autocatalytic polypeptide cleavage site, optionally wherein the autocatalytic polypeptide cleavage site is a P2A autocatalytic polypeptide cleavage site. In some embodiments, the nucleic acid consists essentially of the one or more nucleic acid sequences encoding the RBD polypeptide, the nucleic acid sequence encoding the NP polypeptide, the nucleic acid sequence encoding the IgE leader polypeptide, and the nucleic acid sequence encoding the autocatalytic polypeptide cleavage site. In some embodiments, the nucleic acid encodes for a polypeptide having at least 90%, 91%, 92%, 93%, 94%, 95%, 96%, 97%, 98%, 99%, or 100% sequence identity to SEQ ID NO: 82 or 89. In some embodiments, the variance in sequence identity is not in the RBD polypeptide at K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein. In some embodiments, the variance in sequence identity is not in the RBD polypeptide at K417, N439, L452, T478, E484, or N501, or any combination thereof, with reference to the full SARS-CoV-2 S protein. In some embodiments, the nucleic acid shares or comprises at least 90%, 91%, 92%, 93%, 94%, 95%, 96%, 97%, 98%, 99%, or 100% sequence identity to SEQ ID NO: 80, 81, or 88. In some embodiments, the variance in sequence identity is not in the RBD polypeptide at sequences encoding for K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein. In some embodiments, the variance in sequence identity is not in the RBD polypeptide at sequences encoding for K417, N439, L452, T478, E484, or N501, or any combination thereof, with reference to the full SARS-CoV-2 S protein. In some embodiments, the variance in sequence identity is in the RBD polypeptide at or at sequences encoding for C336, C361, C379, C391, C432, C480, C488, or C525, or any combination thereof, with reference to the full SARS-CoV-2 S protein.

In some embodiments, the nucleic acid further comprises the nucleic acid sequence encoding the M polypeptide. In some embodiments, the nucleic acid consists essentially of the one or more nucleic acid sequences encoding the RBD polypeptide, the nucleic acid sequence encoding the NP polypeptide, the nucleic acid sequence encoding the IgE leader polypeptide, the nucleic acid sequence encoding the autocatalytic polypeptide cleavage site, and the nucleic acid sequence encoding the M polypeptide. In some embodiments, the nucleic acid encodes for a polypeptide having at least 90%, 91%, 92%, 93%, 94%, 95%, 96%, 97%, 98%, 99%, or 100% sequence identity to SEQ ID NO: 85. In some embodiments, the variance in sequence identity is not in the RBD polypeptide at K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein. In some embodiments, the variance in sequence identity is not at K417, N439, L452, T478, E484, or N501, or any combination thereof. In some embodiments, the nucleic acid shares or comprises at least 90%, 91%, 92%, 93%, 94%, 95%, 96%, 97%, 98%, 99%, or 100% sequence identity to SEQ ID NO: 84, with reference to the full SARS-CoV-2 S protein. In some embodiments, the variance in sequence identity is not in the RBD polypeptide at sequences encoding for K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein. In some embodiments, the variance in sequence identity is not in the RBD polypeptide at sequences encoding for K417, N439, L452, T478, E484, or N501, or any combination thereof, with reference to the full SARS-CoV-2 S protein. In some embodiments, the variance in sequence identity is in the RBD polypeptide at or at sequences encoding for C336, C361, C379, C391, C432, C480, C488, or C525, or any combination thereof, with reference to the full SARS-CoV-2 S protein.

In some embodiments, the nucleic acid further comprises a nucleic acid sequence encoding a CC40.8 epitope. In some embodiments, the nucleic acid comprises, consists of, or consists essentially of the one or more nucleic acid sequences encoding the RBD polypeptide, the nucleic acid sequence encoding the NP polypeptide, the nucleic acid sequence encoding the IgE leader polypeptide, and the nucleic acid sequence encoding the CC40.8 epitope. In some embodiments, the nucleic acid encodes for a polypeptide having at least 90%, 91%, 92%, 93%, 94%, 95%, 96%, 97%, 98%, 99%, or 100% sequence identity to SEQ ID NO: 97 or 99. In some embodiments, the variance in sequence identity is not in the RBD polypeptide at K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein. In some embodiments, the variance in sequence identity is not in the RBD polypeptide at K417, N439, L452, T478, E484, or N501, or any combination thereof, with reference to the full SARS-CoV-2 S protein. In some embodiments, the nucleic acid shares or comprises at least 90%, 91%, 92%, 93%, 94%, 95%, 96%, 97%, 98%, 99%, or 100% sequence identity to SEQ ID NO: 96 or 98. In some embodiments, the variance in sequence identity is not in the RBD polypeptide at sequences encoding for K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein. In some embodiments, the variance in sequence identity is not at sequences encoding for K417, N439, L452, T478, E484, or N501, or any combination thereof, with reference to the full SARS-CoV-2 S protein. In some embodiments, the variance in sequence identity is in the RBD polypeptide at or at sequences encoding for C336, C361, C379, C391, C432, C480, C488, or C525, or any combination thereof, with reference to the full SARS-CoV-2 S protein.

In some embodiments, the nucleic acids prepared as plasmids, viruses, bacteriophages, cosmids, fosmids, phagemids, bacterial artificial chromosomes (BAC), yeast artificial chromosomes (YAC), or human artificial chromosomes (HAC). In some embodiments, the nucleic acids are circular or linear. In some embodiments, the nucleic acids are produced in a biological system, including but not limited to mammalian cells, human cells, bacteria cells, E. coli, yeast, S. cerevisiae, or other appropriate biological system. In some embodiments, the nucleic acids are found in a cassette that comprises elements needed to transcribe and translate the nucleic acids in a biological system.

Any one of the nucleic acids disclosed herein may be used in a medicament or for the manufacture of a medicament. In some embodiments, the medicament is used for the prevention, treatment, or inhibition of SARS-CoV-2 or other coronavirus in a subject. In some embodiments, the subject is a human.

Also disclosed herein are polypeptides that can be used as immunogenic compositions or part of immunogenic product combinations, for example, to generate an immune response against SARS-CoV-2 or other coronavirus, and/or generate neutralizing antibodies against SARS-CoV-2 or other coronavirus in a subject.

In some embodiments, the polypeptide comprises at least one SARS-CoV-2 polypeptide sequence and at least one P2A autocatalytic polypeptide cleavage site. In some embodiments, the at least one SARS-CoV-2 polypeptide sequence comprises an RBD polypeptide sequence and an NP polypeptide sequence. In some embodiments, the polypeptide shares or comprises at least 90%, 91%, 92%, 93%, 94%, 95%, 96%, 97%, 98%, 99%, or 100% homology or sequence identity to SEQ ID NO: 25. In some embodiments, the at least one SARS-CoV-2 polypeptide sequence comprises an RBD polypeptide sequence, an M polypeptide sequence, and an NP polypeptide sequence. In some embodiments, the polypeptide shares or comprises at least 90%, 91%, 92%, 93%, 94%, 95%, 96%, 97%, 98%, 99%, or 100% homology or sequence identity to SEQ ID NO: 26 or 27. In some embodiments, the RBD polypeptide is an RBD tandem repeat single chain dimer polypeptide. In some embodiments, the RBD tandem repeat single chain dimer polypeptide comprises a K417N, N439K, L452R, T478K, E484K, or N501Y mutation with reference to the full SARS-CoV-2 S protein (e.g., as set forth in NCBI Accession No. YP_009724390), or any combination thereof, or none of these mutations. In some embodiments, the RBD tandem repeat single chain dimer polypeptide comprises a C336, C361, C379, C391, C432, C480, C488, or C525 mutation, or any combination thereof, or none of these mutations, with reference to the full SARS-CoV-2 S protein (e.g., as set forth in NCBI Accession No. YP_009724390)). In some embodiments, the RBD tandem repeat single chain dimer polypeptide shares or comprises at least 90%, 91%, 92%, 93%, 94%, 95%, 96%, 97%, 98%, 99%, or 100% homology or sequence identity to any one of SEQ ID NOs: 46, or 52-55. In some embodiments, the polypeptide shares or comprises at least 90%, 91%, 92%, 93%, 94%, 95%, 96%, 97%, 98%, 99%, or 100% homology or sequence identity to SEQ ID NO: 41. In some embodiments, the RBD polypeptide comprises three tandem copies of RBD (or RBDv2). In some embodiments, the three tandem copies of RBD each comprise a K417N, N439K, L452R, T478K, E484K, or N501Y mutation with reference to the full SARS-CoV-2 S protein (e.g., as set forth in NCBI Accession No. YP_009724390), or any combination thereof, or none of these mutations. In some embodiments, the three tandem copies of RBD each comprise a C336, C361, C379, C391, C432, C480, C488, or C525 mutation, or any combination thereof, or none of these mutations, with reference to the full SARS-CoV-2 S protein (e.g., as set forth in NCBI Accession No. YP_009724390)). In any of the embodiments disclosed herein, the RBD polypeptide, NP polypeptide, M polypeptide, or S polypeptide may be derived from the wild-type SARS-CoV-2 strain (Wuhan-hu-1) or a SARS-CoV-2 variant, optionally the Alpha, Beta, Gamma, Delta, Theta, Iota, Kappa, Lambda, Mu, or Omicron (including the BA.1 or BA.2 sub-lineage) variant. In some embodiments, the RBD polypeptide, NP polypeptide, M polypeptide, or S polypeptide comprises one or more mutations found in a SARS-CoV-2 variant, optionally the Alpha, Beta, Gamma, Delta, Theta, Iota, Kappa, Lambda, Mu, or Omicron (including the BA.1 or BA.2 sub-lineage) variant, relative to the wild-type SARS-CoV-2 strain (Wuhan-hu-1). In some embodiments, the RBD polypeptide comprises one or more mutations found in a SARS-CoV-2 variant, optionally the Alpha, Beta, Gamma, Delta, Theta, Iota, Kappa, Lambda, Mu, or Omicron (including the BA.1 or BA.2 sub-lineage) variant, relative to the wild-type SARS-CoV-2 strain (Wuhan-hu-1).

As applied to any of the polypeptides disclosed herein, in some embodiments, the polypeptide further comprises an N-terminal IgE leader polypeptide sequence. In some embodiments, the N-terminal IgE leader polypeptide sequence shares or comprises at least 90%, 91%, 92%, 93%, 94%, 95%, 96%, 97%, 98%, 99%, or 100% homology or sequence identity to SEQ ID NO: 44. In some embodiments, the RBD polypeptide is an RBD tandem repeat single chain dimer polypeptide. In some embodiments, the RBD tandem repeat single chain dimer polypeptide comprises a K417N, N439K, L452R, T478K, E484K, or N501Y mutation with reference to the full SARS-CoV-2 S protein (e.g., as set forth in NCBI Accession No. YP_009724390), or any combination thereof, or none of these mutations. In some embodiments, the RBD tandem repeat single chain dimer polypeptide comprises a C336, C361, C379, C391, C432, C480, C488, or C525 mutation, or any combination thereof, or none of these mutations, with reference to the full SARS-CoV-2 S protein (e.g., as set forth in NCBI Accession No. YP_009724390)). In some embodiments, the RBD tandem repeat single chain dimer polypeptide shares or comprises at least 90%, 91%, 92%, 93%, 94%, 95%, 96%, 97%, 98%, 99%, or 100% homology or sequence identity to any one of SEQ ID NO: 46, or 52-55. In some embodiments, the polypeptide shares or comprises at least 90%, 91%, 92%, 93%, 94%, 95%, 96%, 97%, 98%, 99%, or 100% homology or sequence identity to any one of SEQ ID NO: 42, 64-67, or 69. In some embodiments, the RBD polypeptide comprises three tandem copies of RBD (or RBDv2). In some embodiments, the three tandem copies of RBD each comprises a K417N, N439K, L452R, T478K, E484K, or N501Y mutation with reference to the full SARS-CoV-2 S protein (e.g., as set forth in NCBI Accession No. YP_009724390), or any combination thereof, or none of these mutations. In some embodiments, the three tandem copies of RBD each comprise a C336, C361, C379, C391, C432, C480, C488, or C525 mutation, or any combination thereof, or none of these mutations, with reference to the full SARS-CoV-2 S protein (e.g., as set forth in NCBI Accession No. YP_009724390)). In some embodiments, the polypeptide shares or comprises at least 90%, 91%, 92%, 93%, 94%, 95%, 96%, 97%, 98%, 99%, or 100% homology or sequence identity to SEQ ID NO: 68.

In some embodiments, the at least one SARS-CoV-2 polypeptide sequence comprises an RBD polypeptide sequence and an M polypeptide sequence. In some embodiments, the polypeptide shares or comprises at least 90%, 91%, 92%, 93%, 94%, 95%, 96%, 97%, 98%, 99%, or 100% homology or sequence identity to SEQ ID NO: 28. In some embodiments, the variance in sequence identity is in the RBD polypeptide at C336, C361, C379, C391, C432, C480, C488, or C525, or any combination thereof, with reference to the full SARS-CoV-2 S protein.

In some embodiments, the at least one SARS-CoV-2 polypeptide comprises a spike (S) polypeptide. In some embodiments, the at least one SARS-CoV-2 polypeptide further comprises an NP polypeptide. In some embodiments, the S polypeptide comprises mutations to facilitate improved expression, solubility, and/or immunogenicity. In some embodiments, the S polypeptide comprises a K968P or V987P mutation with reference to the full SARS-CoV-2 S protein (e.g., as set forth in NCBI Accession No. YP_009724390), or both. In some embodiments, the S polypeptide shares or comprises at least 90%, 91%, 92%, 93%, 94%, 95%, 96%, 97%, 98%, 99%, or 100% homology or sequence identity to SEQ ID NO: 56. In some embodiments, the polypeptide further comprises an N-terminal IgE leader polypeptide sequence. In some embodiments, the N-terminal IgE leader polypeptide sequence shares or comprises at least 90%, 91%, 92%, 93%, 94%, 95%, 96%, 97%, 98%, 99%, or 100% homology or sequence identity to SEQ ID NO: 44. In some embodiments, the polypeptide shares or comprises at least 90%, 91%, 92%, 93%, 94%, 95%, 96%, 97%, 98%, 99%, or 100% homology or sequence identity to SEQ ID NO: 70. In some embodiments, the variance in sequence identity is in the RBD polypeptide at C336, C361, C379, C391, C432, C480, C488, or C525, or any combination thereof, with reference to the full SARS-CoV-2 S protein.

In some embodiments, the polypeptide comprises at least one SARS-CoV-2 polypeptide sharing or comprising at least 90%, 91%, 92%, 93%, 94%, 95%, 96%, 97%, 98%, 99%, or 100% homology or sequence identity to any one or more of SEQ ID NO: 29-31, 34-36, 74, or 76. In some embodiments, the variance in sequence identity is in the RBD polypeptide at C336, C361, C379, C391, C432, C480, C488, or C525, or any combination thereof, with reference to the full SARS-CoV-2 S protein.

In some embodiments, the polypeptide comprises at least one SARS-CoV-2 polypeptide and at least one HDAg polypeptide. In some embodiments, the polypeptide shares or comprises at least 90%, 91%, 92%, 93%, 94%, 95%, 96%, 97%, 98%, 99%, or 100% homology or sequence identity to SEQ ID NO: 32. In some embodiments, the polypeptide further comprises at least one P2A autocatalytic polypeptide cleavage site. In some embodiments, the polypeptide shares or comprises at least 90%, 91%, 92%, 93%, 94%, 95%, 96%, 97%, 98%, 99%, or 100% homology or sequence identity to SEQ ID NO: 33. In some embodiments, the variance in sequence identity is in the RBD polypeptide at C336, C361, C379, C391, C432, C480, C488, or C525, or any combination thereof, with reference to the full SARS-CoV-2 S protein.

In some embodiments of any one of the polypeptides disclosed herein, the polypeptide further comprises an N-terminal IgE leader polypeptide sequence. In some embodiments, the N-terminal IgE leader polypeptide sequence shares or comprises at least 90%, 91%, 92%, 93%, 94%, 95%, 96%, 97%, 98%, 99%, or 100% homology or sequence identity to SEQ ID NO: 44. In some embodiments, the polypeptide shares or comprises at least 90%, 91%, 92%, 93%, 94%, 95%, 96%, 97%, 98%, 99%, or 100% homology or sequence identity to SEQ ID NO: 42.

In any of the polypeptides disclosed herein, the polypeptide may comprise any one or more of the SARS-CoV-2 polypeptides disclosed herein or otherwise conventionally known in the art. In some embodiments, the one or more SARS-CoV-2 polypeptides comprise an RBD polypeptide. In some embodiments, the RBD polypeptide is from the SARS-CoV-2 virus or a variant thereof. In some embodiments, the RBD polypeptide comprises a K417N, N439K, L452R, T478K, E484K, or N501Y mutation with reference to the full SARS-CoV-2 S protein (e.g., as set forth in NCBI Accession No. YP_009724390), or any combination thereof, or none of these mutations. In some embodiments, a nucleic acid encoding for the RBD polypeptide is represented by SEQ ID NO: 10 or 22. In some embodiments, the RBD polypeptide is represented by SEQ ID NO: 34. In some embodiments, the RBD polypeptide is an RBD tandem repeat single chain dimer polypeptide. In some embodiments, the RBD tandem repeat single chain dimer polypeptide comprises a K417N, N439K, L452R, T478K, E484K, or N501Y mutation with reference to the full SARS-CoV-2 S protein (e.g., as set forth in NCBI Accession No. YP_009724390), or any combination thereof, or none of these mutations. In some embodiments, a nucleic acid encoding the RBD polypeptide is represented by any one or more of SEQ ID NOs: 45, or 47-50. In some embodiments, the RBD polypeptide is represented by any one or more of SEQ ID NO: 46, or 52-55. In some embodiments, a nucleic acid encoding for an M polypeptide is represented by SEQ ID NOs: 11 or 23. In some embodiments, the M polypeptide is represented by SEQ ID NO: 35. In some embodiments, a nucleic acid encoding for an NP polypeptide is represented by SEQ ID NOs: 12 or 24. In some embodiments, the NP polypeptide is represented by SEQ ID NO: 36.

In some embodiments, the polypeptide comprises at least one SARS-CoV-2 polypeptide, wherein the SARS-CoV-2 polypeptide comprises: i) one or more SARS-CoV-2 spike receptor binding domain (RBD) polypeptide sequences; ii) a SARS-CoV-2 nucleocapsid protein (NP) polypeptide sequence; iii) a SARS-CoV-2 membrane protein (M) polypeptide sequence; iv) a hepatitis D antigen (HDAg) polypeptide sequence; v) an autocatalytic polypeptide cleavage site sequence; vi) an IgE leader polypeptide sequence; vii) a SARS-CoV-2 spike (S) polypeptide sequence; or viii) a CC40.8 epitope; or any combination thereof. In some embodiments, one or more of the RBD polypeptide, NP polypeptide, M polypeptide, or S polypeptide is derived from the wild-type SARS-CoV-2 strain (Wuhan-hu-1) or a SARS-CoV-2 variant, optionally the Alpha, Beta, Gamma, Delta, Theta, Iota, Kappa, Lambda, Mu, or Omicron (including the BA.1 or BA.2 sub-lineage) variant. In some embodiments, one or more of the RBD polypeptide, NP polypeptide, M polypeptide, or S polypeptide comprises one or more mutations found in a SARS-CoV-2 variant, optionally the Alpha, Beta, Gamma, Delta, Theta, Iota, Kappa, Lambda, Mu, or Omicron (including the BA.1 or BA.2 sub-lineage) variant, relative to the wild-type SARS-CoV-2 strain (Wuhan-hu-1). In some embodiments, each of the one or more RBD polypeptide comprises one or more mutations found in a SARS-CoV-2 variant, optionally the Alpha, Beta, Gamma, Delta, Theta, Iota, Kappa, Lambda, Mu, or Omicron (including the BA.1 or BA.2 sub-lineage) variant, relative to the wild-type SARS-CoV-2 strain (Wuhan-hu-1). In some embodiments, each of the one or more RBD polypeptide sequences comprise one or more mutations at K417, N439, L452, T478, E484, N501, G339, S371, S373, S375, T376, D405, R408, N440, G446, S447, Q493, G496, Q498, or Y505 with reference to the full SARS-CoV-2 S protein (e.g. as set forth in NCBI Accession No. YP_009724390 (SEQ ID NO: 83)), or none of these mutations. In some embodiments, each of the one or more RBD polypeptide sequences comprise one or more mutations at K417, N439, L452, T478, E484, or N501 with reference to the full SARS-CoV-2 S protein (e.g., as set forth in NCBI Accession No. YP_009724390 (SEQ ID NO: 83)), or none of these mutations. In some embodiments, at least one of the one or more RBD polypeptide sequences comprises mutations at N439 and N501 with reference to the full SARS-CoV-2 S protein (e.g., as set forth in NCBI Accession No. YP_009724390 (SEQ ID NO: 83)). In some embodiments, at least one of the one or more RBD polypeptide sequences comprises mutations at K417, E484, and N501 with reference to the full SARS-CoV-2 S protein (e.g., as set forth in NCBI Accession No. YP_009724390 (SEQ ID NO: 83)). In some embodiments, at least one of the one or more RBD polypeptide sequences comprises mutations corresponding to L452 and T478 with reference to the full SARS-CoV-2 S protein (e.g. as set forth in NCBI Accession No. YP_009724390 (SEQ ID NO: 83)). In some embodiments, at least one of the one or more RBD polypeptide sequences comprise one or more mutations corresponding to L452R or T478K with reference to the full SARS-CoV-2 S protein (e.g. as set forth in NCBI Accession No. YP_009724390 (SEQ ID NO: 83)). In some embodiments, at least one of the one or more RBD polypeptide sequences comprise one or more mutations corresponding to K417, N439, L452, T478, E484, or N501 with reference to the full SARS-CoV-2 S protein (e.g. as set forth in NCBI Accession No. YP_009724390 (SEQ ID NO: 83)). In some embodiments, each of the one or more RBD polypeptide sequences comprise one or more mutations from K417N, N439K, E484K, or N501Y with reference to the full SARS-CoV-2 S protein (e.g., as set forth in NCBI Accession No. YP_009724390 (SEQ ID NO: 83)), or none of these mutations. In some embodiments, at least one of the one or more RBD polypeptide sequences comprise one or more mutations corresponding to K417N, N439K, L452R, T478K, E484K, or N501Y with reference to the full SARS-CoV-2 S protein (e.g. as set forth in NCBI Accession No. YP_009724390 (SEQ ID NO: 83)). In some embodiments, at least one of the one or more RBD polypeptide sequences comprises one or more mutations corresponding to K417, L452, or T478 with reference to the full SARS-CoV-2 S protein (e.g. as set forth in NCBI Accession No. YP_009724390 (SEQ ID NO: 83)). In some embodiments, at least one of the one or more RBD polypeptide sequences comprise one or more mutations corresponding to K417N, L452R, or T478K with reference to the full SARS-CoV-2 S protein (e.g. as set forth in NCBI Accession No. YP_009724390 (SEQ ID NO: 83)), or none of these mutations. In some embodiments, at least one of the one or more RBD polypeptide sequences comprise one or more mutations corresponding to G339, S371, S373, S375, K417, N440, G446, S477, T478, E484, Q493, G496, Q498, N501, or Y505 with reference to the full SARS-CoV-2 S protein (e.g. as set forth in NCBI Accession No. YP_009724390 (SEQ ID NO: 83)), or none of these mutations. In some embodiments, at least one of the one or more RBD polypeptide sequences comprise one or more mutations corresponding to G339D, S371L, S373P, S375F, K417N, N440K, G446S, S477N, T478K, E484A, Q493R, G496S, Q498R, N501Y, or Y505H with reference to the full SARS-CoV-2 S protein (e.g. as set forth in NCBI Accession No. YP_009724390 (SEQ ID NO: 83)), or none of these mutations. In some embodiments, at least one of the one or more RBD polypeptide sequences comprise one or more mutations corresponding to G339, S371, S373, S375, T376, D405, R408, K417, N440, S477, T478, E484, Q493, Q498, N501, or Y505 with reference to the full SARS-CoV-2 S protein (e.g. as set forth in NCBI Accession No. YP_009724390 (SEQ ID NO: 83)), or none of these mutations. In some embodiments, at least one of the one or more RBD polypeptide sequences comprise one or more mutations corresponding to G339D, S371F, S373P, S375F, T376A, D405N, R408S, K417N, N440K, S477N, T478K, E484A, Q493R, Q498R, N501Y, or Y505H with reference to the full SARS-CoV-2 S protein (e.g. as set forth in NCBI Accession No. YP_009724390 (SEQ ID NO: 83)), or none of these mutations. In some embodiments, each of the one or more RBD polypeptide sequences comprise one or more mutations corresponding to C336, C361, C379, C391, C432, C480, C488, or C525 with reference to the full SARS-CoV-2 S protein (e.g. as set forth in NCBI Accession No. YP_009724390 (SEQ ID NO: 83)), or none of these mutations.

In some embodiments, the polypeptide comprises three tandem RBD polypeptide sequences. In some embodiments, the three tandem RBD polypeptide sequences each comprise one or more mutations at K417, N439, L452, T478, E484, N501, G339, S371, S373, S375, T376, D405, R408, N440, G446, S447, Q493, G496, Q498, or Y505 with reference to the full SARS-CoV-2 S protein (e.g. as set forth in NCBI Accession No. YP_009724390 (SEQ ID NO: 83)), or none of these mutations. In some embodiments, the three tandem RBD polypeptide sequences each comprise one or more mutations at K417, N439, L452, T478, E484, or N501 with reference to the full SARS-CoV-2 S protein (e.g. as set forth in NCBI Accession No. YP_009724390 (SEQ ID NO: 83)), or none of these mutations. In some embodiments, at least one of the three tandem RBD polypeptide sequences comprises mutations at N439 and N501 with reference to the full SARS-CoV-2 S protein (e.g., as set forth in NCBI Accession No. YP_009724390 (SEQ ID NO: 83)). In some embodiments, at least one of the three tandem RBD polypeptide sequences comprise mutations at K417, E484, and N501 with reference to the full SARS-CoV-2 S protein (e.g. as set forth in NCBI Accession No. YP_009724390 (SEQ ID NO: 83)). In some embodiments, at least one of the three tandem RBD polypeptide sequences comprises mutations corresponding to L452 and T478 with reference to the full SARS-CoV-2 S protein (e.g. as set forth in NCBI Accession No. YP_009724390 (SEQ ID NO: 83)). In some embodiments, at least one of the three tandem RBD polypeptide sequences comprises mutations corresponding to K417, N439, L452, T478, and N501 with reference to the full SARS-CoV-2 S protein (e.g. as set forth in NCBI Accession No. YP_009724390 (SEQ ID NO: 83)). In some embodiments, each of the three tandem RBD polypeptide sequences comprise one or more mutations from K417N, N439K, L452R, T478K, E484K, or N501Y with reference to the full SARS-CoV-2 S protein (e.g., as set forth in NCBI Accession No. YP_009724390 (SEQ ID NO: 83)), or none of these mutations. In some embodiments, at least one of the three tandem RBD polypeptide sequences comprises mutations corresponding to K417, L452, or T478 with reference to the full SARS-CoV-2 S protein (e.g. as set forth in NCBI Accession No. YP_009724390 (SEQ ID NO: 83)). In some embodiments, each of the three tandem RBD polypeptide sequences comprises one or more mutations from K417N, L452R, or T478K with reference to the full SARS-CoV-2 S protein (e.g. as set forth in NCBI Accession No. YP_009724390 (SEQ ID NO: 83)), or none of these mutations. In some embodiments, at least one of the three tandem RBD polypeptide sequences comprises mutations corresponding to G339, S371, S373, S375, K417, N440, G446, S477, T478, E484, Q493, G496, Q498, N501, or Y505 with reference to the full SARS-CoV-2 S protein (e.g. as set forth in NCBI Accession No. YP_009724390 (SEQ ID NO: 83)). In some embodiments, each of the three tandem RBD polypeptide sequences comprises one or more mutations from G339D, S371L, S373P, S375F, K417N, N440K, G446S, S477N, T478K, E484A, Q493R, G496S, Q498R, N501Y, or Y505H with reference to the full SARS-CoV-2 S protein (e.g. as set forth in NCBI Accession No. YP_009724390 (SEQ ID NO: 83)), or none of these mutations. In some embodiments, at least one of the three tandem RBD polypeptide sequences comprises mutations corresponding to G339, S371, S373, S375, T376, D405, R408, K417, N440, S477, T478, E484, Q493, Q498, N501, or Y505with reference to the full SARS-CoV-2 S protein (e.g. as set forth in NCBI Accession No. YP_009724390 (SEQ ID NO: 83)). In some embodiments, each of the three tandem RBD polypeptide sequences comprises one or more mutations from G339D, S371F, S373P, S375F, T376A, D405N, R408S, K417N, N440K, S477N, T478K, E484A, Q493R, Q498R, N501Y, or Y505H with reference to the full SARS-CoV-2 S protein (e.g. as set forth in NCBI Accession No. YP_009724390 (SEQ ID NO: 83)), or none of these mutations. In some embodiments, each of the one or more RBD polypeptide sequences comprise one or more mutations corresponding to C336, C361, C379, C391, C432, C480, C488, or C525 with reference to the full SARS-CoV-2 S protein (e.g. as set forth in NCBI Accession No. YP_009724390 (SEQ ID NO: 83)), or none of these mutations.

In some embodiments, the three tandem RBD polypeptide sequences comprise 1) one RBD polypeptide corresponding to the RBD polypeptide from the wild-type SARS-CoV-2 strain (Wuhan-hu-1), 2) one RBD polypeptide with mutations at N439 and N501, and 3) one RBD polypeptide with mutations at K417, E484, and N501. In some embodiments, the three tandem RBD polypeptide sequences comprise 1) one RBD polypeptide corresponding to the RBD polypeptide from the wild-type SARS-CoV-2 strain (Wuhan-hu-1), 2) one RBD polypeptide with mutations at K417, N439, L452, T478, and N501, and 3) one RBD polypeptide with mutations at K417, E484, and N501. In some embodiments, the three tandem RBD polypeptide sequences comprise 1) one RBD polypeptide with mutations at G339, S371, S373, S375, K417, N440, G446, S477, T478, E484, Q493, G496, Q498, N501, or Y505, 2) one RBD polypeptide with mutations at G339, S371, S373, S375, T376, D405, R408, K417, N440, S477, T478, E484, Q493, Q498, N501, or Y505, and 3) one RBD polypeptide with mutations at K417, L452, or T478. In some embodiments, the three tandem RBD polypeptide sequences comprise 1) one RBD polypeptide with mutations at G339, S371, S373, S375, K417, N440, G446, S477, T478, E484, Q493, G496, Q498, N501, or Y505, 2) one RBD polypeptide with mutations at G339, S371, S373, S375, T376, D405, R408, K417, N440, S477, T478, E484, Q493, Q498, N501, or Y505, and 3) one RBD polypeptide with a mutation at N501. In some embodiments, each of the one or more RBD polypeptide sequences comprise one or more mutations corresponding to C336, C361, C379, C391, C432, C480, C488, or C525 with reference to the full SARS-CoV-2 S protein (e.g. as set forth in NCBI Accession No. YP_009724390 (SEQ ID NO: 83)), or none of these mutations.

In some embodiments, the polypeptide comprises the one or more RBD polypeptide sequences and the NP polypeptide sequence. In some embodiments, the polypeptide further comprises the IgE leader polypeptide sequence. In some embodiments, the polypeptide consists essentially of the one or more RBD polypeptide sequences, the NP polypeptide sequence, and the IgE leader polypeptide sequence. In some embodiments, the polypeptide comprises a sequence having at least 90%, 91%, 92%, 93%, 94%, 95%, 96%, 97%, 98%, 99%, or 100% sequence identity to SEQ ID NO: 79, 87, 93, or 95. In some embodiments, the variance in sequence identity is not in the RBD polypeptide at K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein. In some embodiments, the variance in sequence identity is not in the RBD polypeptide at K417, N439, L452, T478, E484, or N501, or any combination thereof, with reference to the full SARS-CoV-2 S protein. In some embodiments, the polypeptide is encoded by a nucleic acid comprising at least 90%, 91%, 92%, 93%, 94%, 95%, 96%, 97%, 98%, 99%, or 100% sequence identity to SEQ ID NO: 77, 78, 86, 92, or 94. In some embodiments, the variance in sequence identity is not in the RBD polypeptide at sequences encoding for K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein. In some embodiments, the variance in sequence identity is not in the RBD polypeptide at sequences encoding for K417, N439, L452, T478, E484, or N501, or any combination thereof, with reference to the full SARS-CoV-2 S protein. In some embodiments, the variance in sequence identity is in the RBD polypeptide at or at sequences encoding for C336, C361, C379, C391, C432, C480, C488, or C525, or any combination thereof, with reference to the full SARS-CoV-2 S protein.

In some embodiments, the polypeptide further comprises the autocatalytic polypeptide cleavage site sequence, optionally wherein the autocatalytic polypeptide cleavage site sequence is a P2A autocatalytic polypeptide cleavage site sequence. In some embodiments, the polypeptide consists essentially of the one or more RBD polypeptide sequences, the NP polypeptide sequence, the IgE leader polypeptide sequence, and the autocatalytic polypeptide cleavage site sequence. In some embodiments, the polypeptide comprises a sequence having at least 90%, 91%, 92%, 93%, 94%, 95%, 96%, 97%, 98%, 99%, or 100% sequence identity to SEQ ID NO: 82 or 89. In some embodiments, the variance in sequence identity is not in the RBD polypeptide at K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein. In some embodiments, the variance in sequence identity is not in the RBD polypeptide at K417, N439, L452, T478, E484, or N501, or any combination thereof, with reference to the full SARS-CoV-2 S protein. In some embodiments, the polypeptide is encoded by a nucleic acid comprising at least 90%, 91%, 92%, 93%, 94%, 95%, 96%, 97%, 98%, 99%, or 100% sequence identity to SEQ ID NO: 80, 81, or 88. In some embodiments, the variance in sequence identity is not in the RBD polypeptide at sequences encoding for K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein. In some embodiments, the variance in sequence identity is not in the RBD polypeptide at sequences encoding for K417, N439, L452, T478, E484, or N501, or any combination thereof, with reference to the full SARS-CoV-2 S protein. In some embodiments, the variance in sequence identity is in the RBD polypeptide at or at sequences encoding for C336, C361, C379, C391, C432, C480, C488, or C525, or any combination thereof, with reference to the full SARS-CoV-2 S protein.

In some embodiments, the polypeptide further comprises the M polypeptide sequence. In some embodiments, the polypeptide consists essentially of the one or more RBD polypeptide sequences, the NP polypeptide sequence, the IgE leader polypeptide sequence, the autocatalytic polypeptide cleavage site sequence, and the M polypeptide sequence. In some embodiments, the polypeptide comprises a sequence having at least 90%, 91%, 92%, 93%, 94%, 95%, 96%, 97%, 98%, 99%, or 100% sequence identity to SEQ ID NO: 85. In some embodiments, the variance in sequence identity is not in the RBD polypeptide at K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein. In some embodiments, the variance in sequence identity is not in the RBD polypeptide at K417, N439, L452, T478, E484, or N501, or any combination thereof, with reference to the full SARS-CoV-2 S protein. In some embodiments, the polypeptide is encoded by a nucleic acid comprising at least 90%, 91%, 92%, 93%, 94%, 95%, 96%, 97%, 98%, 99%, or 100% sequence identity to SEQ ID NO: 84. In some embodiments, the variance in sequence identity is not in the RBD polypeptide at sequences encoding for K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein. In some embodiments, the variance in sequence identity is not in the RBD polypeptide at sequences encoding for K417, N439, L452, T478, E484, or N501, or any combination thereof. In some embodiments, the variance in sequence identity is in the RBD polypeptide at or at sequences encoding for C336, C361, C379, C391, C432, C480, C488, or C525, or any combination thereof, with reference to the full SARS-CoV-2 S protein.

In some embodiments, the polypeptide further comprises the CC40.8 epitope. In some embodiments, the polypeptide consists essentially of the one or more RBD polypeptide sequences, the NP polypeptide sequence, the IgE leader polypeptide sequence, and the CC40.8 epitope. In some embodiments, the polypeptide comprises a sequence having at least 90%, 91%, 92%, 93%, 94%, 95%, 96%, 97%, 98%, 99%, or 100% sequence identity to SEQ ID NO: 97 or 99. In some embodiments, the variance in sequence identity is not in the RBD polypeptide at K417, N439, L452, T478, E484, N501, G339, S371, S373, S375, T376, D405, R408, N440, G446, S447, Q493, G496, Q498, or Y505, or any combination thereof, with reference to the full SARS-CoV-2 S protein. In some embodiments, the variance in sequence identity is not in the RBD polypeptide at K417, N439, L452, T478, E484, or N501, or any combination thereof, with reference to the full SARS-CoV-2 S protein. In some embodiments, the polypeptide is encoded by a nucleic acid comprising at least 90%, 91%, 92%, 93%, 94%, 95%, 96%, 97%, 98%, 99%, or 100% sequence identity to SEQ ID NO: 96 or 98. In some embodiments, the variance in sequence identity is not in the RBD polypeptide at sequences encoding for K417, N439, L452, T478, E484, N501, G339, S371, S373, S375, T376, D405, R408, N440, G446, S447, Q493, G496, Q498, or Y505, or any combination thereof. In some embodiments, the variance in sequence identity is not in the RBD polypeptide at sequences encoding for K417, N439, L452, T478, E484, or N501, or any combination thereof, with reference to the full SARS-CoV-2 S protein. In some embodiments, the variance in sequence identity is in the RBD polypeptide at or at sequences encoding for C336, C361, C379, C391, C432, C480, C488, or C525, or any combination thereof, with reference to the full SARS-CoV-2 S protein.

Any one of the polypeptides disclosed herein may be used in a medicament or for the manufacture of a medicament. In some embodiments, the medicament is used for the prevention, treatment, or inhibition of SARS-CoV-2 or other coronavirus in a subject. In some embodiments, the subject is a human.

Any one of the polypeptides disclosed herein may be recombinantly expressed. In some embodiments, the polypeptide is recombinantly expressed in a mammalian, bacterial, yeast, insect, or cell-free system. In some embodiments, the polypeptide is produced in mammalian, human, primary, immortalized, cancer, stem, fibroblasts, human embryonic kidney (HEK) 293, Chinese Hamster Ovary (CHO), bacterial, Escherichia coli, yeast, Saccharomyces cerevisiae, Pichia pastoris, insect, Spodoptera frugiperda Sƒ9, or S. frugiperda Sf21 cells, or in a cell-free system. In some embodiments, the polypeptide is purified using techniques known in the art, including but not limited to extraction, freeze-thawing, homogenization, permeabilization, centrifugation, density gradient centrifugation, ultracentrifugation, precipitation, SDS-PAGE, native PAGE, size exclusion chromatography, liquid chromatography, gas chromatography, hydrophobic interaction chromatography, ion exchange chromatography, anion exchange chromatography, cation exchange chromatography, affinity chromatography, immunoaffinity chromatography, metal binding chromatography, nickel column chromatography, epitope tag purification, or lyophilization. In some embodiments, the polypeptide is properly folded or denatured.

### Exemplary Immunogenic Composition Embodiments

The terms “prime” and “boost” as used herein related to separate immunogenic compositions used in a heterologous prime-boost immunization approach. Immunizations or vaccines commonly require more than one administration of an immunogenic composition to induce a successful immunity against a target pathogen in a host. Compared to this homologous approach where the same composition is provided for all administrations, a heterologous prime-boost administration may be more effective in establishing robust immunity with greater antibody levels and improved clearing or resistance against some pathogens such as viruses, coronaviruses, SARS-CoV-2, bacteria, parasites, protozoa, helminths. In a heterologous prime-boost administration, at least one prime dose comprising one type of immunogenic composition is first provided. After the at least one prime dose is provided, at least one boost dose comprising another type of immunogenic composition is then provided. Administration of the at least one boost dose is performed at least 1, 2, 3, 4, 5, 6, 7, 8, 9, 10, 11, 12, 24, 36, or 48 days or weeks after the at least one prime dose is administered or within a range of time defined by any two of the aforementioned time points e.g., within 1-48 days or 1-48 weeks. In some embodiments, the prime dose comprises a nucleic acid (e.g., DNA or RNA) that encodes for one or more antigens or epitopes, and the boost dose comprises a polypeptide that comprises one or more antigens or epitopes. In the host, the nucleic acid prime is translated in vivo to elicit an immune reaction and causes a greater response against the subsequent polypeptide boost.

In some embodiments, the nucleic acid prime comprises, consists essentially of, or consists of sequences from SARS-CoV-2 or other coronaviruses, including variants thereof. In some embodiments, the sequences from SARS-CoV-2 or other coronaviruses encode for an S, RBD, M, E, or NP polypeptide, including mutated or variant polypeptides thereof. In some embodiments, the nucleic acid prime also includes an IgE leader sequence. In some embodiments, the nucleic acid prime also comprises a CC40.8 epitope sequence. In some embodiments, the nucleic acid prime also includes at least one HDAg sequence. In some embodiments, the nucleic acid sequences are codon optimized for expression in humans. In some embodiments, the polypeptide boost comprises, consists essentially of, or consists of polypeptides from SARS-CoV-2 or other coronaviruses, including variants thereof. In some embodiments, the polypeptides from SARS-CoV-2 or other coronaviruses are S, RBD, M, E, or NP polypeptides. In some embodiments, the prime dose is a polypeptide, and the boost dose is a nucleic acid. General information about heterologous prime-boost approaches can be found in PCT Publications WO 2006/013106, WO 2006/040334, WO 2008/094188, each of which are hereby expressly incorporated by reference for the purpose of describing prime-boost methods.

Disclosed herein are immunogenic compositions or product combinations. In some embodiments, these immunogenic compositions or product combinations may be used in a prime-boost approach. In some embodiments, the immunogenic composition or product combination comprises (a) a nucleic acid comprising at least one nucleic acid sequence encoding a SARS-CoV-2 polypeptide, or (b) a polypeptide comprising at least one SARS-CoV-2 polypeptide, or both. In some embodiments, the immunogenic compositions comprise a nucleic acid and polypeptide, wherein the nucleic acid is any one of the nucleic acids disclosed herein and/or the polypeptide is any one of the polypeptides disclosed herein.

In some embodiments of any one of the immunogenic compositions or product combinations disclosed herein, the at least one nucleic acid sequence encoding for a SARS-CoV-2 polypeptide comprises i) a nucleic acid sequence encoding an RBD polypeptide; ii) a nucleic acid sequence encoding an NP polypeptide; iii) a nucleic acid sequence encoding an M polypeptide; iv) a nucleic acid sequence encoding an HDAg polypeptide; v) a nucleic acid sequence encoding a P2A autocatalytic polypeptide cleavage site; vi) a nucleic acid sequence encoding an IgE leader polypeptide; vii) a nucleic acid sequence encoding a S polypeptide; or viii) a nucleic acid sequence encoding a CC40.8 epitope; or any combination thereof. In some embodiments, the nucleic acid is any one of the nucleic acids disclosed herein. In some embodiments, the nucleic acid shares or comprises at least 90%, 91%, 92%, 93%, 94%, 95%, 96%, 97%, 98%, 99%, or 100% homology or sequence identity to any one or more of SEQ ID NO: 1-12, which is optionally used in a medicament, such as for the prevention, treatment, or inhibition of SARS-CoV-2 in a subject, such as a mammal, preferably a human. In other embodiments, the nucleic acid is codon optimized for expression in a human. In some embodiments, the nucleic acid shares or comprises at least 90%, 91%, 92%, 93%, 94%, 95%, 96%, 97%, 98%, 99%, or 100% homology or sequence identity to any one or more of SEQ ID NO: 13-24, 39-40, 57-63, 71, 73, 75, 77-78, 80-81, 84, 86, 88, 92, 94, 96, or 98, which is optionally used in a medicament, such as for the prevention, treatment, or inhibition of SARS-CoV-2 in a subject, such as a mammal, preferably a human. In some embodiments, the RBD polypeptide is an RBD tandem repeat single chain dimer. In some embodiments, the RBD polypeptide is from the SARS-CoV-2 virus or a variant thereof. In some embodiments, the RBD polypeptide comprises a K417N, N439K, L452R, T478K, E484K, N501Y, G339D, S371L, S371F, S373P, S375F, T376A, D405N, R408S, N440K, G446S, S447N, Q493R, G496S, Q498R, or Y505H mutation with reference to the full SARS-CoV-2 S protein (e.g., as set forth in NCBI Accession No. YP_009724390), or any combination thereof, or none of these mutations. In some embodiments, the RBD polypeptide comprises a K417N, N439K, L452R, T478K, E484K, or N501Y mutation with reference to the full SARS-CoV-2 S protein (e.g., as set forth in NCBI Accession No. YP_009724390), or any combination thereof, or none of these mutations. In some embodiments, the RBD polypeptide shares or comprises at least 90%, 91%, 92%, 93%, 94%, 95%, 96%, 97%, 98%, 99%, or 100% homology or sequence identity to any one or more of SEQ ID NO: 46, or 52-55. In some embodiments, the nucleic acid is provided in a recombinant vector. In some embodiments, the recombinant vector is pVAX1. In some embodiments, each of the one or more nucleic acid sequences encoding the RBD polypeptide comprise one or more mutations corresponding to C336, C361, C379, C391, C432, C480, C488, or C525 with reference to the full SARS-CoV-2 S protein (e.g. as set forth in NCBI Accession No. YP_009724390 (SEQ ID NO: 83)), or none of these mutations.

In some embodiments of any one of the immunogenic compositions or product combinations disclosed herein, the at least one SARS-CoV-2 polypeptide comprises i) an RBD polypeptide sequence; ii) an NP polypeptide sequence; iii) an M polypeptide sequence; iv) an HDAg polypeptide sequence; v) a P2A autocatalytic polypeptide cleavage site sequence; vi) an IgE leader polypeptide sequence; vii) an S polypeptide sequence; or viii) a CC40.8 epitope; or any combination thereof. In some embodiments, the polypeptide is any one of the polypeptides disclosed herein. In some embodiments, the polypeptide shares or comprises at least 90%, 91%, 92%, 93%, 94%, 95%, 96%, 97%, 98%, 99%, or 100% homology or sequence identity to any one or more of SEQ ID NO: 25-36, 41-42, 64-70, 72, 74, 76, 79, 82, 85, 87, 89, 93, 95, 97, or 99, which is optionally used in a medicament, such as for the prevention, treatment, or inhibition of SARS-CoV-2 in a subject, such as a mammal, preferably a human. In some embodiments, the RBD polypeptide is an RBD tandem repeat single chain dimer. In some embodiments, the RBD polypeptide is from the SARS-CoV-2 virus or a variant thereof. In some embodiments, the RBD polypeptide comprises a K417N, N439K, L452R, T478K, E484K, N501Y, G339D, S371L, S371F, S373P, S375F, T376A, D405N, R408S, N440K, G446S, S447N, Q493R, G496S, Q498R, or Y505H mutation with reference to the full SARS-CoV-2 S protein (e.g., as set forth in NCBI Accession No. YP_009724390), or any combination thereof, or none of these mutations. In some embodiments, the RBD polypeptide comprises a K417N, N439K, L452R, T478K, E484K, or N501Y mutation with reference to the full SARS-CoV-2 S protein (e.g., as set forth in NCBI Accession No. YP_009724390), or any combination thereof, or none of these mutations. In some embodiments, the RBD polypeptide shares or comprises at least 90%, 91%, 92%, 93%, 94%, 95%, 96%, 97%, 98%, 99%, or 100% homology or sequence identity to any one of SEQ ID NO: 46, or 52-55. In some embodiments, the polypeptide is recombinantly expressed. In some embodiments, the polypeptide is recombinantly expressed in a mammalian, bacterial, yeast, insect, or cell-free system. In some embodiments, the RBD polypeptide sequence comprises one or more mutations corresponding to C336, C361, C379, C391, C432, C480, C488, or C525 with reference to the full SARS-CoV-2 S protein (e.g. as set forth in NCBI Accession No. YP_009724390 (SEQ ID NO: 83)), or none of these mutations.

In some embodiments, any one of the immunogenic compositions or product combinations disclosed herein further comprise an adjuvant. In some embodiments, the adjuvant is any adjuvant conventionally known in the art. In some embodiments, the adjuvant is Matrix-M, alum and/or QS21.

### Methods of Therapy or Use

Disclosed herein are methods of generating an immune response and/or generating neutralizing antibodies in a subject. In some embodiments, the methods comprise administering any one of the nucleic acids and/or any one of the polypeptides disclosed herein to the subject.

Also disclosed herein are methods of generating an immune response and/or generating neutralizing antibodies in a subject using any one of the immunogenic compositions or product combinations disclosed herein. In some embodiments, these methods comprise administering to the subject at least one prime dose comprising the nucleic acid of any one of the immunogenic compositions or product combinations; and administering to the subject at least one boost dose comprising the polypeptide of any one of the immunogenic compositions or product combinations. In some embodiments, the immune response and/or neutralizing antibodies are against SARS-CoV-2 or other coronavirus. In some embodiments, the subject is a mammal, such as a mouse, rat, monkey, cat, dog, or human. In some embodiments, the at least one boost dose further comprises an adjuvant. In some embodiments, the adjuvant is any adjuvant conventionally known in the art. In some embodiments, the adjuvant is Matrix-M, alum and/or QS21. In some embodiments, the at least one boost dose is administered at least 1, 2, 3, 4, 5, 6, 7, 8, 9, 10, 11, 12, 24, 36, or 48 days or weeks after the at least one prime dose is administered or within a range of time defined by any two of the aforementioned time points e.g., within 1-48 days or 1-48 weeks. In some embodiments, the administration is provided enterally, orally, intranasally, parenterally, subcutaneously, intramuscularly, intradermally, or intravenously or any combination thereof, and optionally with in vivo electroporation. In some embodiments, the administration is performed in conjunction with an antiviral therapy. In some embodiments, the antiviral therapy comprises administration of dexamethasone, favipiravir, favilavir, remdesivir, tocilizumab, galidesivir, sarilumab, lopinavir, ritonavir, darunavir, ribavirin, interferon-α, pegylated interferon-α, interferon alfa-2b, convalescent serum, or any combination thereof.

In some embodiments, administration of the nucleic acid prime and polypeptide boost comprising components of SARS-CoV-2 or other coronaviruses, including variants thereof, in a subject (e.g. mouse, rabbit, monkey, human) of any one of the immunogenic compositions or product combinations disclosed herein results in greater anti-S, anti-RBD, anti-M, anti-E, anti-NP, anti-SARS-CoV-2, or anti-coronavirus antibody titer at a ratio of 1, 2, 3, 4, 5, 6, 7, 8, 9, 10, 50, 100, 150, 200, 300, 400, 500, 600, 700, 800, 900, 1000, 5000, 10000, 100000, or 1000000 or any ratio within a range defined by any two of the aforementioned ratios compared to nucleic acid-only or polypeptide-only immunized, or unimmunized control organisms, quantified by techniques known in the art such as ELISA. In some embodiments, administration of the nucleic acid prime and polypeptide boost comprising components of SARS-CoV2 or other coronaviruses in a subject results in serum that neutralizes the in vitro or in vivo infectivity of SARS-CoV2 or other coronaviruses more effectively and reduces the incidence of infection or multiplicity of infection (MOI) to a ratio of 0.00001, 0.00005, 0.0001, 0.0005, 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1.0 or any ratio within a range defined by any two of the aforementioned ratios compared to sera from nucleic acid-only or polypeptide-only immunized, or unimmunized control organisms. In some embodiments, administration of the nucleic acid prime and polypeptide boost comprising components of SARS-CoV2 or other coronaviruses in a subject results in a greater number of interferon gamma (IFNγ)-positive cells (e.g. T cells, macrophages, natural killer (NK) cells) at a ratio of 1, 2, 3, 4, 5, 6, 7, 8, 9, 10, 20, 30, 40, 50, 60, 70, 80, 90, 100, 110, 120, 130, 140, 150, 200, 250, 300, 350, 400, 450, 500, 550, 600, 650, 700, 750, 800, 850, 900, 950, 1000, 5000, or 10000, or any ratio within a range defined by any two of the aforementioned ratios compared to nucleic acid-only or polypeptide-only immunized, or unimmunized control organisms.

Also disclosed herein are immunogenic compositions or product combinations for use in the treatment or inhibition of SARS-CoV-2 or other coronavirus, including variants thereof. In some embodiments, the immunogenic compositions or product combinations comprise (a) a nucleic acid comprising at least one nucleic acid sequence encoding a SARS-CoV-2 polypeptide, or (b) a polypeptide comprising at least one SARS-CoV-2 polypeptide, or both. In some embodiments, the at least one nucleic acid sequence encoding a SARS-CoV-2 polypeptide comprises: i) a nucleic acid sequence encoding an RBD polypeptide; ii) a nucleic acid sequence encoding an NP polypeptide; iii) a nucleic acid sequence encoding an M polypeptide; iv) a nucleic acid sequence encoding an HDAg polypeptide; v) a nucleic acid sequence encoding a P2A autocatalytic polypeptide cleavage site; vi) a nucleic acid sequence encoding an IgE leader polypeptide; vii) a nucleic acid sequence encoding a S polypeptide; or viii) a nucleic acid sequence encoding a CC40.8 epitope; or any combination thereof. In some embodiments, the nucleic acid is any one of the nucleic acids disclosed herein. In some embodiments, the nucleic acid shares or comprises at least 90%, 91%, 92%, 93%, 94%, 95%, 96%, 97%, 98%, 99%, or 100% homology or sequence identity to any one or more of SEQ ID NO: 1-12, 77, or 80. In some embodiments, the nucleic acid is codon optimized for expression in a human. In some embodiments, the nucleic acid shares or comprises at least 90%, 91%, 92%, 93%, 94%, 95%, 96%, 97%, 98%, 99%, or 100% homology or sequence identity to any one or more of SEQ ID NO: 13-24, 39-40, 57-63, 71, 73, 75, 78, 81, 84, 86, 88, 92, 94, 96, or 98. In some embodiments, the at least one SARS-CoV-2 polypeptide comprises: i) an RBD polypeptide sequence; ii) an NP polypeptide sequence; iii) an M polypeptide sequence; iv) an HDAg polypeptide sequence; v) a P2A autocatalytic polypeptide cleavage site sequence; vi) an IgE leader polypeptide sequence; vii) an S polypeptide sequence; or viii) a CC40.8 epitope; or any combination thereof. In some embodiments, the polypeptide is any one of the polypeptides disclosed herein. In some embodiments, the polypeptide shares or comprises at least 90%, 91%, 92%, 93%, 94%, 95%, 96%, 97%, 98%, 99%, or 100% homology or sequence identity to any one or more of SEQ ID NO: 25-36, 41-42, 64-70, 72, 74, 76, 79, 82, 85, 87, 89, 93, 95, 97, or 99. In some embodiments, the RBD polypeptide is an RBD tandem repeat single chain dimer. In some embodiments, the RBD polypeptide shares or comprises at least 90%, 91%, 92%, 93%, 94%, 95%, 96%, 97%, 98%, 99%, or 100% homology or sequence identity to SEQ ID NO: 46. In some embodiments, the polypeptide is recombinantly expressed. In some embodiments, the polypeptide is recombinantly expressed in a mammalian, bacterial, yeast, insect, or cell-free system. In some embodiments, the immunogenic composition or product combination further comprises an adjuvant. In some embodiments, the adjuvant is any adjuvant conventionally known in the art. In some embodiments, the adjuvant is Matrix-M, alum and/or QS21. In some embodiments, the nucleic acid is provided in a recombinant vector. In some embodiments, the recombinant vector is pVAX1. In some embodiments, each of the one or more nucleic acid sequences encoding the RBD polypeptide comprise one or more mutations corresponding to C336, C361, C379, C391, C432, C480, C488, or C525 with reference to the full SARS-CoV-2 S protein (e.g. as set forth in NCBI Accession No. YP_009724390 (SEQ ID NO: 83)), or none of these mutations. In some embodiments, the RBD polypeptide sequence comprises one or more mutations corresponding to C336, C361, C379, C391, C432, C480, C488, or C525 with reference to the full SARS-CoV-2 S protein (e.g. as set forth in NCBI Accession No. YP_009724390 (SEQ ID NO: 83)), or none of these mutations.

In some embodiments, the nucleic acids and/or polypeptides are administered to an animal, including but not limited to humans, mice, rats, rabbits, cats, dogs, horses, cows, pigs, sheep, monkeys, primates, or chickens. In some embodiments, the nucleic acids and/or polypeptides are administered 1, 2, 3, 4, 5, 6, or 7 days, or 1, 2, 3, 4, 5, 6, 7, 8, 9, or 10 weeks, or 1, 2, 3, 4, 5, 6, 7, 8, 9, 10, 11, 12 months or any time within a range defined by any two of the aforementioned times between each dose. In some embodiments, the nucleic acids are administered before the polypeptides are administered. In some embodiments, the polypeptides are administered before the nucleic acids.

In some embodiments, the nucleic acids and/or polypeptides are administered in an amount of 1, 10, 100, 1000 ng, or 1, 10, 100, 200, 300, 400, 500, 600, 700, 800, 900, 1000 µg, or 1, 10, 100, or 1000 mg or any amount within a range defined by any two of the aforementioned amounts. In some embodiments, the nucleic acids and/or polypeptides are administered with excipients. In some embodiments, the nucleic acids and/or polypeptides are administered with adjuvants. In some embodiments, the nucleic acids are administered with in vivo electroporation.

In some embodiments, the administration of the nucleic acids and/or polypeptides provide transient, lasting, or permanent protection against a coronavirus infection, such as that caused by the SARS-CoV-2 virus, or a variant thereof. In some embodiments, the transient, lasting, or permanent protection against the coronavirus infection is superior to other immunogenic compositions. In some embodiments, the administration of the nucleic acids and/or polypeptides is performed in conjunction with an antiviral therapy. In some embodiments, the administration of the nucleic acids and/or polypeptides to provide transient, lasting, or permanent protection against a coronavirus infection is effective in humans. In some embodiments, the nucleic acids and/or polypeptides are used as vaccines or immunogens for treating, inhibiting, or ameliorating a coronavirus infection or for providing protection against a coronavirus infection.

The invention is generally disclosed herein using affirmative language to describe the numerous embodiments. The invention also includes embodiments in which subject matter is excluded, in full or in part, such as substances or materials, method steps and conditions, protocols, or procedures.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1: Design of SARS-CoV-2 Immunogenic Composition Constructs

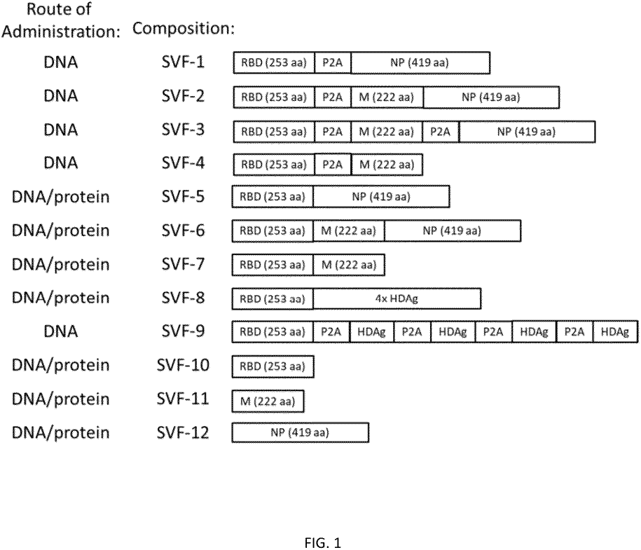

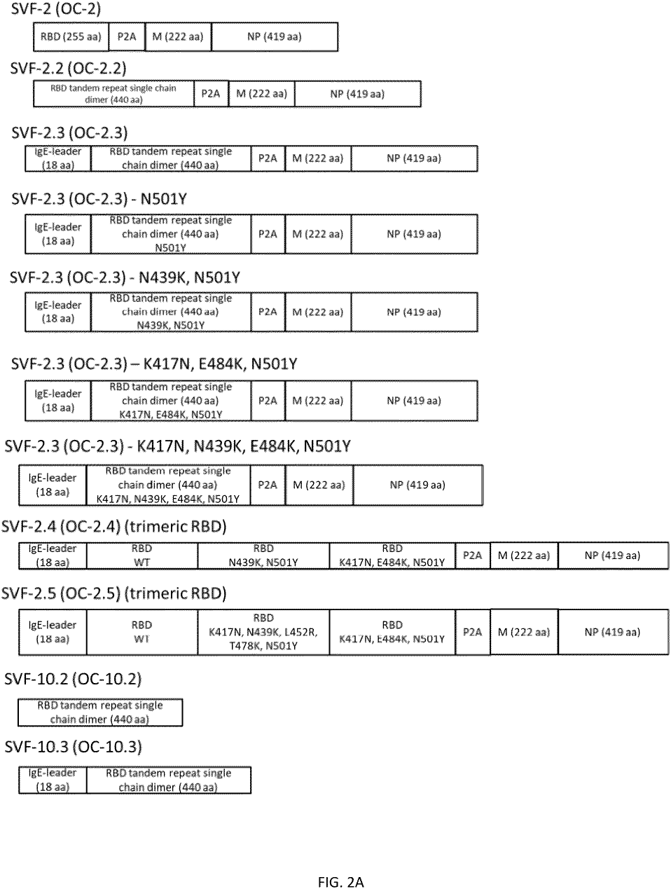

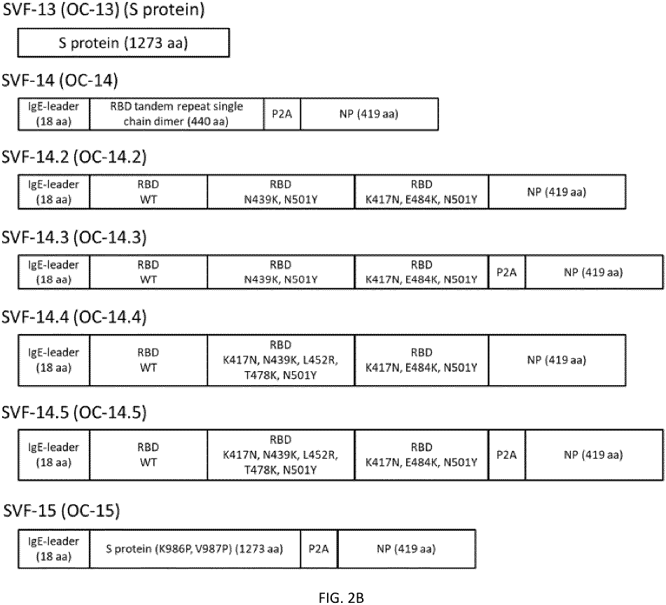

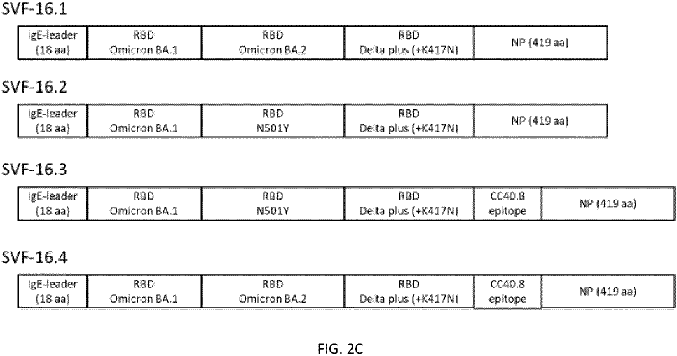

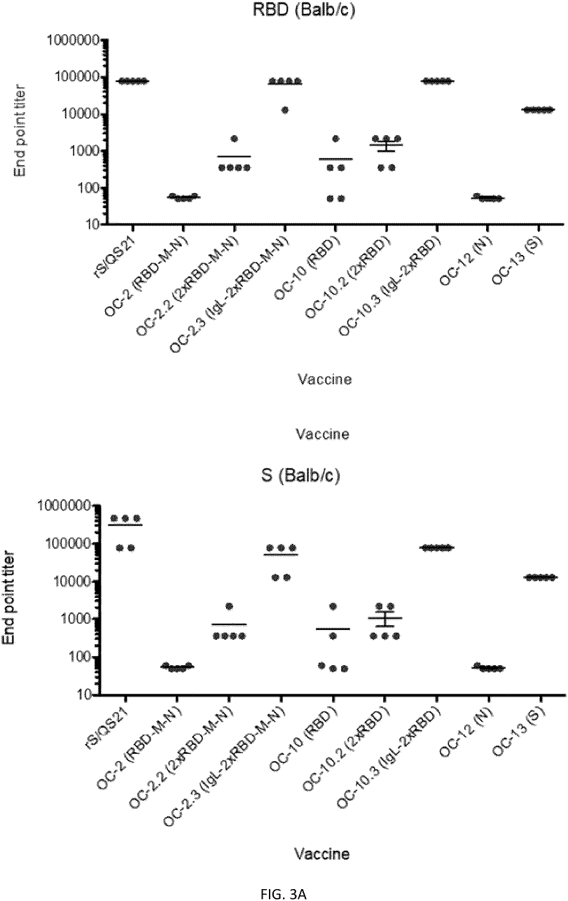

Several recombinant constructs containing components of the SARS-CoV-2 virus are prepared and are depicted in Table 1 and FIGS. 1, 2A-C. As the RBD of the S protein is known to be highly immunogenic, the majority of the constructs comprise an RBD sequence. In some cases, the RBD sequence is an RBD tandem repeat single chain dimer sequence. However, it is envisioned that a construct can have any combination of encoding sequences, in any order, from the SARS-CoV-2 virus or any other coronavirus. This includes constructs lacking an RBD sequence. This also includes sequences for coronavirus replication proteins or hemagglutinin esterase.

An RBD sequence can be found in SVF-1 (OC-1), SVF-2 (OC-2), SVF-3 (OC-3), SVF-4 (OC-4), SVF-5 (OC-4), SVF-6 (OC-6), SVF-7 (OC-7), SVF-8 (OC-8), SVF-9 (OC-9), SVF-10 (OC-10), SVF-14 (OC-14), SVF-14.2 (OC-14.2), SVF-14.3 (OC-14.3), SVF-2.2 (OC-2.2), SVF-2.3 (OC-2.3), SVF-2.4 (OC-2.4), SVF-2.5 (OC-2.5), SVF-16.1, SVF-16.2, SVF-16.3, and SVF-16.4, including any derivatives and/or mutants thereof.

An RBD tandem repeat single chain dimer is found in SVF-2.2 and SVF-2.3, and SVF-14 (OC-14), including any derivatives and/or mutants thereof.

A trimeric RBD construct is found in SVF-2.4, SVF-2.5, SVF-14.2, SVF-14.3, SVF-14.4, SVF-14.5, SVF-16.1, SVF-16.2, SVF-16.3, and SVF-16.4, including any derivatives and/or mutants thereof.

A S protein sequence (which necessarily also contains an RBD sequence) is found in SVF-13 (OC-13) and SVF-15 (OC-15), including any derivatives and/or mutants thereof.

An NP protein sequence is found in SVF-1, SVF-2, SVF-3, SVF-5, SVF-6, SVF-12 (OC-12), SVF-14, SVF-14.2, SVF-14.3, SVF-14.4, SVF-14.5, SVF-15, SVF-2.2, SVF-2.3, SVF-2.4, SVF-2.5, SVF-16.1, SVF-16.2, SVF-16.3, and SVF-16.4, including any derivatives and/or mutants thereof.

An M protein sequence is found in SVF-2, SVF-3, SVF-4, SVF-6, SVF-7, SVF-11 (OC-11), SVF-2.2, SVF-2.3, SVF-2.4, and SVF-2.5, including any derivatives and/or mutants thereof.

A CC40.8 epitope sequence is found in SVF-16.3 and SVF-16.4, including any derivatives and/or mutants thereof.

At least one P2A autocatalytic peptide cleavage site is found in SVF-1, SVF-2, SVF-3, SVF-4, SVF-9, SVF-14, SVF-14.3, SVF-14.5, SVF-15, SVF-2.2, SVF-2.3, and SVF-2.4, including any derivatives and/or mutants thereof. The presence of this P2A autocatalytic peptide cleavage site (which may trivially be substituted with another autocatalytic peptide cleavage site), allows for translation of separate proteins in the target cell from one or more contiguous nucleic acid gene or cassette. The presence of the autocatalytic peptide cleavage site also suggests that recombinant protein expression and purification of said constructs will lead to separate polypeptide components, which will be difficult to purify. While still possible (e.g. with the same or different epitope tags), using the other constructs for producing protein for immunogenic administration is more feasible.

In some embodiments, the recombinant constructs further contain components of the hepatitis B virus or hepatitis D virus. This is seen with SVF-8 and SVF-9, where HDAg copies of 4 different consensus sequences (genotypes 1A, 1B, 2A, and 2B) are provided. HDAg is also a highly immunogenic polypeptide, and it is envisioned that inclusion of the HDAg sequences improves immunogenic response to the RBD or other coronavirus sequences. It is also envisioned that these constructs will provide dual immunogenic response against SARS-CoV-2 (or other coronavirus) and hepatitis B or D.

Constructs SVF-10 (RBD), SVR-11 (M), SVF-12 (NP), and SVF-13 (S) are provided as single SARS-CoV-2 sequence compositions to assess relative immunogenicity of the different components.

TABLE 1

SARS-CoV-2 immunogenic composition candidates

| | | | |
|---|---|---|---|
| Composition | Wild-type DNA sequence | Human codon optimized sequence | Polypeptide sequence |
| SVF-1 (OC-1) | SEQ ID NO: 1 | SEQ ID NO: 13 | SEQ ID NO: 25 |
| SVF-2 (OC-2) | SEQ ID NO: 2 | SEQ ID NO: 14 | SEQ ID NO: 26 |
| SVF-3 (OC-3) | SEQ ID NO: 3 | SEQ ID NO: 15 | SEQ ID NO: 27 |
| SVF-4 (OC-4) | SEQ ID NO: 4 | SEQ ID NO: 16 | SEQ ID NO: 28 |
| SVF-5 (OC-5) | SEQ ID NO: 5 | SEQ ID NO: 17 | SEQ ID NO: 29 |
| SVF-6 (OC-6) | SEQ ID NO: 6 | SEQ ID NO: 18 | SEQ ID NO: 30 |
| SVF-7 (OC-7) | SEQ ID NO: 7 | SEQ ID NO: 19 | SEQ ID NO: 31 |
| SVF-8 (OC-8) | SEQ ID NO: 8 | SEQ ID NO: 20 | SEQ ID NO: 32 |
| SVF-9 (OC-9) | SEQ ID NO: 9 | SEQ ID NO: 21 | SEQ ID NO: 33 |
| SVF-10 (OC-10) | SEQ ID NO: 10 | SEQ ID NO: 22 | SEQ ID NO: 34 |
| SVF-11 (OC-11) | SEQ ID NO: 11 | SEQ ID NO: 23 | SEQ ID NO: 35 |
| SVF-12 (OC-12) | SEQ ID NO: 12 | SEQ ID NO: 24 | SEQ ID NO: 36 |
| SVF-2.2 (OC-2.2) | N/A | SEQ ID NO: 39 | SEQ ID NO: 41 |
| SVF-2.3 (OC-2.3) | N/A | SEQ ID NO: 40 | SEQ ID NO: 42 |
| SVF-2.3 (OC-2.3) -N501Y | N/A | SEQ ID NO: 57 | SEQ ID NO: 64 |
| SVF-2.3 (OC-2.3) -N439K, N501Y | N/A | SEQ ID NO: 58 | SEQ ID NO: 65 |
| SVF-2.3 (OC-2.3) -K417N, E484K, N501Y | N/A | SEQ ID NO: 59 | SEQ ID NO: 66 |
| SVF-2.3 (OC-2.3) -K417N, N439K, E484K, N501Y | N/A | SEQ ID NO: 60 | SEQ ID NO: 67 |
| SVF-2.4 (OC-2.4) | N/A | SEQ ID NO: 61 | SEQ ID NO: 68 |
| SVF-14 | N/A | SEQ ID NO: 62 | SEQ ID NO: 69 |
| SVF-15 | N/A | SEQ ID NO: 63 | SEQ ID NO: 70 |
| SVF-13 (OC-13) | N/A | SEQ ID NO: 71 | SEQ ID NO: 72 |
| SVF-10.2 (OC-10.2) | N/A | SEQ ID NO: 73 | SEQ ID NO: 74 |
| SVF-10.3 (OC-10.3) | N/A | SEQ ID NO: 75 | SEQ ID NO: 76 |
| SVF-14.2 (OC-14.2) | SEQ ID NO: 77 | SEQ ID NO: 78 | SEQ ID NO: 79 |
| SVF-14.3 (OC-14.3) | SEQ ID NO: 80 | SEQ ID NO: 81 | SEQ ID NO: 82 |
| SVF-2.5 (OC-2.5) | N/A | SEQ ID NO: 84 | SEQ ID NO: 85 |
| SVF-14.4 (OC-14.4) | N/A | SEQ ID NO: 86 | SEQ ID NO: 87 |
| SVF-14.5 (OC-14.5) | N/A | SEQ ID NO: 88 | SEQ ID NO: 89 |
| SVF-16.1 | N/A | SEQ ID NO: 92 | SEQ ID NO: 93 |
| SVF-16.2 | N/A | SEQ ID NO: 94 | SEQ ID NO: 95 |
| SVF-16.3 | N/A | SEQ ID NO: 96 | SEQ ID NO: 97 |
| SVF-16.4 | N/A | SEQ ID NO: 98 | SEQ ID NO: 99 |

### Example 2: Methodology

### Animals

BALB/c, C57BL/6 and K18-hACE2 (B6.Cg-Tg(K18-ACE2)2Prlmn/J) mice can be obtained from the Jackson Laboratory. All mice are 8-10 weeks old at the start of the experiments and maintained under standard conditions. New Zealand White rabbits are purchased from commercial vendors.

### Recombinant Vectors

Sequences for SARS-CoV-2 are obtained from NCBI GenBank accession number: MN908947.3 (e.g. complete genome), YP_009724390 (e.g. surface glycoprotein), YP_009724393.1 (e.g. membrane glycoprotein), and YP_009724397.2 (e.g. nucleocapsid phosphoprotein). The HDAg sequences of genotypes 1 and 2 are obtained from four different clinical isolates; US-2 and CB, and 7/18/83 and TW2476, respectively, and codon optimized for expression in human.

For the DNA immunogenic compositions, genes are cloned into the pVAX1 backbone (ThermoFisher) using restriction sites BamHI and XbaI. Plasmids are grown in TOP10 E. coli cells (ThermoFisher) and purified for in vivo injections using Qiagen Endofree DNA purification kit (Qiagen GmbH) following manufacturer’s instructions. The correct gene size is confirmed by restriction enzyme digests. In addition, all cloned gene sequences were sequenced to confirm the correct nucleotide sequence.

For protein expression constructs, genes are cloned into the pET100 E. coli T7 expression vector (ThermoFisher). Other commercially available expression vectors can be used. Expression vectors are transformed into BL21(DE3) E. coli (or other T7 expression E. coli strain) and induced for purification according to protocols known in the art.

### Western Blot

Western blot is performed as known in the art. HeLa cells are transfected with each pVAX1construct using Lipofectamine 3000 Transfection Reagent (ThermoFisher). A pVAX1plasmid with a GFP reporter gene is used as a control. For protein detection, serum from rabbits immunized with one of the SARS-CoV-2 pVAX1compositions or commercially available anti-SARS-CoV-2 antibodies, and an appropriate HRP secondary antibody, are used. Chemiluminescence is induced with the Pierce TM ECL Plus Western Blotting Substrate and images are collected with a Gel Doc XR+ System (BioRad).

### Immunization Protocols

To evaluate the immunogenicity of the constructs in vivo, mice and rabbits are immunized at monthly intervals and sacrificed two weeks later for spleen and blood collection. In brief, mice (five to ten per group) are immunized intramuscularly (i.m.) in the tibialis cranialis anterior (TA) muscle with 1-50 µg plasmid DNA in a volume of 30-50 µL in sterile PBS by regular needle (27G) injection followed by in vivo electroporation (EP) using the Cliniporator2 device (IGEA, Carpi, Italy). During in vivo electroporation, a 1 ms 600 V/cm pulse followed by a 400 ms 60 V/cm pulse pattern is used to facilitate better uptake of the DNA. Prior to vaccine injections, mice are given analgesic and kept under isoflurane anesthesia during the vaccinations. For studies in rabbits, 2-4 New Zealand White rabbits per group are immunized with 100 µg to 900 µg plasmid DNA. Vaccines are administered by i.m. injection in 300µL sterile PBS to the right TA muscle followed by in vivo EP.

### Detection of IFNγ Cells by Enzyme-Linked Immunospot Assay (ELISpot)

Two weeks after the last immunization, splenocytes from each immunized group of mice pooled are collected and tested for their ability to induce SARS-CoV-2-specific T cells based on IFN-γ secretion for 48h as known in the art using SARS-CoV-2 derived peptides and/or proteins in a commercially available ELISpot assay (Mabtech, Nacka Strand, Sweden).

### Antibody Detection by ELISA

Detection of mouse and rabbit IgG against various SARS-CoV-2 peptides and/or proteins is performed using protocols known in the art. Antibody titers are determined as endpoint serum dilutions at which the OD value (e.g. at 405 nm or 492 nm) is at least twice the OD of the negative control (non-immunized or control animal serum) at the same dilution.

### In Vitro SARS-CoV-2 Neutralization Assay

Neutralization ability of immunization sera from animals is assessed in vitro. Vero E6 cells are grown to confluence on a culture plate. Media containing either sera from animals immunized with the SARS-CoV-2 compositions, or sera from control animals, is added to the cells. The cells are then infected with SARS-CoV-2 virus particles. Viral infectivity and serum neutralization are assessed by counting viral plaques or viral titer by detection of the viral genome/gene(s).

### In Vivo SARS-CoV-2 Neutralization Assay in hACE2 Mouse Model

Wild-type or K18-hACE2 mice are immunized with the SARS-CoV-2 immunogenic compositions or a control. Different combinations are employed, including but not limited to DNA-only compositions, protein-only compositions, DNA prime/protein boost compositions, or protein-prime/DNA-boost compositions. K18-hACE2 mice are then infected with SARS-CoV-2 virus particles. For wild-type mice, they were made transiently transgenic for hACE2 by hydrodynamic injection, or other relevant techniques, 1-5 days prior to infection with SARS-CoV-2. Effect of the viral infection, including mouse weight, symptoms, morbidity and mortality, and viral load, are assessed.

### Statistical Analysis

Data are analyzed using GraphPad Prism V.5 and V.8 software and Microsoft Excel V.16.13.1.

### Example 3: SARS-CoV2 DNA and Protein Compositions Are Immunogenic in Animals

While immunogenic compositions and vaccines have traditionally been either whole organisms or antigenic proteins, it has been recently shown that in vivo administration of DNA to living tissue and the subsequent transcription and translation of antigenic proteins are also highly effective in triggering an immune response. These DNA prime/protein boost immunogenic compositions are being explored as potential vaccine candidates against various diseases.

Mice are immunized with (1) a DNA composition comprising one of the compositions disclosed herein (3 sequential doses of 50 µg DNA), (2) a polypeptide composition comprising one of the compositions disclosed herein (3 sequential does of 20 µg protein with alum adjuvant), or (3) a DNA composition comprising one of the compositions disclosed herein followed by a polypeptide composition comprising one of the compositions disclosed herein (2 doses of 50 µg DNA then 2 doses of 20 µg protein with alum).

Following 1, 2, 3, 4, 5, 6, or 7 days, or 1, 2, 3, 4, 5, 6, 7, 8, 9, or 10 weeks, or 1, 2, 3, 4, 5, 6, 7, 8, 9, 10, 11, 12 months or any time within a range defined by any two of the aforementioned times as the duration of administration of the DNA prime/protein boost compositions, immunity of the mice against SARS-CoV-2 antigens is assessed. White blood cells are purified from mouse whole blood samples and incubated with purified polypeptide antigens, including S protein, RBD, M protein, and NP protein. Cells are also incubated with Concanavalin A (“ConA”) as a positive control, and two ovalbumin peptides (“OVA Th” and “OVA CTL”) as negative controls. The population frequency of interferon gamma (IFNγ) producing cells in response to antigen exposure is assessed by enzyme-linked immunospot assay (ELISpot). Briefly, white blood cells are incubated with antigen in wells coated with IFNy antibodies. The cells are then removed, and biotinylated IFNy antibodies, alkaline phosphatase-crosslinked streptavidin, and alkaline phosphatase substrate colorimetric reagents are added to the wells in succession with thorough washing in between. The plate is then allowed to dry and the remaining colored spots that correspond to IFNy-secreting cells are counted by microscopy.

Treated mice show a comparatively stronger immune cell response overall. This demonstrates that this DNA prime/protein boost approach may be effective at inducing a robust immunogenic response greater than traditional protein or organism-based compositions for certain pathogens.

Corresponding experiments are also performed in rabbits (Oryctolagus cuniculus). New Zealand white rabbits are immunized with (1) a DNA-only composition comprising one of the compositions disclosed herein, (2) a protein-only composition comprising one of the compositions disclosed herein, or (3) a DNA prime/protein boost composition comprising one or more of the compositions disclosed herein. Compositions are administered four times as weeks 0, 4, 8, and 12, with either 900 µg DNA im/EP or 300 µg protein with alum administered for each dose. For DNA-protein compositions (3), 900 µg DNA im/EP is administered for the first dose at week 0, and 300 µg protein with alum is administered for the second, third, and fourth doses at weeks 4, 8, and 12. Anti-RBD titers in sera are assessed at weeks 0, 2, 10, and 14 (i.e. 2 weeks after each dosage). Not only does the DNA prime/protein boost composition (3) result in greater overall titers compared to DNA-only (1) and protein-only (2) compositions, but also induces robust antibody production more rapidly, by week 2, relative to the protein-only composition.

Active immunization using the immunogenic compositions described herein is able to induce functional T cells to SARS-CoV-2 or coronavirus antigens.

### Example 4: Immunogenic DNA Compositions Induce Production of SARS-CoV-2 Neutralizing Antibodies in Animals

A single 50 µg dose of DNA expression cassettes comprising compositions SVF-2, SVF-2.2, SVF-2.3, or only spike protein (as a control) was administered to BALC/c and C57BL/6 mice. Serum samples from the test mice were obtained two weeks following administration, and the presence of neutralizing antibodies specific for SARS-CoV-2 protein components was assessed by ELISA (end point titer) and in vitro neutralization assay. Results are shown below in Table 2 (BALB/c) and Table 3 (C57BL/6). Composition SVF-2.3 resulted in the production of anti-SARS-CoV-2 spike protein antibodies comparable with the composition of only spike protein, but also conferred immunogenicity against SARS-CoV-2 nucleocapsid protein in BALB/c mice. Serum from BALB/c mice treated with composition SVF-2.3 also successfully neutralized SARS-CoV-2 infection in an in vitro assay. (S=spike protein; RBD=receptor binding domain; NP=nucleocapsid protein). The same responses are shown two weeks after a second immunization administered three weeks after the first immunization (Tables 4 and 5).

TABLE 2

Quantification of BALB/c mice serum after DNA composition administration

| Composition | Anti-S ELISA | Anti-RBD ELISA | Anti-NP ELISA | Neutralization SARS-CoV-2 |
|---|---|---|---|---|
| SVF-2 | <60 | <60 | 2160 | <8 |
| SVF-2.2 | 60 | 60 | 1080 | <8 |
| SVF-2.3 | 1440 | 720 | 1080 | 8 |
| S DNA | 1440 | 360 | <60 | 8 |

TABLE 3

Quantification of C57BL/6 mice serum after DNA composition administration

| Composition | Anti-S ELISA | Anti-RBD ELISA | Anti-NP ELISA | Neutralization SARS-CoV-2 |
|---|---|---|---|---|
| SVF-2 | <60 | <60 | 360 | <8 |
| SVF-2.2 | <60 | <60 | 135 | <8 |
| SVF-2.3 | 360 | 210 | <60 | <8 |
| S DNA | 360 | 360 | <60 | 8 |

TABLE 4

Quantification of BALB/c mice serum after 2 rounds of DNA composition administration

| Composition | Anti-S ELISA | Anti-RBD ELISA | Anti-NP ELISA | Neutralization SARS-CoV-2 |
|---|---|---|---|---|
| SVF-2 | <60 | <60 | not tested | <8 |
| SVF-2.2 | 60 | 720 | not tested | <8 |
| SVF-2.3 | 51480 | 64800 | not tested | 256 |
| S DNA | 12960 | 12960 | not tested | 256 |

TABLE 5

Quantification of C57BL/6 mice serum after 2 rounds of DNA composition administration

| Composition | Anti-S ELISA | Anti-RBD ELISA | Anti-NP ELISA | Neutralization SARS-CoV-2 |
|---|---|---|---|---|
| SVF-2 | <60 | <60 | not tested | <8 |
| SVF-2.2 | <60 | <60 | not tested | <8 |
| SVF-2.3 | 36360 | 8280 | not tested | 128 |
| S DNA | 25960 | 25960 | not tested | 512 |

### Example 5: Additional Exemplary Constructs Are Immunogenic in Mice

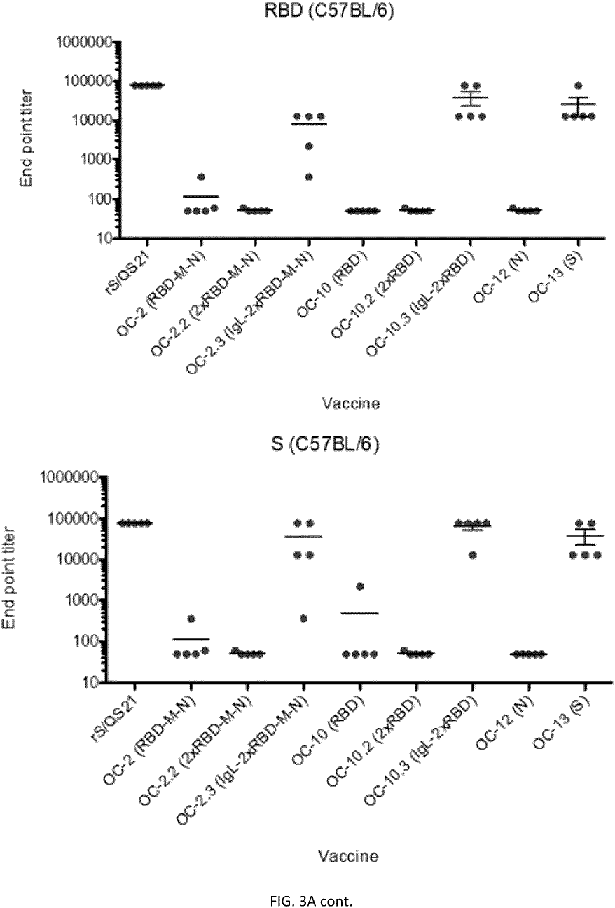

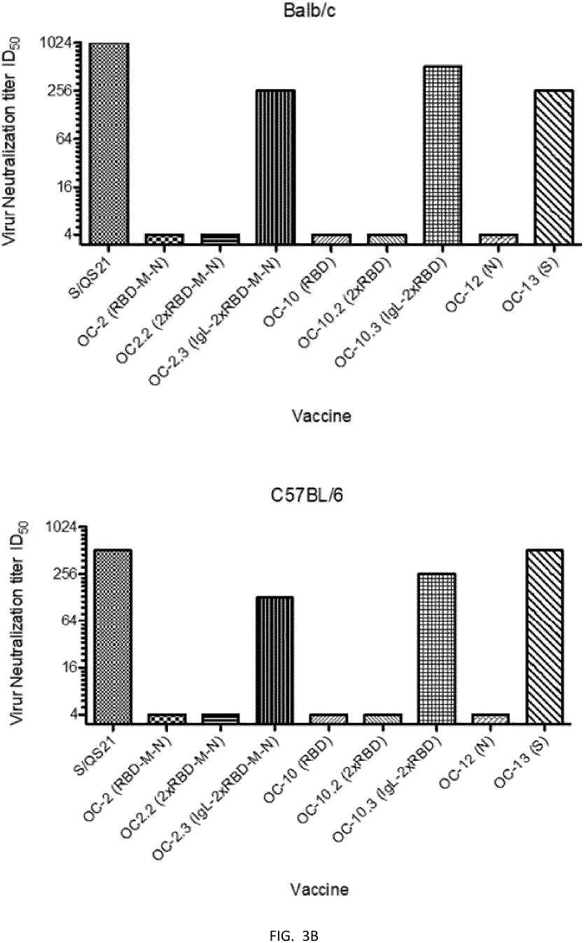

BALB/c and C57BL/6 mice were immunized at weeks 0 and 3 with 50 µg of plasmid construct DNA using in vivo EP. The constructs used were OC-2, OC-2.2, OC-2.3, OC-10, OC-10.2, OC-10.3, OC-12, and OC-13, with recombinant S protein with QS21 adjuvant used as control. Serum samples from the test mice were obtained two weeks following administration of the second dose, and the presence of neutralizing antibodies specific for SARS-CoV-2 RBD and S protein was assessed by ELISA (FIG. 3A). Levels are given as the end point titer defined as the highest dilution giving an optical density at 450 nm of twice the negative control at the same dilution. Constructs OC-2.3, OC-10.3, and OC-13 exhibited robust immunogenic properties in both BALB/c and C57BL/6 mice.

The in vitro neutralization of immunized mice serum against SARS-CoV-2 was assessed. Pooled serum samples from each group of mice were incubated with SARS-CoV-2 and then added to Vero-E6 cells. The level of viral cytopathic effect (CPE) was determined by inspection under microscope and the virus neuralization titer ID₅₀ was determined as the dilution of serum giving 50% inhibition of CPE (FIG. 3B). Mice immunized with constructs OC-2.3, OC-10.3, and OC-13 resulted in serum that robustly neutralized SARS-CoV-2 infectivity.

### Example 6: The Immunogenic Compositions Induce T Cell Responses in Mice

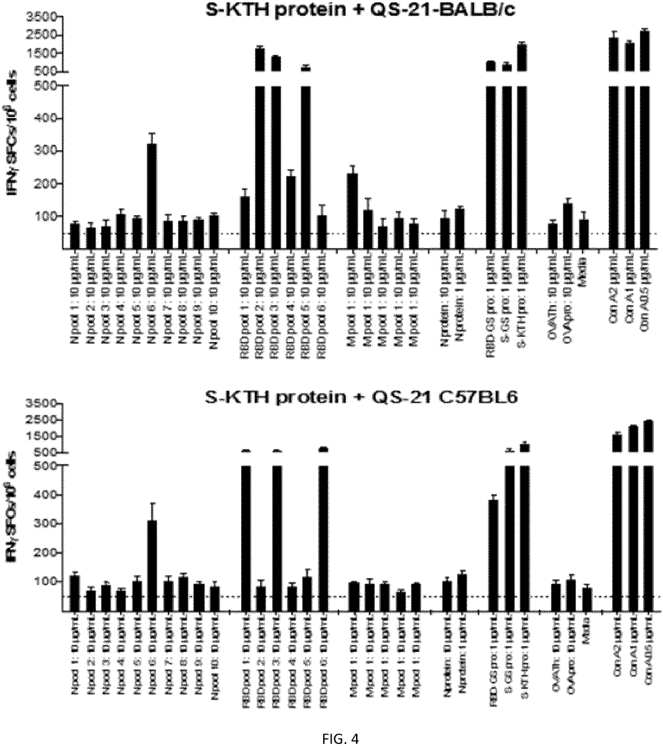

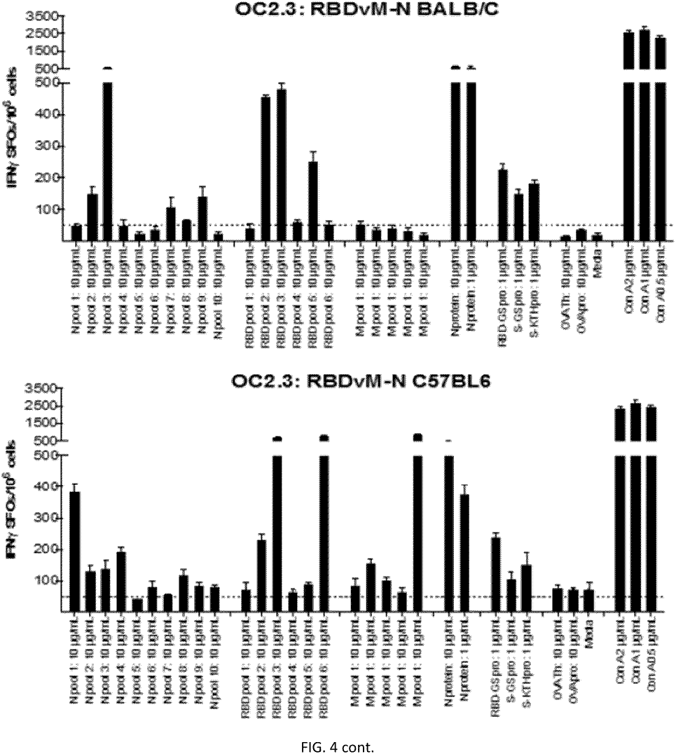

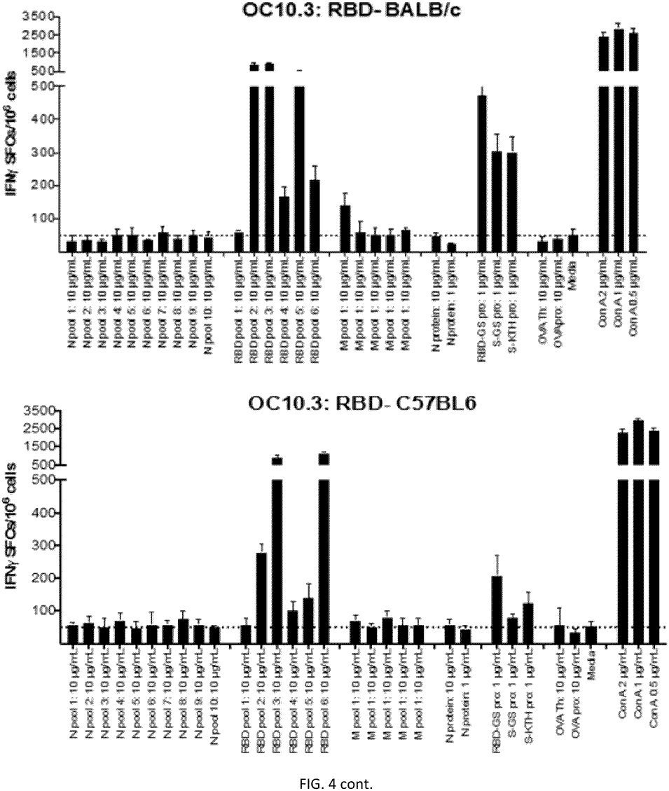

BALB/c and C57BL/6 mice were immunized at weeks 0 and 3 with 50 µg of OC-2.3 and OC-10.3 construct DNA using in vivo EP, with recombinant S protein with QS21 adjuvant as control. Responses of the mice T cells against peptide pools spanning the RBD, M, and NP proteins was detected by interferon gamma ELISpot (FIG. 4). “S-KTH” indicates recombinant S protein provided by Royal Technical University (KTH). “S-GS” indicates recombinant S protein obtained from Genscript (#Z03501). “RBD-GS” indicates recombinant RBD of S protein obtained from Genscript (#Z03479). These peptide pools were generated as 20 amino acid long peptides with 10 amino acids overlap. Ovalbumin peptides were used as negative control, and concanavalin A was used as positive control. Mice immunized with OC-2.3, which contains sequences for RBD, M, and NP protein, resulted in robust T cell activation against RBD and N peptides and protein, while M peptides were less reactive. Mice immunized with OC-10.3, which comprises only RBD, resulted in robust T cell activation only against RBD peptides and protein.

### Example 7: Sera From Immunized Animals Are Effective at Neutralizing SARS-CoV-2 Infection

The ability of induced antibodies to neutralize SARS-CoV-2 infection in vivo is further determined using the K18-hACE2 mice model or transiently hACE2-transgenic wild-type mice. Total IgG is purified from immunized and non-immunized rabbits and is injected in mice. The DNA prime/protein boost-induced antibodies protects, or significantly delays peak viremia in all challenged mice better than the DNA-only or protein-only compositions.

### Example 8: T Cell Response Against SARS-CoV-2 Epitopes Can Be Enhanced With a Prime/Boost Approach

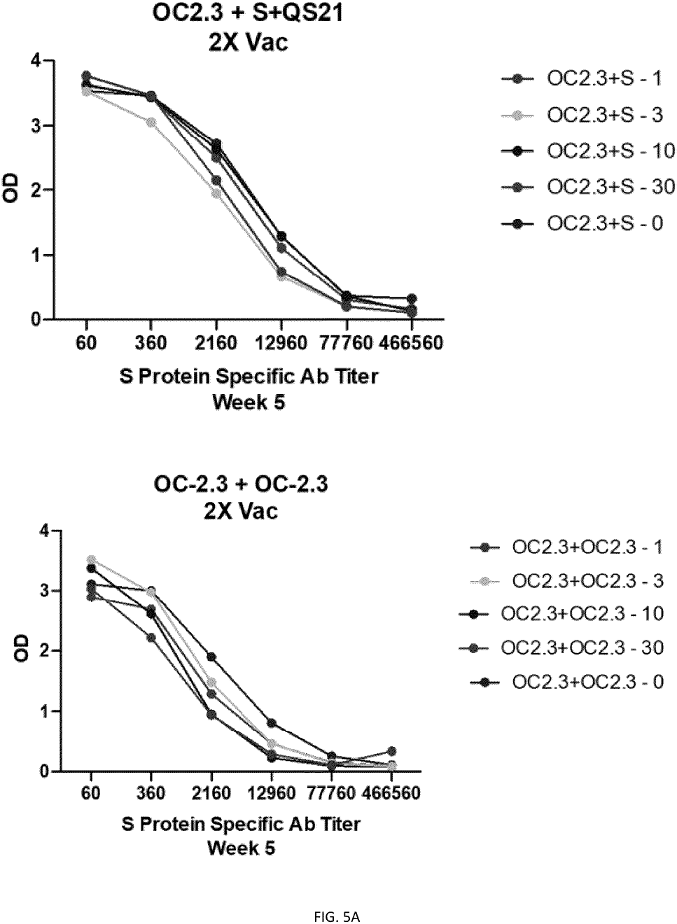

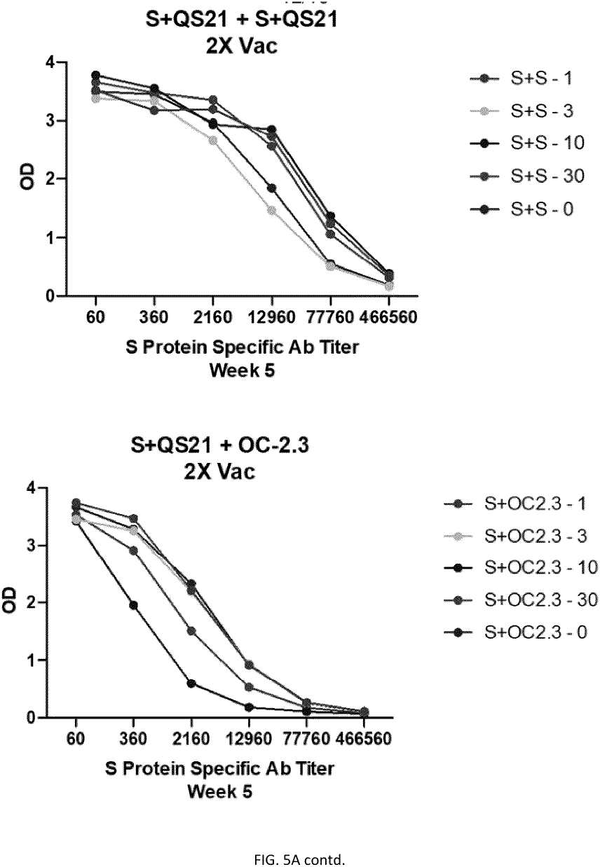

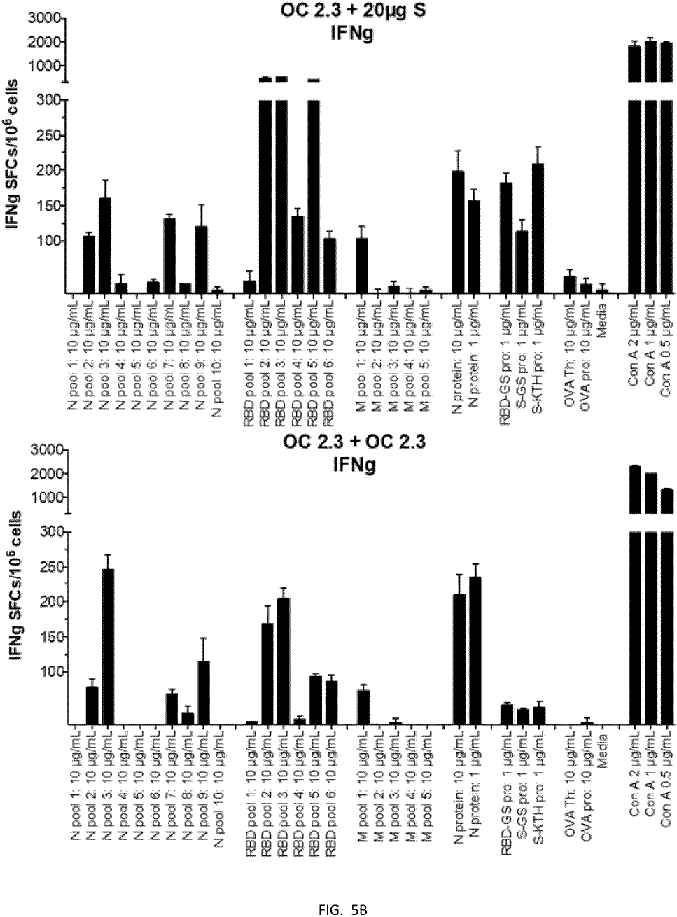

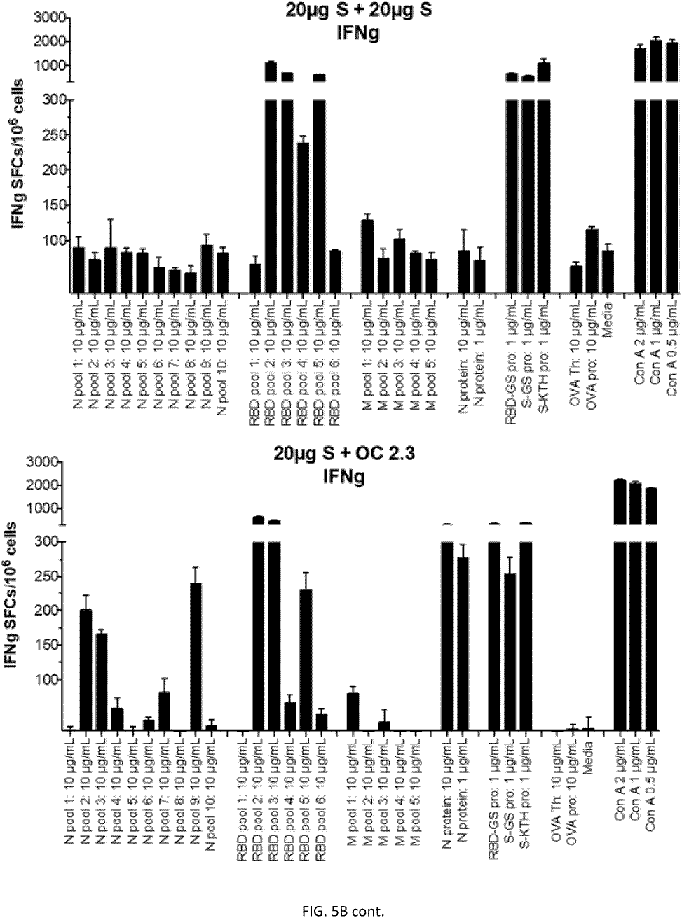

The effects of homologous (DNA only prime and boost; or protein only prime and boost) and heterologous priming (DNA prime, protein boost; or protein prime, DNA boost) with the OC-2.3 DNA construct and recombinant S protein with QS21 adjuvant (rS/QS21) was tested in BALB/c mice. The mice were immunized at weeks 0 and 3 with either 50 µg of plasmid construct DNA using in vivo EP, or recombinant S protein with QS21 adjuvant. FIG. 5A shows the anti-S protein titers in serum from in immunized mice (5 mice tested, labeled “0”, “1”, “3”, “10”, and “30”). Each of the 4 conditions (i.e. different combinations of S/QS21 peptide and OC-2.3 DNA as either prime or boost, or both). FIG. 5B shows T cell responses from the immunized mice towards peptide pools spanning the SARS-CoV-2 RBD, M, and NP proteins as detected by ELISpot. These peptide pools were generated as 20 amino acid long peptides with 10 amino acids overlap. Ovalbumin peptides were used as negative control, and concanavalin A was used as positive control. As seen for both the OC-2.3 DNA prime and rS/QS21 boost approach and the rS/QS21 prime and OC-2.3 DNA prime approach, the heterologous combination results in robust immunogenicity against RBD protein and peptides while also resulting in reactivity towards NP peptides and protein. This improved coverage of the SARS-CoV-2 viral components will provide improved protection against the virus as well as various strains or mutants where a certain component is conserved.

### Example 9: The Immunogenic Compositions Are Immunogenic in Rabbits and Non-Human Primates

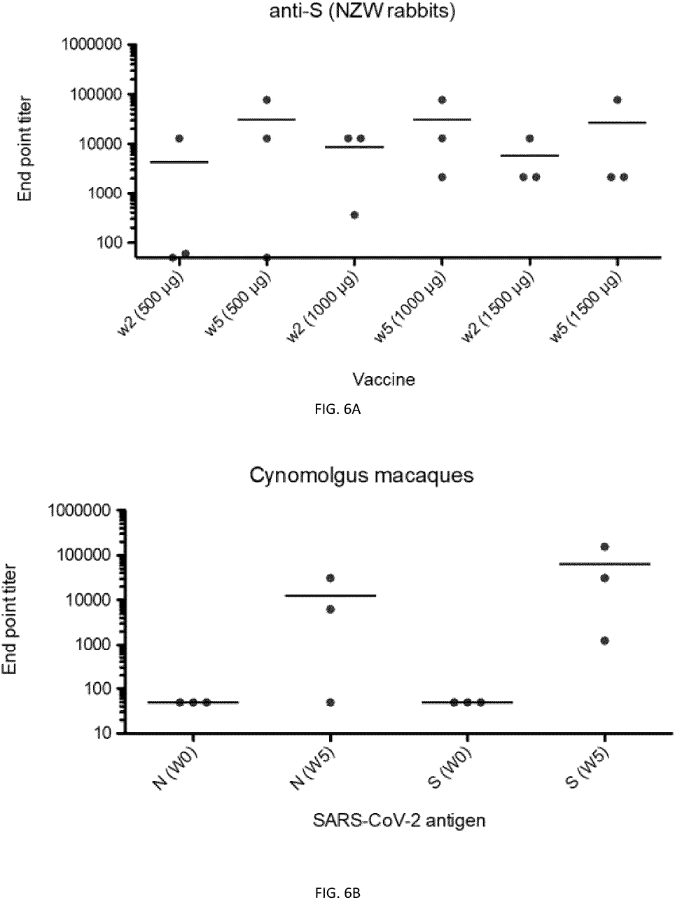

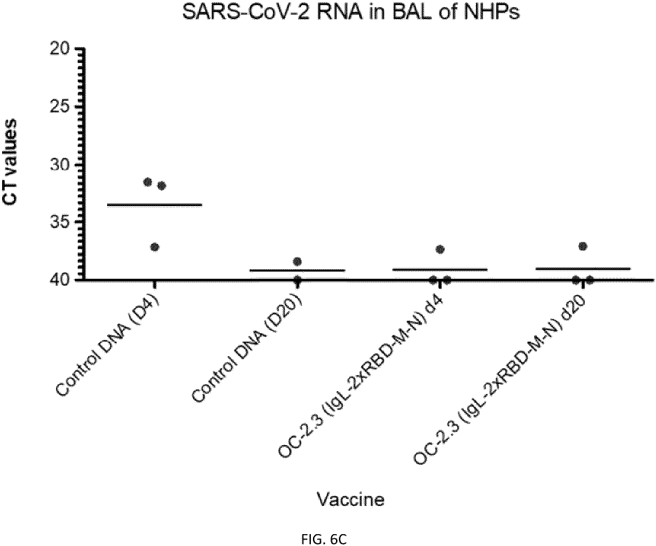

The immunogenic abilities of the OC-2.3 DNA construct in rabbits and cynomolgus macaques was assessed. The rabbits were administered with either 500, 1000, or 1500 µg of OC-2.3 DNA using in vivo EP at weeks 0 and 3. The macaques were administered with 1000 µg of OC-2.3 DNA using in vivo EP at weeks 0 and 3. The injection was performed using a single step procedure using a custom injection device. The anti-S antibody levels in the animals were assessed after the second administration (FIGS. 6A-B). Levels are given as the end point titer defined as the highest dilution giving an optical density at 450 nm of twice the negative control at the same dilution.

Cynomolgus macaques (groups of 3) were immunized with 1000 µg OC-2.3 or control DNA (HBV DNA) as two doses at weeks 0 and 3, and subsequently challenged with SARS-CoV-2 (0.5 mL intranasally and 4.5 mL intratracheally with 10⁶ pfu/mL). Bronchoalveolar lavage (BAL) samples were taken at days 4 and 20 post-challenge, and SARS-CoV-2 RNA was quantified by qPCR (FIG. 6C). A Ct value greater than 40 represents RNA levels below the detection limit. Monkeys immunized with OC-2.3 showed essentially undetectable levels of SARS-CoV-2 RNA at both days 4 and 20, whereas monkeys immunized with control DNA exhibited a detectable SARS-CoV-2 infection at day 4, and clearance of the infection by day 20. Quantification of antibody titers and presence of SARS-CoV-2 RNA in BAL is provided in Table 6. Leakage was noted with immunizations on subjects 4 and 5.

TABLE 6

Quantification of tested Cynomolgus macaques

| Histological finding | Control DNA (HBV) | | | SARS-CoV-2 DNA (IgE Leader-2xRBD-M-N; OC-2.3) | | |
|---|---|---|---|---|---|---|
| | Subject 1 | Subject 2 | Subject 3 | Subject 4 | Subject 5 | Subject 6 |
| Anti-S titer | < 50 | < 50 | < 50 | 31250 | 1250 | 31250 |
| Anti-HB V PreS 1 titer | 50 | 31250 | 31250 | < 50 | < 50 | < 50 |
| SARS-CoV-2 RNA in BAL day 4 (Ct value) | 31.52 | 31.86 | 37.14 | 37.34 | > 40 | > 40 |
| SARS-CoV-2 RNA in BAL day 20 (Ct value) | 38.39 | > 40 | Not tested | > 40 | 37.04 | > 40 |

### Example 10: Human Clinical Trials With an Exemplary Immunogenic Composition Candidate

The following example describes embodiments of using an immunogenic composition or product combination, optionally comprised of a nucleic acid component and a polypeptide component, used to treat or prevent viral infections caused by coronaviruses such as SARS-CoV-2.

The DNA prime/protein boost compositions are administered to human patients enterally, orally, intranasally, parenterally, subcutaneously, intramuscularly, intradermally, or intravenously. These human patients may be currently infected with SARS-CoV-2, previously infected with SARS-CoV-2, at risk of being infected with SARS-CoV-2, or uninfected with SARS-CoV-2.

The DNA prime doses are administered first, at an amount of 1, 10, 100, 1000 ng, or 1, 10, 50, 100, 200, 300, 400, 500, 600, 700, 800, 900, 1000 µg, or 1, 10, 100, 200, 300, 400, 500, 600, 700, 800, 900, 1000 mg, or any amount within a range defined by any two of the aforementioned amounts, or any other amount appropriate for optimal efficacy in humans. After the first DNA prime dose, 1, 2, 3, 4, or 5 additional DNA prime doses can be administered 1, 2, 3, 4, 5, 6, 7, 8, 9, 10, 11, 12, 24, 36, or 48 days or weeks or any time within a range defined by any two of the aforementioned times after administration of the previous DNA prime dose, e.g., within 1-48 days or 1-48 weeks. The protein boost doses are administered following the DNA prime doses, at an amount of 1, 10, 100, 1000 ng, or 1, 10, 50, 100, 200, 300, 400, 500, 600, 700, 800, 900, 1000 µg, or 1, 10, 100, 200, 300, 400, 500, 600, 700, 800, 900, 1000 mg, or any amount within a range defined by any two of the aforementioned amounts, or any other amount appropriate for optimal efficacy in humans. The first protein boost dose is administered 1, 2, 3, 4, 5, 6, 7, 8, 9, 10, 11, 12, 24, 36, or 48 days or weeks or any time within a range defined by any two of the aforementioned times after administration of the final DNA prime dose. After the first protein boost dose, 1, 2, 3, 4, or 5 additional protein boost doses can be administered 1, 2, 3, 4, 5, 6, 7, 8, 9, 10, 11, 12, 24, 36, or 48 days or weeks or any time within a range defined by any two of the aforementioned times after administration of the previous protein boost dose.

Patients will be monitored for successful response against SARS-CoV-2, for example, production of anti-S protein, anti-RBD, anti-M protein, anti-NP protein, anti-SARS-CoV2 or anti-coronavirus antibodies. In the conditions where HDAg sequences are included, anti-HDAg antibodies in sera is also tested. Also expected is the rapid activation of T cells and other immune cells when exposed to SARS-CoV-2 or coronavirus antigens, and protection against future infections by SARS-CoV-2 or coronavirus.

In patients currently infected, previously infected, or at risk for infection SARS-CoV-2 or coronavirus, administration of the DNA prime/protein boost compositions may be performed in conjunction with antiviral therapy. Potential antiviral therapy therapeutics include but are not limited to dexamethasone, favipiravir, favilavir, remdesivir, tocilizumab, galidesivir, sarilumab, lopinavir, ritonavir, darunavir, ribavirin, interferon-α, pegylated interferon-α, interferon alfa-2b, convalescent serum, or any combination thereof. Patients will be monitored for side effects such as dizziness, nausea, diarrhea, depression, insomnia, headaches, itching, rashes, fevers, or other known side effects of the provided antiviral therapeutics.

### Example 11: Materials and Methods

### Animals

Female BALB/c (H-2d ) mice were obtained from Charles River Laboratories, Sulzfeld, Germany. Female B6.Cg-Tg(K18-ACE2)2Prlmn/J mice (JAX stock #034860) were purchased from Jackson Laboratory, USA. All mice were 8-12 weeks old at the start of the experiments and maintained under standard conditions at Preclinical laboratory (PKL), Karolinska University Hospital Huddinge, Sweden or Astrid Fagraeus Laboratory (AFL), Karolinska Institutet, Solna, Sweden. Nine New Zealand White rabbits were purchased from Charles River, France and kept at AFL. All animal procedures were granted by regional animal ethics committees (approvals Dnr. 03634-2020, 17114-2020 and 16676-2020). Challenge experiments were performed in a biosafety level (BSL)-3 animal facility approved by the Swedish Board of Work Environment Safety.

### DNA Plasmids and Recombinant Proteins

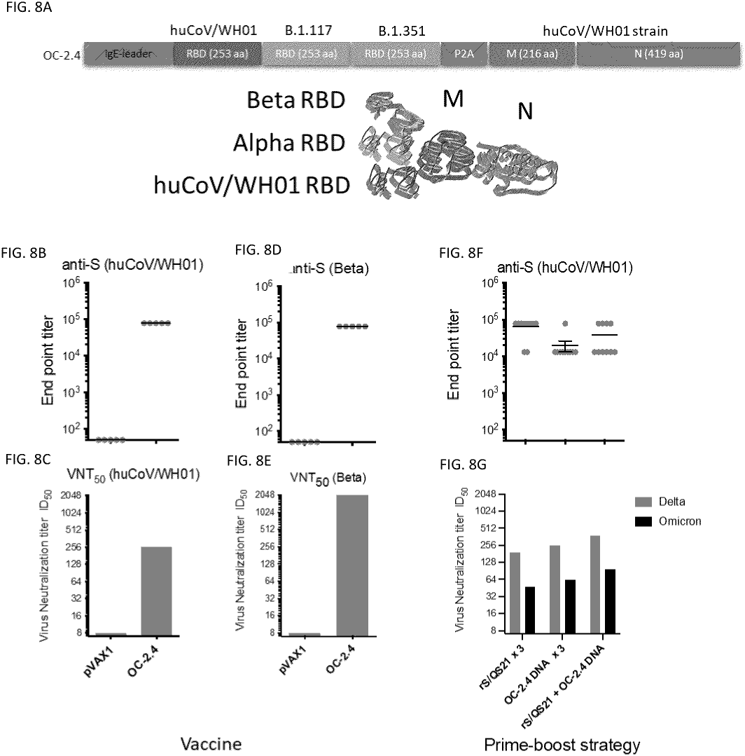

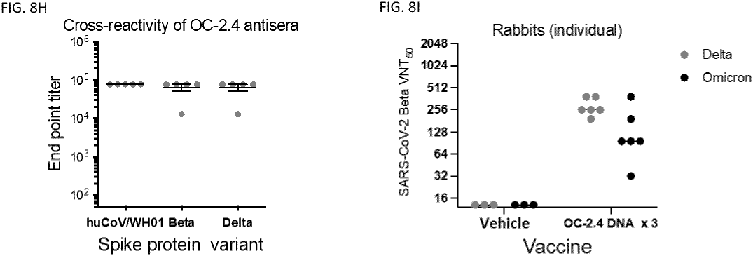

Vaccine candidate genes were generated based on the sequence from the huCoV-19/WH01 strain (FIG. 8A). The genes contained the full Spike protein or a combination of the RBD, N, and M proteins, with a autoproteolytic P2A sequence. All sequences were codon optimized for expression in human cells and were synthesized by a commercial vendor (Genscript). Plasmids were grown in TOP10 Escherichia coli cells (Life Technologies) and purified for in vivo injections using Qiagen Endofree DNA purification kit (Qiagen) according to the manufacturer’s instructions. The correct gene size was confirmed by restriction enzyme digests using BamHI and XbaI (Fast Digest; Thermo Fisher Scientific), and sequencing. Recombinant N protein was designed in-house and produced by Genscript (32878912; Ahlen et al, 2020). Recombinant S, RBD, and M was purchased from Genscript. The spike proteins were produced as full length by transient protein production in mammalian cells (Expi293). To facilitate trimerization of the full-length spike, a C-terminal T4 fibritin trimerization motif was included according to Wrapp et al (2020). Further a strep-tag, fused to the C-terminal was used for purification (Hober et al, 2021). The beta version (B1.351) of the spike was produced with three mutations in the RBD part (K417N E484K N501Y).

### Peptides

A total of 42 20-mer peptides with 10 aa overlap, corresponding to the huCoV-19/WH01 RBD (25 peptides), M (22 peptides), and N (41 peptides) and Bat N (42 peptides) were purchased from Sigma-Aldrich (St. Louis, MO). The peptides were divided in pools of 4-5 or 8-10 peptides/pool depending on experimental setup.

### Immunization and Infection Schedules in Mice and Rabbits

BALB/c (H-2d ) or C57BL/6 (H-2b ) (n = 5) mice were immunized up to three times with 3-week intervals, and sacrificed 2 weeks after the last immunization for spleen and blood collection as described previously (Ahlen et al, 2020; Maravelia et al, 2021). Twenty K18-hACE2 mice were divided into four groups (n = 5) and immunized with indicated vaccines. Each K18-hACE mouse received three immunizations with 3 weeks between each injection. Two weeks after the last immunization, the K18-hACE2 mice were infected with SARS-CoV-2 Beta. Immunization method in brief, BALB/c or K18- hACE2 mice (five per group) were immunized intramuscularly in the Tibialis cranialis anterior muscle with 50 µg plasmid DNA in a volume of 50 µl sterile phosphate-buffered saline (PBS) by regular needle (27G) injection followed by in vivo electroporation using a Cliniporator2 device (IGEA) using two needle electrodes. Prior to vaccine injections, mice were given analgesic and kept under isoflurane anesthesia during the vaccinations. During in vivo electroporation (in both mice and rabbits), a 1-ms 600-V/cm pulse followed by a 400-ms 60-V/cm pulse pattern was used to facilitate better uptake of the DNA. In addition, groups of mice were injected subcutaneously at the base of the tail with recombinant SARS-CoV-2 spike (S) or nucleo (N) protein mixed (1:1) with QS21 adjuvant (GMP grade, Alpha diagnostics). Each New Zealand white rabbit was immunized with 84 µg or 840 µg of OC-2.4 DNA vaccine or only formulation buffer (Tris-EDTA, pH 7.4) vehicle. Injection were administered in the right Quadriceps muscle in 500 µl followed by in vivo electroporation under anesthesia using the GeneDriVe (IGEA) device and a 4 needle electrode array at a depth of 21 mm.

### Mouse Challenge Model

Two weeks post the last immunization, the K18-hACE2 mice were challenged with 1 × 10⁵ pfu SARS-CoV-2 Beta variant via intranasal administration in a volume of 40 µl in a BSL-3 facility. The health of the animals was assessed daily for up to 13 days and evaluated based on several parameters, including body weight, general condition, piloerection, as well as movement and posture. At the time of euthanization, blood, nasal lavage, lungs, and spleen were collected.

### Detection of IgG-specific Antibodies

Serum from mice and rabbits were used for detection of immunoglobulins against S or N protein. In brief, plates were coated with 1 µg/ml of recombinant S or N protein (Genscript) in 50 mM of Sodium Carbonate buffer pH 9.6 overnight at 4° C. Plates were blocked by incubation with dilution buffer (phosphate-buffered saline, 2% goat serum, 1% BSA) for 1 h at 37° C. Serum was added in serial dilutions with a starting dilution of 1:60 and then in serial dilution of 1:6. Serum antibodies were detected by an alkaline phosphatase conjugated goat anti-mouse IgG (Sigma A1047) 1:1,000 or mouse anti-rabbit IgG (Sigma A2556) 1:1,000 and visualized using p-nitrophenyl phosphate substrate solution. Optical density (OD) was read at 405 nm with a 620 nm background. Antibody titers were determined as endpoint serum dilutions at which the OD value was at least three times the OD of the negative control (nonimmunized or control animal serum) at the same dilution.

### Detection of Specific IFN- Γ Producing T Cells and Antibodies

Two weeks post last vaccination, splenocytes from each group of wild-type mice or peripheral blood mononuclear cells (PBMCs) from rabbits were harvested and tested for their ability to induce specific T cells based on IFN- y secretion after peptide or protein stimulation for 48 h essentially as described (Hawman et al, 2021; Maravelia et al, 2021) using a commercially available enzyme-linked immunospot (ELISpot) assay (Mabtech).

### Virus Propagation

The SARS-CoV-2 huCoV-19/WH01, Beta, and Omicron strains were isolated from patient samples at the Public Health Agency of Sweden and confirmed by sequencing. The SARS-CoV-2 Delta variant was provided by Dr. Charlotta Polacek Strandh, Statens Serum Institute, Copenhagen, Denmark. All variants were propagated on VeroE6 cells and titered using a plaque assay as previously described (Varnaite_ et al, 2020), with fixation after 72 h. The huCoV-19/ WH01, Delta, and Omicron strains used in this study was passaged three times and the Beta strain two times. All infectious experiments were performed in a BSL-3 facility approved by the Swedish Board of Work Environment Safety.

### Neutralization of SARS-CoV-2 in Vitro

Titer of neutralizing antibodies in serum from mice and rabbits were determined by CPE-based microneutralization assay. For mice, serum from each vaccination group was pooled, while for rabbits serum from each individual was tested. Briefly, serum was heat inactivated at 56° C. for 30 min before serial diluted 2- fold. Each dilution was conducted in quadruplets and mixed with 500 pfu of SARS-CoV-2 huCoV-19/WH01, Beta, Delta, or Omicron in a 1:1 dilution. After 1 h of incubation at 37° C., 5% CO2 100 µl of serum-virus mix were added to Vero E6 cells on a 96-well plate (20 × 104 cells/well) and incubated for 72 h at 37° C., 5% CO2. CPE for each well was determined using a Nicon Eclipse TE300 microscope. As controls, wells with medium only, diluted serum only, virus only, and serum known to contain SARS-CoV-2 neutralizing antibodies mixed with virus were included in each experiment. All infectious experiments were performed in a BSL-3 facility approved by the Swedish Board of Work Environment Safety.

### PCR/Viral RNA

Trizol (Sigma-Aldrich) in a ratio of 1:3 was used to inactivate potential virus in nasal lavage samples (50 11) from SARS-CoV-2-infected K18-hACE2 mice. For lung and spleen, PBS was added to each sample (1 g/ml) and pestles were used to crush the organs. Thereafter, the samples were centrifuged (5 min at 7,000 rpm) and 50 µl of each lung or spleen sample was added to Trizol (1:3). Total RNA was extracted using the Direct-zol RNA Miniprep kit (Zymo Research) according to the manufacturer’s instructions. Viral RNA were thereafter measured by quantitative real-time polymerase chain reaction (qRT-PCR) using TagMan Fast Virus 1-Step master mix (Thermo Fisher Scientific) with primers and probe for the SARS-CoV-2 E gene.

Forward: 50 - ACAGGTACGTTAATAGTTAATAGCGT -30

Reverse: 50 - ATATTGCAGCAGTACGCACACA -30

Probe: FAM- ACACTA GCC ATC CTT ACT GCG CTT CG MGB

For lung and spleen samples, mouse ACTB mix (Thermo Fisher Scientific) was used as endogenous control. The PCR reaction was performed using a capillary Roche LightCycler 2.0 system.

### Histological Analysis

The lungs were formalin fixed, embedded in paraffin, and sectioned for H&E staining. The sections were analyzed by an independent veterinary pathologist blinded to the treatment groups. All sections were scored according to bronchial and alveolar signs of inflammation and disease.

### Statistical Analysis

Data were analyzed with use of on GraphPad Prism V.5 software and Microsoft Excel V.16.13.1. The group size of animals were kept to a minimum (5 female mice or 3 + 3 male and female rabbits) to allow for a meaningful statistical analysis size. All experiments included replicate determinations of three or five replicates, which relates to both the number of animals and the number of replicates in the in vitro assay.

### Example 12: Immunogenicity of the Universal SARS-CoV-2 Vaccine

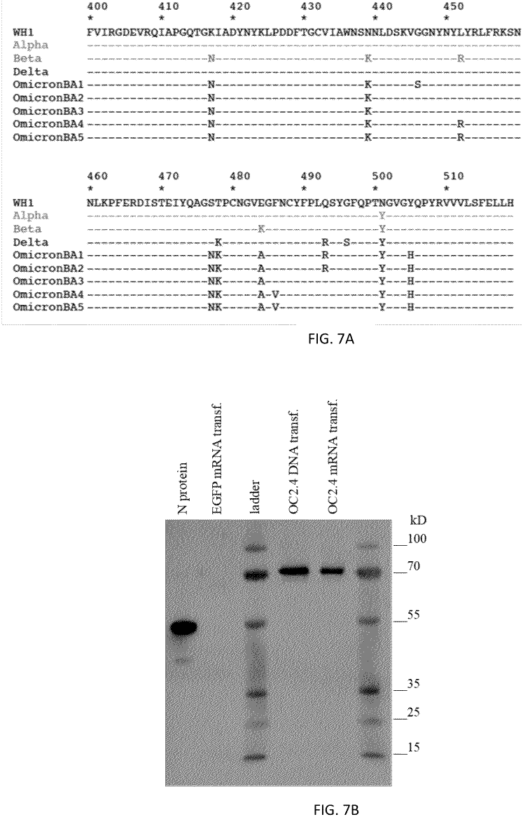

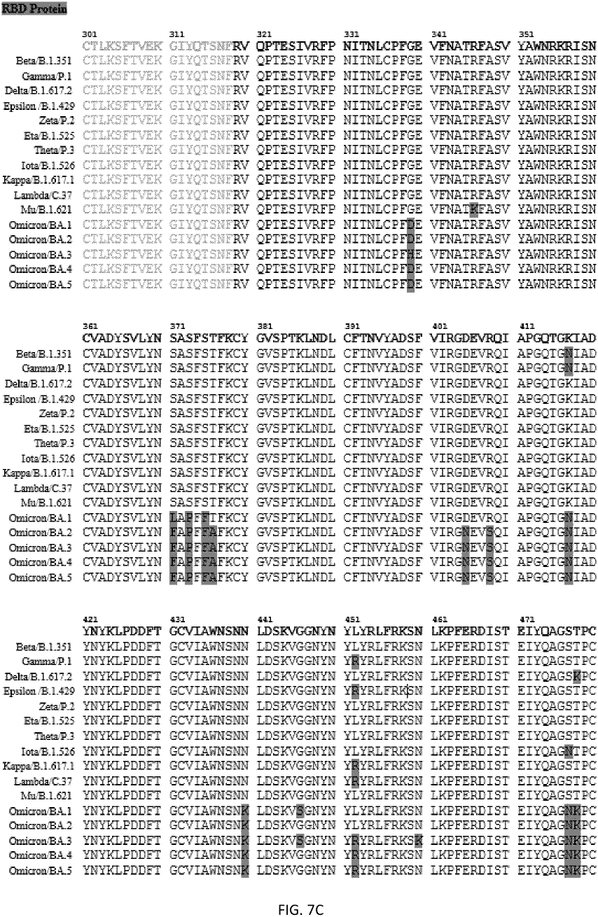

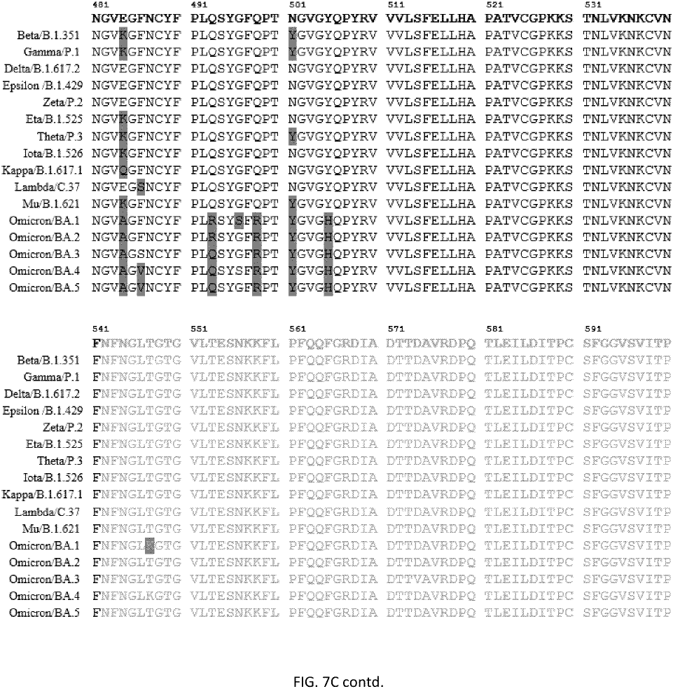

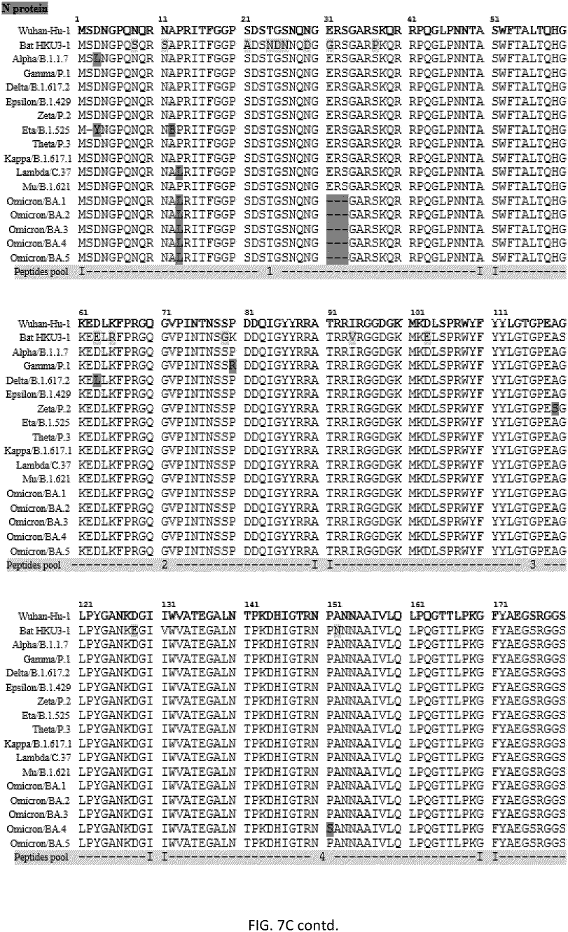

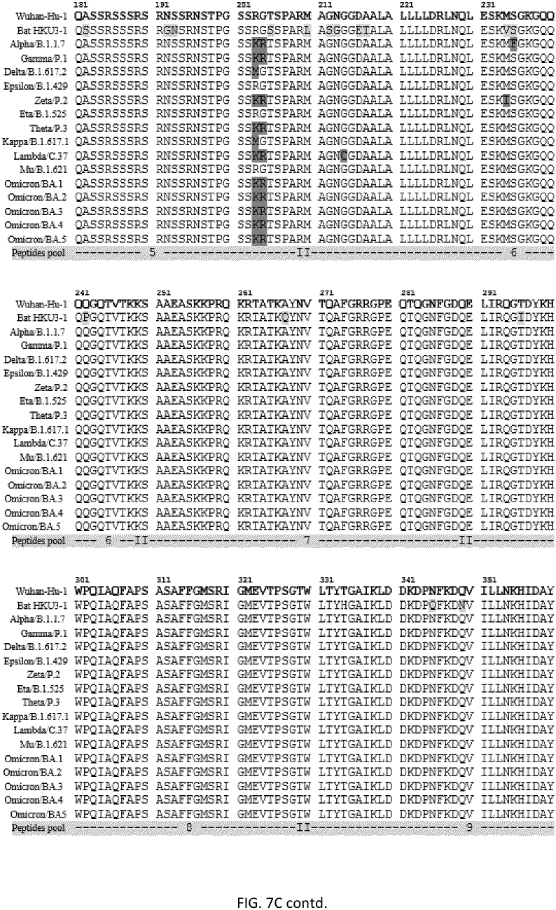

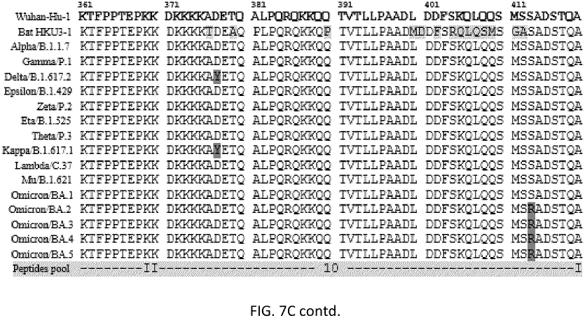

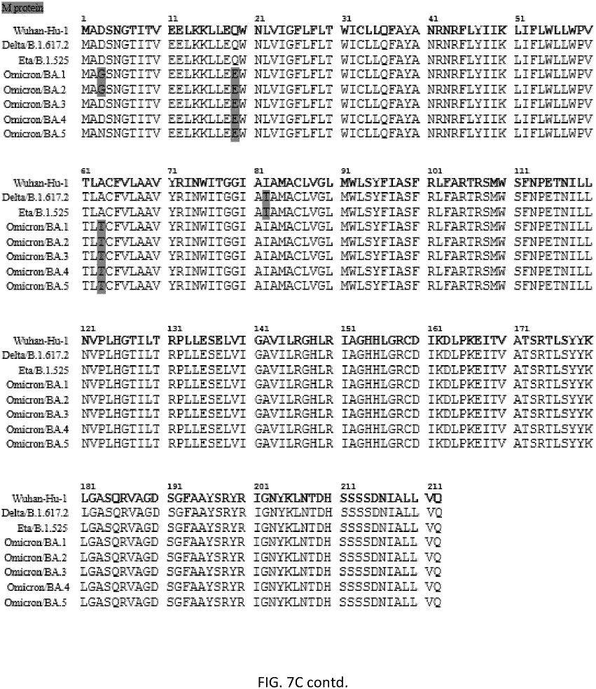

Although SARS-CoV-2 can infect multiple species (Shi et al, 2020), it is clear that bats are reservoir for the origin of most human coronaviruses (Hu et al, 2015). The spike proteins of SARS-CoV and SARS-CoV-2 induce cross-reactive T cells, but poorly cross-reactive Nabs (Le Bert et al, 2020). Among the structural proteins of SARSCoV and SARS-CoV-2, the envelope protein M and the nucleocapsid protein N have a higher genetic similarity to other animal SARS-CoV viruses than the receptor-binding S envelope protein (Hu et al, 2015; Latinne et al, 2020). T cells reactive to these two antigens show a higher cross-reactivity across betacoronaviruses (Ahlen et al, 2020; Le Bert et al, 2020). To take advantage of this, we designed a more universal SARS-CoV (OC-2.4) vaccine containing receptor-binding domain (RBD) loops of the S protein corresponding to the huCoV-19/WH01, Alpha, and Beta variants, combined with the M and N proteins of the huCoV-19/WH01 variant (OC2.4) (Dai et al, 2020; FIG. 7, FIG. 8A). In particular, Beta variant share some of the spike mutations with the recently emerged Omicron variant BA.5, which supports the use of the Beta variant. An autoproteolytic P2A sequence was inserted between the RBD and the M and N proteins to avoid interference with the folding of the RBD. The M and N proteins were expressed as a fusion protein (FIG. 7). Alignments between RBD, M, and N proteins between SARS-CoV strains and variants are shown in FIG. 7C. FIG. 8A illustrates the vaccine design and the concept of inducing both NAbs and broadly cross-reactive T cells. As control vaccines, we used either a pVAX plasmid without insert, or a recombinant S protein (huCoV-19/WH01) mixed with QS21 adjuvant. The aim of a universal vaccine is to induce broadly reactive antibodies and T cells, that complements the COVID-19 vaccines already in use. As the S protein differs more and more between SARS-CoV strains and variants (FIG. 7), cross-reactive T cells targeting other regions may be of a growing importance.

We first immunized Balb/c mice with the OC-2.4 DNA using in vivo electroporation (EP) and found that they developed high levels of antibodies binding to recombinant S proteins of the huCoV19/WH01, Beta, and Delta variants (FIGS. 8B, D, F and H) as well as to the N protein (mean ± SD anti-N endpoint titer: 25,920 ± 28,979). The S-specific antibodies effectively neutralized both the huCoV-19/WH01 and Beta variants of SARS-CoV-2 in vitro (FIGS. 8C and E).

In mice, priming with recombinant huCoV-19/WH01 S protein in adjuvant and boosting with the universal SARS-CoV DNA vaccine OC-2.4 effectively enhanced anti-S levels by 10- to100-fold to huCoV-19/WH01 S protein (FIG. 8F). Importantly, heterologous boosting with the universal DNA vaccine OC-2.4 induced higher neutralization levels than homologous boosting (FIG. 8G). Also, homologous boosting with the universal DNA vaccine OC-2.4 seemed superior in inducing NAbs to the Omicron variant (FIG. 8G).

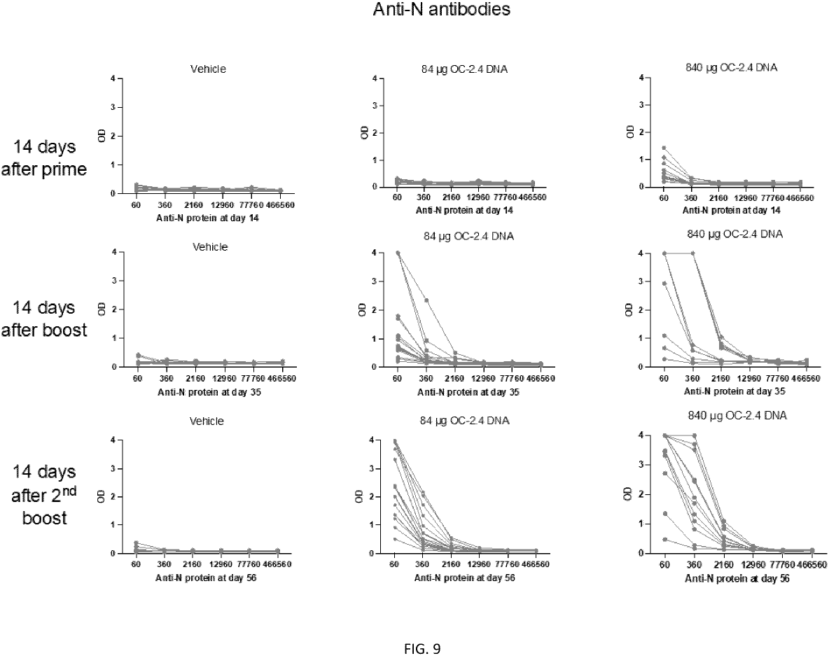

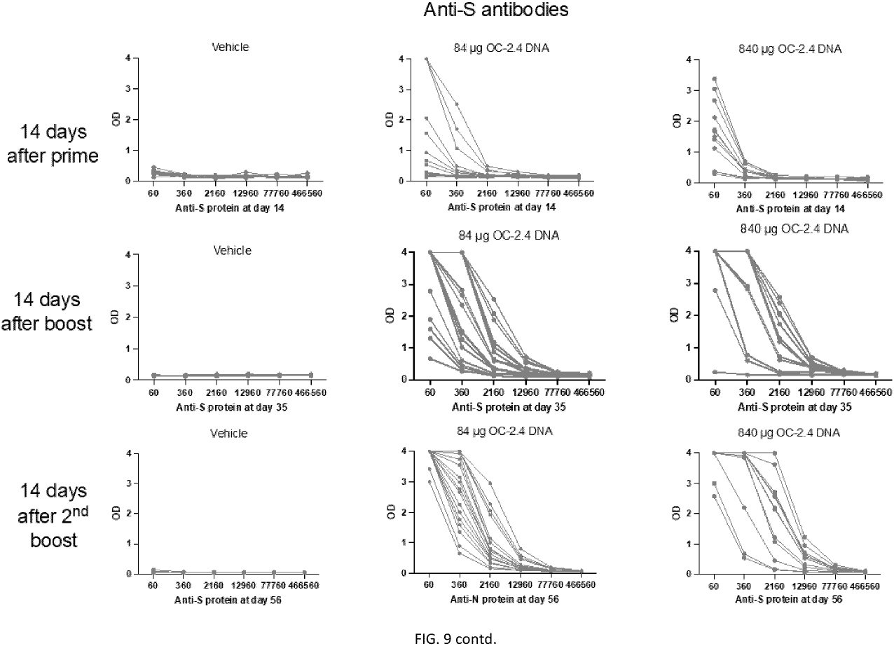

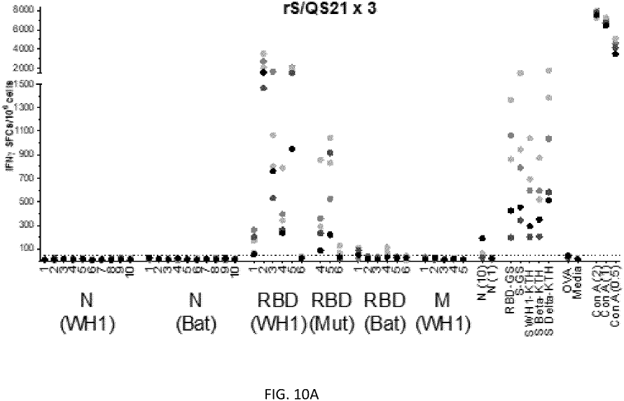

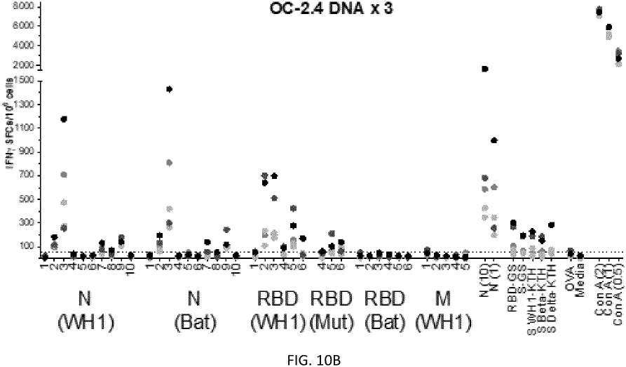

In rabbits, we found that one, two, and three doses of the OC-2.4 DNA vaccine induced high levels of anti-S antibody that effectively neutralized both Delta and Omicron variants of SARSCoV-2 (FIG. 8I, FIG. 9). Thus, the inclusion of three RBD loops was effective at inducing broadly neutralizing antibodies to the huCoV-19/WH01, Beta, Delta, and Omicron variants. As a comparison, three doses of a recombinant spike protein in adjuvant corresponding to the huCoV-19/WH01 variant, was generally less effective in priming NAbs to the Beta, Delta, and Omicron variants (FIG. 8).

### Example 13: The Universal SARS-CoV-2 Vaccine Induces Broadly Cross-Reactive T Cell Responses

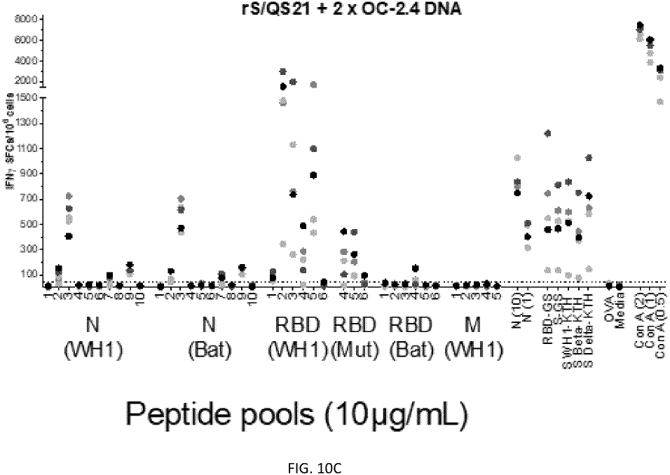

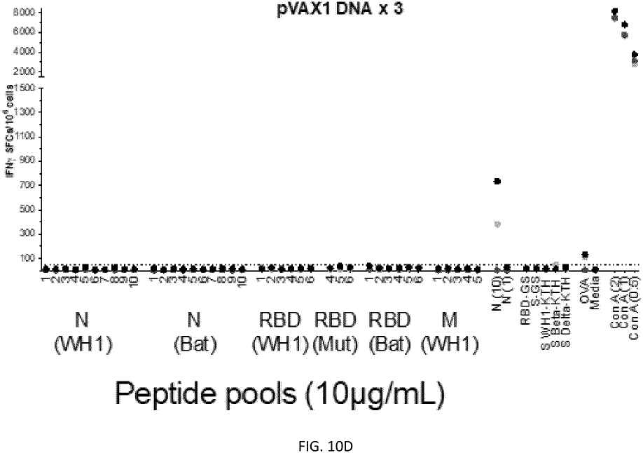

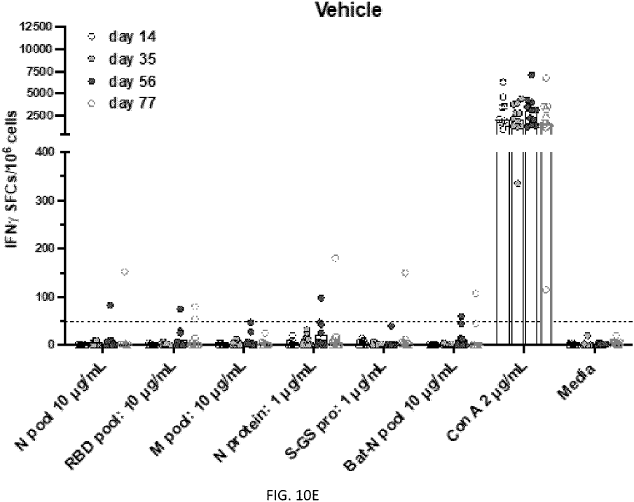

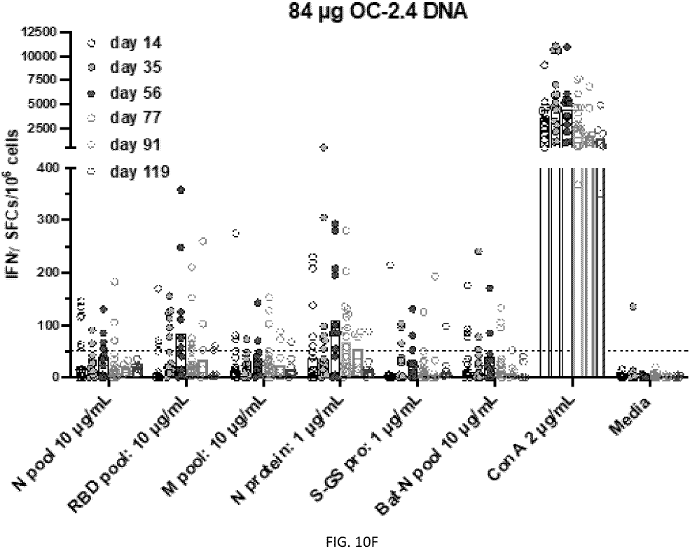

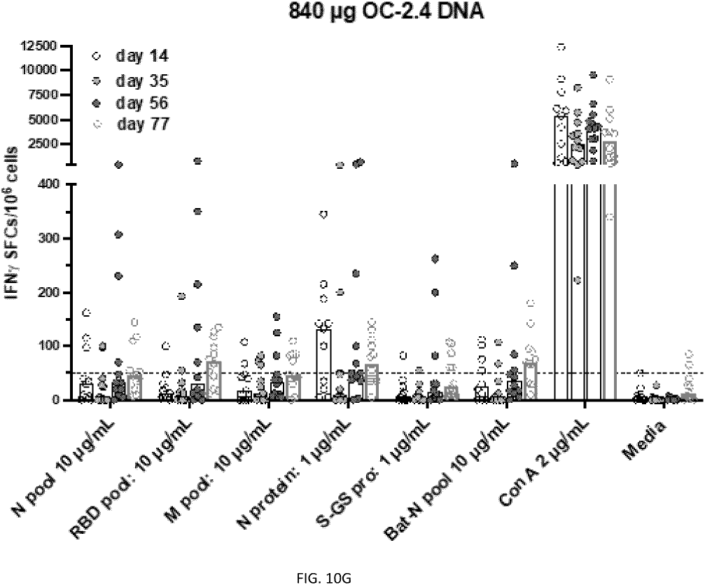

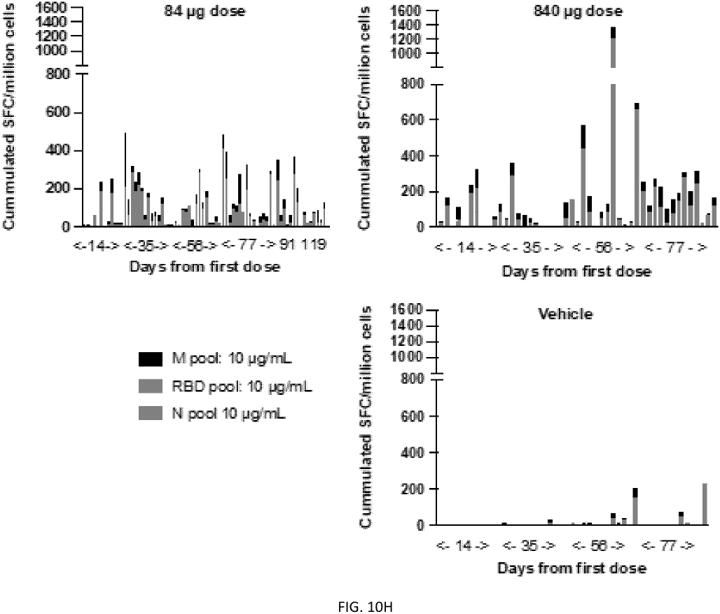

Next, splenocytes (mice) and peripheral mononuclear cells (rabbits) from animals immunized with the universal SARS-CoV DNA vaccine OC-2.4 were analyzed for reactivity to S, M and M peptides and antigens, and cross reactivity of T cells to the Bat CoV N sequences (FIG. 10). Mice develop T cells reactive to all components of the vaccine depending on the mouse strain, with Balb/c mice primarily developing T cells to S and N (FIGS. 10A-D). Importantly, the mean number of spot-forming cells (SFCs) per million splenocytes to both WH1 N and Bat N peptide pools number 3 (FIG. 10), were significantly higher in the group that was primed with rS/QS21 and boosted with OC-2.4 DNA, as compared to their group only vaccinated with rS/QS21 (569 vs. 15, and 573 vs. 19, respectively, SFCs/ 10⁶ PBMC, P < 0.0001, Student’s t-test, Graph Pad Prism). Thus, boosting with OC-2.4 DNA broadens the T cell reactivity in a host primed with a S-based vaccine. We could detect T cells to one or more components of the vaccine in PBMC from a majority of vaccinated rabbits at one or more time points, although some rabbits failed to develop detectable T cell responses at any time point (FIGS. 10E-H). In the vehicle group, none of the rabbits were reactive (> 50 SFC_{S}/10⁶ PBMCs) at days 14 (0/9) or 35 (0/11), whereas 1/11 at day 56 and 1/12 at day 77 were reactive to the N protein, as compared to 6/15 (40%; P = 0.0519, Fisher’s exact test), 7/15 (47%; P < 0.05, Fisher’s exact test), 9/13 (69%; P < 0.01, Fisher’s exact test), and 10/18 (56%; P < 0.05, Fisher’s exact test), respectively, at the same days in the group receiving 84 µg doses of OC-2.4 (FIG. 10). The heterologous prime-boost strategy with priming with a spike-based vaccine and boosting with the OC-2.4 DNA vaccine effectively broadened the T cell response and introduced new T cell specificities to the spike-based vaccine (FIGS. 10A-D). This is a situation that is anticipated for the initial human use of this vaccine as a booster vaccine.

Importantly, the T cells reactive to the N protein were, unlike T cells reactive to the spike/RBD protein, highly cross-reactive to sequences corresponding to the Bat-CoV N in both mice and rabbits (FIG. 10). Thus, these data shows that the vaccine induces highly cross-reactive T cells that also cross react with animal SARS-CoV proteins.

We also performed a full toxicological analysis of the rabbits following four doses of the vehicle or the OC-2.4 vaccine delivered by in vivo EP. At the injection site, degeneration and inflammation were seen 2 days after the last vaccination with vehicle for both the 84 µg and 840 µg dose. At 14 days after the last vaccination, these changes had healed completely in all DNA-vaccinated animals, except in one female that had a slight inflammation and degeneration. Also, no changes in biochemical or hematological markers could be linked to the treatments. Thus, apart from the OC-2.4 vaccine being immunogenic it also seems safe without any signs of pathology, except for immediately after vaccination at the treatment site, which is to be expected from a vaccine.

### Example 14: The Universal SARS-CoV-2 Vaccine Protects Against K18 Mice Against Lethal Challenge With SARS-CoV-2 Beta Variant.

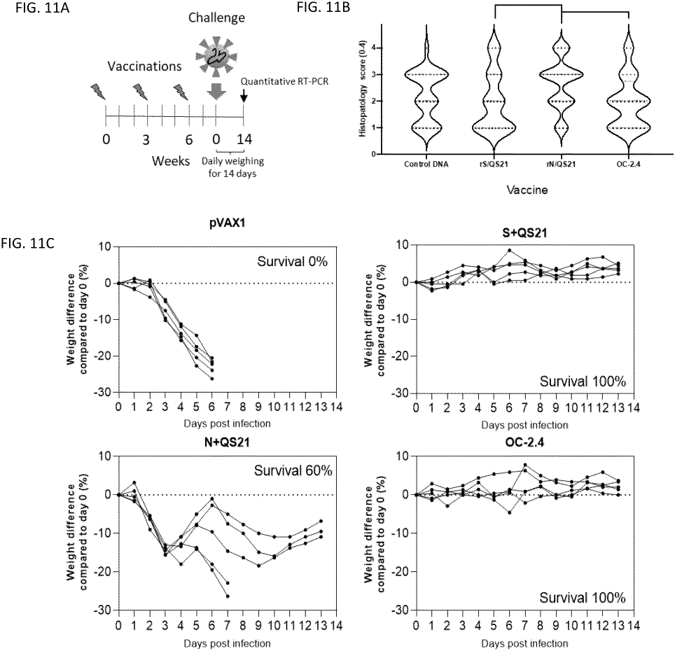

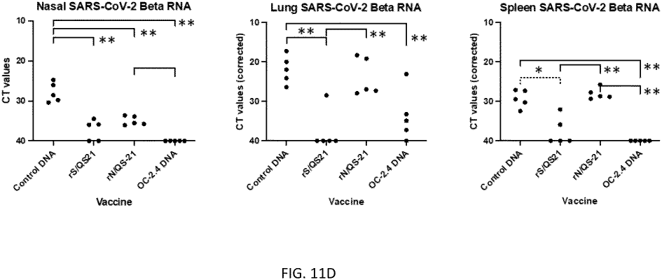

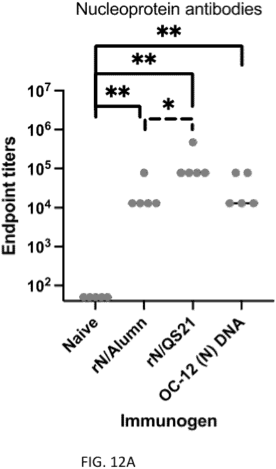

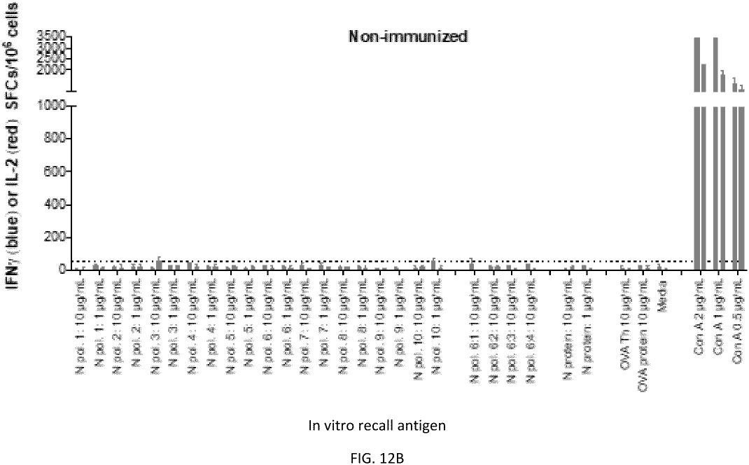

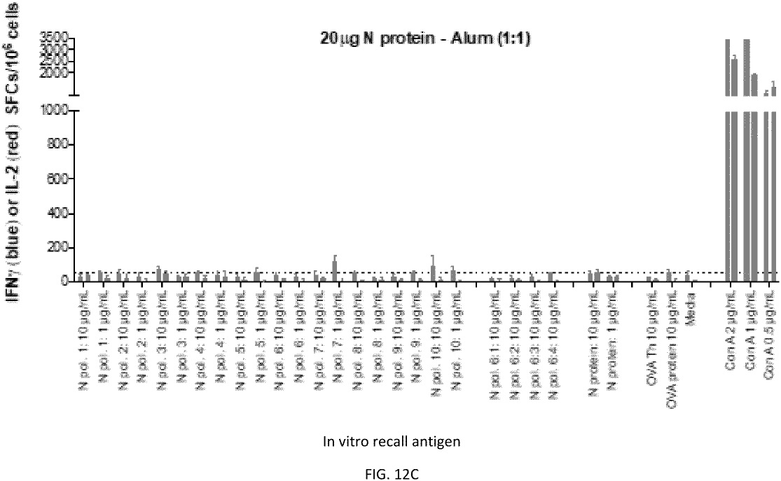

Finally, we analyzed the ability of the universal DNA vaccine OC-2.4 to induce protective immune responses against a lethal challenge with the SARS-CoV-2 Beta variant in human ACE2 transgenic K18 mice. Groups of mice were immunized three times, 3 weeks apart, and 2 weeks after the last dose the mice were challenged with 1 x 10⁵ pfu of the SARS-CoV-2 Beta variant intranasally (FIG. 11A), and then followed closely for symptoms and weight changes for 13 days. The universal DNA vaccine OC-2.4 fully protected these mice against lethal infection and showed complete protection against viral replication in the upper airways and in the spleen (FIGS. 11C and D). However, these histopathological signs of disease were lower, albeit not significantly different from the mock-vaccinated group (FIG. 11B). The viral replication in the lungs of the OC-2.4-vaccinated mice was also significantly reduced as compared to controls (FIG. 11D). In contrast, vaccination with recombinant S in QS21 adjuvant also protected against lethal infection, and resulted in low infection in the lungs, but viral replication could still be detected in the upper airways and in the spleen (FIG. 11C). Also, this group did not show significantly lower histopathological signs of disease as compared to the mock-vaccinated group (FIG. 11B). Interestingly, vaccination with a recombinant N protein (WH1 variant) in QS21 adjuvant (rN/QS21) that only activates antibodies and T cell responses to N, showed a 60% protection against lethal disease and viral replication in the upper airways, but less so in the lungs (FIGS. 11C and D). However, this group had a significantly more pronounced histological disease as compared to the groups vaccinated with recombinant S and OC-2.4 (FIG. 10B). This strongly support the notion that T cells alone have a key role against protection against severe disease (Gao et al, 2022; Pardieck et al, 2022), and that the role of T cells may differ in their function in the upper and the lower airways.

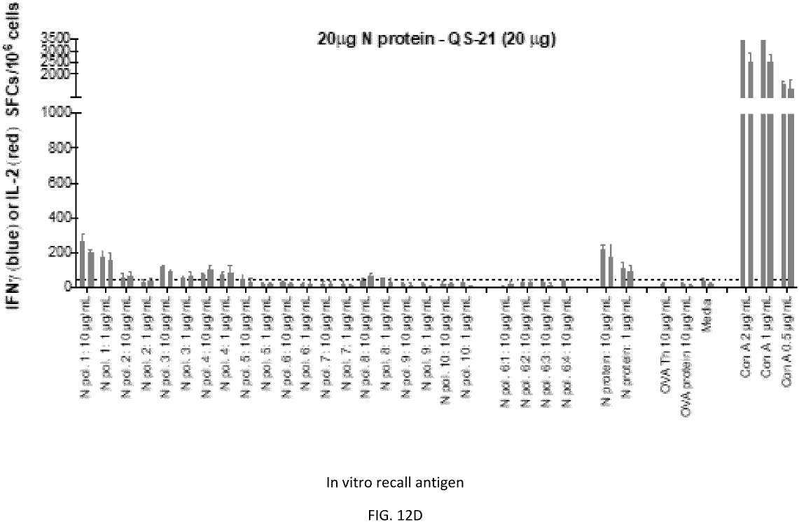

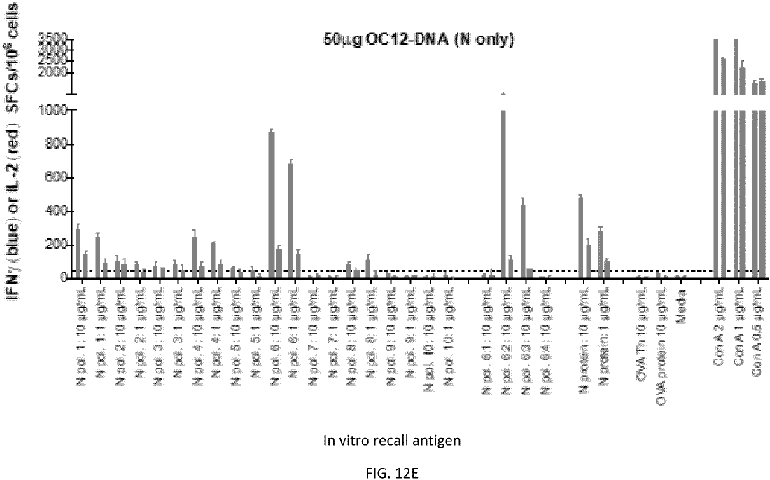

To compare the priming of N-specific antibodies and IFNy-producing T cells between DNA delivered by in vivo EP and a recombinant N protein in adjuvant, we immunized C57BL/6 mice (same background as the K18 mice) twice with either a plasmid encoding N alone, rN in alumn, or rN/QS21 (FIG. 12). A naive group was included as negative control. This showed that rN/QS21 was most potent in inducing N antibodies, whereas DNA was most effective in priming IFNy-producing N-specific T cells, but comparable to rN/QS21 to priming IL-2-producing N-specific T cells (FIG. 12). Also, the reactivity to pool 6 peptides was most likely due to a CD8-restricted response, as it was only seen after immunization with DNA (FIG. 12). Overall, QS21 was superior to alumn in priming N-specific antibody and T cell responses (FIG. 12), which helps to explain its T-cell dependent and partially protective effect in the K18 model (FIG. 11).

### Example 15: Discussion

In this study, we herein designed and evaluated a completely new vaccine design strategy. We combined the RBD loops from three different variants of SARS-CoV-2, with the highly conserved M and N proteins. The concept was to induce broadly reactive and neutralizing antibodies, as well as broader and new T cell specificities as compared to any of the vaccines against SARS-CoV-2 used globally. We could show that when delivered as a DNA, the vaccine effectively induced broadly reactive and neutralizing antibodies as well as completely new T cell specificities. Importantly, we could show that this vaccine design could be used as a booster vaccine for the currently used spike-based vaccine, by improving anti-spike antibody levels, as well as broadening the T cell responses. Lastly, we could show that our vaccine effectively protected K18 hACE2 mice against a lethal disease caused by SARS-CoV-2 Beta variant, a variant sharing many mutations with the currently circulating Omicron variants. Thus, taking all data together with the favorable safety profile obtained in a toxicological study in rabbits, this supports a clinical development of this vaccine. A very interesting observation was that mice vaccinated with an N protein alone, despite being protected against lethal disease, had a more pronounced damage as compared to the mice with priming of both Nabs and T cells. This supports the concept that T cells indeed protect against severe disease and death (Pardieck et al, 2022). However, the absence of NAbs most likely allows for a viral spread and subsequent T cell-mediated killing resulting in a more pronounced histological disease.

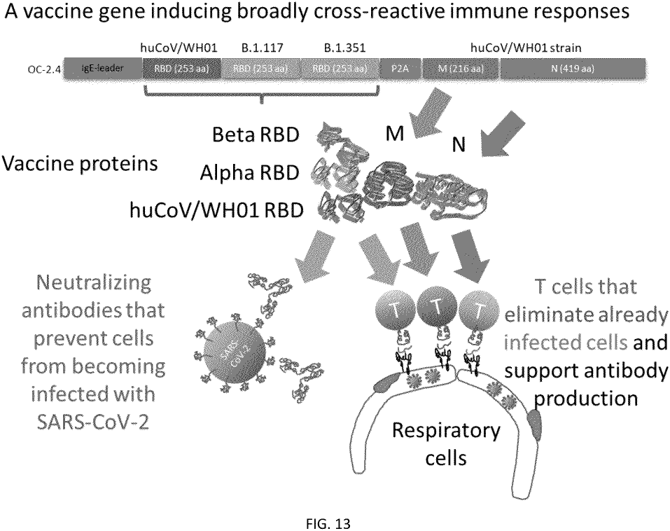

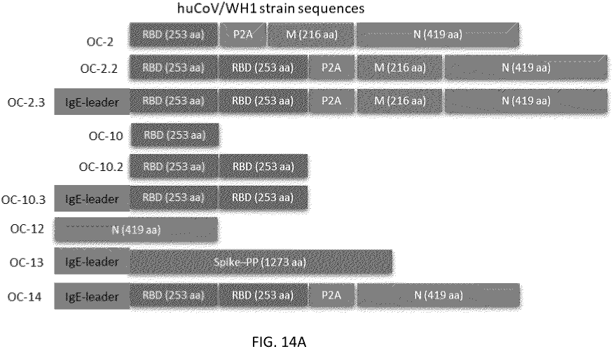

In conclusion, with a virus like SARS-CoV-2 that shows an impressive ability to mutate and to spread also among a population with high vaccine coverage, new vaccine designs are needed. Here, we describe a unique universal SARS-CoV DNA vaccine that induces more broadly neutralizing activity against the huCoV-19/WH01, Beta, Delta, and Omicron variants than standard spike-based vaccines (FIG. 13). Our data supports clinical development of this completely new approach to vaccines against SARS-CoV-2 to complement existing vaccines as a heterologous booster, and potentially also protect against future SARS-CoV viruses that have replaced, or greatly changed the spike protein of SARS-CoV-2.

### Example 17: Materials and Methods

### Animals

Female C57BL/6 (H-2^{b}) and BALB/c (H-2^{d}) mice were obtained from Charles River Laboratories, Sulzfeld, Germany. All mice were 8-12 weeks old at the start of the experiments and maintained under standard conditions at Preclinical laboratory (PKL), Karolinska University Hospital Huddinge, Sweden. Nine New Zealand White rabbits were purchased from commercial vendors and kept at Astrid Fagreus Facility at Karolinska Institutet. A total of 18 ferrets were purchased from Marshall Bioresources (NY) and acclimatized for 21 days. They were housed three animals per two-level cage at the Biosafety level-3 (BSL-3) facility at Astrid Fagraeus Laboratory (AFL), Karolinska Institutet, according to the guidelines for ferrets. A total of six non-human primates (NHP; cynomolgus fasucularis) were purchased. The animals were housed in pairs at the Biosafety level-3 (BSL-3) facility at AFL, Karolinska Institutet, according to guidelines for NHPs.

All animal procedures were granted by regional animal ethics committees (approvals 03634-2020, 16171-2020, 7602-2020), 17114-2020.

### DNA Plasmids and Recombinant Proteins

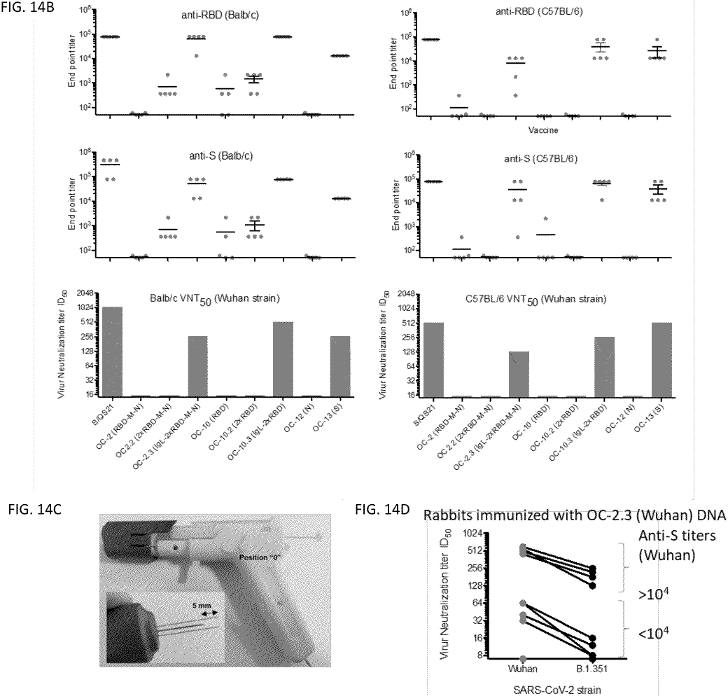

A total of eight genes were generated based on the sequence from the Wuhan strain (FIG. 14). The genes contained a combination of, or alone, the RBD, N and M proteins, with or without autoproteolytic P2A sequences. All sequences were codon optimized for expression in human cells and were synthesized by a commercial source (Genscript). Plasmids were grown in TOP10 Escherichia coli cells (Life Technologies) and purified for in vivo injections using Qiagen Endofree DNA purification kit (Qiagen) following manufacturer’s instructions. The correct gene size was confirmed by restriction enzyme digests using BamHI and XbaI (Fast Digest; Thermo Fisher Scientific). Recombinant N protein was designed in-house and produced by Genscript (32878912). Recombinant S, RBD, and M was purchased Genscript. The spike proteins were produced as full length by transient protein production in mammalian cells (Expi293). To facilitate trimerization of the full-length spike, a C-terminal T4 fibritin trimerization motif was included according to Wrapp et al. Further a strep-tag, fused to the C-terminal was used for purification (Hober et al. 2021). The beta version (B1.351) of the spike was produced with three mutations in the RBD part (K417N E484K N501Y).

### Peptides

A total of 42 20-mer peptides with 10 aa overlap, corresponding to the Wuhan RBD (25 peptides), M (22 peptides) and N (41 peptides) and Bat N (42 peptides) were purchased from Sigma-Aldrich (St. Louis, MO). The peptides were divided in pools of 4-5 or 8-10 peptides/pool depending on experimental setup.

### Immunization Schedules in Mice, Ferrets and NHPs

Mice were immunized two times with three weeks intervals, and sacrificed 2 weeks after the second immunization for spleens and blood collection. In brief, female C57BL/6 or BALB/c mice (5 per group) were immunized intramuscularly in the tibialis cranialis anterior muscle with 50 µg plasmid DNA in a volume of 50 µL in sterile phosphate-buffered saline (PBS) by regular needle (27G) injection followed by in vivo electroporation using a Cliniporator2 device (IGEA) using 2 electrodes. Prior to vaccine injections, mice were given analgesic and kept under isoflurane anesthesia during the vaccinations. During in vivo electroporation (in mice, rabbit, ferret and NHP) a 1-ms 600-V/cm pulse followed by a 400-ms 60-V/cm pulse pattern was used to facilitate better uptake of the DNA. Groups of mice were injected subcutaneously at the base of the tail with recombinant protein mixed (1:1) with QS21 adjuvant (GMP grade, Alpha diagnostics).

For studies in rabbits, 3 New Zealand White rabbits per group were immunized with 250, 500 or 750 µg OC-2.3 DNA vaccine. Vaccines were administered in right tibialis anterior muscle in 500, 1000 or 1500 µL sterile PBS followed by in vivo electroporation using the GeneDriVe (IGEA) device and GeneGun electrode (IGEA) with a 4 electrode array at a depth of 1 cm.

Ferrets, in groups of 3, were immunized in the tibialis anterior muscle with 300 µg DNA vaccine followed by in vivo electroporation using EPSGun. One group of ferrets were immunized subcutaneously with recombinant protein in QS-21.

Six NHP were immunized intramuscularly in right quadriceps muscle with 1 mg SARS-CoV-2 OC-2.3 DNA or control DNA (HBV) in 500 µL followed by in vivo electroporation using EPSGun.

### Ferret Challenge Model

Ferrets were challenged with 1 × 10⁶ pfu SARS-CoV-2 via intranasal administration in a volume of 0.5 mL. Animals were assessed with regards to weight, general health status and body temperature daily following infection. Nasal lavage (NAL) was collected 2, 4, 7 and 9 days post-infection (PI). Ten days PI, animals were euthanised, where after broncheoalveolar lavage (BAL) was performed, and trachea and lungs were excised for histopathological analysis.

### Non Human Primate Challenge Model

NHP were challenged with 5 × 10⁶ pfu SARS-CoV-2 via intranasal administration in a volume of 0.5 mL and intrathecal in a volume of 4.5 mL. Animals were assessed with regards to health status daily following infection. Blood samples were collected day -35, -21 and 0 prior infection and day 4, 10, 14 and 21 post infection. Bronchoalveolar lavage (BAL) was performed day 4 and 10 post infection to for detection of virus.

### Detection of IgG Specific Antibodies

Detection of mouse, rabbit, ferret and NHP immunoglobulins against S, RBD or N was performed according to previously described protocol (Ahlen et al. 2007). Serum antibodies were detected by an alkaline phosphatase conjugated goat anti-mouse IgG (Sigma A1047) 1:1 000 or mouse anti-rabbit IgG (Sigma A2556) 1:1 000 and visualized using p-nitrophenyl phosphate substrate solution. Optical density (OD) was read at 405 nm with a 620 nm background. Antibody titers were determined as endpoint serum dilutions at which the OD value was at least three times the OD of the negative control (nonimmunized or control animal serum) at the same dilution.

### Detection of Specific IFN-γ Producing T Cells

Two weeks post last vaccination, splenocytes or peripheral blood mononuclear cells (PBMCs) from each group of immunized mice, ferrets, or non-human primates were harvested and tested for their ability to induce specific T cells based on IFN-y, and IL-2 (mouse), secretion after peptide stimulation for 36 to 48h essentially as described (Maravelia et al. 2021). Detection of mouse and rabbit IgG against PreS 1 consensus sequences and overlapping 20mer-peptides was performed following previously described protocol (Maravelia et al. 2021, Hawman et al. 2021).

### Virus Propagation

The SARS-CoV-2 huCoV-19/WH01, Beta, and Omicron strains were isolated from patient samples at the Public Health Agency of Sweden and confirmed by sequencing. The SARS-CoV-2 Delta variant was provided by Dr. Charlotta Polacek Strandh, Statens Serum Institute, Copenhagen, Denmark. All variants were propagated on Vero-E6 cells and titered using a plaque assay as previously described (Varnaite et al. 2021), with fixation after 72 hrs. The huCoV-19/WH01, Delta and Omicron strains used in this study was passaged 3 times and the Beta strain 2 times.

### Neutralization of SARS-CoV-2 in Vitro

Titer of neutralizing antibodies in serum from mice and rabbits were determined by CPE based microneutralization assay. For mice, serum from each vaccination group was pooled, while for rabbits serum from each individual was tested. Briefly, serum was heat inactivated at 56° C. for 30 min before serial diluted 2-fold. Each dilution was conducted in quadruplets and mixed with 500 pfu of SARS-CoV-2 huCoV-19/WH01, Beta, Delta or Omicron in a 1:1 dilution. After 1 hour of incubation at 37° C., 5% CO₂ 100 uL of serum-virus mix was added to Vero E6 cells on a 96-well plate (20×10⁴ cells/well) and incubated for 72 hrs at 37° C., 5% CO₂. CPE for each well was determined using a Nicon Eclipse TE300 microscope. As controls, wells with medium only, diluted serum only, virus only and serum known to contain SARS-CoV-2 neutralizing antibodies mixed with virus was included in each experiment.

### PCR/Viral RNA

Trizol (Sigma-Aldrich) in a ratio of 1:3 was used to inactivate potential virus in nasal lavage samples (50 µL) from SARS-CoV-2 infected K18-hACE2 mice. For lung and spleen, PBS was added to each sample (1 g/ml) and pestles were used to crush the organs. Thereafter, the samples were centrifuged (5 min at 7000 rpm) and 50 µL of each lung or spleen sample was added to Trizol (1:3). Total RNA was extracted using the Direct-zol RNA Miniprep kit (Zymo Research) according to the manufacturer’s instructions. Viral RNA were thereafter measured by quantitative real-time polymerase chain reaction (qRT-PCR) using TagMan Fast Virus 1-Step master mix (Thermo Fisher Scientific) with primers and probe for the SARS-CoV-2 E gene.

Forward:5′-ACAGGTACGTTAATAGTTAATAGCGT-3′

Reverese:5′-ATATTGCAGCAGTACGCACACA-3′

Probe: FAM- ACACTA GCC ATC CTT ACT GCG CTT CG MGB

For lung and spleen samples, mouse ACTB mix (Thermo Fisher Scientific) was used as endogenous control. The PCR reaction was performed using a capillary Roche LightCycler 2.0 system.

### Histological Analysis

The lungs were formalin fixed, embedded in paraffin, and sectioned for H&E staining. The sections were analyzed by an independent veterinary pathologist blinded to the treatment groups. All sections were scored according to bronchial and alveolar signs of inflammation and disease.

### Statistical Analysis

Data analyzed on GraphPad Prism V.5 software and Microsoft Excel V.16.13.1.

### Example 18: Generation of Experimental Vaccines

It is clear that human coronaviruses stem from Bats (Hu et al. 2015). The spike proteins of SARS-CoV and SARS-CoV-2 induces cross reactive T cells but poorly cross reactive Nabs (Le Burt et al. 2020). Out of the structural proteins of SARS-CoV and SARS-CoV-2, the M and N proteins have a higher genetic homology to other animal SARS-CoV viruses (Hu et al 2015, Latinne et al. 2020), and that T cells to these show a better cross reactivity across beta coronaviruses (Le Burt et al. 2020). We recently showed that these proteins induce T cell that can recognize sequences from both pangolin and Bat SARS-CoV viruses (EMBO Mol Med). Thus, we could show that a universal SARS-CoV vaccine containing the receptor binding domain in different versions combined with the M and N proteins induce broad responses (EMBO Mol Med). As an extension of the previous study, we here generated multiple chimeric vaccine genes that either induced T cells, or both NAbs and T cells to better define the role of T cells in protection and disease (FIG. 14).

### Example 19: Priming Antibodies

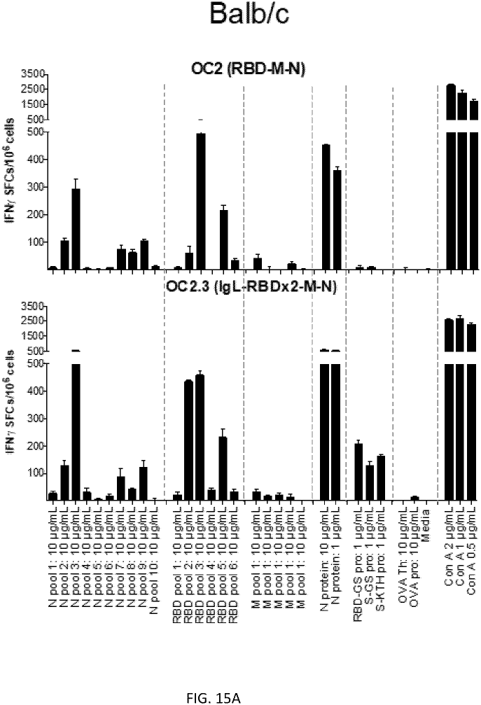

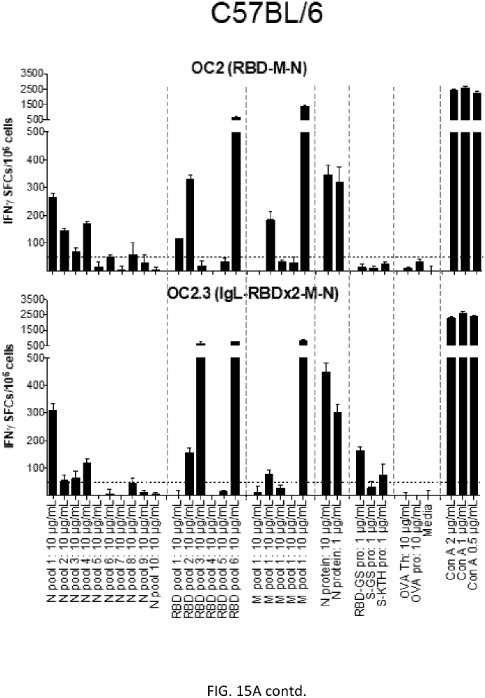

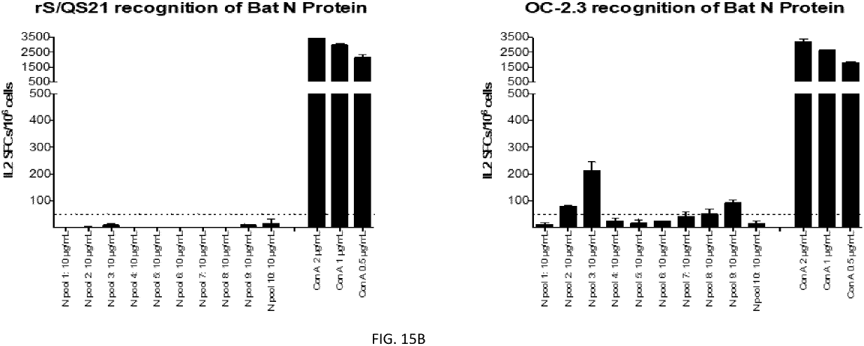

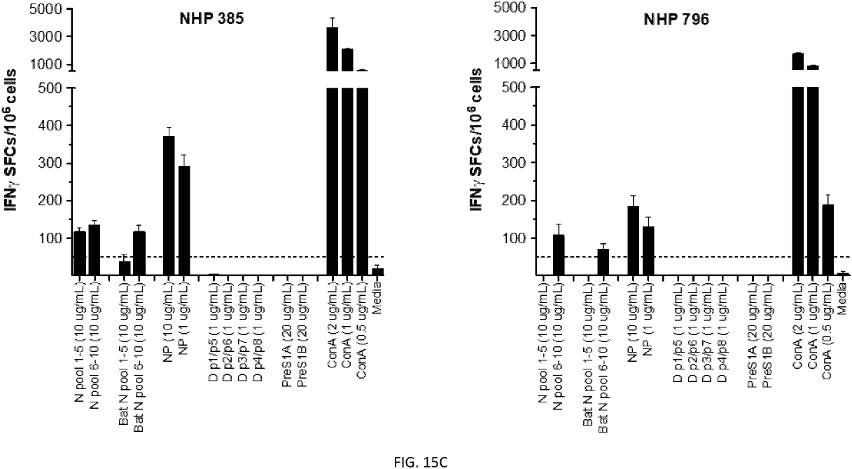

As the spike protein differs significantly between SARS-CoV strains, the need for cross-reactive T cells increase. The design of the RBD sequence affected the ability to induce NAbs to RBD and S similar to previous observations (Dai et al. 2020) (FIG. 14). However, this did not affect the ability to induce T cells to RBD, M and N (FIG. 15). Constructs containing a single linear RBD loop without an Ig leader sequence (OC-2 and OC-10) failed to induce detectable antibodies to recombinant RBD or S, despite an effective priming of T cells (FIGS. s 14-15). Constructs containing a double RBD loop (OC-2.2) slightly improved B cell immunogenicity (FIG. 14). As expected, the addition of an Ig leader sequence followed by a double RBD loop was highly effective in inducing antibodies to recombinant RBD or S, either alone or in combination with other SARS-CoV-2 proteins (OC-2.3 and OC-10.3; FIG. 14). These vaccines also effectively induced T cells to all vaccine components (FIG. 15).

The antibody responses to RBD and S, but not T cell responses, were weaker in C57BL/6 as compared to Balb/c mice (FIG. 14A). The N containing constructs induced antibodies and T cells to N (FIGS. 14B, 15A). The M containing constructs induced T cells to M (FIG. 15A). No antibodies to M could be reproducibly detected regardless of the antigen used, possibly reflecting that the sequences were not exposed to B cells due to the construct design, or that the antigens used in the detection simply failed to detect these antibodies (data not shown).

A central question regarding DNA vaccines is the ability to retain immunogenicity in larger animals. One SARS-CoV DNA vaccine (OC-2.3) was therefore used to immunize rabbits using a newly developed device named the Egun intended for human use (FIG. 14C). The device allows for a single step delivery of DNA together with in vivo EP. Rabbits receiving two DNA doses of 250, 500, or 750 µg developed anti-S levels between 10³-10⁵ (Wuhan strain; FIG. 14D). These were evaluated for the ability to neutralize Wuhan and Beta/B.1.351 SARS-CoV-2 VOCs in vitro (FIG. 14D). This showed that a priming of high levels of anti-S to Wuhan virus was required for a good cross-neutralization of the Beta VOC. Thus, we could confirm that high levels of antibodies are essential for effective cross-neutralization of different SARS-CoV VOC.

### Example 20: T Cell Induction by Universal SARS-CoV Vaccines

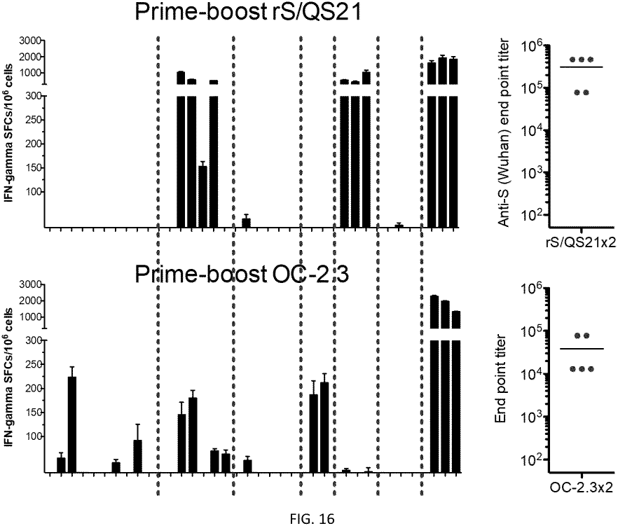

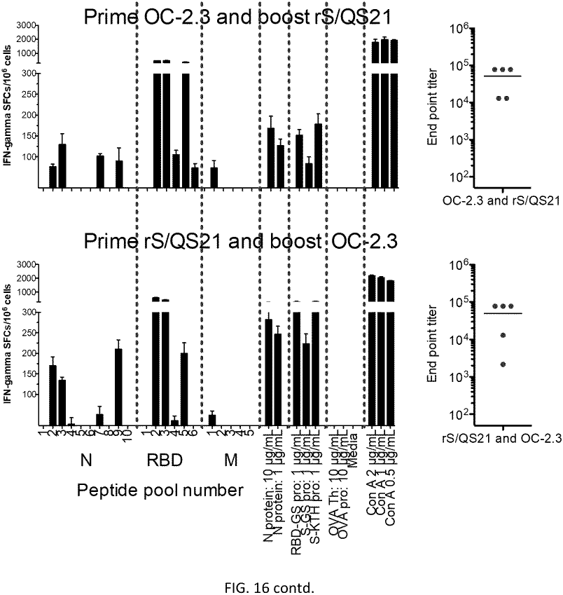

The DNA vaccines containing a single or double RBD loop affectively primed T cells to the different components in the respective vaccine (OC-2 and OC-2.3; FIG. 15A). The recognition of the different components differed slightly between Balb/c and C57BL/6 mice, with Balb/c mice recognizing mainly RBD and N, whereas C57BL/6 also showed a response to M (FIG. 16). This illustrates the advantage of including multiple SARS-CoV-2 proteins in priming broadly reactive T cell responses in genetically diverse hosts. Importantly, we found that the SARS-CoV-2-specific T cells primed by the universal vaccine cross-reacted with peptides corresponding to N protein sequence from Bat SARS-CoV (FIG. 16B). This is consistent with the observation that murine N-specific T cells recognize regions that have a 100% homology with Pangolin-derived SARS-CoV sequences (Ahlen et al. 2020), and supports the concept of universal SARS-CoV vaccines. In addition, in two out of three NHPs immunized twice with the same vaccine (described in detail later in the text), had SARS-CoV-2 N-specific T cells were cross reactive with Bat-SARS-CoV N sequences (FIG. 15C). Thus, the addition of M and N sequences ensures cross reactivity to animal SARS-CoVs.

The majority of approved COVID-19 vaccines are based on the spike protein (Golob et al. 2021). Thus, any new vaccine candidate needs to work well in combination with these, and add new properties not present in the current vaccines. We therefore combined our universal SARS-CoV vaccine in heterologous prime boost strategies with a spike protein-based vaccine. Immunization with recombinant Spike (rS) in QS21 is effective in inducing antibodies, NAbs and T cells to S (FIGS. 14B and 16). However, to induce broader T cell reactivities, additional SARS-CoV proteins are required. In heterologous prime-boost regimens with the universal SARS-CoV DNA vaccine OC-2.3 broadened the T cell reactivity (FIG. 16). Regardless of the combination, high levels of anti-S antibodies were combined with a broader T cell reactivity to M and N. This suggest that a universal SARS-CoV DNA vaccine can effectively be used in heterologous prime-boost strategies to boost and broaden the responses generated by existing spike-based vaccines.

### Example 21: Role of Antibodies and T Cells in the Protection Against SARS-CoV-2 Infection in Ferrets

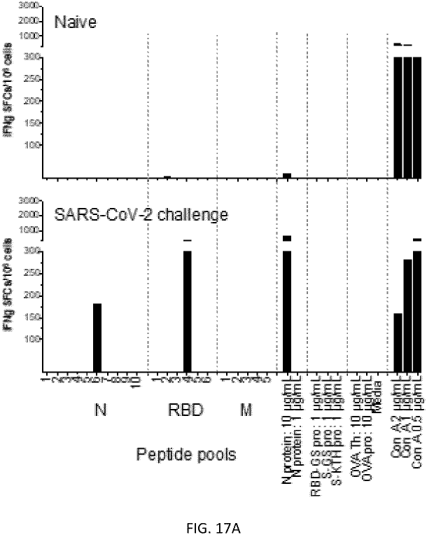

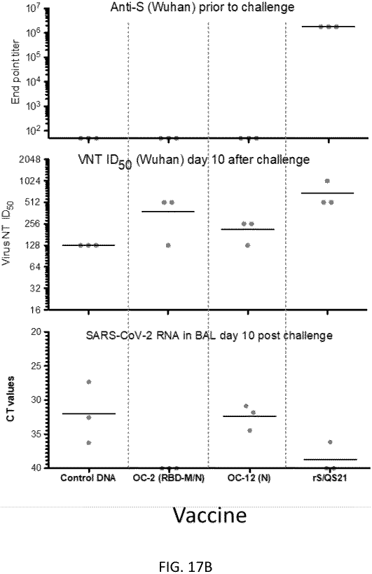

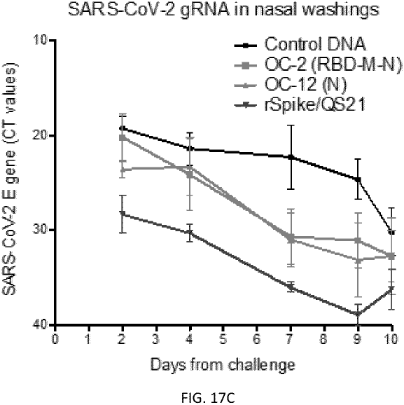

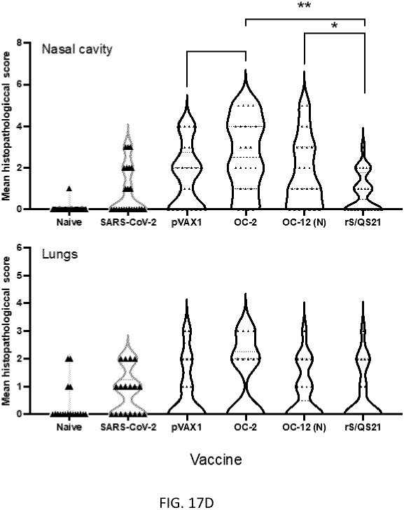

The role of T cells in the protection or control of SARS-CoV-2 infection is poorly understood. We used the ferret model to take advantage of two of our DNA vaccines that induced SARS-CoV-2-specific T cells, but not NAbs or memory B cells, to better separate the role of these responses in the protection against SARS-CoV-2. In a pilot experiment where three ferrets were left naive and three were infected with 10⁶ pfus of SARS-CoV-2, the infected animals developed SARS-CoV-2 specific antibodies and T cells at 10 days post infection (FIG. 17A and data not shown). Next, groups of ferrets were immunized with a control DNA vaccine (Maravelia et al. 2021), the OC-2 construct (RBD-M-N; FIG. 14A), the OC-12 (N; FIG. 14A) construct, and rS/QS21, at weeks zero and three. Two weeks after the last vaccination all ferrets were challenged with SARS-CoV-2. Consistent with the murine data, did the groups vaccinated with OC-2 (RBD/M/N) or OC-12 (N) DNA vaccines not develop antibodies to S but to N prior to challenge (FIG. 17B and data not shown). In contrast, vaccination with rS/QS21 induced high levels of S antibodies prior to challenge (FIG. 17B). All ferrets except two (one in the OC-2 and one in the OC-12 group) displayed no or mild clinical symptoms such as slight drowsiness at days two to four. The remaining two were significantly affected at day two to four with thick mucus in the nose, loss of appetite, and drowsiness. They recovered by day four to five. The control vaccine group had high levels of SARS-CoV-2 RNA in nasal washings at days two to nine, and all had RNA in BAL at day 10 (FIG. 17C). In contrast, the group vaccinated with rS/QS21 had rapidly dropping levels of SARS-CoV-2 RNA in nasal washings, and all but one had cleared virus in BAL by day 10. Interestingly, the two groups with vaccine induced SARS-CoV-2-specific T cells prior to challenge, had mean nasal SARS-CoV-2 RNA levels in between the control DNA group and the rS/QS21 vaccine group. The two animals vaccinated with OC-2.3 or OC-12 that developed clinical symptoms and, had nasal SARS-CoV-2 RNA levels similar to those in the control group (data not shown). Finally, only the OC-2 (RBD-M-N) DNA vaccinated group was completely negative for SARS-CoV-2 RNA in BAL at day 10 (FIG. 17B).

The control DNA group had slowly appearing S and N antibody levels, including NAbs reflecting the kinetics of the natural infection (FIG. 17B and data not shown). In contrast, the animals with vaccine induced SARS-CoV-2-specific T cells showed anamnestic responses with respect to N antibodies and NAbs (FIG. 17B and data not shown). Finally, with respect to histological findings at necropsy where the most pronounced changes were multifocal intraluminal cellular debris in the trachea and carina in all animals vaccinated with OC-2 (RBD-M-N) or OC-12 (N) DNA, and in one in the group vaccinated with rS/QS21 (FIG. 17D). In nasal turbinates varying degrees of inflammation was noted in all groups. In the trachea and carina there seemed to be more inflammation in the OC-2 (RBD-M-N) or OC-12 (N) DNA vaccinated groups, than in the other groups. Thus, although the vaccine primed T cells can help to control SARS-CoV-2 replication, the histological examination suggest that this control may be exerted by elimination of SARS-CoV-2 infected cells resulting in a slightly more pronounced pathology. Overall, the histological findings suggested that none of the vaccine strategies completely protected against histological signs of disease (FIG. 17D), which may be explained by the 2-10 fold higher dose used for challenge than often used in infection experiments (Shi et al. 2020). Taken together, this study suggests a role for SARS-CoV-2-specific T cells in controlling SARS-CoV-2 replication, either directly by eliminating infected cells, or indirectly, by promoting anamnestic antibody responses, or a combination of both. The increased pathology in respiratory tissue seen in the absence of NAbs prior to a high dose challenge, suggest that the elimination of infected cells by T cells may contribute to both viral control and to the pathology. Thus, this supports a central role of NAbs in limiting the spread of the SARS-CoV-2 and suggest a coordinated action of B cells and T cells in control and clearance of SARS-CoV-2.

### Example 22: Role of Antibodies and T Cells in the Protection Against SARS-CoV-2 Infection in a Lethal Disease Model

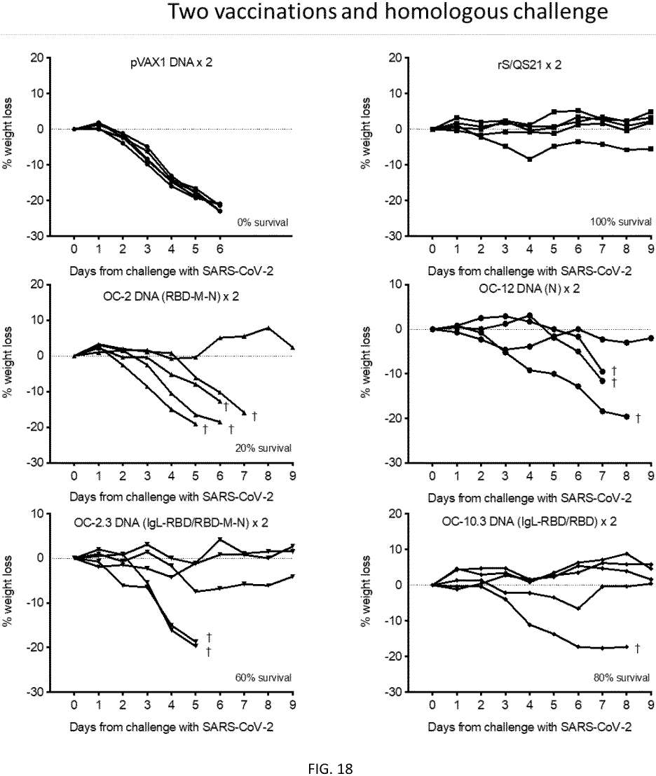

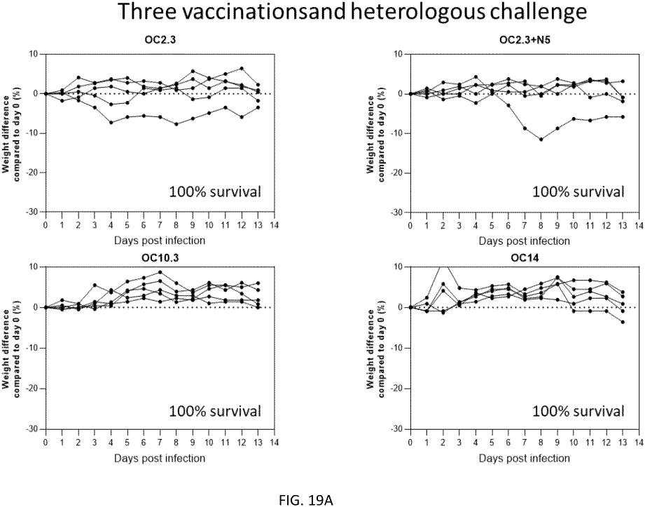

To evaluate the role of NAbs and T cells in a lethal disease model, we immunized K18, mice expressing the hACE2 receptor, twice with different immunogens (FIG. 18). Again, groups vaccinated with rS/QS21 were protected against lethal disease. In contrast, the groups were only T cells were primed, similar to the ferrets, partially protected against lethal disease (20-25%; FIG. 18). However, the weight loss was significantly delayed in the T cell vaccine group as compared to controls (>10% weight loss at day four; 5/5 in controls vs 2/9 in the OC-2 and OC-12 groups combined, p<0.05, Fisher’s exact test). In the two groups OC-2.3 and OC-10.3 with increasing levels of spike antibodies the protection was 60% and 80%, respectively. Thus, this confirms the key role of NAbs in protection against disease, as well as confirming a partially protective role of T cells against disease.

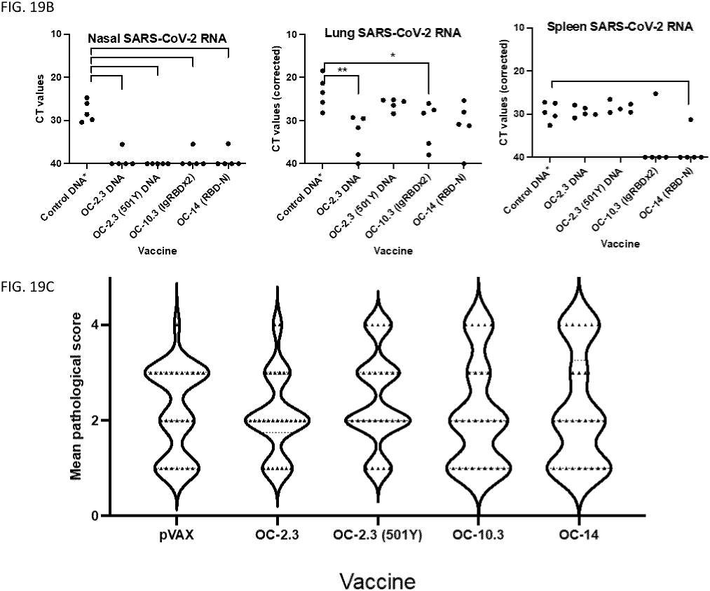

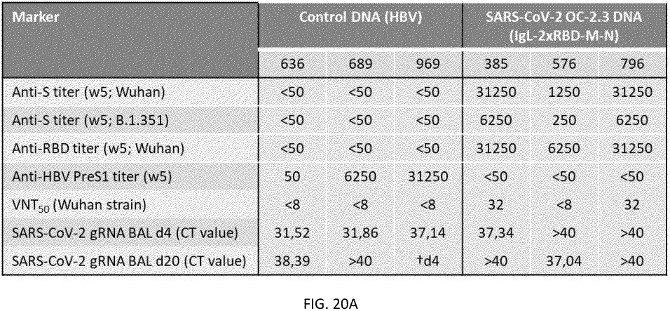

Next, we evaluated different immunogens for heterologous challenge with the Beta variant. Groups of K18 mice were immunized three times, 3 weeks apart, and 2 weeks after the last dose the mice were challenged with 1 × 10⁵ pfu of the SARS-CoV-2 Beta variant intranasally (FIG. 19) and then followed closely for symptoms and weight changes for 13 days. All groups expressing the RBD had a 100% survival evidencing cross protection between the Wuhan and Beta strain.

### Example 23: Role of Antibodies and T Cells in the Protection Against SARS-CoV-2 in NHPs

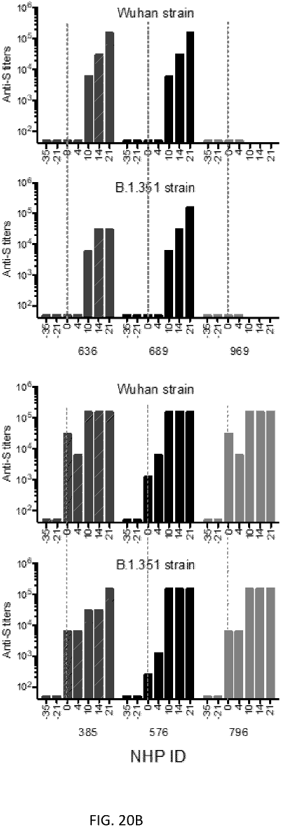

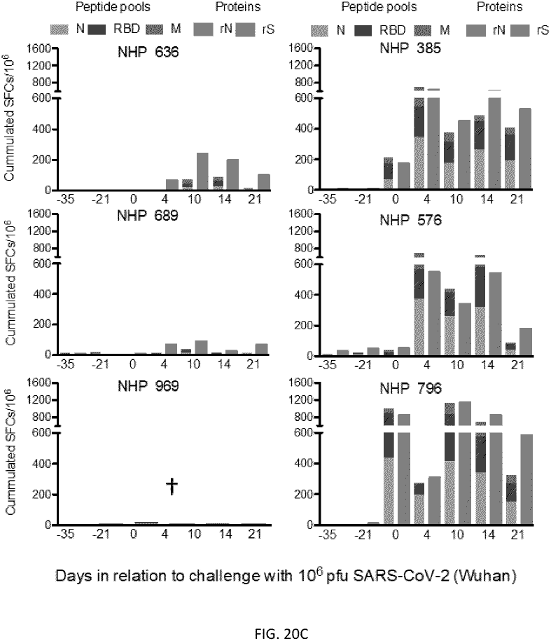

We evaluated one universal SARS-CoV DNA vaccine OC-2.3 (IgL-RBDx2-M-N) in a pilot experiment in cynomolgus macaques. Groups of three NHPs were immunized with a control DNA encoding a HBV vaccine (Maravelia et al. 2020) or the universal SARS-CoV DNA vaccine OC-2.3, twice at days -35 and -14, and then challenged on day zero with a total dose of 5 × 10⁶ pfu SARS-CoV-2 in the right nostril and intra tracheally (FIG. 20). This resulted in infection in all six animals. One of the control vaccine animals died under anaesthesia during the BAL sampling at day 4, possibly as a reaction to anaesthesia, or a combination of the anaesthesia and the infection. All control animals had RNA in BAL at day 4, and one (no 636) out of two had virus in BAL at day 20 (FIG. 20). The two surviving control animals had a mild to moderate histological disease, and both developed weak T cell responses mainly to recombinant N and S proteins after challenge. The antibody responses to S were not detectable until day 10 after challenge.

The group immunized with SARS-CoV DNA vaccine all developed anti-S and anti-RBD prior to challenge (FIGS. 20A-B). The two animals with anti-S/RBD levels of >10⁴ both had low levels of NAbs to SARS-CoV-2 Wuhan strain, but not to the beta VOC, prior to challenge (FIG. 20B). All vaccinated animals showed strong anamnestic responses and had by day 10 reached anti-S titers of >10⁵ to Wuhan strain, and >10⁴to the beta VOC. Importantly, all vaccinated animals had detectable SARS-CoV-2-specific T cell responses prior to challenge, and the strength of these correlated well with the anti-S levels (FIG. 20C). There was no difference in SARS-CoV-2 RNA levels in nasal washings between the control and vaccine groups (data not shown), highlighting the importance of high levels of NAbs. In contrast, two out of three SARS-CoV-2 vaccinated animals cleared SARS-CoV-2 RNA in BAL at day 4, and two at day 20 (FIG. 20A), and all vaccinated animals developed a strong multi specific anamnestic T cell response after challenge (FIG. 20C). It was noted that the animal with the lowest anti-S and T cell responses prior to challenge had a more pronounced histological changes as compared to the two animals with the strongest vaccine induced response (FIG. 20). Overall, this limited pilot study, support the notion that, a coordinated activation of NAbs and T cells is required for a rapid clearance of SARS-CoV-2 RNA and control of infection in the airways.

### Example 24: Discussion

There are numerous vaccines against SARS-CoV-2 in clinical development, and more than 10 have already been approved for human use in several countries (Golob et al 2021). All these, except two killed whole virus vaccines, are based on the spike protein of SARS-CoV-2 with the aim of inducing Nabs (Golob et al. 2021). A recent finding show the ability of SARS-CoV-2 VOCs carrying mutations in position 484 to reduce the effectivity of at least three different S-based vaccines in clinical trials (Madti et al 2021, Shinde et al. 2021, Sadoff et al. 2021, Sheikh et al 2021). This strongly suggest a high dependence of NAbs in protection against mild and moderate disease, but possibly less so against severe disease and death. SARS-CoV-2 infection in macaques induces both humoral and cellular immune responses (Shaan Lakshmanappa et al. 2021), and a prior infection has a protective effect upon rechallenge (Chandrashekar et al. 2020). The individual contribution of antibodies and T cells are still not fully understood. Thus, the respective contribution of NAbs and T cells needs to be further investigation.

To further our understanding of NAbs and T cells we herein generated templates for universal DNA vaccines encoding RBD, membrane protein, and/or nucleoprotein that effectively induced T cells but not NAbs, or both, to elucidate how T cells can help controlling the COVID-19 infection. These universal SARS-CoV vaccine candidates effectively induced neutralizing antibodies and primed broadly reactive T cells. When high levels of Nabs were reached, then these antibodies neutralized both Wuhan and Beta VOCs in vitro. However, when using vaccines that only induced T cells we could show that pre-existing T cells resulted in anamnestic antibody responses, and a better control of SARS-CoV-2 replication as evidenced by both lower nasal SARS-CoV-2 levels and clearance in the lungs in the ferret challenge model. Thus, vaccine induced SARS-CoV-specific T cells promote control and clearance of SARS-CoV-2, which may be relevant for both infection and vaccination. However, it is important to note that in the absence of NAbs, the viral control may come at a prize of a more pronounced histological damage possibly caused by the elimination of infected cells. Here it may differ between different vaccine technologies in how effective they induce different types of T cells. Thus, it was equally clear that NAbs were essential for a rapid control and containment of the infection in the ferret model. These data were reiterated in the K18 huACE2 challenge model where T cells alone offered a limited protection, wheras control improved with increasing antibody levels to S and RBD. Finally, vaccination of NHPs with a universal SARS-CoV DNA vaccine resulted in the presence of anti-S and RBD antibodies, low levels of NAbs, and broadly reactive T cells that together mediated a rapid elimination of virus in the lungs. The recall responses after challenge were very strong with respect to both S/RBD antibodies and broadly reactive T cells. There was a tentative association between the anti-S response, NAB levels, and the T cell response prior to challenge. The observation that a lower anti-S response protects the lungs but not the upper airways are fully consistent with other vaccine studies in NHPs (Feng et al. 2020, Guebre-Xabier et al. 2020, Mercado et al. 2020, van Doremalen et al. 2020, Vogel et al. 2020, Yu et al 2020).

In conclusion, we could show for the first time that vaccine induced SARS-CoV-2-specific T cells alone can promote control and clearance of SARS-CoV-2. This protection was mainly seen in the lower respiratory tract, and to a lesser extent in the upper airways. However, a full protection against disease required both NAbs and T cells. Universal SARS-CoV vaccines will most likely have the ability to induce a T cell memory that is cross reactive to diverse SARS-CoVs. These T cells may in the absence of cross reactive NAbs help to control infection from future SARS-CoV outbreaks, and minimize the burden on the healthcare. In addition, the presence of vaccine-primed S-specific T cells that partially control viral replication help to explain why VOCs carrying mutations that escape NAbs still cause mild to moderate disease, but prevent hospitalization or death.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C″ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description or claims, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

### References

Dai et al. “A Universal Design of Betacoronavirus Vaccines against COVID-19, MERS, and SARS” Cell. (2020);182(3):722-733.

Fehr AR & Perlman S. “Coronaviruses: An Overview of Their Replication and Pathogenesis” Methods Mol. Biol. (2015); 1282:1-23.

Starr et al. “Deep Mutational Scanning of SARS-CoV-2 Receptor Binding Domain Reveals Constraints on Folding and ACE2 Binding” Cell; (2020) 182(5);1295-1310.

Vijayachari et al. “Immunogenicity of a novel enhanced consensus DNA vaccine encoding the leptospiral protein LipL45” Hum. Vaccin. Immunother. (2015);11(8):1945-53.

Wu A et al. “Genome Compositions and Divergence of the Novel Coronavirus (2019-nCoV) Originating in China” Cell Host Microbe. (2020); 27(3):325-328.

Zhou et al. A human antibody reveals a conserved site on beta-coronavirus spike proteins and confers protection against SARS-CoV-2 infection. Science. Transl. Med. (2022) doi:10.1126/scitranslmed.abi9215.

What is claimed is:

1. A nucleic acid comprising at least one nucleic acid sequence encoding a SARS-CoV-2 polypeptide, wherein the at least one nucleic acid sequence encoding a SARS-CoV-2 polypeptide comprises:
i) one or more nucleic acid sequences encoding a SARS-CoV-2 spike receptor binding domain (RBD) polypeptide;
ii) a nucleic acid sequence encoding a SARS-CoV-2 nucleocapsid protein (NP) polypeptide;
iii) a nucleic acid sequence encoding a SARS-CoV-2 membrane (M) polypeptide;
iv) a nucleic acid sequence encoding a hepatitis D antigen (HDAg) polypeptide;
v) a nucleic acid sequence encoding an autocatalytic polypeptide cleavage site;
vi) a nucleic acid sequence encoding an IgE leader polypeptide;
vii) a nucleic acid sequence encoding a SARS-CoV-2 spike (S) polypeptide; or
viii) a nucleic acid sequence encoding a CC40.8 epitope;
or any combination thereof.

2-120. (canceled)

121. The nucleic acid of claim 1, wherein one or more of the RBD polypeptide, NP polypeptide, M polypeptide, or S polypeptide is derived from the wild-type SARS-CoV-2 strain (Wuhan-hu-1) or a SARS-CoV-2 variant.

122. The nucleic acid of claim 1, wherein one or more of the RBD polypeptide, NP polypeptide, M polypeptide, or S polypeptide comprises one or more mutations found in a SARS-CoV-2 variant.

123. The nucleic acid of claim 1, wherein each of the one or more nucleic acid sequences encoding the RBD polypeptide comprise one or more mutations corresponding to K417, N439, L452, T478, E484, N501, G339, S371, S373, S375, T376, D405, R408, N440, G446, S447, Q493, G496, Q498, or Y505 with reference to the full SARS-CoV-2 S protein set forth in SEQ ID NO: 83.

124. The nucleic acid of claim 123, wherein the nucleic acid comprises three tandem nucleic acid sequences encoding an RBD polypeptide.

125. The nucleic acid of claim 124, wherein the nucleic acid encodes a polypeptide having at least 90%, 91%, 92%, 93%, 94%, 95%, 96%, 97%, 98%, 99%, or 100% sequence identity to SEQ ID NO: 79, 82, 85, 87, 89, 93, 95, 97, or 99.

126. The nucleic acid of claim 125, wherein the nucleic acid comprises at least 90%, 91%, 92%, 93%, 94%, 95%, 96%, 97%, 98%, 99%, or 100% sequence identity to SEQ ID NO: 77, 78, 80, 81, 84, 86, 88, 92, 94, 96, or 98.

127. A product combination comprising the nucleic acid of claim 1 and a polypeptide, wherein the polypeptide comprises:
i) one or more SARS-CoV-2 spike receptor binding domain (RBD) polypeptide sequences;
ii) a SARS-CoV-2 nucleocapsid protein (NP) polypeptide sequence;
iii) a SARS-CoV-2 membrane protein (M) polypeptide sequence;
iv) a hepatitis D antigen (HDAg) polypeptide sequence;
v) an autocatalytic polypeptide cleavage site sequence;
vi) an IgE leader polypeptide sequence; or
vii) a SARS-CoV- spike (S) polypeptide sequence; or
viii) a CC40.8 epitope;
or any combination thereof.

128. The product combination of claim 127, wherein one or more of the RBD polypeptide, NP polypeptide, M polypeptide, or S polypeptide is derived from the wild-type SARS-CoV-2 strain (Wuhan-hu-1) or a SARS-CoV-2 variant.

129. The product combination of claim 127, wherein one or more of the RBD polypeptide, NP polypeptide, M polypeptide, or S polypeptide comprises one or more mutations found in a SARS-CoV-2 variant.

130. The product combination of claim 129, wherein each of the one or more RBD polypeptide sequences comprise one or more mutations at K417, N439, L452, T478, E484, N501, G339, S371, S373, S375, T376, D405, R408, N440, G446, S447, Q493, G496, Q498, or Y505 with reference to the full SARS-CoV-2 S protein set forth in SEQ ID NO: 83.

131. The product combination of claim 130, wherein the polypeptide comprises three tandem RBD polypeptide sequences.

132. The product combination of claim 131, wherein the polypeptide comprises a sequence having at least 90%, 91%, 92%, 93%, 94%, 95%, 96%, 97%, 98%, 99%, or 100% sequence identity to SEQ ID NO: 79, 82, 85, 87, 89, 93, 95, 97, or 99.

133. The product combination of claim 132, wherein the polypeptide is encoded by a nucleic acid comprising at least 90%, 91%, 92%, 93%, 94%, 95%, 96%, 97%, 98%, 99%, or 100% sequence identity to SEQ ID NO: 77, 78, 80, 81, 84, 86, 88, 92, 94, 96, or 98.

134. The product combination of claim 133, wherein the polypeptide is recombinantly expressed, optionally in a mammalian, bacterial, yeast, insect, or cell-free system.


SEQUENCE LISTING (100 sequences)

SEQ ID NO: 1 - DNA, 2091 bp, synthetic construct
  misc_feature 1..2091: note = SVF-1 wild-type DNA sequence
  source 1..2091: mol_type = other DNA
atggccttaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagggaagcggagctactaacttcagcctgctgaagcaggctggagacgtggaggagaaccctggacctatggcaagcgacaatggaccacagaatcagaggaacgcaccaagaatcactttcggcggcccaagcgactcaaccggcagcaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaataaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggcagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgagccggatcggaatggaggtgaccccaagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctcaaaacagctgcagcagtcaatgtcaagcgccgattcaactcaggca

SEQ ID NO: 2 - DNA, 2754 bp, synthetic construct
  misc_feature 1..2754: note = SVF-2 wild-type DNA sequence
  source 1..2754: mol_type = other DNA
atggccttaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagggaagcggagctactaacttcagcctgctgaagcaggctggagacgtggaggagaaccctggacctatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacaggcaagcgacaatggaccacagaatcagaggaacgcaccaagaatcactttcggcggcccaagcgactcaaccggcagcaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaataaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggcagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgagccggatcggaatggaggtgaccccaagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctcaaaacagctgcagcagtcaatgtcaagcgccgattcaactcaggca

SEQ ID NO: 3 - DNA, 2823 bp, synthetic construct
  misc_feature 1..2823: note = SVF-3 wild-type DNA sequence
  source 1..2823: mol_type = other DNA
atggccttaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagggaagcggagctactaacttcagcctgctgaagcaggctggagacgtggaggagaaccctggacctatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagggaagcggagctactaacttcagcctgctgaagcaggctggagacgtggaggagaaccctggacctatggcaagcgacaatggaccacagaatcagaggaacgcaccaagaatcactttcggcggcccaagcgactcaaccggcagcaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaataaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggcagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgagccggatcggaatggaggtgaccccaagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctcaaaacagctgcagcagtcaatgtcaagcgccgattcaactcaggca

SEQ ID NO: 4 - DNA, 1497 bp, synthetic construct
  misc_feature 1..1497: note = SVF-4 wild-type DNA sequence
  source 1..1497: mol_type = other DNA
atggccttaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagggaagcggagctactaacttcagcctgctgaagcaggctggagacgtggaggagaaccctggacctatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacag

SEQ ID NO: 5 - DNA, 2022 bp, synthetic construct
  misc_feature 1..2022: note = SVF-5 wild-type DNA sequence
  source 1..2022: mol_type = other DNA
atggccttaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaaggcaagcgacaatggaccacagaatcagaggaacgcaccaagaatcactttcggcggcccaagcgactcaaccggcagcaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaataaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggcagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgagccggatcggaatggaggtgaccccaagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctcaaaacagctgcagcagtcaatgtcaagcgccgattcaactcaggca

SEQ ID NO: 6 - DNA, 2685 bp, synthetic construct
  misc_feature 1..2685: note = SVF-6 wild-type DNA sequence
  source 1..2685: mol_type = other DNA
atggccttaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaaggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacaggcaagcgacaatggaccacagaatcagaggaacgcaccaagaatcactttcggcggcccaagcgactcaaccggcagcaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaataaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggcagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgagccggatcggaatggaggtgaccccaagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctcaaaacagctgcagcagtcaatgtcaagcgccgattcaactcaggca

SEQ ID NO: 7 - DNA, 1428 bp, synthetic construct
  misc_feature 1..1428: note = SVF-7 wild-type DNA sequence
  source 1..1428: mol_type = other DNA
atggccttaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaaggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacag

SEQ ID NO: 8 - DNA, 3321 bp, synthetic construct
  misc_feature 1..3321: note = SVF-8 wild-type DNA sequence
  source 1..3321: mol_type = other DNA
atggccttaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagctgccgcccgcagagccgcagcgaaagcaaaaaaaaccgcggcggccgcgaagaagtgctggaacagtgggtgaacggccgcaaaaaactggaagaactggaacgcgaactgcgccgcgcgcgcaaaaaaattaaaaaactggaagatgataacccgtggctgggcaacgtgaaaggcattctgggcaaaaaagataaagatggcgaaggcgcgccgccggcgaaacgcgcgcgcaccgatcagatggaaattgatagcggcccgcgcaaacgcccgctgcgcggcggctttaccgatcgcgaacgccaggatcatcgccgccgcaaagcgctgaaaaacaaaaaaaaacagctgagcgcgggcggcaaaagcctgagcaaagaagaagaagaagaactgaaacgcctgacccgcgaagatgaagaacgcaaaaaagaagaacatggcccgagccgcctgggcgtgaacccgagcgaaggcggcccgcgcggcgcgccgggcggcggctttgtgccgagcatgcagggcattccggaaagccgctttacccgcaccggcgaaggcctggatgtgcgcggcagccgcggctttccgcaggatattctgtttccgagcgatccgccgtttagcccgcagagctgccgcccgcagagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaatgcaccccgcagagccagagcgaaagcaaaaaaaaccgccgcggcggccgcgaagatattctggaaaaatggattaccacccgccgcaaagcggaagaactggaaaaagatctgcgcaaagcgcgcaaaaccattaaaaaactggaagatgaaaacccgtggctgggcaacattattggcattattcgcaaaggcaaagatggcgaaggcgcgccgccggcgaaacgcccgcgcaccgatcagatggaaattgatagcggcaccggcaaacgcccgcataaaagcggctttaccgataaagaacgcgaagatcatcgccgccgcaaagcgctggaaaacaaaaaaaaacagctgagcagcggcggcaaaaacctgagccgcgaagaagaagaagaactgggccgcctgaccgtggaagatgaagaacgccgccgccgcgtggcgggcccgcgcaccggcgatgtgaacctgagcggcggcggcccgcgcggcgcgccgggcggcggctttgtgccgcgcatggaaggcgtgccggaaagcccgtttacccgcaccggcgaaggcctggatattcgcggcaaccagggctttccgtgggtgcgcccgagcccgccgcagcagcgcctgccgctgctggaatgcaccccgcag

SEQ ID NO: 9 - DNA, 3597 bp, synthetic construct
  misc_feature 1..3597: note = SVF-9 wild-type DNA sequence
  source 1..3597: mol_type = other DNA
atggccttaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagggaagcggagctactaacttcagcctgctgaagcaggctggagacgtggaggagaaccctggacctatgagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagctgccgcccgcagggaagcggagctactaacttcagcctgctgaagcaggctggagacgtggaggagaaccctggacctatgagccgcagcgaaagcaaaaaaaaccgcggcggccgcgaagaagtgctggaacagtgggtgaacggccgcaaaaaactggaagaactggaacgcgaactgcgccgcgcgcgcaaaaaaattaaaaaactggaagatgataacccgtggctgggcaacgtgaaaggcattctgggcaaaaaagataaagatggcgaaggcgcgccgccggcgaaacgcgcgcgcaccgatcagatggaaattgatagcggcccgcgcaaacgcccgctgcgcggcggctttaccgatcgcgaacgccaggatcatcgccgccgcaaagcgctgaaaaacaaaaaaaaacagctgagcgcgggcggcaaaagcctgagcaaagaagaagaagaagaactgaaacgcctgacccgcgaagatgaagaacgcaaaaaagaagaacatggcccgagccgcctgggcgtgaacccgagcgaaggcggcccgcgcggcgcgccgggcggcggctttgtgccgagcatgcagggcattccggaaagccgctttacccgcaccggcgaaggcctggatgtgcgcggcagccgcggctttccgcaggatattctgtttccgagcgatccgccgtttagcccgcagagctgccgcccgcagggaagcggagctactaacttcagcctgctgaagcaggctggagacgtggaggagaaccctggacctatgagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaatgcaccccgcagggaagcggagctactaacttcagcctgctgaagcaggctggagacgtggaggagaaccctggacctatgagccagagcgaaagcaaaaaaaaccgccgcggcggccgcgaagatattctggaaaaatggattaccacccgccgcaaagcggaagaactggaaaaagatctgcgcaaagcgcgcaaaaccattaaaaaactggaagatgaaaacccgtggctgggcaacattattggcattattcgcaaaggcaaagatggcgaaggcgcgccgccggcgaaacgcccgcgcaccgatcagatggaaattgatagcggcaccggcaaacgcccgcataaaagcggctttaccgataaagaacgcgaagatcatcgccgccgcaaagcgctggaaaacaaaaaaaaacagctgagcagcggcggcaaaaacctgagccgcgaagaagaagaagaactgggccgcctgaccgtggaagatgaagaacgccgccgccgcgtggcgggcccgcgcaccggcgatgtgaacctgagcggcggcggcccgcgcggcgcgccgggcggcggctttgtgccgcgcatggaaggcgtgccggaaagcccgtttacccgcaccggcgaaggcctggatattcgcggcaaccagggctttccgtgggtgcgcccgagcccgccgcagcagcgcctgccgctgctggaatgcaccccgcag

SEQ ID NO: 10 - DNA, 765 bp, synthetic construct
  misc_feature 1..765: note = SVF-10 wild-type DNA sequence
  source 1..765: mol_type = other DNA
atggccttaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaag

SEQ ID NO: 11 - DNA, 666 bp, synthetic construct
  misc_feature 1..666: note = SVF-11 wild-type DNA sequence
  source 1..666: mol_type = other DNA
atggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacag

SEQ ID NO: 12 - DNA, 1260 bp, synthetic construct
  misc_feature 1..1260: note = SVF-12 wild-type DNA sequence
  source 1..1260: mol_type = other DNA
atggcctctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcc

SEQ ID NO: 13 - DNA, 2091 bp, synthetic construct
  misc_feature 1..2091: note = SVF-1 codon optimized DNA sequence
  source 1..2091: mol_type = other DNA
atggctctgaagtataacgagaatgggactattaccgatgctgtggactgtgctctggaccctctgagtgagactaaatgtaccctgaagtcctttacagtggagaagggcatctatcagacctctaacttccgggtgcagcctacagagagcatcgtgcgcttccctaacatcaccaatctgtgcccattcggcgaggtgtttaacgccacacggttcgccagcgtgtacgcctggaatcggaagagaatctccaactgcgtggccgactattctgtgctgtacaattccgcctctttcagcacctttaagtgctatggcgtgtccccaaccaagctgaatgacctgtgcttcacaaacgtgtacgccgactcttttgtgatccggggcgatgaggtgagacagatcgcaccaggacagaccggcaagatcgcagactacaattataagctgcctgacgacttcaccggctgcgtgatcgcctggaactctaacaatctggatagcaaagtgggcggcaactacaattatctgtaccggctgttcagaaagtccaacctgaagccatttgagagggacatcagcaccgagatctaccaggccggctccacaccctgcaatggcgtggagggcttcaactgttattttcccctgcagagctacggctttcagcctaccaatggcgtgggctatcagccataccgcgtggtggtgctgtccttcgagctgctgcacgcaccagcaaccgtgtgcggacccaagaagggcagcggagccacaaacttttccctgctgaagcaggcaggcgacgtggaggagaatccaggacccatggcaagcgacaatggaccacagaatcagaggaacgcaccaagaatcactttcggcggcccaagcgactcaaccggcagcaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaataaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggcagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgagccggatcggaatggaggtgaccccaagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctcaaaacagctgcagcagtcaatgtcaagcgccgattcaactcaggca

SEQ ID NO: 14 - DNA, 2754 bp, synthetic construct
  misc_feature 1..2754: note = SVF-2 (OC2) codon optimized DNA sequence
  source 1..2754: mol_type = other DNA
atggctctgaagtataacgagaatgggactattaccgatgctgtggactgtgctctggaccctctgagtgagactaaatgtaccctgaagtcctttacagtggagaagggcatctatcagacctctaacttccgggtgcagcctacagagagcatcgtgcgcttccctaacatcaccaatctgtgcccattcggcgaggtgtttaacgccacacggttcgccagcgtgtacgcctggaatcggaagagaatctccaactgcgtggccgactattctgtgctgtacaattccgcctctttcagcacctttaagtgctatggcgtgtccccaaccaagctgaatgacctgtgcttcacaaacgtgtacgccgactcttttgtgatccggggcgatgaggtgagacagatcgcaccaggacagaccggcaagatcgcagactacaattataagctgcctgacgacttcaccggctgcgtgatcgcctggaactctaacaatctggatagcaaagtgggcggcaactacaattatctgtaccggctgttcagaaagtccaacctgaagccatttgagagggacatcagcaccgagatctaccaggccggctccacaccctgcaatggcgtggagggcttcaactgttattttcccctgcagagctacggctttcagcctaccaatggcgtgggctatcagccataccgcgtggtggtgctgtccttcgagctgctgcacgcaccagcaaccgtgtgcggacccaagaagggcagcggagccacaaacttttccctgctgaagcaggcaggcgacgtggaggagaatccaggacctatggccgatagcaacggcaccatcacagtggaggagctgaagaagctgctggagcagtggaacctggtcatcggcttcctgtttctgacatggatctgtctgctgcagttcgcctatgccaacaggaatcgctttctgtacatcatcaagctgatcttcctgtggctgctgtggccagtgaccctggcatgcttcgtgctggccgccgtgtatcggatcaactggatcacaggcggcatcgccatcgccatggcctgtctggtgggcctgatgtggctgtcctactttatcgcctctttcagactgtttgcccggaccagatccatgtggtctttcaaccccgagacaaatatcctgctgaacgtgcctctgcacggcaccatcctgacaaggccactgctggagtccgagctggtcatcggagccgtgatcctgaggggacacctgaggatcgcaggacaccacctgggccgctgtgacatcaaggatctgcctaaggagatcaccgtggccacaagccggaccctgtcctactataagctgggagcatctcagagagtggcaggcgattctggcttcgccgcctatagcaggtaccgcatcggcaattacaagctgaacaccgaccacagctcctctagcgataacatcgccctgctggtgcaggcaagcgacaatggaccacagaatcagaggaacgcaccaagaatcactttcggcggcccaagcgactcaaccggcagcaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaataaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggcagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgagccggatcggaatggaggtgaccccaagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctcaaaacagctgcagcagtcaatgtcaagcgccgattcaactcaggca

SEQ ID NO: 15 - DNA, 2823 bp, synthetic construct
  misc_feature 1..2823: note = SVF-3 codon optimized DNA sequence
  source 1..2823: mol_type = other DNA
atggctctgaagtataacgagaatgggactattaccgatgctgtggactgtgctctggaccctctgagtgagactaaatgtaccctgaagtcctttacagtggagaagggcatctatcagacctctaacttccgggtgcagcctacagagagcatcgtgcgcttccctaacatcaccaatctgtgcccattcggcgaggtgtttaacgccacacggttcgccagcgtgtacgcctggaatcggaagagaatctccaactgcgtggccgactattctgtgctgtacaattccgcctctttcagcacctttaagtgctatggcgtgtccccaaccaagctgaatgacctgtgcttcacaaacgtgtacgccgactcttttgtgatccggggcgatgaggtgagacagatcgcaccaggacagaccggcaagatcgcagactacaattataagctgcctgacgacttcaccggctgcgtgatcgcctggaactctaacaatctggatagcaaagtgggcggcaactacaattatctgtaccggctgttcagaaagtccaacctgaagccatttgagagggacatcagcaccgagatctaccaggccggctccacaccctgcaatggcgtggagggcttcaactgttattttcccctgcagagctacggctttcagcctaccaatggcgtgggctatcagccataccgcgtggtggtgctgtccttcgagctgctgcacgcaccagcaaccgtgtgcggacccaagaagggcagcggagccacaaacttttccctgctgaagcaggcaggcgacgtggaggagaatccaggacctatggccgatagcaacggcaccatcacagtggaggagctgaagaagctgctggagcagtggaacctggtcatcggcttcctgtttctgacatggatctgtctgctgcagttcgcctatgccaacaggaatcgctttctgtacatcatcaagctgatcttcctgtggctgctgtggccagtgaccctggcatgcttcgtgctggccgccgtgtatcggatcaactggatcacaggcggcatcgccatcgccatggcctgtctggtgggcctgatgtggctgtcctactttatcgcctctttcagactgtttgcccggaccagatccatgtggtctttcaaccccgagacaaatatcctgctgaacgtgcctctgcacggcaccatcctgacaaggccactgctggagtccgagctggtcatcggagccgtgatcctgaggggacacctgaggatcgcaggacaccacctgggccgctgtgacatcaaggatctgcctaaggagatcaccgtggccacaagccggaccctgtcctactataagctgggagcatctcagagagtggcaggcgattctggcttcgccgcctatagcaggtaccgcatcggcaattacaagctgaacaccgaccacagctcctctagcgataacatcgccctgctggtgcagggcagcggagcaaccaactttagtctgctgaaacaggccggagacgtggaagaaaaccccggacccatggcaagcgacaatggaccacagaatcagaggaacgcaccaagaatcactttcggcggcccaagcgactcaaccggcagcaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaataaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggcagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgagccggatcggaatggaggtgaccccaagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctcaaaacagctgcagcagtcaatgtcaagcgccgattcaactcaggca

SEQ ID NO: 16 - DNA, 1497 bp, synthetic construct
  misc_feature 1..1497: note = SVF-4 codon optimized DNA sequence
  source 1..1497: mol_type = other DNA
atggctctgaagtataacgagaatgggactattaccgatgctgtggactgtgctctggaccctctgagtgagactaaatgtaccctgaagtcctttacagtggagaagggcatctatcagacctctaacttccgggtgcagcctacagagagcatcgtgcgcttccctaacatcaccaatctgtgcccattcggcgaggtgtttaacgccacacggttcgccagcgtgtacgcctggaatcggaagagaatctccaactgcgtggccgactattctgtgctgtacaattccgcctctttcagcacctttaagtgctatggcgtgtccccaaccaagctgaatgacctgtgcttcacaaacgtgtacgccgactcttttgtgatccggggcgatgaggtgagacagatcgcaccaggacagaccggcaagatcgcagactacaattataagctgcctgacgacttcaccggctgcgtgatcgcctggaactctaacaatctggatagcaaagtgggcggcaactacaattatctgtaccggctgttcagaaagtccaacctgaagccatttgagagggacatcagcaccgagatctaccaggccggctccacaccctgcaatggcgtggagggcttcaactgttattttcccctgcagagctacggctttcagcctaccaatggcgtgggctatcagccataccgcgtggtggtgctgtccttcgagctgctgcacgcaccagcaaccgtgtgcggacccaagaagggcagcggagccacaaacttttccctgctgaagcaggcaggcgacgtggaggagaatccaggacctatggccgatagcaacggcaccatcacagtggaggagctgaagaagctgctggagcagtggaacctggtcatcggcttcctgtttctgacatggatctgtctgctgcagttcgcctatgccaacaggaatcgctttctgtacatcatcaagctgatcttcctgtggctgctgtggccagtgaccctggcatgcttcgtgctggccgccgtgtatcggatcaactggatcacaggcggcatcgccatcgccatggcctgtctggtgggcctgatgtggctgtcctactttatcgcctctttcagactgtttgcccggaccagatccatgtggtctttcaaccccgagacaaatatcctgctgaacgtgcctctgcacggcaccatcctgacaaggccactgctggagtccgagctggtcatcggagccgtgatcctgaggggacacctgaggatcgcaggacaccacctgggccgctgtgacatcaaggatctgcctaaggagatcaccgtggccacaagccggaccctgtcctactataagctgggagcatctcagagagtggcaggcgattctggcttcgccgcctatagcaggtaccgcatcggcaattacaagctgaacaccgaccacagctcctctagcgataacatcgccctgctggtgcag

SEQ ID NO: 17 - DNA, 2022 bp, synthetic construct
  misc_feature 1..2022: note = SVF-5 codon optimized DNA sequence
  source 1..2022: mol_type = other DNA
atggctctgaagtataacgagaatgggactattaccgatgctgtggactgtgctctggaccctctgagtgagactaaatgtaccctgaagtcctttacagtggagaagggcatctatcagacctctaacttccgggtgcagcctacagagagcatcgtgcgcttccctaacatcaccaatctgtgcccattcggcgaggtgtttaacgccacacggttcgccagcgtgtacgcctggaatcggaagagaatctccaactgcgtggccgactattctgtgctgtacaattccgcctctttcagcacctttaagtgctatggcgtgtccccaaccaagctgaatgacctgtgcttcacaaacgtgtacgccgactcttttgtgatccggggcgatgaggtgagacagatcgcaccaggacagaccggcaagatcgcagactacaattataagctgcctgacgacttcaccggctgcgtgatcgcctggaactctaacaatctggatagcaaagtgggcggcaactacaattatctgtaccggctgttcagaaagtccaacctgaagccatttgagagggacatcagcaccgagatctaccaggccggctccacaccctgcaatggcgtggagggcttcaactgttattttcccctgcagagctacggctttcagcctaccaatggcgtgggctatcagccataccgcgtggtggtgctgtccttcgagctgctgcacgcaccagcaaccgtgtgcggacccaagaaggcaagcgacaatggaccacagaatcagaggaacgcaccaagaatcactttcggcggcccaagcgactcaaccggcagcaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaataaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggcagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgagccggatcggaatggaggtgaccccaagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctcaaaacagctgcagcagtcaatgtcaagcgccgattcaactcaggca

SEQ ID NO: 18 - DNA, 2685 bp, synthetic construct
  misc_feature 1..2685: note = SVF-6 codon optimized DNA sequence
  source 1..2685: mol_type = other DNA
atggctctgaagtataacgagaatgggactattaccgatgctgtggactgtgctctggaccctctgagtgagactaaatgtaccctgaagtcctttacagtggagaagggcatctatcagacctctaacttccgggtgcagcctacagagagcatcgtgcgcttccctaacatcaccaatctgtgcccattcggcgaggtgtttaacgccacacggttcgccagcgtgtacgcctggaatcggaagagaatctccaactgcgtggccgactattctgtgctgtacaattccgcctctttcagcacctttaagtgctatggcgtgtccccaaccaagctgaatgacctgtgcttcacaaacgtgtacgccgactcttttgtgatccggggcgatgaggtgagacagatcgcaccaggacagaccggcaagatcgcagactacaattataagctgcctgacgacttcaccggctgcgtgatcgcctggaactctaacaatctggatagcaaagtgggcggcaactacaattatctgtaccggctgttcagaaagtccaacctgaagccatttgagagggacatcagcaccgagatctaccaggccggctccacaccctgcaatggcgtggagggcttcaactgttattttcccctgcagagctacggctttcagcctaccaatggcgtgggctatcagccataccgcgtggtggtgctgtccttcgagctgctgcacgcaccagcaaccgtgtgcggacccaagaaggccgatagcaacggcaccatcacagtggaggagctgaagaagctgctggagcagtggaacctggtcatcggcttcctgtttctgacatggatctgtctgctgcagttcgcctatgccaacaggaatcgctttctgtacatcatcaagctgatcttcctgtggctgctgtggccagtgaccctggcatgcttcgtgctggccgccgtgtatcggatcaactggatcacaggcggcatcgccatcgccatggcctgtctggtgggcctgatgtggctgtcctactttatcgcctctttcagactgtttgcccggaccagatccatgtggtctttcaaccccgagacaaatatcctgctgaacgtgcctctgcacggcaccatcctgacaaggccactgctggagtccgagctggtcatcggagccgtgatcctgaggggacacctgaggatcgcaggacaccacctgggccgctgtgacatcaaggatctgcctaaggagatcaccgtggccacaagccggaccctgtcctactataagctgggagcatctcagagagtggcaggcgattctggcttcgccgcctatagcaggtaccgcatcggcaattacaagctgaacaccgaccacagctcctctagcgataacatcgccctgctggtgcaggcaagcgacaatggaccacagaatcagaggaacgcaccaagaatcactttcggcggcccaagcgactcaaccggcagcaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaataaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggcagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgagccggatcggaatggaggtgaccccaagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctcaaaacagctgcagcagtcaatgtcaagcgccgattcaactcaggca

SEQ ID NO: 19 - DNA, 1428 bp, synthetic construct
  misc_feature 1..1428: note = SVF-7 codon optimized DNA sequence
  source 1..1428: mol_type = other DNA
atggctctgaagtataacgagaatgggactattaccgatgctgtggactgtgctctggaccctctgagtgagactaaatgtaccctgaagtcctttacagtggagaagggcatctatcagacctctaacttccgggtgcagcctacagagagcatcgtgcgcttccctaacatcaccaatctgtgcccattcggcgaggtgtttaacgccacacggttcgccagcgtgtacgcctggaatcggaagagaatctccaactgcgtggccgactattctgtgctgtacaattccgcctctttcagcacctttaagtgctatggcgtgtccccaaccaagctgaatgacctgtgcttcacaaacgtgtacgccgactcttttgtgatccggggcgatgaggtgagacagatcgcaccaggacagaccggcaagatcgcagactacaattataagctgcctgacgacttcaccggctgcgtgatcgcctggaactctaacaatctggatagcaaagtgggcggcaactacaattatctgtaccggctgttcagaaagtccaacctgaagccatttgagagggacatcagcaccgagatctaccaggccggctccacaccctgcaatggcgtggagggcttcaactgttattttcccctgcagagctacggctttcagcctaccaatggcgtgggctatcagccataccgcgtggtggtgctgtccttcgagctgctgcacgcaccagcaaccgtgtgcggacccaagaaggccgatagcaacggcaccatcacagtggaggagctgaagaagctgctggagcagtggaacctggtcatcggcttcctgtttctgacatggatctgtctgctgcagttcgcctatgccaacaggaatcgctttctgtacatcatcaagctgatcttcctgtggctgctgtggccagtgaccctggcatgcttcgtgctggccgccgtgtatcggatcaactggatcacaggcggcatcgccatcgccatggcctgtctggtgggcctgatgtggctgtcctactttatcgcctctttcagactgtttgcccggaccagatccatgtggtctttcaaccccgagacaaatatcctgctgaacgtgcctctgcacggcaccatcctgacaaggccactgctggagtccgagctggtcatcggagccgtgatcctgaggggacacctgaggatcgcaggacaccacctgggccgctgtgacatcaaggatctgcctaaggagatcaccgtggccacaagccggaccctgtcctactataagctgggagcatctcagagagtggcaggcgattctggcttcgccgcctatagcaggtaccgcatcggcaattacaagctgaacaccgaccacagctcctctagcgataacatcgccctgctggtgcag

SEQ ID NO: 20 - DNA, 3321 bp, synthetic construct
  misc_feature 1..3321: note = SVF-8 codon optimized DNA sequence
  source 1..3321: mol_type = other DNA
atggctctgaagtataacgagaatgggactattaccgatgctgtggactgtgctctggaccctctgagtgagactaaatgtaccctgaagtcctttacagtggagaagggcatctatcagacctctaacttccgggtgcagcctacagagagcatcgtgcgcttccctaacatcaccaatctgtgcccattcggcgaggtgtttaacgccacacggttcgccagcgtgtacgcctggaatcggaagagaatctccaactgcgtggccgactattctgtgctgtacaattccgcctctttcagcacctttaagtgctatggcgtgtccccaaccaagctgaatgacctgtgcttcacaaacgtgtacgccgactcttttgtgatccggggcgatgaggtgagacagatcgcaccaggacagaccggcaagatcgcagactacaattataagctgcctgacgacttcaccggctgcgtgatcgcctggaactctaacaatctggatagcaaagtgggcggcaactacaattatctgtaccggctgttcagaaagtccaacctgaagccatttgagagggacatcagcaccgagatctaccaggccggctccacaccctgcaatggcgtggagggcttcaactgttattttcccctgcagagctacggctttcagcctaccaatggcgtgggctatcagccataccgcgtggtggtgctgtccttcgagctgctgcacgcaccagcaaccgtgtgcggacccaagaagtccaggtctgagagcaagaagaataggggaggaagggaggagatcctggagcagtgggtgggagcacgcaagaagctggaggagctggagcgggacctgagaaagatcaagaagaagatcaagaagctggaagaggagaacccctggctgggcaatatcaagggcatcctgggcaagaaggatagggagggcgagggagcaccacctgcaaagagggcaagggcagaccagatggaggtggattccggacctaggaagcggcccttccggggagagtttacagacaaggagcggagagatcacaggcgccggaaggccctggagaacaagcggaagcagctgagctccggcggcaagtccctgtctaaggaggaggaggaggagctgagaaagctgaccgaggaggacgagagaagggagaggagggtggcaggaccaagggtgggaggagtgaatcctctggagggaggaacaaggggagcaccaggaggaggcttcgtgcctagcatgcagggcgtgcctgagtccccatttgccaggaccggagagggcctggacgtgagaggaaaccagggcttcccatgggacatcctgtttccagccgatccacccttcagcccacagtcctgcaggcctcagagccggtccgagtctaagaagaatagaggcggaagggaggaggtgctggagcagtgggtgaacggcagaaagaagctggaagaactggagagggagctgagaagggcccgcaagaagatcaagaagttagaggacgataatccttggctgggcaatgtgaaaggcatcctgggcaagaaggacaaggatggagagggagcacctccagcaaagagggcacgcacagaccagatggagatcgatagcggaccaaggaagcggcccctgagaggaggctttaccgacagggagaggcaggatcaccgccggagaaaggccctgaagaacaagaagaagcagctgtccgccggaggcaagagcctgtccaaagaagaggaggaggagctgaagcggctgacaagagaggacgaggagcggaagaaggaggagcacggaccaagccggctgggagtgaatccaagcgagggaggacctagaggcgcccctggcggaggcttcgtgccttccatgcagggcatcccagagtctaggtttaccaggacaggcgaaggcctggacgtgcggggctccagaggctttccccaggacatcctgttcccttctgatccccctttttctccacagagctgtagaccacagtctcagagcgagacaaggaggggccggagaggaaccagggaggagacactggagaagtggatcaccgccagaaagaaggccgaggagctggagaaggacctgaggaagacccgcaagacaatcaagaagctggaagaagagaacccatggctgggcaatatcgtgggcatcatcagaaagggcaaggacggcgagggagcaccaccagcaaagcggcccagaaccgatcagatggaggtggatagcggccctggcaagaggccacacaagtccggcttcaccgacaaggagagggaggaccataggcgccggaaggccctggagaacaagaagaagcaattaagcgccggcggcaagatcctgtccaaagaggaagaggaggagctgagaaggctgaccgacgaggatgaggagaggaaaagaagggtggcaggacctagggtgggcgacgtgaatccaagcaggggaggaccaaggggagcacctgggggcggcttcgtgcctcagatggcaggagtgccagagtcccctttttctcggacaggcgagggcctggatatcagaggaacccagggattcccatgggtgtccccatctcctccacagcagaggctgccactgctggagtgcaccccacagagccagagcgaaagcaaaaaaaaccgccgcggcggccgcgaagatattctggaaaaatggattaccacccgccgcaaagcggaagaactggaaaaagatctgcgcaaagcgcgcaaaaccattaaaaaactggaagatgaaaacccgtggctgggcaacattattggcattattcgcaaaggcaaagatggcgaaggcgcgccgccggcgaaacgcccgcgcaccgatcagatggaaattgatagcggcaccggcaaacgcccgcataaaagcggctttaccgataaagaacgcgaagatcatcgccgccgcaaagcgctggaaaacaaaaaaaaacagctgagcagcggcggcaaaaacctgagccgcgaagaagaagaagaactgggccgcctgaccgtggaagatgaagaacgccgccgccgcgtggcgggcccgcgcaccggcgatgtgaacctgagcggcggcggcccgcgcggcgcgccgggcggcggctttgtgccgcgcatggaaggcgtgccggaaagcccgtttacccgcaccggcgaaggcctggatattcgcggcaaccagggctttccgtgggtgcgcccgagcccgccgcagcagcgcctgccgctgctggaatgcaccccgcag

SEQ ID NO: 21 - DNA, 3597 bp, synthetic construct
  misc_feature 1..3597: note = SVF-9 codon optimized DNA sequence
  source 1..3597: mol_type = other DNA
atggctctgaagtataacgagaatgggactattaccgatgctgtggactgtgctctggaccctctgagtgagactaaatgtaccctgaagtcctttacagtggagaagggcatctatcagacctctaacttccgggtgcagcctacagagagcatcgtgcgcttccctaacatcaccaatctgtgcccattcggcgaggtgtttaacgccacacggttcgccagcgtgtacgcctggaatcggaagagaatctccaactgcgtggccgactattctgtgctgtacaattccgcctctttcagcacctttaagtgctatggcgtgtccccaaccaagctgaatgacctgtgcttcacaaacgtgtacgccgactcttttgtgatccggggcgatgaggtgagacagatcgcaccaggacagaccggcaagatcgcagactacaattataagctgcctgacgacttcaccggctgcgtgatcgcctggaactctaacaatctggatagcaaagtgggcggcaactacaattatctgtaccggctgttcagaaagtccaacctgaagccatttgagagggacatcagcaccgagatctaccaggccggctccacaccctgcaatggcgtggagggcttcaactgttattttcccctgcagagctacggctttcagcctaccaatggcgtgggctatcagccataccgcgtggtggtgctgtccttcgagctgctgcacgcaccagcaaccgtgtgcggacccaagaagggcagcggagccacaaacttttccctgctgaagcaggcaggcgacgtggaggagaatccaggacctatgtccaggtctgagagcaagaagaataggggaggaagggaggagatcctggagcagtgggtgggagcacgcaagaagctggaggagctggagcgggacctgagaaagatcaagaagaagatcaagaagctggaagaggagaacccctggctgggcaatatcaagggcatcctgggcaagaaggatagggagggcgagggagcaccacctgcaaagagggcaagggcagaccagatggaggtggattccggacctaggaagcggcccttccggggagagtttacagacaaggagcggagagatcacaggcgccggaaggccctggagaacaagcggaagcagctgagctccggcggcaagtccctgtctaaggaggaggaggaggagctgagaaagctgaccgaggaggacgagagaagggagaggagggtggcaggaccaagggtgggaggagtgaatcctctggagggaggaacaaggggagcaccaggaggaggcttcgtgcctagcatgcagggcgtgcctgagtccccatttgccaggaccggagagggcctggacgtgagaggaaaccagggcttcccatgggacatcctgtttccagccgatccacccttcagcccacagtcctgcaggcctcagggctccggcgccaccaatttctctctgctgaagcaggccggcgatgtggaagaaaaccctggaccaatgagccggtccgagtctaagaagaatagaggcggaagggaggaggtgctggagcagtgggtgaacggcagaaagaagctggaagaactggagagggagctgagaagggcccgcaagaagatcaagaagttagaggacgataatccttggctgggcaatgtgaaaggcatcctgggcaagaaggacaaggatggagagggagcacctccagcaaagagggcacgcacagaccagatggagatcgatagcggaccaaggaagcggcccctgagaggaggctttaccgacagggagaggcaggatcaccgccggagaaaggccctgaagaacaagaagaagcagctgtccgccggaggcaagagcctgtccaaagaagaggaggaggagctgaagcggctgacaagagaggacgaggagcggaagaaggaggagcacggaccaagccggctgggagtgaatccaagcgagggaggacctagaggcgcccctggcggaggcttcgtgccttccatgcagggcatcccagagtctaggtttaccaggacaggcgaaggcctggacgtgcggggctccagaggctttccccaggacatcctgttcccttctgatccccctttttctccacagagctgtagaccacagggcagcggagcaaccaacttctccctgctgaagcaagccggcgatgtggaggagaatccaggacctatgtctcagagcgagacaaggaggggccggagaggaaccagggaggagacactggagaagtggatcaccgccagaaagaaggccgaggagctggagaaggacctgaggaagacccgcaagacaatcaagaagctggaagaagagaacccatggctgggcaatatcgtgggcatcatcagaaagggcaaggacggcgagggagcaccaccagcaaagcggcccagaaccgatcagatggaggtggatagcggccctggcaagaggccacacaagtccggcttcaccgacaaggagagggaggaccataggcgccggaaggccctggagaacaagaagaagcaattaagcgccggcggcaagatcctgtccaaagaggaagaggaggagctgagaaggctgaccgacgaggatgaggagaggaaaagaagggtggcaggacctagggtgggcgacgtgaatccaagcaggggaggaccaaggggagcacctgggggcggcttcgtgcctcagatggcaggagtgccagagtcccctttttctcggacaggcgagggcctggatatcagaggaacccagggattcccatgggtgtccccatctcctccacagcagaggctgccactgctggagtgcaccccacagggctctggagccacaaactttagcctgctgaagcaggccggcgacgtggaagaaaacccaggacccatgagccagagcgaaagcaaaaaaaaccgccgcggcggccgcgaagatattctggaaaaatggattaccacccgccgcaaagcggaagaactggaaaaagatctgcgcaaagcgcgcaaaaccattaaaaaactggaagatgaaaacccgtggctgggcaacattattggcattattcgcaaaggcaaagatggcgaaggcgcgccgccggcgaaacgcccgcgcaccgatcagatggaaattgatagcggcaccggcaaacgcccgcataaaagcggctttaccgataaagaacgcgaagatcatcgccgccgcaaagcgctggaaaacaaaaaaaaacagctgagcagcggcggcaaaaacctgagccgcgaagaagaagaagaactgggccgcctgaccgtggaagatgaagaacgccgccgccgcgtggcgggcccgcgcaccggcgatgtgaacctgagcggcggcggcccgcgcggcgcgccgggcggcggctttgtgccgcgcatggaaggcgtgccggaaagcccgtttacccgcaccggcgaaggcctggatattcgcggcaaccagggctttccgtgggtgcgcccgagcccgccgcagcagcgcctgccgctgctggaatgcaccccgcag

SEQ ID NO: 22 - DNA, 765 bp, synthetic construct
  misc_feature 1..765: note = SVF-10 codon optimized DNA sequence
  source 1..765: mol_type = other DNA
atggctctgaagtataacgagaatgggactattaccgatgctgtggactgtgctctggaccctctgagtgagactaaatgtaccctgaagtcctttacagtggagaagggcatctatcagacctctaacttccgggtgcagcctacagagagcatcgtgcgcttccctaacatcaccaatctgtgcccattcggcgaggtgtttaacgccacacggttcgccagcgtgtacgcctggaatcggaagagaatctccaactgcgtggccgactattctgtgctgtacaattccgcctctttcagcacctttaagtgctatggcgtgtccccaaccaagctgaatgacctgtgcttcacaaacgtgtacgccgactcttttgtgatccggggcgatgaggtgagacagatcgcaccaggacagaccggcaagatcgcagactacaattataagctgcctgacgacttcaccggctgcgtgatcgcctggaactctaacaatctggatagcaaagtgggcggcaactacaattatctgtaccggctgttcagaaagtccaacctgaagccatttgagagggacatcagcaccgagatctaccaggccggctccacaccctgcaatggcgtggagggcttcaactgttattttcccctgcagagctacggctttcagcctaccaatggcgtgggctatcagccataccgcgtggtggtgctgtccttcgagctgctgcacgcaccagcaaccgtgtgcggacccaagaag

SEQ ID NO: 23 - DNA, 666 bp, synthetic construct
  misc_feature 1..666: note = SVF-11 codon optimized DNA sequence
  source 1..666: mol_type = other DNA
atggccgatagcaacggcaccatcacagtggaggagctgaagaagctgctggagcagtggaacctggtcatcggcttcctgtttctgacatggatctgtctgctgcagttcgcctatgccaacaggaatcgctttctgtacatcatcaagctgatcttcctgtggctgctgtggccagtgaccctggcatgcttcgtgctggccgccgtgtatcggatcaactggatcacaggcggcatcgccatcgccatggcctgtctggtgggcctgatgtggctgtcctactttatcgcctctttcagactgtttgcccggaccagatccatgtggtctttcaaccccgagacaaatatcctgctgaacgtgcctctgcacggcaccatcctgacaaggccactgctggagtccgagctggtcatcggagccgtgatcctgaggggacacctgaggatcgcaggacaccacctgggccgctgtgacatcaaggatctgcctaaggagatcaccgtggccacaagccggaccctgtcctactataagctgggagcatctcagagagtggcaggcgattctggcttcgccgcctatagcaggtaccgcatcggcaattacaagctgaacaccgaccacagctcctctagcgataacatcgccctgctggtgcag

SEQ ID NO: 24 - DNA, 1263 bp, synthetic construct
  misc_feature 1..1263: note = SVF-12 codon optimized DNA sequence
  source 1..1263: mol_type = other DNA
atggcaagcgacaatggaccacagaatcagaggaacgcaccaagaatcactttcggcggcccaagcgactcaaccggcagcaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaataaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggcagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgagccggatcggaatggaggtgaccccaagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctcaaaacagctgcagcagtcaatgtcaagcgccgattcaactcaggcataa

SEQ ID NO: 25 - protein, 697 aa, synthetic construct
  REGION 1..697: note = SVF-1 polypeptide sequence
MALKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKGSGATNFSLLKQAGDVEENPGPMASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 26 - protein, 918 aa, synthetic construct
  REGION 1..918: note = SVF-2 (OC2) polypeptide sequence
MALKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 27 - protein, 941 aa, synthetic construct
  REGION 1..941: note = SVF-3 polypeptide sequence
MALKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQGSGATNFSLLKQAGDVEENPGPMASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 28 - protein, 499 aa, synthetic construct
  REGION 1..499: note = SVF-4 polypeptide sequence
MALKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 29 - protein, 674 aa, synthetic construct
  REGION 1..674: note = SVF-5 polypeptide sequence
MALKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 30 - protein, 895 aa, synthetic construct
  REGION 1..895: note = SVF-6 polypeptide sequence
MALKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 31 - protein, 476 aa, synthetic construct
  REGION 1..476: note = SVF-7 polypeptide sequence
MALKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 32 - protein, 1107 aa, synthetic construct
  REGION 1..1107: note = SVF-8 polypeptide sequence
MALKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQ

SEQ ID NO: 33 - protein, 1199 aa, synthetic construct
  REGION 1..1199: note = SVF-9 polypeptide sequence
MALKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKGSGATNFSLLKQAGDVEENPGPMSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQGSGATNFSLLKQAGDVEENPGPMSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQGSGATNFSLLKQAGDVEENPGPMSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQGSGATNFSLLKQAGDVEENPGPMSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQ

SEQ ID NO: 34 - protein, 255 aa, synthetic construct
  REGION 1..255: note = SVF-10 polypeptide sequence (RBD peptide)
MALKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKK

SEQ ID NO: 35 - protein, 222 aa, synthetic construct
  REGION 1..222: note = SVF-11 polypeptide sequence (M peptide)
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 36 - protein, 420 aa, synthetic construct
  REGION 1..420: note = SVF-12 polypeptide sequence (NP peptide)
MASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 37 - DNA, 66 bp, synthetic construct
  misc_feature 1..66: note = P2A autocatalytic peptide cleavage site DNA sequence
  source 1..66: mol_type = other DNA
ggaagcggagctactaacttcagcctgctgaagcaggctggagacgtggaggagaaccctggacct

SEQ ID NO: 38 - protein, 22 aa, synthetic construct
  REGION 1..22: note = P2A autocatalytic peptide cleavage site polypeptide sequence
GSGATNFSLLKQAGDVEENPGP

SEQ ID NO: 39 - DNA, 3309 bp, synthetic construct
  misc_feature 1..3309: note = OC2.2 codon optimized DNA sequence
  source 1..3309: mol_type = other DNA
atggcccgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaagatcgctgactacaattataaactgcccgacgatttcaccggctgcgtgatcgcctggaacagcaacaatctggattccaaagtgggcgggaactacaattatctgtaccggctgttcagaaagtccaatctgaaaccctttgagagggacatcagtactgaaatctaccaggccgggtcaaccccttgcaatggggtggagggattcaactgttactttccactgcagagttatggatttcagcccaccaacggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaagattgctgactataattacaaactgccagacgatttcactggatgcgtgattgcctggaactctaacaatctggatagtaaagtgggaggcaactataattacctgtataggctgttccgcaagtctaatctgaaaccatttgagcgggacatcagcaccgagatctaccaggccggctccaccccctgcaatggagtggagggcttcaactgctattttccactgcagagctatgggtttcagcccacaaacggggtgggataccagccttatagagtggtggtgctgtccttcgaactgctgcatgcccctgctacagtgtgcggcccaaagaaatctaccaacctggtcaagaacaagggaagcggagctactaacttcagcctgctgaagcaggctggagacgtggaggagaaccctggacctatggccgattccaacggaactattaccgtggaggaactgaagaaactgctggagcagtggaacctggtcatcggcttcctgtttctgacctggatttgtctgctgcagttcgcctacgctaaccggaacagatttctgtacatcattaagctgatcttcctgtggctgctgtggcccgtgactctggcctgcttcgtgctggccgccgtgtacaggatcaactggattaccgggggaatcgccattgctatggcctgtctggtgggcctgatgtggctgtcatacttcatcgccagcttccgcctgtttgctaggacacgcagcatgtggtccttcaaccctgagactaatatcctgctgaacgtgccactgcacggcaccattctgacacggcccctgctggagtccgaactggtcatcggggccgtgattctgaggggacatctgcgcatcgctgggcaccatctgggaagatgcgacatcaaggatctgcctaaagaaattactgtggccacctcaaggacactgagctactataagctgggagctagccagagggtggctggggacagcggatttgctgcttactcccggtatagaattggcaattacaaactgaacacagaccacagctcctctagtgataatatcgccctgctggtgcaggctagcgacaacgggccccagaaccagcggaatgcccctagaatcaccttcggcgggccatccgattctacaggcagcaaccagaatggagagaggtccggagctcgctctaagcagagacggccccagggcctgccaaacaataccgcctcctggtttactgctctgacccagcatgggaaggaagatctgaaattccccaggggacagggcgtgcctatcaacaccaattcaagcccagacgatcagattgggtactataggagggctacaaggagaatccggggaggcgacggaaagatgaaagatctgagccccagatggtacttttactatctgggaacaggaccagaggctggactgccttatggcgctaacaaggacggaatcatttgggtggccactgaaggcgctctgaatacccctaaagatcacattggaacccgcaacccagccaacaatgccgctatcgtgctgcagctgccacagggcaccacactgcccaagggcttctacgctgaggggagtaggggaggatcacaggcttcctctcgcagttcaagcaggtcccgcaattcctctcggaactctacaccagggagttcacggggaactagcccagctagaatggctggaaatggaggggacgccgctctggccctgctgctgctggatagactgaaccagctggagtctaagatgagtggcaaagggcagcagcagcagggacagacagtgactaagaaatccgccgctgaagcctctaagaaaccccggcagaagagaaccgctacaaaagcctacaatgtgacccaggcttttggcaggcgcggacctgagcagacacagggaaacttcggcgaccaggaactgattaggcagggcaccgattataagcactggccccagatcgctcagttcgcccctagtgcttcagccttctttggaatgtctcgcattggcatggaggtgacacctagtggcacttggctgacttacaccggggccatcaagctggacgataaagacccaaacttcaaggatcaggtcatcctgctgaacaagcatattgacgcctataaaaccttcccccctacagagcccaagaaagacaagaaaaagaaagccgatgaaacccaggctctgccccagaggcagaagaaacagcagacagtgactctgctgcctgccgctgatctggacgatttcagtaaacagctgcagcagagtatgagtagtgccgacagtacccaggct

SEQ ID NO: 40 - DNA, 3360 bp, synthetic construct
  misc_feature 1..3360: note = OC2.3 codon optimized DNA sequence
  source 1..3360: mol_type = other DNA
atggactggacctggattctgttcctggtggccgccgccacacgggtgcacagcgcccgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaagatcgctgactacaattataaactgcccgacgatttcaccggctgcgtgatcgcctggaacagcaacaatctggattccaaagtgggcgggaactacaattatctgtaccggctgttcagaaagtccaatctgaaaccctttgagagggacatcagtactgaaatctaccaggccgggtcaaccccttgcaatggggtggagggattcaactgttactttccactgcagagttatggatttcagcccaccaacggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaagattgctgactataattacaaactgccagacgatttcactggatgcgtgattgcctggaactctaacaatctggatagtaaagtgggaggcaactataattacctgtataggctgttccgcaagtctaatctgaaaccatttgagcgggacatcagcaccgagatctaccaggccggctccaccccctgcaatggagtggagggcttcaactgctattttccactgcagagctatgggtttcagcccacaaacggggtgggataccagccttatagagtggtggtgctgtccttcgaactgctgcatgcccctgctacagtgtgcggcccaaagaaatctaccaacctggtcaagaacaagggaagcggagctactaacttcagcctgctgaagcaggctggagacgtggaggagaaccctggacctatggccgattccaacggaactattaccgtggaggaactgaagaaactgctggagcagtggaacctggtcatcggcttcctgtttctgacctggatttgtctgctgcagttcgcctacgctaaccggaacagatttctgtacatcattaagctgatcttcctgtggctgctgtggcccgtgactctggcctgcttcgtgctggccgccgtgtacaggatcaactggattaccgggggaatcgccattgctatggcctgtctggtgggcctgatgtggctgtcatacttcatcgccagcttccgcctgtttgctaggacacgcagcatgtggtccttcaaccctgagactaatatcctgctgaacgtgccactgcacggcaccattctgacacggcccctgctggagtccgaactggtcatcggggccgtgattctgaggggacatctgcgcatcgctgggcaccatctgggaagatgcgacatcaaggatctgcctaaagaaattactgtggccacctcaaggacactgagctactataagctgggagctagccagagggtggctggggacagcggatttgctgcttactcccggtatagaattggcaattacaaactgaacacagaccacagctcctctagtgataatatcgccctgctggtgcaggctagcgacaacgggccccagaaccagcggaatgcccctagaatcaccttcggcgggccatccgattctacaggcagcaaccagaatggagagaggtccggagctcgctctaagcagagacggccccagggcctgccaaacaataccgcctcctggtttactgctctgacccagcatgggaaggaagatctgaaattccccaggggacagggcgtgcctatcaacaccaattcaagcccagacgatcagattgggtactataggagggctacaaggagaatccggggaggcgacggaaagatgaaagatctgagccccagatggtacttttactatctgggaacaggaccagaggctggactgccttatggcgctaacaaggacggaatcatttgggtggccactgaaggcgctctgaatacccctaaagatcacattggaacccgcaacccagccaacaatgccgctatcgtgctgcagctgccacagggcaccacactgcccaagggcttctacgctgaggggagtaggggaggatcacaggcttcctctcgcagttcaagcaggtcccgcaattcctctcggaactctacaccagggagttcacggggaactagcccagctagaatggctggaaatggaggggacgccgctctggccctgctgctgctggatagactgaaccagctggagtctaagatgagtggcaaagggcagcagcagcagggacagacagtgactaagaaatccgccgctgaagcctctaagaaaccccggcagaagagaaccgctacaaaagcctacaatgtgacccaggcttttggcaggcgcggacctgagcagacacagggaaacttcggcgaccaggaactgattaggcagggcaccgattataagcactggccccagatcgctcagttcgcccctagtgcttcagccttctttggaatgtctcgcattggcatggaggtgacacctagtggcacttggctgacttacaccggggccatcaagctggacgataaagacccaaacttcaaggatcaggtcatcctgctgaacaagcatattgacgcctataaaaccttcccccctacagagcccaagaaagacaagaaaaagaaagccgatgaaacccaggctctgccccagaggcagaagaaacagcagacagtgactctgctgcctgccgctgatctggacgatttcagtaaacagctgcagcagagtatgagtagtgccgacagtacccaggct

SEQ ID NO: 41 - protein, 1103 aa, synthetic construct
  REGION 1..1103: note = OC2.2 polypeptide sequence
MARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 42 - protein, 1120 aa, synthetic construct
  REGION 1..1120: note = OC2.3 polypeptide sequence
MDWTWILFLVAAATRVHSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 43 - DNA, 54 bp, synthetic construct
  misc_feature 1..54: note = IgE leader codon optimized DNA sequence
  source 1..54: mol_type = other DNA
atggactggacctggattctgttcctggtggccgccgccacacgggtgcacagc

SEQ ID NO: 44 - protein, 18 aa, synthetic construct
  REGION 1..18: note = IgE leader polypeptide sequence
MDWTWILFLVAAATRVHS

SEQ ID NO: 45 - DNA, 1314 bp, synthetic construct
  misc_feature 1..1314: note = RBDv2 (tandem repeat single chain dimer) codon optimized DNAsequence
  source 1..1314: mol_type = other DNA
cgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaagatcgctgactacaattataaactgcccgacgatttcaccggctgcgtgatcgcctggaacagcaacaatctggattccaaagtgggcgggaactacaattatctgtaccggctgttcagaaagtccaatctgaaaccctttgagagggacatcagtactgaaatctaccaggccgggtcaaccccttgcaatggggtggagggattcaactgttactttccactgcagagttatggatttcagcccaccaacggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaagattgctgactataattacaaactgccagacgatttcactggatgcgtgattgcctggaactctaacaatctggatagtaaagtgggaggcaactataattacctgtataggctgttccgcaagtctaatctgaaaccatttgagcgggacatcagcaccgagatctaccaggccggctccaccccctgcaatggagtggagggcttcaactgctattttccactgcagagctatgggtttcagcccacaaacggggtgggataccagccttatagagtggtggtgctgtccttcgaactgctgcatgcccctgctacagtgtgcggcccaaagaaatctaccaacctggtcaagaacaag

SEQ ID NO: 46 - protein, 438 aa, synthetic construct
  REGION 1..438: note = RBDv2 (tandem repeat single chain dimer) polypeptide sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 47 - DNA, 1314 bp, synthetic construct
  misc_feature 1..1314: note = RBDv2 (E501Y) codon optimized DNA sequence
  source 1..1314: mol_type = other DNA
cgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaagatcgctgactacaattataaactgcccgacgatttcaccggctgcgtgatcgcctggaacagcaacaatctggattccaaagtgggcgggaactacaattatctgtaccggctgttcagaaagtccaatctgaaaccctttgagagggacatcagtactgaaatctaccaggccgggtcaaccccttgcaatggggtggagggattcaactgttactttccactgcagagttatggatttcagcccacctatggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaagattgctgactataattacaaactgccagacgatttcactggatgcgtgattgcctggaactctaacaatctggatagtaaagtgggaggcaactataattacctgtataggctgttccgcaagtctaatctgaaaccatttgagcgggacatcagcaccgagatctaccaggccggctccaccccctgcaatggagtggagggcttcaactgctattttccactgcagagctatgggtttcagcccacatatggggtgggataccagccttatagagtggtggtgctgtccttcgaactgctgcatgcccctgctacagtgtgcggcccaaagaaatctaccaacctggtcaagaacaag

SEQ ID NO: 48 - DNA, 1314 bp, synthetic construct
  misc_feature 1..1314: note = RBDv2 (N439K, N501Y) codon optimized DNA sequence
  source 1..1314: mol_type = other DNA
cgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaagatcgctgactacaattataaactgcccgacgatttcaccggctgcgtgatcgcctggaacagcaacaagctggattccaaagtgggcgggaactacaattatctgtaccggctgttcagaaagtccaatctgaaaccctttgagagggacatcagtactgaaatctaccaggccgggtcaaccccttgcaatggggtggagggattcaactgttactttccactgcagagttatggatttcagcccacctatggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaagattgctgactataattacaaactgccagacgatttcactggatgcgtgattgcctggaactctaacaagctggatagtaaagtgggaggcaactataattacctgtataggctgttccgcaagtctaatctgaaaccatttgagcgggacatcagcaccgagatctaccaggccggctccaccccctgcaatggagtggagggcttcaactgctattttccactgcagagctatgggtttcagcccacatatggggtgggataccagccttatagagtggtggtgctgtccttcgaactgctgcatgcccctgctacagtgtgcggcccaaagaaatctaccaacctggtcaagaacaag

SEQ ID NO: 49 - DNA, 1314 bp, synthetic construct
  misc_feature 1..1314: note = RBDv2 (K417N, E484K, N501Y) codon optimized DNA sequence
  source 1..1314: mol_type = other DNA
cgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaacatcgctgactacaattataaactgcccgacgatttcaccggctgcgtgatcgcctggaacagcaacaacctggattccaaagtgggcgggaactacaattatctgtaccggctgttcagaaagtccaatctgaaaccctttgagagggacatcagtactgaaatctaccaggccgggtcaaccccttgcaatggggtgaagggattcaactgttactttccactgcagagttatggatttcagcccacctatggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaacattgctgactataattacaaactgccagacgatttcactggatgcgtgattgcctggaactctaacaacctggatagtaaagtgggaggcaactataattacctgtataggctgttccgcaagtctaatctgaaaccatttgagcgggacatcagcaccgagatctaccaggccggctccaccccctgcaatggagtgaagggcttcaactgctattttccactgcagagctatgggtttcagcccacatatggggtgggataccagccttatagagtggtggtgctgtccttcgaactgctgcatgcccctgctacagtgtgcggcccaaagaaatctaccaacctggtcaagaacaag

SEQ ID NO: 50 - DNA, 1314 bp, synthetic construct
  misc_feature 1..1314: note = RBDv2 (K417N, N439K, E484K, N501Y codon optimized DNA sequence
  source 1..1314: mol_type = other DNA
cgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaacatcgctgactacaattataaactgcccgacgatttcaccggctgcgtgatcgcctggaacagcaacaagctggattccaaagtgggcgggaactacaattatctgtaccggctgttcagaaagtccaatctgaaaccctttgagagggacatcagtactgaaatctaccaggccgggtcaaccccttgcaatggggtgaagggattcaactgttactttccactgcagagttatggatttcagcccacctatggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaacattgctgactataattacaaactgccagacgatttcactggatgcgtgattgcctggaactctaacaagctggatagtaaagtgggaggcaactataattacctgtataggctgttccgcaagtctaatctgaaaccatttgagcgggacatcagcaccgagatctaccaggccggctccaccccctgcaatggagtgaagggcttcaactgctattttccactgcagagctatgggtttcagcccacatatggggtgggataccagccttatagagtggtggtgctgtccttcgaactgctgcatgcccctgctacagtgtgcggcccaaagaaatctaccaacctggtcaagaacaag

SEQ ID NO: 51 - DNA, 3816 bp, synthetic construct
  misc_feature 1..3816: note = SARS-CoV-2 S protein (K986P, V987P) codon optimized DNA sequence
  source 1..3816: mol_type = other DNA
ttcgtctttctggtgctgctgcctctggtcagctcccagtgcgtgaacctgaccacaaggacacagctgccccctgcctatacaaattccttcactcggggcgtgtactatcctgacaaagtgtttagatctagtgtgctgcactccacacaggatctgtttctgccattcttttctaacgtgacttggttccacgccatccacgtgagcgggaccaatggaacaaagaggttcgacaatccagtgctgccctttaacgatggcgtgtacttcgccagtaccgagaagtcaaacatcattcgcgggtggatctttggaactaccctggacagcaaaacccagtccctgctgatcgtgaacaatgccacaaacgtggtcatcaaggtgtgcgaattccagttttgtaatgatcccttcctgggcgtgtactatcataagaacaacaagtcttggatggagagtgaatttcgcgtgtattcaagcgccaacaattgcacatttgagtacgtgagccagcctttcctgatggacctggaaggcaagcaggggaatttcaaaaacctgcgggagttcgtgtttaagaatattgatggctacttcaagatctactctaaacacacccccatcaacctggtgcgggacctgcctcaggggttcagtgccctggagcctctggtggatctgccaatcggaattaacatcacccggtttcagacactgctggccctgcatagatcctacctgacaccaggcgactcctctagtggctggactgctggggccgctgcctactatgtggggtatctgcagccccggaccttcctgctgaaatacaacgagaatggaactattaccgacgctgtggattgcgccctggaccccctgtccgaaactaagtgtaccctgaaatcttttaccgtggagaagggaatctatcagacaagcaatttcagggtgcagccaactgaatccattgtgcgctttccaaatatcaccaacctgtgcccctttggcgaggtgttcaacgctacaagattcgccagcgtgtacgcttggaataggaagcgcatcagcaactgcgtggccgactattccgtgctgtacaactccgcttctttcagtacctttaagtgctatggagtgtcacccaccaaactgaatgacctgtgctttacaaacgtgtacgccgattccttcgtgattaggggcgacgaggtgcgccagatcgctcctggacagaccggcaagattgctgactacaattataaactgccagacgatttcacaggctgcgtgatcgcctggaactctaacaatctggatagtaaagtgggcgggaactacaattatctgtacaggctgtttcgcaagagcaatctgaaacccttcgagcgggacattagtactgaaatctaccaggccggctcaaccccttgcaatggggtggagggcttcaactgttatttcccactgcagtcctacgggttccagcccacaaacggagtgggctatcagccttacagagtggtggtgctgtcttttgaactgctgcacgctccagctacagtgtgcggacccaagaaaagtactaatctggtgaagaacaaatgcgtgaacttcaacttcaacggactgacagggactggagtgctgaccgagagcaacaagaaattcctgccatttcagcagttcggaagggacatcgccgatacaactgacgctgtgcgcgacccccagaccctggaaattctggatatcacaccttgctcattcggaggcgtgagcgtgattacccccggcaccaatacatccaaccaggtggccgtgctgtatcaggacgtgaattgtacagaggtgccagtggctatccacgccgatcagctgactcccacctggcgggtgtacagcactggctccaacgtgtttcagaccagagccggatgcctgattggcgctgagcatgtgaacaatagttatgaatgcgacattccaatcggcgccgggatctgtgcttcataccagacacagactaactctccccggagagccaggagtgtggcttcacagagcatcattgcctataccatgtcactgggggccgaaaatagcgtggcttactctaacaatagtattgctatccccacaaacttcactatttctgtgaccacagagatcctgcccgtgagcatgaccaagacatctgtggactgcaccatgtatatttgtggcgattcaacagaatgcagcaacctgctgctgcagtacggaagcttttgtacacagctgaatcgcgccctgactggcatcgctgtggagcaggacaaaaacactcaggaagtgttcgcccaggtgaagcagatctacaaaaccccacccatcaaggactttgggggcttcaacttcagccagattctgcctgatccatcaaagcccagcaaaaggtcctttatcgaggacctgctgttcaacaaggtgaccctggctgatgccggcttcatcaaacagtatggggattgcctgggagacattgctgcccgcgacctgatctgtgcccagaagtttaatgggctgaccgtgctgcctccactgctgacagatgaaatgattgcccagtacacatctgctctgctggccgggactatcaccagtggatggactttcggagccggcgctgccctgcagattccttttgccatgcagatggcttataggttcaacgggatcggagtgacccagaatgtgctgtacgagaaccagaagctgatcgccaatcagtttaacagcgccattggcaaaatccaggactccctgtcaagcactgcttctgccctggggaaactgcaggatgtggtgaatcagaacgctcaggccctgaataccctggtgaagcagctgtcctctaacttcggggccattagttcagtgctgaatgatatcctgtccaggctggacccccctgaggctgaagtgcagattgaccggctgatcacaggaagactgcagagcctgcagacctacgtgacacagcagctgattagggctgccgaaatccgcgcttccgccaatctggctgccaccaagatgtctgagtgcgtgctggggcagagtaagcgcgtggacttttgtggcaaagggtatcacctgatgtccttcccccagtctgcccctcacggagtggtgtttctgcatgtgacctacgtgcctgctcaggagaagaacttcactaccgctccagccatctgccacgatggcaaagcccattttccccgggaaggcgtgttcgtgtccaacgggacccattggtttgtgacacagagaaatttctacgagcctcagatcattacaactgacaataccttcgtgagcggaaactgtgacgtggtcatcggcatcgtgaacaataccgtgtacgatcctctgcagccagagctggacagctttaaggaggaactggataagtacttcaaaaatcacacctcccccgacgtggatctgggcgacattagtgggatcaatgcctcagtggtgaacattcagaaggagatcgacagactgaacgaagtggctaaaaatctgaacgagagcctgattgatctgcaggagctgggcaagtatgaacagtacatcaaatggccttggtatatttggctgggattcatcgccggcctgattgctatcgtgatggtgactatcatgctgtgctgtatgacctcttgctgtagttgcctgaagggctgctgttcatgtgggagctgctgtaaattcgacgaggacgattccgaaccagtgctgaagggcgtgaaactgcactacact

SEQ ID NO: 52 - protein, 438 aa, synthetic construct
  REGION 1..438: note = RBDv2 (E501Y) polypeptide sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNK

SEQ ID NO: 53 - protein, 438 aa, synthetic construct
  REGION 1..438: note = RBDv2 (N439K, N501Y) polypeptide sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNKLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNKLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNK

SEQ ID NO: 54 - protein, 438 aa, synthetic construct
  REGION 1..438: note = RBDv2 (K417N, E484K, N501Y) polypeptide sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNK

SEQ ID NO: 55 - protein, 438 aa, synthetic construct
  REGION 1..438: note = RBDv2 (K417N, N439K, E484K, N501Y polypeptide sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNKLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNKLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNK

SEQ ID NO: 56 - protein, 1272 aa, synthetic construct
  REGION 1..1272: note = SARS-CoV-2 S protein (K986P, V987P) polypeptide sequence
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 57 - DNA, 3360 bp, synthetic construct
  misc_feature 1..3360: note = OC2.3 (N501Y) codon optimized DNA sequence
  source 1..3360: mol_type = other DNA
atggactggacctggattctgttcctggtggccgccgccacacgggtgcacagcgcccgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaagatcgctgactacaattataaactgcccgacgatttcaccggctgcgtgatcgcctggaacagcaacaatctggattccaaagtgggcgggaactacaattatctgtaccggctgttcagaaagtccaatctgaaaccctttgagagggacatcagtactgaaatctaccaggccgggtcaaccccttgcaatggggtggagggattcaactgttactttccactgcagagttatggatttcagcccacctatggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaagattgctgactataattacaaactgccagacgatttcactggatgcgtgattgcctggaactctaacaatctggatagtaaagtgggaggcaactataattacctgtataggctgttccgcaagtctaatctgaaaccatttgagcgggacatcagcaccgagatctaccaggccggctccaccccctgcaatggagtggagggcttcaactgctattttccactgcagagctatgggtttcagcccacatatggggtgggataccagccttatagagtggtggtgctgtccttcgaactgctgcatgcccctgctacagtgtgcggcccaaagaaatctaccaacctggtcaagaacaagggaagcggagctactaacttcagcctgctgaagcaggctggagacgtggaggagaaccctggacctatggccgattccaacggaactattaccgtggaggaactgaagaaactgctggagcagtggaacctggtcatcggcttcctgtttctgacctggatttgtctgctgcagttcgcctacgctaaccggaacagatttctgtacatcattaagctgatcttcctgtggctgctgtggcccgtgactctggcctgcttcgtgctggccgccgtgtacaggatcaactggattaccgggggaatcgccattgctatggcctgtctggtgggcctgatgtggctgtcatacttcatcgccagcttccgcctgtttgctaggacacgcagcatgtggtccttcaaccctgagactaatatcctgctgaacgtgccactgcacggcaccattctgacacggcccctgctggagtccgaactggtcatcggggccgtgattctgaggggacatctgcgcatcgctgggcaccatctgggaagatgcgacatcaaggatctgcctaaagaaattactgtggccacctcaaggacactgagctactataagctgggagctagccagagggtggctggggacagcggatttgctgcttactcccggtatagaattggcaattacaaactgaacacagaccacagctcctctagtgataatatcgccctgctggtgcaggctagcgacaacgggccccagaaccagcggaatgcccctagaatcaccttcggcgggccatccgattctacaggcagcaaccagaatggagagaggtccggagctcgctctaagcagagacggccccagggcctgccaaacaataccgcctcctggtttactgctctgacccagcatgggaaggaagatctgaaattccccaggggacagggcgtgcctatcaacaccaattcaagcccagacgatcagattgggtactataggagggctacaaggagaatccggggaggcgacggaaagatgaaagatctgagccccagatggtacttttactatctgggaacaggaccagaggctggactgccttatggcgctaacaaggacggaatcatttgggtggccactgaaggcgctctgaatacccctaaagatcacattggaacccgcaacccagccaacaatgccgctatcgtgctgcagctgccacagggcaccacactgcccaagggcttctacgctgaggggagtaggggaggatcacaggcttcctctcgcagttcaagcaggtcccgcaattcctctcggaactctacaccagggagttcacggggaactagcccagctagaatggctggaaatggaggggacgccgctctggccctgctgctgctggatagactgaaccagctggagtctaagatgagtggcaaagggcagcagcagcagggacagacagtgactaagaaatccgccgctgaagcctctaagaaaccccggcagaagagaaccgctacaaaagcctacaatgtgacccaggcttttggcaggcgcggacctgagcagacacagggaaacttcggcgaccaggaactgattaggcagggcaccgattataagcactggccccagatcgctcagttcgcccctagtgcttcagccttctttggaatgtctcgcattggcatggaggtgacacctagtggcacttggctgacttacaccggggccatcaagctggacgataaagacccaaacttcaaggatcaggtcatcctgctgaacaagcatattgacgcctataaaaccttcccccctacagagcccaagaaagacaagaaaaagaaagccgatgaaacccaggctctgccccagaggcagaagaaacagcagacagtgactctgctgcctgccgctgatctggacgatttcagtaaacagctgcagcagagtatgagtagtgccgacagtacccaggct

SEQ ID NO: 58 - DNA, 3360 bp, synthetic construct
  misc_feature 1..3360: note = OC2.3 (N439K, N501Y) codon optimized DNA sequence
  source 1..3360: mol_type = other DNA
atggactggacctggattctgttcctggtggccgccgccacacgggtgcacagcgcccgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaagatcgctgactacaattataaactgcccgacgatttcaccggctgcgtgatcgcctggaacagcaacaagctggattccaaagtgggcgggaactacaattatctgtaccggctgttcagaaagtccaatctgaaaccctttgagagggacatcagtactgaaatctaccaggccgggtcaaccccttgcaatggggtggagggattcaactgttactttccactgcagagttatggatttcagcccacctatggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaagattgctgactataattacaaactgccagacgatttcactggatgcgtgattgcctggaactctaacaagctggatagtaaagtgggaggcaactataattacctgtataggctgttccgcaagtctaatctgaaaccatttgagcgggacatcagcaccgagatctaccaggccggctccaccccctgcaatggagtggagggcttcaactgctattttccactgcagagctatgggtttcagcccacatatggggtgggataccagccttatagagtggtggtgctgtccttcgaactgctgcatgcccctgctacagtgtgcggcccaaagaaatctaccaacctggtcaagaacaagggaagcggagctactaacttcagcctgctgaagcaggctggagacgtggaggagaaccctggacctatggccgattccaacggaactattaccgtggaggaactgaagaaactgctggagcagtggaacctggtcatcggcttcctgtttctgacctggatttgtctgctgcagttcgcctacgctaaccggaacagatttctgtacatcattaagctgatcttcctgtggctgctgtggcccgtgactctggcctgcttcgtgctggccgccgtgtacaggatcaactggattaccgggggaatcgccattgctatggcctgtctggtgggcctgatgtggctgtcatacttcatcgccagcttccgcctgtttgctaggacacgcagcatgtggtccttcaaccctgagactaatatcctgctgaacgtgccactgcacggcaccattctgacacggcccctgctggagtccgaactggtcatcggggccgtgattctgaggggacatctgcgcatcgctgggcaccatctgggaagatgcgacatcaaggatctgcctaaagaaattactgtggccacctcaaggacactgagctactataagctgggagctagccagagggtggctggggacagcggatttgctgcttactcccggtatagaattggcaattacaaactgaacacagaccacagctcctctagtgataatatcgccctgctggtgcaggctagcgacaacgggccccagaaccagcggaatgcccctagaatcaccttcggcgggccatccgattctacaggcagcaaccagaatggagagaggtccggagctcgctctaagcagagacggccccagggcctgccaaacaataccgcctcctggtttactgctctgacccagcatgggaaggaagatctgaaattccccaggggacagggcgtgcctatcaacaccaattcaagcccagacgatcagattgggtactataggagggctacaaggagaatccggggaggcgacggaaagatgaaagatctgagccccagatggtacttttactatctgggaacaggaccagaggctggactgccttatggcgctaacaaggacggaatcatttgggtggccactgaaggcgctctgaatacccctaaagatcacattggaacccgcaacccagccaacaatgccgctatcgtgctgcagctgccacagggcaccacactgcccaagggcttctacgctgaggggagtaggggaggatcacaggcttcctctcgcagttcaagcaggtcccgcaattcctctcggaactctacaccagggagttcacggggaactagcccagctagaatggctggaaatggaggggacgccgctctggccctgctgctgctggatagactgaaccagctggagtctaagatgagtggcaaagggcagcagcagcagggacagacagtgactaagaaatccgccgctgaagcctctaagaaaccccggcagaagagaaccgctacaaaagcctacaatgtgacccaggcttttggcaggcgcggacctgagcagacacagggaaacttcggcgaccaggaactgattaggcagggcaccgattataagcactggccccagatcgctcagttcgcccctagtgcttcagccttctttggaatgtctcgcattggcatggaggtgacacctagtggcacttggctgacttacaccggggccatcaagctggacgataaagacccaaacttcaaggatcaggtcatcctgctgaacaagcatattgacgcctataaaaccttcccccctacagagcccaagaaagacaagaaaaagaaagccgatgaaacccaggctctgccccagaggcagaagaaacagcagacagtgactctgctgcctgccgctgatctggacgatttcagtaaacagctgcagcagagtatgagtagtgccgacagtacccaggct

SEQ ID NO: 59 - DNA, 3360 bp, synthetic construct
  misc_feature 1..3360: note = OC2.3 (K417N, E484K, N501Y) codon optimized DNA sequence
  source 1..3360: mol_type = other DNA
atggactggacctggattctgttcctggtggccgccgccacacgggtgcacagcgcccgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaacatcgctgactacaattataaactgcccgacgatttcaccggctgcgtgatcgcctggaacagcaacaacctggattccaaagtgggcgggaactacaattatctgtaccggctgttcagaaagtccaatctgaaaccctttgagagggacatcagtactgaaatctaccaggccgggtcaaccccttgcaatggggtgaagggattcaactgttactttccactgcagagttatggatttcagcccacctatggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaacattgctgactataattacaaactgccagacgatttcactggatgcgtgattgcctggaactctaacaacctggatagtaaagtgggaggcaactataattacctgtataggctgttccgcaagtctaatctgaaaccatttgagcgggacatcagcaccgagatctaccaggccggctccaccccctgcaatggagtgaagggcttcaactgctattttccactgcagagctatgggtttcagcccacatatggggtgggataccagccttatagagtggtggtgctgtccttcgaactgctgcatgcccctgctacagtgtgcggcccaaagaaatctaccaacctggtcaagaacaagggaagcggagctactaacttcagcctgctgaagcaggctggagacgtggaggagaaccctggacctatggccgattccaacggaactattaccgtggaggaactgaagaaactgctggagcagtggaacctggtcatcggcttcctgtttctgacctggatttgtctgctgcagttcgcctacgctaaccggaacagatttctgtacatcattaagctgatcttcctgtggctgctgtggcccgtgactctggcctgcttcgtgctggccgccgtgtacaggatcaactggattaccgggggaatcgccattgctatggcctgtctggtgggcctgatgtggctgtcatacttcatcgccagcttccgcctgtttgctaggacacgcagcatgtggtccttcaaccctgagactaatatcctgctgaacgtgccactgcacggcaccattctgacacggcccctgctggagtccgaactggtcatcggggccgtgattctgaggggacatctgcgcatcgctgggcaccatctgggaagatgcgacatcaaggatctgcctaaagaaattactgtggccacctcaaggacactgagctactataagctgggagctagccagagggtggctggggacagcggatttgctgcttactcccggtatagaattggcaattacaaactgaacacagaccacagctcctctagtgataatatcgccctgctggtgcaggctagcgacaacgggccccagaaccagcggaatgcccctagaatcaccttcggcgggccatccgattctacaggcagcaaccagaatggagagaggtccggagctcgctctaagcagagacggccccagggcctgccaaacaataccgcctcctggtttactgctctgacccagcatgggaaggaagatctgaaattccccaggggacagggcgtgcctatcaacaccaattcaagcccagacgatcagattgggtactataggagggctacaaggagaatccggggaggcgacggaaagatgaaagatctgagccccagatggtacttttactatctgggaacaggaccagaggctggactgccttatggcgctaacaaggacggaatcatttgggtggccactgaaggcgctctgaatacccctaaagatcacattggaacccgcaacccagccaacaatgccgctatcgtgctgcagctgccacagggcaccacactgcccaagggcttctacgctgaggggagtaggggaggatcacaggcttcctctcgcagttcaagcaggtcccgcaattcctctcggaactctacaccagggagttcacggggaactagcccagctagaatggctggaaatggaggggacgccgctctggccctgctgctgctggatagactgaaccagctggagtctaagatgagtggcaaagggcagcagcagcagggacagacagtgactaagaaatccgccgctgaagcctctaagaaaccccggcagaagagaaccgctacaaaagcctacaatgtgacccaggcttttggcaggcgcggacctgagcagacacagggaaacttcggcgaccaggaactgattaggcagggcaccgattataagcactggccccagatcgctcagttcgcccctagtgcttcagccttctttggaatgtctcgcattggcatggaggtgacacctagtggcacttggctgacttacaccggggccatcaagctggacgataaagacccaaacttcaaggatcaggtcatcctgctgaacaagcatattgacgcctataaaaccttcccccctacagagcccaagaaagacaagaaaaagaaagccgatgaaacccaggctctgccccagaggcagaagaaacagcagacagtgactctgctgcctgccgctgatctggacgatttcagtaaacagctgcagcagagtatgagtagtgccgacagtacccaggct

SEQ ID NO: 60 - DNA, 3360 bp, synthetic construct
  misc_feature 1..3360: note = OC2.3 (K417N, N439K, E484K, N501Y) codon optimized DNA sequence
  source 1..3360: mol_type = other DNA
atggactggacctggattctgttcctggtggccgccgccacacgggtgcacagcgcccgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaacatcgctgactacaattataaactgcccgacgatttcaccggctgcgtgatcgcctggaacagcaacaagctggattccaaagtgggcgggaactacaattatctgtaccggctgttcagaaagtccaatctgaaaccctttgagagggacatcagtactgaaatctaccaggccgggtcaaccccttgcaatggggtgaagggattcaactgttactttccactgcagagttatggatttcagcccacctatggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaacattgctgactataattacaaactgccagacgatttcactggatgcgtgattgcctggaactctaacaagctggatagtaaagtgggaggcaactataattacctgtataggctgttccgcaagtctaatctgaaaccatttgagcgggacatcagcaccgagatctaccaggccggctccaccccctgcaatggagtgaagggcttcaactgctattttccactgcagagctatgggtttcagcccacatatggggtgggataccagccttatagagtggtggtgctgtccttcgaactgctgcatgcccctgctacagtgtgcggcccaaagaaatctaccaacctggtcaagaacaagggaagcggagctactaacttcagcctgctgaagcaggctggagacgtggaggagaaccctggacctatggccgattccaacggaactattaccgtggaggaactgaagaaactgctggagcagtggaacctggtcatcggcttcctgtttctgacctggatttgtctgctgcagttcgcctacgctaaccggaacagatttctgtacatcattaagctgatcttcctgtggctgctgtggcccgtgactctggcctgcttcgtgctggccgccgtgtacaggatcaactggattaccgggggaatcgccattgctatggcctgtctggtgggcctgatgtggctgtcatacttcatcgccagcttccgcctgtttgctaggacacgcagcatgtggtccttcaaccctgagactaatatcctgctgaacgtgccactgcacggcaccattctgacacggcccctgctggagtccgaactggtcatcggggccgtgattctgaggggacatctgcgcatcgctgggcaccatctgggaagatgcgacatcaaggatctgcctaaagaaattactgtggccacctcaaggacactgagctactataagctgggagctagccagagggtggctggggacagcggatttgctgcttactcccggtatagaattggcaattacaaactgaacacagaccacagctcctctagtgataatatcgccctgctggtgcaggctagcgacaacgggccccagaaccagcggaatgcccctagaatcaccttcggcgggccatccgattctacaggcagcaaccagaatggagagaggtccggagctcgctctaagcagagacggccccagggcctgccaaacaataccgcctcctggtttactgctctgacccagcatgggaaggaagatctgaaattccccaggggacagggcgtgcctatcaacaccaattcaagcccagacgatcagattgggtactataggagggctacaaggagaatccggggaggcgacggaaagatgaaagatctgagccccagatggtacttttactatctgggaacaggaccagaggctggactgccttatggcgctaacaaggacggaatcatttgggtggccactgaaggcgctctgaatacccctaaagatcacattggaacccgcaacccagccaacaatgccgctatcgtgctgcagctgccacagggcaccacactgcccaagggcttctacgctgaggggagtaggggaggatcacaggcttcctctcgcagttcaagcaggtcccgcaattcctctcggaactctacaccagggagttcacggggaactagcccagctagaatggctggaaatggaggggacgccgctctggccctgctgctgctggatagactgaaccagctggagtctaagatgagtggcaaagggcagcagcagcagggacagacagtgactaagaaatccgccgctgaagcctctaagaaaccccggcagaagagaaccgctacaaaagcctacaatgtgacccaggcttttggcaggcgcggacctgagcagacacagggaaacttcggcgaccaggaactgattaggcagggcaccgattataagcactggccccagatcgctcagttcgcccctagtgcttcagccttctttggaatgtctcgcattggcatggaggtgacacctagtggcacttggctgacttacaccggggccatcaagctggacgataaagacccaaacttcaaggatcaggtcatcctgctgaacaagcatattgacgcctataaaaccttcccccctacagagcccaagaaagacaagaaaaagaaagccgatgaaacccaggctctgccccagaggcagaagaaacagcagacagtgactctgctgcctgccgctgatctggacgatttcagtaaacagctgcagcagagtatgagtagtgccgacagtacccaggct

SEQ ID NO: 61 - DNA, 4017 bp, synthetic construct
  misc_feature 1..4017: note = OC2.4 codon optimized DNA sequence
  source 1..4017: mol_type = other DNA
atggactggacttggattctgttcctggtcgccgccgccacccgagtgcatagcgcccgcgtgcagcccaccgagagcattgtgaggtttcccaacatcacaaatctgtgccctttcggcgaggtgtttaatgctactaggttcgccagcgtgtacgcttggaatcggaagagaatttctaactgcgtggccgactatagtgtgctgtacaacagcgcctccttctctacctttaagtgctatggggtgtctcccaccaaactgaatgacctgtgcttcacaaacgtgtacgccgacagttttgtgatccggggagatgaggtgagacagattgccccaggccagaccgggaagatcgctgactacaattataaactgcccgacgatttcacaggctgcgtgatcgcctggaacagtaacaatctggattcaaaagtgggcgggaactacaattatctgtacaggctgttccgcaagagcaacctgaaaccatttgagagggacatcagcactgaaatctaccaggccggctccaccccctgcaatggagtggagggcttcaactgttattttccactgcagagctacgggtttcagcccaccaacggggtgggatatcagccttaccgcgtggtggtgctgtccttcgaactgctgcacgctccagctaccgtgtgcggacccaagaaaagtactaacctggtgaagaacaagagggtgcagccaactgagtcaatcgtgcgctttccaaacattaccaatctgtgccccttcggggaagtgtttaatgccacacgcttcgcttccgtgtacgcctggaataggaagcgcatctcaaattgcgtggctgactatagcgtgctgtacaacagtgcttcattcagcacttttaagtgctatggcgtgtcacctactaaactgaatgacctgtgctttaccaacgtgtacgctgacagctttgtgattaggggggatgaggtgcgccagatcgctcctggacagactggaaagattgctgactataactacaaactgccagacgatttcaccggatgcgtgattgcctggaacagcaataagctggattccaaagtgggaggcaactataattacctgtatcggctgttcagaaagtctaacctgaaaccttttgagcgggacatctctaccgagatctaccaggccggaagtaccccatgcaatggcgtggaggggttcaactgctactttccactgcagtcttacggctttcagcccacctatggcgtggggtatcagccttacagagtggtggtgctgagcttcgaactgctgcatgctcctgctaccgtgtgcggaccaaagaaaagcaccaacctggtcaagaacaagcgggtgcagcctacagagtccatcgtgagatttcctaacattactaatctgtgcccattcggcgaagtgtttaatgctacaagatttgctagcgtgtacgcctggaatcggaagagaatctccaattgcgtggcagactattctgtgctgtacaactccgcttctttcagtacctttaagtgttacggagtgtccccaacaaaactgaatgacctgtgcttcacgaacgtgtacgccgactcttttgtgatccggggcgatgaggtgagacagatcgcccccggacagacaggcaatatcgctgactacaactataagctgcctgacgatttcactggatgcgtgattgcttggaattctaacaatctggatagtaaagtggggggaaattataactatctgtataggctgttccgcaagtctaacctgaaaccctttgagagggacatctcaaccgagatctaccaggctgggagcaccccttgcaatggggtgaagggattcaactgttactttccactgcagagctatggatttcagcccacatacggagtgggctaccagccatatagagtggtggtgctgagtttcgagctgctgcatgccccagctacagtgtgcggacctaagaaaagcacaaacctggtcaaaaacaagggaagcggagctactaacttcagcctgctgaagcaggctggagacgtggaggagaaccctggacctatggccgatagcaacgggactatcaccgtggaggaactgaagaaactgctggaacagtggaacctggtcatcggcttcctgtttctgacctggatctgtctgctgcagttcgcctatgctaacaggaatcgctttctgtacatcattaagctgattttcctgtggctgctgtggcctgtgactctggcctgcttcgtgctggccgccgtgtacaggatcaactggattaccggcgggatcgccattgctatggcctgtctggtgggcctgatgtggctgtcatactttatcgccagcttccgcctgtttgctcggaccagaagtatgtggtcattcaaccctgagacaaatattctgctgaacgtgccactgcacggcaccatcctgacacggcccctgctggagagcgaactggtcatcggcgccgtgattctgagggggcatctgcgcatcgctggccaccatctggggagatgcgacatcaaggatctgcccaaagaaattactgtggccacctctaggacactgagttactataagctgggagcttcccagagggtggctggcgacagcggatttgctgcttattccaggtaccgcatcgggaattacaaactgaacacagaccacagctcctctagtgataatattgccctgctggtgcaggctagcgacaacggaccccagaaccagcggaatgcccctagaatcaccttcggaggcccatcagatagcacaggctccaaccagaatggagagaggtcaggagctcgcagcaagcagagacggccccagggactgccaaacaataccgcctcttggtttactgctctgacccagcatggcaaggaagatctgaaattcccacggggccagggggtgcccattaacaccaattcaagccccgacgatcagatcggctactataggagggctacaaggagaattcggggaggagacgggaagatgaaagatctgagccctagatggtacttctactatctgggaacaggaccagaggctgggctgccttacggagctaacaaggacgggatcatttgggtggccactgaaggagctctgaatacccctaaagatcacatcgggaccagaaacccagccaacaatgccgctattgtgctgcagctgcctcagggaaccacactgccaaagggattctacgctgagggctcccggggaggcagccaggcttccagccggagctcaagccggagtagaaattcctctcggaactcaacaccaggcagttcaagggggacttccccagctaggatggctggaaatggaggcgacgccgctctggccctgctgctgctggatagactgaaccagctggagagcaagatgtccgggaaaggacagcagcagcagggacagacagtgactaagaaatccgccgctgaagcctctaagaaacctcggcagaagagaaccgctacaaaagcctataatgtgactcaggcttttggaaggcgcggaccagagcagacccagggaaacttcggcgaccaggaactgatcaggcagggcaccgattacaagcactggccccagattgctcagttcgccccttccgcttctgccttctttgggatgtcccgcatcggaatggaggtgacaccatctggaacttggctgacttataccggcgccattaagctggacgataaagaccccaacttcaaggatcaggtcatcctgctgaacaagcatattgacgcctacaaaaccttcccccctacagagcccaagaaagacaagaaaaagaaagccgatgaaacccaggctctgccccagagacagaagaaacagcagacagtgactctgctgcctgccgctgatctggacgatttctcaaaacagctgcagcagagcatgtcaagtgccgattcaactcaggct

SEQ ID NO: 62 - DNA, 2697 bp, synthetic construct
  misc_feature 1..2697: note = SVF-14 codon optimized DNA sequence
  source 1..2697: mol_type = other DNA
atggattggacttggattctgtttctggtcgccgccgccacacgagtgcattccgcccgagtgcagcctaccgagagtattgtccgattccctaacattactaatctgtgcccattcggcgaggtgtttaacgccaccaggtttgctagcgtgtacgcctggaaccggaagagaatcagcaattgcgtggccgactactccgtgctgtataattccgcttctttcagtacctttaagtgctacggcgtgtcacccactaaactgaacgacctgtgcttcaccaacgtgtacgccgacagctttgtgatccggggggatgaagtgagacagattgcccctggccagacagggaagatcgctgactacaactataaactgccagacgatttcactggctgcgtgatcgcctggaattctaacaatctggatagtaaagtgggcgggaactacaattatctgtacaggctgttccgcaagagcaacctgaaaccatttgagcgggacatctccacagaaatctaccaggccggatctactccctgcaacggagtggagggcttcaattgttactttcctctgcagagctatggcttccagccaaccaatggcgtggggtaccagccctatagagtggtggtgctgagctttgaactgctgcacgctccagctaccgtgtgcggacccaagaaatctacaaacctggtgaagaacaagcgggtgcagcccacagagagtatcgtgagattccccaatatcactaatctgtgccctttcggcgaagtgtttaacgctacacgctttgcctccgtgtacgcttggaacaggaagcgcatttctaactgcgtggctgactacagtgtgctgtataattcagctagcttctccacttttaagtgctacggagtgtcccctaccaaactgaacgacctgtgctttactaacgtgtacgctgactcttttgtgattaggggcgatgaggtgcgccagatcgctccaggacagaccggaaagattgctgactataactacaaactgcctgacgatttcacaggatgcgtgattgcctggaattcaaacaatctggatagcaaagtgggaggcaactataattacctgtatcggctgttcagaaagagcaacctgaaaccttttgagagggacatcagtaccgagatctaccaggccgggtcaactccatgcaacggcgtggaggggttcaattgctatttcccactgcagagctatggattccagccaaccaacggagtgggctaccagccctatcgcgtggtggtgctgagttttgaactgctgcatgctcctgctaccgtgtgcggaccaaagaaatctaccaacctggtcaaaaacaagggaagcggagctactaacttcagcctgctgaagcaggctggagacgtggaggagaaccctggacctatggcttccgataatggcccacagaaccagcggaatgctcccagaatcacttttgggggaccttctgacagtaccggctctaaccagaatggcgagaggagtggagctcgctcaaagcagagacggccccaggggctgccaaacaataccgcctcctggttcacagctctgactcagcacggaaaggaagatctgaaatttccccggggccagggggtgcctatcaacactaatagctcccctgacgatcagattggatactataggagggctaccaggagaatcaggggaggggacggcaagatgaaagatctgtctccacgctggtacttctactatctgggaaccggaccagaggctggactgccttatggagctaacaaggacggcatcatttgggtggccacagaaggggctctgaacactcccaaagatcatattggcacaagaaatcctgccaacaatgccgctatcgtgctgcagctgccacaggggaccacactgcccaagggcttttacgctgaggggagcaggggaggctcccaggcttctagtcgctcaagctcccggtcaagaaactctagtaggaatagcacccctgggtcaagccggggaacaagcccagccagaatggctggaaacggaggagacgccgctctggccctgctgctgctggatcgcctgaatcagctggagtccaagatgtctgggaaaggacagcagcagcagggacagactgtgaccaagaaatccgccgctgaagcctctaagaaaccaaggcagaagcgcacagccactaaagcttacaacgtgacccaggctttcggaaggcgcggaccagagcagacacaggggaattttggcgaccaggaactgattcggcagggcaccgattataagcactggccacagatcgcccagttcgctccctcagccagcgccttcttcggaatgagcagaattggcatggaggtgaccccctccgggacatggctgacctacacaggagccatcaagctggacgataaagaccctaacttcaaagatcaggtcatcctgctgaacaagcatattgacgcctataaaacttttccccctaccgagcccaagaaagacaagaaaaagaaagccgatgaaacacaggctctgccacagcggcagaagaaacagcagactgtgaccctgctgcccgccgctgatctggacgatttcagtaaacagctgcagcagtcaatgtcttccgccgattcaactcaggca

SEQ ID NO: 63 - DNA, 5199 bp, synthetic construct
  misc_feature 1..5199: note = SVF-15 codon optimized DNA sequence
  source 1..5199: mol_type = other DNA
atggactggacatggattctgtttctggtcgctgctgctacccgagtgcattctgccttcgtctttctggtgctgctgcctctggtcagctcccagtgcgtgaacctgaccacaaggacacagctgccccctgcctatacaaattccttcactcggggcgtgtactatcctgacaaagtgtttagatctagtgtgctgcactccacacaggatctgtttctgccattcttttctaacgtgacttggttccacgccatccacgtgagcgggaccaatggaacaaagaggttcgacaatccagtgctgccctttaacgatggcgtgtacttcgccagtaccgagaagtcaaacatcattcgcgggtggatctttggaactaccctggacagcaaaacccagtccctgctgatcgtgaacaatgccacaaacgtggtcatcaaggtgtgcgaattccagttttgtaatgatcccttcctgggcgtgtactatcataagaacaacaagtcttggatggagagtgaatttcgcgtgtattcaagcgccaacaattgcacatttgagtacgtgagccagcctttcctgatggacctggaaggcaagcaggggaatttcaaaaacctgcgggagttcgtgtttaagaatattgatggctacttcaagatctactctaaacacacccccatcaacctggtgcgggacctgcctcaggggttcagtgccctggagcctctggtggatctgccaatcggaattaacatcacccggtttcagacactgctggccctgcatagatcctacctgacaccaggcgactcctctagtggctggactgctggggccgctgcctactatgtggggtatctgcagccccggaccttcctgctgaaatacaacgagaatggaactattaccgacgctgtggattgcgccctggaccccctgtccgaaactaagtgtaccctgaaatcttttaccgtggagaagggaatctatcagacaagcaatttcagggtgcagccaactgaatccattgtgcgctttccaaatatcaccaacctgtgcccctttggcgaggtgttcaacgctacaagattcgccagcgtgtacgcttggaataggaagcgcatcagcaactgcgtggccgactattccgtgctgtacaactccgcttctttcagtacctttaagtgctatggagtgtcacccaccaaactgaatgacctgtgctttacaaacgtgtacgccgattccttcgtgattaggggcgacgaggtgcgccagatcgctcctggacagaccggcaagattgctgactacaattataaactgccagacgatttcacaggctgcgtgatcgcctggaactctaacaatctggatagtaaagtgggcgggaactacaattatctgtacaggctgtttcgcaagagcaatctgaaacccttcgagcgggacattagtactgaaatctaccaggccggctcaaccccttgcaatggggtggagggcttcaactgttatttcccactgcagtcctacgggttccagcccacaaacggagtgggctatcagccttacagagtggtggtgctgtcttttgaactgctgcacgctccagctacagtgtgcggacccaagaaaagtactaatctggtgaagaacaaatgcgtgaacttcaacttcaacggactgacagggactggagtgctgaccgagagcaacaagaaattcctgccatttcagcagttcggaagggacatcgccgatacaactgacgctgtgcgcgacccccagaccctggaaattctggatatcacaccttgctcattcggaggcgtgagcgtgattacccccggcaccaatacatccaaccaggtggccgtgctgtatcaggacgtgaattgtacagaggtgccagtggctatccacgccgatcagctgactcccacctggcgggtgtacagcactggctccaacgtgtttcagaccagagccggatgcctgattggcgctgagcatgtgaacaatagttatgaatgcgacattccaatcggcgccgggatctgtgcttcataccagacacagactaactctccccggagagccaggagtgtggcttcacagagcatcattgcctataccatgtcactgggggccgaaaatagcgtggcttactctaacaatagtattgctatccccacaaacttcactatttctgtgaccacagagatcctgcccgtgagcatgaccaagacatctgtggactgcaccatgtatatttgtggcgattcaacagaatgcagcaacctgctgctgcagtacggaagcttttgtacacagctgaatcgcgccctgactggcatcgctgtggagcaggacaaaaacactcaggaagtgttcgcccaggtgaagcagatctacaaaaccccacccatcaaggactttgggggcttcaacttcagccagattctgcctgatccatcaaagcccagcaaaaggtcctttatcgaggacctgctgttcaacaaggtgaccctggctgatgccggcttcatcaaacagtatggggattgcctgggagacattgctgcccgcgacctgatctgtgcccagaagtttaatgggctgaccgtgctgcctccactgctgacagatgaaatgattgcccagtacacatctgctctgctggccgggactatcaccagtggatggactttcggagccggcgctgccctgcagattccttttgccatgcagatggcttataggttcaacgggatcggagtgacccagaatgtgctgtacgagaaccagaagctgatcgccaatcagtttaacagcgccattggcaaaatccaggactccctgtcaagcactgcttctgccctggggaaactgcaggatgtggtgaatcagaacgctcaggccctgaataccctggtgaagcagctgtcctctaacttcggggccattagttcagtgctgaatgatatcctgtccaggctggacccccctgaggctgaagtgcagattgaccggctgatcacaggaagactgcagagcctgcagacctacgtgacacagcagctgattagggctgccgaaatccgcgcttccgccaatctggctgccaccaagatgtctgagtgcgtgctggggcagagtaagcgcgtggacttttgtggcaaagggtatcacctgatgtccttcccccagtctgcccctcacggagtggtgtttctgcatgtgacctacgtgcctgctcaggagaagaacttcactaccgctccagccatctgccacgatggcaaagcccattttccccgggaaggcgtgttcgtgtccaacgggacccattggtttgtgacacagagaaatttctacgagcctcagatcattacaactgacaataccttcgtgagcggaaactgtgacgtggtcatcggcatcgtgaacaataccgtgtacgatcctctgcagccagagctggacagctttaaggaggaactggataagtacttcaaaaatcacacctcccccgacgtggatctgggcgacattagtgggatcaatgcctcagtggtgaacattcagaaggagatcgacagactgaacgaagtggctaaaaatctgaacgagagcctgattgatctgcaggagctgggcaagtatgaacagtacatcaaatggccttggtatatttggctgggattcatcgccggcctgattgctatcgtgatggtgactatcatgctgtgctgtatgacctcttgctgtagttgcctgaagggctgctgttcatgtgggagctgctgtaaattcgacgaggacgattccgaaccagtgctgaagggcgtgaaactgcactacactggaagcggagctactaacttcagcctgctgaagcaggctggagacgtggaggagaaccctggacctatggcctctgataacggccctcagaatcagaggaacgctccacgcatcactttcggcggaccatccgactctaccggcagcaatcagaacggggagcggagtggagccagatcaaagcagaggcgcccacagggcctgcccaacaataccgccagctggttcactgctctgacccagcatgggaaggaagatctgaaatttcctcgggggcagggagtgccaatcaatactaacagctccccagacgatcagattggatactataggagagctaccaggcgcatcaggggaggcgatggcaagatgaaagacctgtccccccgctggtatttctactatctgggaacaggaccagaagctggcctgccttacggggctaacaaagacggcatcatttgggtggccacagagggggctctgaacactcctaaggatcacattggaaccagaaatccagccaacaatgctgctatcgtgctgcagctgccacagggaaccacactgcctaagggattctacgctgagggcagcaggggaggcagccaggcttctagtcgctcaagctcccggtcaagaaattctagtcggaacagcacaccaggatcaagccggggcactagcccagctagaatggctggaaatggaggggacgctgccctggccctgctgctgctggatagactgaaccagctggaatccaagatgtctggaaaaggccagcagcagcaggggcagacagtgaccaagaagagcgccgctgaggcttccaagaaacctaggcagaagagaaccgctacaaaagcctataatgtgactcaggccttcggccggagaggaccagagcagacccagggaaactttggcgaccaggaactgattaggcagggcaccgattacaagcactggcctcagatcgctcagttcgccccaagtgcttcagccttctttgggatgtcacgcattggaatggaagtgactcccagcgggacctggctgacttataccggagccatcaagctggacgataaagatcctaacttcaaggaccaggtcatcctgctgaacaagcatatcgacgcctacaaaacatttccacccactgaacccaagaaagataagaaaaagaaagccgacgagacacaggctctgccacagcggcagaagaaacagcagacagtgactctgctgcccgctgccgatctggacgatttctcaaaacagctgcagcagtcaatgtcttccgccgattcaactcaggca

SEQ ID NO: 64 - protein, 1120 aa, synthetic construct
  REGION 1..1120: note = OC2.3 (N501Y) polypeptide sequence
MDWTWILFLVAAATRVHSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 65 - protein, 1120 aa, synthetic construct
  REGION 1..1120: note = OC2.3 (N439K, N501Y) polypeptide sequence
MDWTWILFLVAAATRVHSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNKLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNKLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 66 - protein, 1120 aa, synthetic construct
  REGION 1..1120: note = OC2.3 (K417N, E484K, N501Y) polypeptide sequence
MDWTWILFLVAAATRVHSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 67 - protein, 1120 aa, synthetic construct
  REGION 1..1120: note = OC2.3 (K417N, N439K, E484K, N501Y) polypeptide sequence
MDWTWILFLVAAATRVHSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNKLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNKLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 68 - protein, 1339 aa, synthetic construct
  REGION 1..1339: note = OC2.4 polypeptide sequence
MDWTWILFLVAAATRVHSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNKLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 69 - protein, 899 aa, synthetic construct
  REGION 1..899: note = SVF-14 polypeptide sequence
MDWTWILFLVAAATRVHSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKGSGATNFSLLKQAGDVEENPGPMASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 70 - protein, 1733 aa, synthetic construct
  REGION 1..1733: note = SVF-15 polypeptide sequence
MDWTWILFLVAAATRVHSAFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTGSGATNFSLLKQAGDVEENPGPMASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 71 - DNA, 3819 bp, synthetic construct
  misc_feature 1..3819: note = SVF-13 (OC-13) codon optimized DNA sequence (S protein)
  source 1..3819: mol_type = other DNA
atgtttgtctttctggtcctgctgcccctggtctcatcacagtgcgtcaacctgactacacgaacccagctgccacctgcttatacaaattccttcacccggggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaattgcacatttgagtacgtgtcccagcctttcctgatggacctggagggcaagcagggcaatttcaagaacctgagggagttcgtgtttaagaatatcgatggctacttcaagatctactctaagcacacccccatcaacctggtgcgcgacctgcctcagggcttcagcgccctggagccactggtggatctgcctatcggcatcaacatcacccggtttcagacactgctggccctgcacagaagctacctgacacccggcgactcctctagcggatggaccgcaggagcagcagcctactatgtgggctatctgcagcctaggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaaccaggttcgcaagcgtgtacgcatggaataggaagcgcatctctaactgcgtggccgactatagcgtgctgtacaactccgcctctttcagcacctttaagtgctatggcgtgtcccccacaaagctgaatgacctgtgctttaccaacgtgtacgccgattctttcgtgatcaggggcgacgaggtgcgccagatcgcacctggacagacaggcaagatcgccgactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagagatctaccaggccggcagcaccccttgcaatggcgtggagggctttaactgttatttcccactgcagtcctacggcttccagcccacaaacggcgtgggctatcagccttaccgcgtggtggtgctgagctttgagctgctgcacgcaccagcaacagtgtgcggacccaagaagtccaccaatctggtgaagaacaagtgcgtgaacttcaacttcaacggcctgaccggaacaggcgtgctgaccgagtccaacaagaagttcctgccatttcagcagttcggcagggacatcgcagataccacagacgccgtgcgcgacccacagaccctggagatcctggatatcacaccctgctctttcggcggcgtgagcgtgatcacaccaggaaccaatacaagcaaccaggtggccgtgctgtatcaggacgtgaattgtaccgaggtgcctgtggccatccacgccgatcagctgaccccaacatggcgggtgtacagcaccggctccaacgtgttccagacaagagcaggatgcctgatcggagcagagcacgtgaacaattcctatgagtgcgacatcccaatcggcgccggcatctgtgcctcttaccagacccagacaaactctccaaggagagcacggagcgtggcatcccagtctatcatcgcctataccatgtccctgggcgccgagaattctgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctgcagtacggcagcttttgtacccagctgaatagagccctgacaggcatcgccgtggagcaggataagaacacacaggaggtgttcgcccaggtgaagcagatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagcacgggacctgatctgtgcccagaagtttaatggcctgaccgtgctgccacccctgctgacagatgagatgatcgcacagtacacaagcgccctgctggcaggaaccatcacatccggatggaccttcggcgcaggagccgccctgcagatcccctttgccatgcagatggcctataggttcaacggcatcggcgtgacccagaatgtgctgtacgagaaccagaagctgatcgccaatcagtttaactccgccatcggcaagatccaggacagcctgtcctctacagcctccgccctgggcaagctgcaggatgtggtgaatcagaacgcccaggccctgaataccctggtgaagcagctgagctccaacttcggcgccatctctagcgtgctgaatgatatcctgagccggctggacaaggtggaggcagaggtgcagatcgaccggctgatcacaggcagactgcagtctctgcagacctatgtgacacagcagctgatcagggcagcagagatcagggcaagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaataccttcgtgagcggcaactgtgacgtggtcatcggcatcgtgaacaataccgtgtatgatccactgcagcccgagctggacagctttaaggaggagctggataagtacttcaagaatcacacctcccctgacgtggatctgggcgacatcagcggcatcaatgcctccgtggtgaacatccagaaggagatcgaccgcctgaacgaggtggccaagaatctgaacgagagcctgatcgatctgcaggagctgggcaagtatgagcagtacatcaagtggccatggtacatctggctgggcttcatcgccggcctgatcgccatcgtgatggtgaccatcatgctgtgctgtatgacatcctgctgttcttgcctgaagggctgctgtagctgtggctcctgctgtaagtttgatgaggacgattctgagcccgtgctgaagggagtgaagctgcattacacc

SEQ ID NO: 72 - protein, 1273 aa, synthetic construct
  REGION 1..1273: note = SVF-13 (OC-13) polypeptide sequence (S protein)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 73 - DNA, 1320 bp, synthetic construct
  misc_feature 1..1320: note = SVF-10.2 (OC-10.2) codon optimized DNA sequence
  source 1..1320: mol_type = other DNA
atggcccgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaagatcgctgactacaattataaactgcccgacgatttcaccggctgcgtgatcgcctggaacagcaacaatctggattccaaagtgggcgggaactacaattatctgtaccggctgttcagaaagtccaatctgaaaccctttgagagggacatcagtactgaaatctaccaggccgggtcaaccccttgcaatggggtggagggattcaactgttactttccactgcagagttatggatttcagcccaccaacggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaagattgctgactataattacaaactgccagacgatttcactggatgcgtgattgcctggaactctaacaatctggatagtaaagtgggaggcaactataattacctgtataggctgttccgcaagtctaatctgaaaccatttgagcgggacatcagcaccgagatctaccaggccggctccaccccctgcaatggagtggagggcttcaactgctattttccactgcagagctatgggtttcagcccacaaacggggtgggataccagccttatagagtggtggtgctgtccttcgaactgctgcatgcccctgctacagtgtgcggcccaaagaaatctaccaacctggtcaagaacaag

SEQ ID NO: 74 - protein, 440 aa, synthetic construct
  REGION 1..440: note = SVF-10.2 (OC-10.2) polypeptide sequence
MARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 75 - DNA, 1371 bp, synthetic construct
  misc_feature 1..1371: note = SVF-10.3 (OC-10.3) codon optimized DNA sequence
  source 1..1371: mol_type = other DNA
atggactggacctggattctgttcctggtggccgccgccacacgggtgcacagcgcccgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaagatcgctgactacaattataaactgcccgacgatttcaccggctgcgtgatcgcctggaacagcaacaatctggattccaaagtgggcgggaactacaattatctgtaccggctgttcagaaagtccaatctgaaaccctttgagagggacatcagtactgaaatctaccaggccgggtcaaccccttgcaatggggtggagggattcaactgttactttccactgcagagttatggatttcagcccaccaacggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaagattgctgactataattacaaactgccagacgatttcactggatgcgtgattgcctggaactctaacaatctggatagtaaagtgggaggcaactataattacctgtataggctgttccgcaagtctaatctgaaaccatttgagcgggacatcagcaccgagatctaccaggccggctccaccccctgcaatggagtggagggcttcaactgctattttccactgcagagctatgggtttcagcccacaaacggggtgggataccagccttatagagtggtggtgctgtccttcgaactgctgcatgcccctgctacagtgtgcggcccaaagaaatctaccaacctggtcaagaacaag

SEQ ID NO: 76 - protein, 457 aa, synthetic construct
  REGION 1..457: note = SVF-10.3 (OC-10.3) polypeptide sequence
MDWTWILFLVAAATRVHSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 77 - DNA, 3288 bp, synthetic construct
  misc_feature 1..3288: note = SVF-14.2 (OC-14.2) DNA sequence
  source 1..3288: mol_type = other DNA
atggactggacctggattctgttcctggtggccgccgccacacgggtgcacagcgcccgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaagatcgctgactacaattataaactgcccgacgatttcaccggctgcgtgatcgcctggaacagcaacaatctggattccaaagtgggcgggaactacaattatctgtaccggctgttcagaaagtccaatctgaaaccctttgagagggacatcagtactgaaatctaccaggccgggtcaaccccttgcaatggggtggagggattcaactgttactttccactgcagagttatggatttcagcccaccaacggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaagattgctgactataattacaaactgccagacgatttcactggatgcgtgattgcctggaactctaacaagctggatagtaaagtgggaggcaactataattacctgtataggctgttccgcaagtctaatctgaaaccatttgagcgggacatcagcaccgagatctaccaggccggctccaccccctgcaatggagtggagggcttcaactgctattttccactgcagagctatgggtttcagcccacatatggggtgggataccagccttatagagtggtggtgctgtccttcgaactgctgcatgcccctgctacagtgtgcggcccaaagaaatctaccaacctggtcaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaatattgctgactataattacaaactgccagacgatttcactggatgcgtgattgcctggaactctaacaatctggatagtaaagtgggaggcaactataattacctgtataggctgttccgcaagtctaatctgaaaccatttgagcgggacatcagcaccgagatctaccaggccggctccaccccctgcaatggagtgaagggcttcaactgctattttccactgcagagctatgggtttcagcccacatatggggtgggataccagccttatagagtggtggtgctgtccttcgaactgctgcatgcccctgctacagtgtgcggcccaaagaaatctaccaacctggtcaagaacaagatggctagcgacaacgggccccagaaccagcggaatgcccctagaatcaccttcggcgggccatccgattctacaggcagcaaccagaatggagagaggtccggagctcgctctaagcagagacggccccagggcctgccaaacaataccgcctcctggtttactgctctgacccagcatgggaaggaagatctgaaattccccaggggacagggcgtgcctatcaacaccaattcaagcccagacgatcagattgggtactataggagggctacaaggagaatccggggaggcgacggaaagatgaaagatctgagccccagatggtacttttactatctgggaacaggaccagaggctggactgccttatggcgctaacaaggacggaatcatttgggtggccactgaaggcgctctgaatacccctaaagatcacattggaacccgcaacccagccaacaatgccgctatcgtgctgcagctgccacagggcaccacactgcccaagggcttctacgctgaggggagtaggggaggatcacaggcttcctctcgcagttcaagcaggtcccgcaattcctctcggaactctacaccagggagttcacggggaactagcccagctagaatggctggaaatggaggggacgccgctctggccctgctgctgctggatagactgaaccagctggagtctaagatgagtggcaaagggcagcagcagcagggacagacagtgactaagaaatccgccgctgaagcctctaagaaaccccggcagaagagaaccgctacaaaagcctacaatgtgacccaggcttttggcaggcgcggacctgagcagacacagggaaacttcggcgaccaggaactgattaggcagggcaccgattataagcactggccccagatcgctcagttcgcccctagtgcttcagccttctttggaatgtctcgcattggcatggaggtgacacctagtggcacttggctgacttacaccggggccatcaagctggacgataaagacccaaacttcaaggatcaggtcatcctgctgaacaagcatattgacgcctataaaaccttcccccctacagagcccaagaaagacaagaaaaagaaagccgatgaaacccaggctctgccccagaggcagaagaaacagcagacagtgactctgctgcctgccgctgatctggacgatttcagtaaacagctgcagcagagtatgagtagtgccgacagtacccaggct

SEQ ID NO: 78 - DNA, 3288 bp, synthetic construct
  misc_feature 1..3288: note = SVF-14.2 (OC-14.2) codon optimized DNA sequence
  source 1..3288: mol_type = other DNA
atggactggacatggattctgtttctggtcgcagccgctacaagggtgcattccgcccgcgtgcagcccactgagagcatcgtgaggttccccaacatcacaaatctgtgccctttcggcgaggtgtttaacgctactcggtttgcatccgtctacgcctggaaccgcaagcgaattagcaattgcgtggccgactattccgtgctgtacaattccgcttctttcagtacctttaagtgctatggggtgagccccactaaactgaacgacctgtgcttcaccaatgtctacgctgactcctttgtgatccgcggagatgaagtccgacagattgccccagggcagaccggaaagatcgctgactacaactataaactgcccgacgatttcacaggctgcgtgatcgcctggaatagcaacaatctggattccaaggtcggcgggaactacaattatctgtacagactgttcaggaagtctaacctgaaaccatttgagcgggacatcagtacagaaatctaccaggccgggtcaactccctgcaacggcgtggaggggttcaattgttattttccactgcagagttacggattccagcccaccaacggagtcggctatcagccttacagagtggtcgtgctgtcctttgaactgctgcacgcaccagcaaccgtgtgcggacccaagaaatccacaaacctggtcaagaacaagcgcgtgcagccaacagagtctatcgtgcgattcccaaacattactaatctgtgccccttcggggaagtgtttaacgctactagattcgcctccgtctacgcttggaatcggaagagaatctctaattgcgtggcagactatagtgtgctgtacaattcagccagcttctccacctttaagtgctatggcgtgtctcctaccaaactgaacgacctgtgctttaccaacgtgtacgccgacagttttgtcattcggggggatgaggtgagacagatcgcacctggacagactggcaagattgccgactataactacaaactgccagacgatttcaccggatgcgtgattgcctggaactctaataagctggatagtaaagtcggaggcaactataattacctgtatcgcctgttccgaaagagcaacctgaaaccttttgagagggacatcagcaccgagatctaccaggccggctccactccatgcaacggggtggagggattcaattgctactttccactgcagtcttacgggttccagcccacctatggggtgggatatcagccttaccgcgtcgtggtcctgagctttgaactgctgcatgctcctgcaaccgtgtgcggcccaaagaaaagtaccaacctggtgaagaacaagcgggtgcagcctaccgagtcaatcgtgagattccctaacattacaaatctgtgcccattcggcgaagtgtttaacgctacaaggtttgcaagcgtctacgcctggaacaggaagcgcatctcaaattgcgtggctgactatagcgtgctgtacaattctgcaagtttctcaacttttaagtgctatggagtgtcacctacaaaactgaacgacctgtgcttcacgaatgtctacgccgacagctttgtgatcaggggcgatgaggtccgccagattgctcccggccagacagggaacatcgcagactacaattataagctgcctgacgatttcactggatgcgtgattgcttggaactcaaacaatctggatagcaaagtcgggggaaattataattatctgtaccggctgttcagaaagtctaatctgaaaccctttgagcgggacatctccaccgaaatctaccaggccggaagtactccttgcaacggagtgaagggcttcaattgttactttccactgcagagctatggcttccagcccacctacggcgtggggtaccagccatatagggtcgtcgtgctgtcctttgagctgctgcacgcaccagctaccgtgtgcggacctaagaaatctaccaacctggtcaaaaacaagatggctagcgataatggaccccagaaccagcgaaatgcacctcggatcacttttggcggaccaagcgactccaccggctccaaccagaatggagagagatccggagcaaggtctaagcagagacggccccagggcctgccaaacaataccgccagctggttcacagctctgactcagcacgggaaggaagatctgaaatttccaagggggcagggagtgcccattaacaccaatagctcccccgacgatcagatcgggtactataggcgagcaacacgacggattaggggaggcgacggaaagatgaaagatctgtcccctcgctggtatttctactatctgggaaccggaccagaggctgggctgccttacggagcaaacaaggacggaatcatttgggtggctacagaaggcgcactgaacactcctaaagatcatatcggaacacgcaatccagccaacaatgccgctattgtgctgcagctgcctcagggcaccacactgccaaagggcttctacgcagagggcagtagagggggatcacaggcctctagtaggtcaagctcccgatcccggaactctagtaggaattctaccccaggctcaagccgagggacaagcccagctcgaatggcaggaaacggaggggacgcagccctggccctgctgctgctggatcgcctgaatcagctggagtcaaagatgagcggaaaaggccagcagcagcagggacagactgtgaccaagaaatccgctgcagaagcttctaagaaacctaggcagaagcgcacagccactaaagcttataacgtgactcaggccttcgggagaaggggaccagagcagacccagggcaattttggggaccaggaactgatccggcagggcaccgattacaagcactggccccagattgcacagttcgccccttctgcaagtgccttctttgggatgtcaagaatcggaatggaggtgaccccaagcggcacatggctgacctatacaggggctattaagctggacgataaagaccccaacttcaaggatcaggtcatcctgctgaacaagcatattgacgcctacaaaacttttccccctaccgagcccaagaaagacaagaaaaagaaagcagatgaaacacaggccctgccccagcgccagaagaaacagcagactgtgaccctgctgcctgccgctgatctggatgatttcagtaaacagctgcagcagtccatgagttccgccgattccacccaggca

SEQ ID NO: 79 - protein, 1096 aa, synthetic construct
  REGION 1..1096: note = SVF-14.2 (OC-14.2) polypeptide sequence
MDWTWILFLVAAATRVHSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNKLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKMASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 80 - DNA, 3354 bp, synthetic construct
  misc_feature 1..3354: note = SVF-14.3 (OC-14.3) DNA sequence
  source 1..3354: mol_type = other DNA
atggactggacctggattctgttcctggtggccgccgccacacgggtgcacagcgcccgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaagatcgctgactacaattataaactgcccgacgatttcaccggctgcgtgatcgcctggaacagcaacaatctggattccaaagtgggcgggaactacaattatctgtaccggctgttcagaaagtccaatctgaaaccctttgagagggacatcagtactgaaatctaccaggccgggtcaaccccttgcaatggggtggagggattcaactgttactttccactgcagagttatggatttcagcccaccaacggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaagattgctgactataattacaaactgccagacgatttcactggatgcgtgattgcctggaactctaacaagctggatagtaaagtgggaggcaactataattacctgtataggctgttccgcaagtctaatctgaaaccatttgagcgggacatcagcaccgagatctaccaggccggctccaccccctgcaatggagtggagggcttcaactgctattttccactgcagagctatgggtttcagcccacatatggggtgggataccagccttatagagtggtggtgctgtccttcgaactgctgcatgcccctgctacagtgtgcggcccaaagaaatctaccaacctggtcaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaatattgctgactataattacaaactgccagacgatttcactggatgcgtgattgcctggaactctaacaatctggatagtaaagtgggaggcaactataattacctgtataggctgttccgcaagtctaatctgaaaccatttgagcgggacatcagcaccgagatctaccaggccggctccaccccctgcaatggagtgaagggcttcaactgctattttccactgcagagctatgggtttcagcccacatatggggtgggataccagccttatagagtggtggtgctgtccttcgaactgctgcatgcccctgctacagtgtgcggcccaaagaaatctaccaacctggtcaagaacaagggaagcggagctactaacttcagcctgctgaagcaggctggagacgtggaggagaaccctggacctatggctagcgacaacgggccccagaaccagcggaatgcccctagaatcaccttcggcgggccatccgattctacaggcagcaaccagaatggagagaggtccggagctcgctctaagcagagacggccccagggcctgccaaacaataccgcctcctggtttactgctctgacccagcatgggaaggaagatctgaaattccccaggggacagggcgtgcctatcaacaccaattcaagcccagacgatcagattgggtactataggagggctacaaggagaatccggggaggcgacggaaagatgaaagatctgagccccagatggtacttttactatctgggaacaggaccagaggctggactgccttatggcgctaacaaggacggaatcatttgggtggccactgaaggcgctctgaatacccctaaagatcacattggaacccgcaacccagccaacaatgccgctatcgtgctgcagctgccacagggcaccacactgcccaagggcttctacgctgaggggagtaggggaggatcacaggcttcctctcgcagttcaagcaggtcccgcaattcctctcggaactctacaccagggagttcacggggaactagcccagctagaatggctggaaatggaggggacgccgctctggccctgctgctgctggatagactgaaccagctggagtctaagatgagtggcaaagggcagcagcagcagggacagacagtgactaagaaatccgccgctgaagcctctaagaaaccccggcagaagagaaccgctacaaaagcctacaatgtgacccaggcttttggcaggcgcggacctgagcagacacagggaaacttcggcgaccaggaactgattaggcagggcaccgattataagcactggccccagatcgctcagttcgcccctagtgcttcagccttctttggaatgtctcgcattggcatggaggtgacacctagtggcacttggctgacttacaccggggccatcaagctggacgataaagacccaaacttcaaggatcaggtcatcctgctgaacaagcatattgacgcctataaaaccttcccccctacagagcccaagaaagacaagaaaaagaaagccgatgaaacccaggctctgccccagaggcagaagaaacagcagacagtgactctgctgcctgccgctgatctggacgatttcagtaaacagctgcagcagagtatgagtagtgccgacagtacccaggct

SEQ ID NO: 81 - DNA, 3354 bp, synthetic construct
  misc_feature 1..3354: note = SVF-14.3 (OC-14.3) codon optimized DNA sequence
  source 1..3354: mol_type = other DNA
atggactggacatggattctgtttctggtcgcagccgctacaagggtgcattccgcccgcgtgcagcccactgagagcatcgtgaggttccccaacatcacaaatctgtgccctttcggcgaggtgtttaacgctactcggtttgcatccgtctacgcctggaaccgcaagcgaattagcaattgcgtggccgactattccgtgctgtacaattccgcttctttcagtacctttaagtgctatggggtgagccccactaaactgaacgacctgtgcttcaccaatgtctacgctgactcctttgtgatccgcggagatgaagtccgacagattgccccagggcagaccggaaagatcgctgactacaactataaactgcccgacgatttcacaggctgcgtgatcgcctggaatagcaacaatctggattccaaggtcggcgggaactacaattatctgtacagactgttcaggaagtctaacctgaaaccatttgagcgggacatcagtacagaaatctaccaggccgggtcaactccctgcaacggcgtggaggggttcaattgttattttccactgcagagttacggattccagcccaccaacggagtcggctatcagccttacagagtggtcgtgctgtcctttgaactgctgcacgcaccagcaaccgtgtgcggacccaagaaatccacaaacctggtcaagaacaagcgcgtgcagccaacagagtctatcgtgcgattcccaaacattactaatctgtgccccttcggggaagtgtttaacgctactagattcgcctccgtctacgcttggaatcggaagagaatctctaattgcgtggcagactatagtgtgctgtacaattcagccagcttctccacctttaagtgctatggcgtgtctcctaccaaactgaacgacctgtgctttaccaacgtgtacgccgacagttttgtcattcggggggatgaggtgagacagatcgcacctggacagactggcaagattgccgactataactacaaactgccagacgatttcaccggatgcgtgattgcctggaactctaataagctggatagtaaagtcggaggcaactataattacctgtatcgcctgttccgaaagagcaacctgaaaccttttgagagggacatcagcaccgagatctaccaggccggctccactccatgcaacggggtggagggattcaattgctactttccactgcagtcttacgggttccagcccacctatggggtgggatatcagccttaccgcgtcgtggtcctgagctttgaactgctgcatgctcctgcaaccgtgtgcggcccaaagaaaagtaccaacctggtgaagaacaagcgggtgcagcctaccgagtcaatcgtgagattccctaacattacaaatctgtgcccattcggcgaagtgtttaacgctacaaggtttgcaagcgtctacgcctggaacaggaagcgcatctcaaattgcgtggctgactatagcgtgctgtacaattctgcaagtttctcaacttttaagtgctatggagtgtcacctacaaaactgaacgacctgtgcttcacgaatgtctacgccgacagctttgtgatcaggggcgatgaggtccgccagattgctcccggccagacagggaacatcgcagactacaattataagctgcctgacgatttcactggatgcgtgattgcttggaactcaaacaatctggatagcaaagtcgggggaaattataattatctgtaccggctgttcagaaagtctaatctgaaaccctttgagcgggacatctccaccgaaatctaccaggccggaagtactccttgcaacggagtgaagggcttcaattgttactttccactgcagagctatggcttccagcccacctacggcgtggggtaccagccatatagggtcgtcgtgctgtcctttgagctgctgcacgcaccagctaccgtgtgcggacctaagaaatctaccaacctggtcaaaaacaagggaagcggagctactaacttcagcctgctgaagcaggctggagacgtggaggagaaccctggacctatggctagcgataatggaccccagaaccagcgaaatgcacctcggatcacttttggcggaccaagcgactccaccggctccaaccagaatggagagagatccggagcaaggtctaagcagagacggccccagggcctgccaaacaataccgccagctggttcacagctctgactcagcacgggaaggaagatctgaaatttccaagggggcagggagtgcccattaacaccaatagctcccccgacgatcagatcgggtactataggcgagcaacacgacggattaggggaggcgacggaaagatgaaagatctgtcccctcgctggtatttctactatctgggaaccggaccagaggctgggctgccttacggagcaaacaaggacggaatcatttgggtggctacagaaggcgcactgaacactcctaaagatcatatcggaacacgcaatccagccaacaatgccgctattgtgctgcagctgcctcagggcaccacactgccaaagggcttctacgcagagggcagtagagggggatcacaggcctctagtaggtcaagctcccgatcccggaactctagtaggaattctaccccaggctcaagccgagggacaagcccagctcgaatggcaggaaacggaggggacgcagccctggccctgctgctgctggatcgcctgaatcagctggagtcaaagatgagcggaaaaggccagcagcagcagggacagactgtgaccaagaaatccgctgcagaagcttctaagaaacctaggcagaagcgcacagccactaaagcttataacgtgactcaggccttcgggagaaggggaccagagcagacccagggcaattttggggaccaggaactgatccggcagggcaccgattacaagcactggccccagattgcacagttcgccccttctgcaagtgccttctttgggatgtcaagaatcggaatggaggtgaccccaagcggcacatggctgacctatacaggggctattaagctggacgataaagaccccaacttcaaggatcaggtcatcctgctgaacaagcatattgacgcctacaaaacttttccccctaccgagcccaagaaagacaagaaaaagaaagcagatgaaacacaggccctgccccagcgccagaagaaacagcagactgtgaccctgctgcctgccgctgatctggatgatttcagtaaacagctgcagcagtccatgagttccgccgattccacccaggca

SEQ ID NO: 82 - protein, 1118 aa, synthetic construct
  REGION 1..1118: note = SVF-14.3 (OC-14.3) polypeptide sequence
MDWTWILFLVAAATRVHSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNKLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKGSGATNFSLLKQAGDVEENPGPMASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 83 - protein, 1273 aa, synthetic construct; identical to SEQ ID NO: 72
  REGION 1..1273: note = SARS-CoV-2 S protein (NCBI Reference YP_009724390.1)

SEQ ID NO: 84 - DNA, 4017 bp, synthetic construct
  misc_feature 1..4017: note = SVF-2.5 (OC-2.5) codon optimized DNA sequence
  source 1..4017: mol_type = other DNA
atggactggacttggattctgttcctggtcgccgccgccacccgagtgcatagcgcccgcgtgcagcccaccgagagcattgtgaggtttcccaacatcacaaatctgtgccctttcggcgaggtgtttaatgctactaggttcgccagcgtgtacgcttggaatcggaagagaatttctaactgcgtggccgactatagtgtgctgtacaacagcgcctccttctctacctttaagtgctatggggtgtctcccaccaaactgaatgacctgtgcttcacaaacgtgtacgccgacagttttgtgatccggggagatgaggtgagacagattgccccaggccagaccgggaagatcgctgactacaattataaactgcccgacgatttcacaggctgcgtgatcgcctggaacagtaacaatctggattcaaaagtgggcgggaactacaattatctgtacaggctgttccgcaagagcaacctgaaaccatttgagagggacatcagcactgaaatctaccaggccggctccaccccctgcaatggagtggagggcttcaactgttattttccactgcagagctacgggtttcagcccaccaacggggtgggatatcagccttaccgcgtggtggtgctgtccttcgaactgctgcacgctccagctaccgtgtgcggacccaagaaaagtactaacctggtgaagaacaagagggtgcagccaactgagtcaatcgtgcgctttccaaacattaccaatctgtgccccttcggggaagtgtttaatgccacacgcttcgcttccgtgtacgcctggaataggaagcgcatctcaaattgcgtggctgactatagcgtgctgtacaacagtgcttcattcagcacttttaagtgctatggcgtgtcacctactaaactgaatgacctgtgctttaccaacgtgtacgctgacagctttgtgattaggggggatgaggtgcgccagatcgctcctggacagactggaaatattgctgactataactacaaactgccagacgatttcaccggatgcgtgattgcctggaacagcaataagctggattccaaagtgggaggcaactataattacaggtatcggctgttcagaaagtctaacctgaaaccttttgagcgggacatctctaccgagatctaccaggccggaagtaagccatgcaatggcgtggaggggttcaactgctactttccactgcagtcttacggctttcagcccacctatggcgtggggtatcagccttacagagtggtggtgctgagcttcgaactgctgcatgctcctgctaccgtgtgcggaccaaagaaaagcaccaacctggtcaagaacaagcgggtgcagcctacagagtccatcgtgagatttcctaacattactaatctgtgcccattcggcgaagtgtttaatgctacaagatttgctagcgtgtacgcctggaatcggaagagaatctccaattgcgtggcagactattctgtgctgtacaactccgcttctttcagtacctttaagtgttacggagtgtccccaacaaaactgaatgacctgtgcttcacgaacgtgtacgccgactcttttgtgatccggggcgatgaggtgagacagatcgcccccggacagacaggcaatatcgctgactacaactataagctgcctgacgatttcactggatgcgtgattgcttggaattctaacaatctggatagtaaagtggggggaaattataactatctgtataggctgttccgcaagtctaacctgaaaccctttgagagggacatctcaaccgagatctaccaggctgggagcaccccttgcaatggggtgaagggattcaactgttactttccactgcagagctatggatttcagcccacatacggagtgggctaccagccatatagagtggtggtgctgagtttcgagctgctgcatgccccagctacagtgtgcggacctaagaaaagcacaaacctggtcaaaaacaagggaagcggagctactaacttcagcctgctgaagcaggctggagacgtggaggagaaccctggacctatggccgatagcaacgggactatcaccgtggaggaactgaagaaactgctggaacagtggaacctggtcatcggcttcctgtttctgacctggatctgtctgctgcagttcgcctatgctaacaggaatcgctttctgtacatcattaagctgattttcctgtggctgctgtggcctgtgactctggcctgcttcgtgctggccgccgtgtacaggatcaactggattaccggcgggatcgccattgctatggcctgtctggtgggcctgatgtggctgtcatactttatcgccagcttccgcctgtttgctcggaccagaagtatgtggtcattcaaccctgagacaaatattctgctgaacgtgccactgcacggcaccatcctgacacggcccctgctggagagcgaactggtcatcggcgccgtgattctgagggggcatctgcgcatcgctggccaccatctggggagatgcgacatcaaggatctgcccaaagaaattactgtggccacctctaggacactgagttactataagctgggagcttcccagagggtggctggcgacagcggatttgctgcttattccaggtaccgcatcgggaattacaaactgaacacagaccacagctcctctagtgataatattgccctgctggtgcaggctagcgacaacggaccccagaaccagcggaatgcccctagaatcaccttcggaggcccatcagatagcacaggctccaaccagaatggagagaggtcaggagctcgcagcaagcagagacggccccagggactgccaaacaataccgcctcttggtttactgctctgacccagcatggcaaggaagatctgaaattcccacggggccagggggtgcccattaacaccaattcaagccccgacgatcagatcggctactataggagggctacaaggagaattcggggaggagacgggaagatgaaagatctgagccctagatggtacttctactatctgggaacaggaccagaggctgggctgccttacggagctaacaaggacgggatcatttgggtggccactgaaggagctctgaatacccctaaagatcacatcgggaccagaaacccagccaacaatgccgctattgtgctgcagctgcctcagggaaccacactgccaaagggattctacgctgagggctcccggggaggcagccaggcttccagccggagctcaagccggagtagaaattcctctcggaactcaacaccaggcagttcaagggggacttccccagctaggatggctggaaatggaggcgacgccgctctggccctgctgctgctggatagactgaaccagctggagagcaagatgtccgggaaaggacagcagcagcagggacagacagtgactaagaaatccgccgctgaagcctctaagaaacctcggcagaagagaaccgctacaaaagcctataatgtgactcaggcttttggaaggcgcggaccagagcagacccagggaaacttcggcgaccaggaactgatcaggcagggcaccgattacaagcactggccccagattgctcagttcgccccttccgcttctgccttctttgggatgtcccgcatcggaatggaggtgacaccatctggaacttggctgacttataccggcgccattaagctggacgataaagaccccaacttcaaggatcaggtcatcctgctgaacaagcatattgacgcctacaaaaccttcccccctacagagcccaagaaagacaagaaaaagaaagccgatgaaacccaggctctgccccagagacagaagaaacagcagacagtgactctgctgcctgccgctgatctggacgatttctcaaaacagctgcagcagagcatgtcaagtgccgattcaactcaggct

SEQ ID NO: 85 - protein, 1339 aa, synthetic construct
  REGION 1..1339: note = SVF-2.5 (OC-2.5) polypeptide sequence
MDWTWILFLVAAATRVHSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNKLDSKVGGNYNYRYRLFRKSNLKPFERDISTEIYQAGSKPCNGVEGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 86 - DNA, 3288 bp, synthetic construct
  misc_feature 1..3288: note = SVF-14.4 (OC-14.4) codon optimized DNA sequence
  source 1..3288: mol_type = other DNA
atggactggacatggattctgtttctggtcgcagccgctacaagggtgcattccgcccgcgtgcagcccactgagagcatcgtgaggttccccaacatcacaaatctgtgccctttcggcgaggtgtttaacgctactcggtttgcatccgtctacgcctggaaccgcaagcgaattagcaattgcgtggccgactattccgtgctgtacaattccgcttctttcagtacctttaagtgctatggggtgagccccactaaactgaacgacctgtgcttcaccaatgtctacgctgactcctttgtgatccgcggagatgaagtccgacagattgccccagggcagaccggaaagatcgctgactacaactataaactgcccgacgatttcacaggctgcgtgatcgcctggaatagcaacaatctggattccaaggtcggcgggaactacaattatctgtacagactgttcaggaagtctaacctgaaaccatttgagcgggacatcagtacagaaatctaccaggccgggtcaactccctgcaacggcgtggaggggttcaattgttattttccactgcagagttacggattccagcccaccaacggagtcggctatcagccttacagagtggtcgtgctgtcctttgaactgctgcacgcaccagcaaccgtgtgcggacccaagaaatccacaaacctggtcaagaacaagcgcgtgcagccaacagagtctatcgtgcgattcccaaacattactaatctgtgccccttcggggaagtgtttaacgctactagattcgcctccgtctacgcttggaatcggaagagaatctctaattgcgtggcagactatagtgtgctgtacaattcagccagcttctccacctttaagtgctatggcgtgtctcctaccaaactgaacgacctgtgctttaccaacgtgtacgccgacagttttgtcattcggggggatgaggtgagacagatcgcacctggacagactggcaatattgccgactataactacaaactgccagacgatttcaccggatgcgtgattgcctggaactctaataagctggatagtaaagtcggaggcaactataattacaggtatcgcctgttccgaaagagcaacctgaaaccttttgagagggacatcagcaccgagatctaccaggccggctccaagccatgcaacggggtggagggattcaattgctactttccactgcagtcttacgggttccagcccacctatggggtgggatatcagccttaccgcgtcgtggtcctgagctttgaactgctgcatgctcctgcaaccgtgtgcggcccaaagaaaagtaccaacctggtgaagaacaagcgggtgcagcctaccgagtcaatcgtgagattccctaacattacaaatctgtgcccattcggcgaagtgtttaacgctacaaggtttgcaagcgtctacgcctggaacaggaagcgcatctcaaattgcgtggctgactatagcgtgctgtacaattctgcaagtttctcaacttttaagtgctatggagtgtcacctacaaaactgaacgacctgtgcttcacgaatgtctacgccgacagctttgtgatcaggggcgatgaggtccgccagattgctcccggccagacagggaacatcgcagactacaattataagctgcctgacgatttcactggatgcgtgattgcttggaactcaaacaatctggatagcaaagtcgggggaaattataattatctgtaccggctgttcagaaagtctaatctgaaaccctttgagcgggacatctccaccgaaatctaccaggccggaagtactccttgcaacggagtgaagggcttcaattgttactttccactgcagagctatggcttccagcccacctacggcgtggggtaccagccatatagggtcgtcgtgctgtcctttgagctgctgcacgcaccagctaccgtgtgcggacctaagaaatctaccaacctggtcaaaaacaagatggctagcgataatggaccccagaaccagcgaaatgcacctcggatcacttttggcggaccaagcgactccaccggctccaaccagaatggagagagatccggagcaaggtctaagcagagacggccccagggcctgccaaacaataccgccagctggttcacagctctgactcagcacgggaaggaagatctgaaatttccaagggggcagggagtgcccattaacaccaatagctcccccgacgatcagatcgggtactataggcgagcaacacgacggattaggggaggcgacggaaagatgaaagatctgtcccctcgctggtatttctactatctgggaaccggaccagaggctgggctgccttacggagcaaacaaggacggaatcatttgggtggctacagaaggcgcactgaacactcctaaagatcatatcggaacacgcaatccagccaacaatgccgctattgtgctgcagctgcctcagggcaccacactgccaaagggcttctacgcagagggcagtagagggggatcacaggcctctagtaggtcaagctcccgatcccggaactctagtaggaattctaccccaggctcaagccgagggacaagcccagctcgaatggcaggaaacggaggggacgcagccctggccctgctgctgctggatcgcctgaatcagctggagtcaaagatgagcggaaaaggccagcagcagcagggacagactgtgaccaagaaatccgctgcagaagcttctaagaaacctaggcagaagcgcacagccactaaagcttataacgtgactcaggccttcgggagaaggggaccagagcagacccagggcaattttggggaccaggaactgatccggcagggcaccgattacaagcactggccccagattgcacagttcgccccttctgcaagtgccttctttgggatgtcaagaatcggaatggaggtgaccccaagcggcacatggctgacctatacaggggctattaagctggacgataaagaccccaacttcaaggatcaggtcatcctgctgaacaagcatattgacgcctacaaaacttttccccctaccgagcccaagaaagacaagaaaaagaaagcagatgaaacacaggccctgccccagcgccagaagaaacagcagactgtgaccctgctgcctgccgctgatctggatgatttcagtaaacagctgcagcagtccatgagttccgccgattccacccaggca

SEQ ID NO: 87 - protein, 1096 aa, synthetic construct
  REGION 1..1096: note = SVF-14.4 (OC-14.4) polypeptide sequence
MDWTWILFLVAAATRVHSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNKLDSKVGGNYNYRYRLFRKSNLKPFERDISTEIYQAGSKPCNGVEGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKMASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 88 - DNA, 3354 bp, synthetic construct
  misc_feature 1..3354: note = SVF-14.5 (OC-14.5) codon optimized DNA sequence
  source 1..3354: mol_type = other DNA
atggactggacatggattctgtttctggtcgcagccgctacaagggtgcattccgcccgcgtgcagcccactgagagcatcgtgaggttccccaacatcacaaatctgtgccctttcggcgaggtgtttaacgctactcggtttgcatccgtctacgcctggaaccgcaagcgaattagcaattgcgtggccgactattccgtgctgtacaattccgcttctttcagtacctttaagtgctatggggtgagccccactaaactgaacgacctgtgcttcaccaatgtctacgctgactcctttgtgatccgcggagatgaagtccgacagattgccccagggcagaccggaaagatcgctgactacaactataaactgcccgacgatttcacaggctgcgtgatcgcctggaatagcaacaatctggattccaaggtcggcgggaactacaattatctgtacagactgttcaggaagtctaacctgaaaccatttgagcgggacatcagtacagaaatctaccaggccgggtcaactccctgcaacggcgtggaggggttcaattgttattttccactgcagagttacggattccagcccaccaacggagtcggctatcagccttacagagtggtcgtgctgtcctttgaactgctgcacgcaccagcaaccgtgtgcggacccaagaaatccacaaacctggtcaagaacaagcgcgtgcagccaacagagtctatcgtgcgattcccaaacattactaatctgtgccccttcggggaagtgtttaacgctactagattcgcctccgtctacgcttggaatcggaagagaatctctaattgcgtggcagactatagtgtgctgtacaattcagccagcttctccacctttaagtgctatggcgtgtctcctaccaaactgaacgacctgtgctttaccaacgtgtacgccgacagttttgtcattcggggggatgaggtgagacagatcgcacctggacagactggcaatattgccgactataactacaaactgccagacgatttcaccggatgcgtgattgcctggaactctaataagctggatagtaaagtcggaggcaactataattacaggtatcgcctgttccgaaagagcaacctgaaaccttttgagagggacatcagcaccgagatctaccaggccggctccaagccatgcaacggggtggagggattcaattgctactttccactgcagtcttacgggttccagcccacctatggggtgggatatcagccttaccgcgtcgtggtcctgagctttgaactgctgcatgctcctgcaaccgtgtgcggcccaaagaaaagtaccaacctggtgaagaacaagcgggtgcagcctaccgagtcaatcgtgagattccctaacattacaaatctgtgcccattcggcgaagtgtttaacgctacaaggtttgcaagcgtctacgcctggaacaggaagcgcatctcaaattgcgtggctgactatagcgtgctgtacaattctgcaagtttctcaacttttaagtgctatggagtgtcacctacaaaactgaacgacctgtgcttcacgaatgtctacgccgacagctttgtgatcaggggcgatgaggtccgccagattgctcccggccagacagggaacatcgcagactacaattataagctgcctgacgatttcactggatgcgtgattgcttggaactcaaacaatctggatagcaaagtcgggggaaattataattatctgtaccggctgttcagaaagtctaatctgaaaccctttgagcgggacatctccaccgaaatctaccaggccggaagtactccttgcaacggagtgaagggcttcaattgttactttccactgcagagctatggcttccagcccacctacggcgtggggtaccagccatatagggtcgtcgtgctgtcctttgagctgctgcacgcaccagctaccgtgtgcggacctaagaaatctaccaacctggtcaaaaacaagggaagcggagctactaacttcagcctgctgaagcaggctggagacgtggaggagaaccctggacctatggctagcgataatggaccccagaaccagcgaaatgcacctcggatcacttttggcggaccaagcgactccaccggctccaaccagaatggagagagatccggagcaaggtctaagcagagacggccccagggcctgccaaacaataccgccagctggttcacagctctgactcagcacgggaaggaagatctgaaatttccaagggggcagggagtgcccattaacaccaatagctcccccgacgatcagatcgggtactataggcgagcaacacgacggattaggggaggcgacggaaagatgaaagatctgtcccctcgctggtatttctactatctgggaaccggaccagaggctgggctgccttacggagcaaacaaggacggaatcatttgggtggctacagaaggcgcactgaacactcctaaagatcatatcggaacacgcaatccagccaacaatgccgctattgtgctgcagctgcctcagggcaccacactgccaaagggcttctacgcagagggcagtagagggggatcacaggcctctagtaggtcaagctcccgatcccggaactctagtaggaattctaccccaggctcaagccgagggacaagcccagctcgaatggcaggaaacggaggggacgcagccctggccctgctgctgctggatcgcctgaatcagctggagtcaaagatgagcggaaaaggccagcagcagcagggacagactgtgaccaagaaatccgctgcagaagcttctaagaaacctaggcagaagcgcacagccactaaagcttataacgtgactcaggccttcgggagaaggggaccagagcagacccagggcaattttggggaccaggaactgatccggcagggcaccgattacaagcactggccccagattgcacagttcgccccttctgcaagtgccttctttgggatgtcaagaatcggaatggaggtgaccccaagcggcacatggctgacctatacaggggctattaagctggacgataaagaccccaacttcaaggatcaggtcatcctgctgaacaagcatattgacgcctacaaaacttttccccctaccgagcccaagaaagacaagaaaaagaaagcagatgaaacacaggccctgccccagcgccagaagaaacagcagactgtgaccctgctgcctgccgctgatctggatgatttcagtaaacagctgcagcagtccatgagttccgccgattccacccaggca

SEQ ID NO: 89 - protein, 1118 aa, synthetic construct
  REGION 1..1118: note = SVF-14.5 (OC-14.5) polypeptide sequence
MDWTWILFLVAAATRVHSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNKLDSKVGGNYNYRYRLFRKSNLKPFERDISTEIYQAGSKPCNGVEGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKGSGATNFSLLKQAGDVEENPGPMASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 90 - DNA, 75 bp, synthetic construct
  misc_feature 1..75: note = SARS-CoV-2 CC40.8 epitope codon optimized DNA sequence
  source 1..75: mol_type = other DNA
cctctgcagcctgagctggacagcttcaaagaggaactggataagtacttcaagaaccacacctctcccgacgtg

SEQ ID NO: 91 - protein, 25 aa, synthetic construct
  REGION 1..25: note = SARS-CoV-2 CC40.8 epitope polypeptide sequence
PLQPELDSFKEELDKYFKNHTSPDV

SEQ ID NO: 92 - DNA, 3288 bp, synthetic construct
  misc_feature 1..3288: note = SVF-16.1 codon optimized DNA sequence
  source 1..3288: mol_type = other DNA
atggactggacctggatcctgttcctggtggctgctgctacacgggtgcacagcgccagagtgcagcctaccgaaagcatcgtgcggttcccgaacatcaccaacctgtgcccctttgacgaggtgttcaacgccaccagattcgctagcgtgtacgcctggaatagaaagagaatcagcaactgcgtcgccgactacagcgtcctgtacaatctggccccgttcttcaccttcaagtgctacggcgtgtcacctaccaagctgaacgacctgtgtttcaccaatgtgtacgccgattccttcgtgattagaggcgacgaggtgcggcagatcgcccccggacagaccggcaacattgccgattacaactacaaactccctgacgattttaccggctgcgtgatcgcttggaattctaacaaactggattctaaggtgagtggaaactacaactacctgtacagactgtttcggaagagcaacctgaagcctttcgagagagacatctctacagagatctaccaggccggcaataaaccctgcaacggcgtggccggcttcaattgctacttccctctgcggagctatagcttccggcctacctacggcgtgggccaccagccttaccgggttgtggtgctgagcttcgagctgctgcacgccccagccacagtgtgcggccctaagaagagcaccaacctggtgaagaacaagcgggtgcagcctacagaaagcatcgtgagattccccaacatcacaaacctgtgccctttcgatgaagtgttcaacgctacacggtttgccagcgtgtatgcctggaatcggaaaagaatcagcaattgtgtggcagattacagcgtgctgtacaacttcgccccttttttcgccttcaagtgctacggcgtgtctcctaccaagctgaatgacctgtgttttaccaacgtgtacgccgactctttcgtgatcagagggaacgaggtgtcccagatcgccccaggccagacaggaaacatcgcctcttataactacaagctgcccgatgacttcaccggatgcgtgatcgcttggaacagcaacaagctcgacagcaaggtgggaggcaactacaactacctgtaccggctgttccggaaaagcaacctgaagccttttgaacgggatatctctaccgaaatctatcaggccggcaacaagccttgtaatggagtggccggcttcaattgctatttccccctgagatcttacggcttcagacctacatacggtgttggacaccagccttatagagtggttgtgctgagcttcgagctgctgcacgcccccgccacggtgtgtggccctaagaaaagcacaaacctcgtgaagaacaagagagtccagccaaccgaatctatcgttagattccctaatattacaaacctgtgtccttttggagaggtgtttaacgctaccagatttgccagcgtgtacgcctggaacagaaaaagaatcagcaactgcgtggccgactactccgtcctgtacaacagcgccagcttctccaccttcaagtgctacggcgtgtcccctaccaagctgaacgacctgtgctttaccaacgtgtacgccgatagtttcgtgatccggggcgatgaggtgcgacagatcgcccctggccagaccggaaacatcgccgactacaactacaaactgcccgacgactttaccggctgtgtgatcgcctggaacagcaacaacctggactctaaagtgggcggaaactacaattaccggtacaggctgttcagaaagagcaatctgaagcccttcgagcgggacatcagcaccgagatctaccaggccggaagcaagccctgcaacggcgtcgagggcttcaattgctatttcccactgcaaagctacggattccagcctaccaatggcgtgggctaccagccatacagggtggtggtgctgagcttcgaactgctgcatgctccggccaccgtgtgcggccctaagaagtctacgaacctggtcaagaacaagatggcctctgacaacggcccccaaaaccagagaaacgcgcctagaatcacatttggcggtcccagcgactccaccggctctaaccagaacggcgaaagatccggcgccagaagcaagcaacggagaccccagggcctgccaaacaacaccgccagttggttcacagcccttacacaacacggcaaagaggacctgaagttccctagaggccagggcgttcctatcaacacaaactcttctcctgacgaccagattggatactacagaagagccacaagaagaatcagaggcggcgacggaaagatgaaagacctgagccccaggtggtacttctactacctgggaaccggccctgaagccggcctgccttacggcgctaacaaggacggcatcatctgggttgcgaccgaaggcgccctgaacacccctaaggaccacatcggcaccagaaaccccgccaacaacgcggctatcgtgcttcagctgcctcagggcacaacactgcctaagggcttctacgccgagggcagcagagggggcagccaggcctcatccagaagcagctcccggagcagaaatagcagccggaactcgacccctggcagcagcagaggaaccagccctgcccggatggccggaaacggcggcgacgccgccctggccctgctgctcctggataggctgaaccagctggaatccaagatgagcggcaaaggccagcaacagcaaggccagaccgtgaccaagaaatctgcagctgaggcctctaagaagcctagacagaaacggaccgccacaaaggcttataatgtgacccaagcttttggtagaagaggaccagagcagacccagggcaacttcggcgaccaggagctgatcagacagggcactgattacaagcactggccccagatcgcccaattcgcccccagcgctagcgccttcttcggtatgagccggatcggcatggaagtgaccccttccggcacatggctgacctacacaggcgccatcaagctggacgacaaggaccccaacttcaaggatcaggtgatcctgctgaacaagcacatcgacgcctacaagaccttcccccccacagagcctaagaaagacaagaagaaaaaggccgatgagacacaggcattgccccagagacaaaagaaacagcagaccgtgaccctgctgccagccgccgatctggacgacttcagcaagcagctgcagcagagcatgagcagcgccgacagcacacaggcc

SEQ ID NO: 93 - protein, 1096 aa, synthetic construct
  REGION 1..1096: note = SVF-16.1 polypeptide sequence
MDWTWILFLVAAATRVHSARVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIASYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKMASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 94 - DNA, 3288 bp, synthetic construct
  misc_feature 1..3288: note = SVF-16.2 codon optimized DNA sequence
  source 1..3288: mol_type = other DNA
atggactggacctggatcctgttcctggtggcggctgcaacccgggtgcacagcgccagagtgcagcctaccgagtccatcgtgagattccccaacatcaccaatctgtgccccttcgacgaggtgttcaacgctacaagattcgccagcgtgtacgcctggaaccggaagagaatcagcaactgcgtggctgactacagcgtgctgtacaacctggccccttttttcaccttcaagtgctacggcgtgtctcctaccaagctgaatgacctgtgctttacaaatgtttacgccgacagcttcgtcatcagaggcgacgaggtgcggcagatcgccccaggtcagaccggcaacatcgctgactacaactacaagctgcccgacgacttcaccggctgtgttatcgcctggaatagcaacaaactcgacagcaaggtcagcggcaattacaattacctgtacagactgttcagaaagtccaacctgaagcctttcgagagagatatctccacagagatctaccaggcaggcaacaagccttgcaacggggtggccggcttcaactgttatttccctctgagatcctacagctttcggcccacgtacggcgtgggccaccagccctacagagtcgtggtgctgtctttcgagctgcttcacgcccctgctaccgtgtgtggccctaagaagtctacaaacctggtcaaaaacaagcgggtgcagccaacagaatctatcgtgagattccctaatatcacaaatctgtgccccttcggcgaggtgtttaacgccacacggttcgccagcgtgtacgcctggaaccggaagagaatcagcaactgcgtggccgattactctgtgctgtacaactctgccagctttagcaccttcaagtgctacggcgtgtcccctacaaagctgaacgacctgtgcttcaccaatgtgtacgccgattcttttgtgatccggggcgacgaggtgcggcaaatcgctcccggacagaccggaaagatcgccgactacaactacaagctgcctgatgactttaccggctgcgtgatcgcttggaattctaacaacctggactctaaggtgggcggaaactacaattacctgtacaggctcttcagaaagagcaacctcaagccttttgagagagacatctccaccgagatctatcaggccggtagcaccccttgtaatggcgtggaaggcttcaactgctacttccccctgcagagctacggtttccaacctacttatggcgtgggctaccagccttatagagtggtcgtgctgagcttcgagctgctgcatgcccccgccaccgtgtgcggccctaagaaatcaaccaacctggtgaagaacaaacgagtgcagcccaccgagagcattgtgcggtttccaaacattacaaacctgtgccctttcggcgaagtgttcaacgccaccagattcgccagcgtttacgcctggaacagaaagagaatcagcaattgcgtggccgactacagcgttctgtacaactctgccagcttcagcaccttcaagtgctacggcgtttccccaacaaagctgaacgatttgtgtttcacaaacgtgtacgcagactccttcgtgatcaggggcgacgaggtgcggcagattgcgcccggacagacaggaaacatcgctgactacaactataagctgccagacgacttcaccggctgcgtgattgcctggaacagcaataatctcgactctaaggtgggcggcaactacaactacagatacagactgttcagaaagagcaatctgaaacctttcgagcgggacatctccaccgaaatctaccaggccggcagcaaaccctgtaacggcgtggaaggcttcaactgctacttccccctgcagagctacggatttcagcctaccaacggcgttggttaccagccttaccgtgtggtggtgctgagctttgagctgctgcacgcccctgccaccgtgtgtggacctaagaagagcacaaacctggtgaagaataagatggccagcgacaacggccctcaaaaccagcggaacgccccacgtatcaccttcggcggccctagcgacagcaccggcagcaaccagaacggagaaagatctggagctagaagcaagcagagaaggcctcagggcctgcctaataacacagcttcttggtttacagccctgacccaacacggcaaggaagatctgaagttccctagaggacagggcgtccccatcaacaccaacagcagccccgacgatcagatcggatattaccggcgggccaccagaagaatcagaggcggagatggaaagatgaaggatctcagcccccggtggtacttctactacctgggcaccggccctgaggccggcctgccttacggcgccaataaagatggcatcatctgggtggccaccgaaggagccctgaacaccccaaaggaccacatcggcactcggaatcctgccaacaacgcggctatcgtgctgcagctgcctcagggaaccacactgcctaagggcttctacgccgagggctctagaggcggctctcaggccagcagcagaagtagcagcagaagcagaaactccagccgaaacagcacacccggcagctctagaggcaccagccccgcccgtatggccggcaatggcggcgacgccgccctggccctgctgctgctggatagactgaaccagctggaaagcaagatgtccggcaagggccagcagcagcaaggccagaccgtgaccaaaaagagcgccgctgaagccagcaagaagcctagacagaagcggaccgctacaaaggcctacaacgtgacacaggcttttggaagaagaggccccgagcagacccagggcaacttcggcgatcaggagctgatcaggcaaggcactgattacaagcactggcctcagatcgcccagttcgcccctagtgcctcagctttcttcggcatgagcagaatcggaatggaagtgacacctagtggcacctggctgacatacactggcgccatcaagctggacgacaaggaccctaacttcaaagaccaggtgatcctgctgaacaagcacatcgacgcctataagacattcccacccacagagccaaagaaggacaagaaaaaaaaggccgatgaaacccaggccctgccacaaagacagaagaagcagcagacagtgaccctgctgcccgccgccgatctggatgatttttctaaacagctccaacaaagcatgagcagcgccgactccacccaggcc

SEQ ID NO: 95 - protein, 1096 aa, synthetic construct
  REGION 1..1096: note = SVF-16.2 polypeptide sequence
MDWTWILFLVAAATRVHSARVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKMASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 96 - DNA, 3363 bp, synthetic construct
  misc_feature 1..3363: note = SVF-16.3 codon optimized DNA sequence
  source 1..3363: mol_type = other DNA
atggactggacctggatcctgttcctggtggccgctgcaacgcgggtgcacagcgccagagtgcagcccaccgagagcatcgtgcggttccctaacatcaccaatctgtgtccattcgacgaggtctttaacgccacaagattcgctagcgtgtatgcctggaacagaaagagaatcagcaactgcgtggctgactacagcgtgctgtataacctggcccctttcttcacattcaagtgctacggcgtgtcccctaccaagctgaacgacctgtgcttcacaaacgtctacgccgacagcttcgtgatcagaggcgatgaggtgcggcagatcgcaccaggccagaccggaaacatcgccgactacaactacaagctgcctgatgacttcacaggctgtgtgatcgcttggaacagcaataagctggacagcaaggtgtcaggaaactataactacctgtacagactgtttagaaagagcaaccttaagccttttgaacgggacatcagcaccgagatctaccaggccggaaacaagccttgcaacggcgtggccggcttcaactgctacttccctctgagaagctactccttccggcctacatacggcgtgggccaccagccttaccgggtggtggtgctgagcttcgagctgctgcacgcccccgccaccgtgtgcggccctaagaaaagcaccaacctggtgaagaacaagcgtgtgcagcccaccgagtctatcgtgcgatttcctaatatcaccaacctgtgccctttcggcgaagtgttcaacgccacacggttcgccagcgtgtacgcctggaatcggaagcggattagcaattgcgtggccgactactctgtgctgtacaacagcgcctctttttccacattcaagtgttatggagtgtcccctaccaagctgaacgacctgtgcttcacaaacgtgtacgccgattcattcgtgatcagaggcgacgaagtgcgccagatcgccccaggccagacaggcaagatcgccgattacaactacaagctgcctgacgacttcaccggctgcgtgatcgcctggaacagtaacaacctggactctaaggtgggcggtaactacaattacctgtacagactgttccggaagtccaacctgaagcctttcgagcgggacatcagcacagaaatctaccaggccggcagcacaccttgcaacggtgttgagggcttcaactgctacttcccactgcagagctacggctttcagccaacgtacggcgtcggctaccagccttaccgggtggttgtgctgtcctttgagctgctgcacgcccccgccaccgtgtgtggacccaagaagagcacaaacctggtcaagaacaaaagagttcaacccaccgagtccatcgtgcggttccctaacatcacaaatctgtgcccctttggcgaggtgttcaatgccacaagattcgcctcagtttacgcctggaatagaaagagaatctccaactgcgtggctgattacagcgtcctctacaacagcgctagctttagcacatttaagtgctacggcgtgtcacccaccaagcttaatgatctgtgtttcaccaacgtgtacgccgattctttcgtgatcagaggcgacgaggtgcggcagatcgcccctgggcaaaccggcaacattgccgactataactataagctgcctgatgatttcaccggctgtgtcatcgcctggaacagcaacaacttggactccaaggtaggcggcaactacaactaccgctaccggctgttcagaaagagcaacttgaaacctttcgagagagatatctctaccgagatctaccaggcgggatctaagccttgtaatggcgtggaaggcttcaattgctacttccccctgcagtcttacggcttccagcctaccaacggcgtgggctaccagccctacagagtcgtggtactgtcttttgagctgctacatgcccctgccaccgtgtgcggcccaaaaaagagcaccaatctggtgaagaacaagcctctgcagcctgagctggacagcttcaaagaggaactggataagtacttcaagaaccacacctctcccgacgtgatggcctccgacaacggcccacaaaaccagcggaatgcccctagaatcaccttcggcggaccttcggatagcaccggatccaaccagaacggcgaacggtccggcgccagaagcaaacagcgcagacctcagggcctgcctaacaacaccgccagctggttcaccgctctcacccaacacggcaaagaagatctgaagttccctagaggccagggagtgcccatcaacacaaacagttctcctgatgaccagatcggctactacagacgggccaccaggaggatcagaggcggcgacggcaagatgaaagacctgagccctagatggtacttctactacctgggcacaggaccagaagccggactgccctacggcgccaacaaggacggcattatctgggtggccacagagggcgctctgaatacccctaaggatcacattggaaccagaaaccccgctaataacgccgcaatcgtgctgcagctgccccagggcaccaccctgcctaaaggcttctacgccgaaggcagccggggtggctcccaggcctcttctagatcgagcagcagatctagaaacagcagccggaacagcactcctggctcttctagaggaacatctcccgcccggatggccggcaatggaggcgatgccgctctggccctgctgctgctggatagactcaaccagctggaatctaagatgagcggcaaaggacagcagcagcagggccagaccgtcaccaagaagagcgcagctgaggcctctaagaagcccagacaaaagcggaccgccacaaaggcctataacgtgacccaggccttcggcagaagagggcctgagcagacccagggcaacttcggcgatcaggagctgatcagacagggcactgattacaagcactggcctcagatcgcccagttcgcccctagcgcctctgcttttttcggcatgagcagaatcggcatggaagtgacacccagcggaacatggctgacttacaccggcgccatcaagctcgatgacaaggaccccaacttcaaggaccaagtgatcctgctgaacaagcacatcgacgcctacaaaaccttcccccccacagaacccaagaaggacaaaaaaaagaaggccgacgagacacaggccctgccccagagacagaaaaagcagcagaccgtgaccctgctgcctgccgccgacctggacgactttagcaaacagctgcaacaaagcatgagctctgccgacagcacccaggcc

SEQ ID NO: 97 - protein, 1121 aa, synthetic construct
  REGION 1..1121: note = SVF-16.3 polypeptide sequence
MDWTWILFLVAAATRVHSARVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKPLQPELDSFKEELDKYFKNHTSPDVMASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 98 - DNA, 3363 bp, synthetic construct
  misc_feature 1..3363: note = SVF-16.4 codon optimized DNA sequence
  source 1..3363: mol_type = other DNA
atggactggacctggatcctgttcctggttgctgccgccacaagagtgcacagcgctagagtgcagcctacagagagcatcgtgaggttccccaacatcacaaacctctgccctttcgatgaagtgttcaacgccacaagattcgcctctgtgtatgcctggaacaggaagcgcatcagcaactgcgtggccgactacagcgtgctgtacaacctcgccccttttttcacattcaagtgctacggcgtgtccccaacaaaactgaacgacctatgcttcaccaacgtgtacgccgattcctttgtgatcagaggcgacgaggtgcggcagatcgcgcctggccagacaggcaacattgccgactacaattacaaactgccagacgacttcaccggctgtgtgatcgcctggaatagcaacaagctggatagcaaagtgtccggcaactacaactacctgtacagactgtttagaaagtctaacctgaaaccctttgagagagatatctcgaccgagatctaccaggccggaaacaagccttgcaatggagtcgctggcttcaactgctacttccccctgagatcttatagcttcagacccacctacggcgtgggccaccagccctatagagtggtggtcctgagctttgaactgctgcatgcccctgctaccgtgtgcggcccaaagaagagcacaaacctggtgaagaataagcgggtgcagcccaccgaaagcatcgtgcggttccccaacataaccaatctgtgccccttcgacgaggtgttcaatgccacacggttcgcctccgtgtacgcttggaaccggaagcggatctctaattgtgtggccgactacagcgtgctgtataacttcgcccctttcttcgctttcaagtgctacggggtgtcccctaccaagctgaacgacctgtgcttcacgaacgtttacgccgacagctttgtgatcagaggcaacgaggtgtcccagatcgcccctggacagaccggaaacatcgccagctacaactacaagctgcctgacgactttacaggctgcgtgatcgcctggaacagcaataaactggatagcaaagtgggcggcaactataactacctgtacagactgttcagaaagtccaacctgaagccttttgaacgggatatctctaccgagatctaccaagccggaaacaagccatgtaatggcgtggccggctttaactgctactttcctctgagaagctacggcttcagacccacctacggcgtgggacaccagccatacagagtggtcgtgctgagcttcgagctgctacacgccccagccaccgtgtgcggccccaagaagagcaccaacctggtcaagaacaagagagttcaaccaacagagagcatcgttagatttcctaacatcactaacctgtgtcctttcggcgaagtgtttaatgcaacccgcttcgcctcagtgtacgcgtggaatagaaaaagaattagcaactgcgtcgctgattactcggtgctgtataacagcgccagcttcagcaccttcaagtgctacggcgtcagccctacaaagctcaacgacctgtgcttcacaaacgtttacgctgacagcttcgtgatcagaggcgacgaggtgcggcagatcgcccctggccagacgggcaacatcgccgattacaactacaagctccctgatgatttcaccggctgtgtgatcgcctggaattctaacaacctggactctaaggtgggcggcaattacaactacagatacagactgttccggaagtctaacctgaagccattcgagcgggacatcagtaccgaaatctaccaggccggctctaagccctgcaacggagtggaaggattcaactgttattttcctctgcagagctacggctttcagcctaccaatggcgtgggctaccagccctaccgggtggtggtgctgagcttcgagctgctgcatgcccctgccaccgtgtgtggccctaagaagagcacaaacctggtgaaaaacaagcctctgcagcctgagctggacagcttcaaagaggaactggataagtacttcaagaaccacacctctcccgacgtgatggccagcgataacggccctcagaaccagagaaacgcccctagaattaccttcggcggcccttctgatagcacggggtcgaaccagaacggcgagcggtccggagcccggtcgaagcagagaagacctcagggactgcccaataataccgccagctggttcaccgctctgacccaacacggcaaggaagatctgaaattccctagaggccagggcgtgcccatcaacaccaattctagccctgatgaccagatcggctactaccgacgggcgacacggagaatccggggtggcgacggaaagatgaaggacctgagcccccggtggtacttctactacctgggcacaggccctgaggccggcctgccatacggagctaacaaggacggcatcatctgggtggccacagagggcgccctgaacacccctaaggaccacatcggcacacggaatcctgccaataacgccgccattgtactgcagctgccccagggcacaaccctgcctaagggcttctacgccgagggcagccggggaggaagccaggcctccagcagatccagctctagaagcagaaacagcagcagaaacagcacacctggctcatctcggggcacaagccctgctagaatggccggcaacgggggcgacgccgctctggccctgctgctgctagacagactgaaccagctcgagagcaagatgagcggcaagggacagcagcagcagggccaaaccgtcaccaaaaaatctgccgctgaggcctccaagaagcctagacagaaaaggaccgccaccaaagcctacaacgtgacccaagctttcggcaggcgtggacccgagcagactcagggcaacttcggtgatcaggagctgatccggcagggcaccgactacaagcactggcctcaaatcgctcagttcgccccttcagcctctgccttcttcggcatgagcaggatcggcatggaagtgacccctagcggcacatggctgacctacaccggcgccattaagctggacgataaggaccccaatttcaaggaccaggtgatcctgctgaacaagcacatcgacgcttacaaaaccttcccgcctacagaacctaagaaagacaagaaaaagaaggccgatgaaacccaggccctgccacaacggcagaagaagcagcaaaccgtgaccctgctgcccgccgccgatctggatgacttcagcaagcagctgcaacagagcatgtcatccgccgacagcacccaggct

SEQ ID NO: 99 - protein, 1121 aa, synthetic construct
  REGION 1..1121: note = SVF-16.4 polypeptide sequence
MDWTWILFLVAAATRVHSARVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIASYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKPLQPELDSFKEELDKYFKNHTSPDVMASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 100 - protein, 219 aa, synthetic construct
  REGION 1..219: note = SARS-CoV-2 Wuhan-Hu-1 RBD polypeptide sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK